(19) United States
(12) Patent Application Publication
(10) Pub. No.: US 2026/0274795 A1
(43) Pub. Date: Sep. 17, 2026

(54) HETEROCYCLIC COMPOUND AND ORGANIC LIGHT-EMITTING DEVICE COMPRISING SAME

(71) Applicant: LT MATERIALS CO., LTD., Yongin-si, Gyeonggi-do (KR)

(72) Inventors: Nam-Jin LEE, Yongin-si (KR); Won-Jang JEONG, Yongin-si (KR); Dong-Jun KIM, Yongin-si (KR); Dae-Hyuk CHOI, Yongin-si (KR)

(73) Assignee: LT MATERIALS CO., LTD., Yongin-si, Gyeonggi-do (KR)

(21) Appl. No.: 18/873,657

(22) PCT Filed: Jun. 5, 2023
(86) PCT No.: PCT/KR2023/007709

§ 371 Date: Dec. 10, 2024

(30) Foreign Application Priority Data
Jun. 17, 2022 (KR) 10-2022-0074274

(51) Int. Cl. C07D 209/86; C07B 59/00; H10K 50/15; H10K 50/16; H10K 50/17; H10K 50/18; H10K 85/60

(52) U.S. Cl. CPC C07D 209/86; C07B 59/002; H10K 85/622; H10K 85/626; H10K 85/633; H10K 85/636; H10K 85/6572; H10K 85/6574; H10K 85/6576; C07B 2200/05; H10K 50/156; H10K 50/16; H10K 50/17; H10K 50/171; H10K 50/18; H10K 50/181

(57) ABSTRACT

The present specification provides a heterocyclic compound and an organic light emitting device including the same.

### TECHNICAL FIELD

The present specification relates to a heterocyclic compound and an organic light emitting device including the same.

This application claims priority to and the benefit of Korean Patent Application No. 10-2022-0074274 filed in the Korean Intellectual Property Office on Jun. 17, 2022, the entire contents of which are incorporated herein by reference.

### BACKGROUND ART

An electroluminescence device is a kind of self-emitting type display device, and has an advantage in that the viewing angle is wide, the contrast is excellent, and the response speed is fast.

An organic light emitting device is composed of a structure in which an organic thin film is disposed between two electrodes. When a voltage is applied to an organic light emitting device having such a structure, electrons and holes injected from the two electrodes are combined with each other in the organic thin film to make a pair, and then, the paired electrons and holes emit light while being annihilated. The organic thin film may be composed of a single layer or multiple layers, if necessary.

A material for the organic thin film may have a light emitting function, if necessary. For example, as the material for the organic thin film, it is also possible to use a compound, which may itself constitute a light emitting layer alone, or it is also possible to use a compound, which may serve as a host or a dopant of a host-dopant-based light emitting layer. In addition, as a material for the organic thin film, it is also possible to use a compound, which may perform a function such as hole injection, hole transport, electron blocking, hole blocking, electron transport or electron injection.

In order to improve the performance, efficiency and service life of the organic light emitting device, there is a continuous need for developing a material for an organic thin film.

### DISCLOSURE

### Technical Problem

The present invention has been made in an effort to provide a heterocyclic compound and an organic light emitting device including the same.

### Technical Solution

In an exemplary embodiment of the present application, provided is a heterocyclic compound represented by the following Chemical Formula 1.

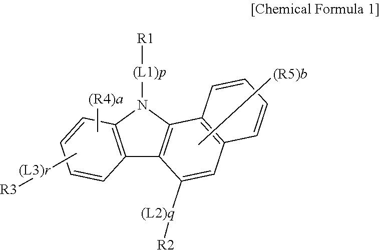

In Chemical Formula 1,
L1 to L3 are the same as or different from each other, and are each independently a direct bond; a substituted or unsubstituted C6 to C60 arylene group; or a substituted or unsubstituted C2 to C60 heteroarylene group,
R1 is a substituted or unsubstituted C6 to C60 aryl group; or a substituted or unsubstituted C2 to C60 heteroaryl group,
R2 and R3 are the same as or different from each other, and are each independently a substituted or unsubstituted C6 to C60 aryl group; a substituted or unsubstituted C2 to C60 heteroaryl group; or —N(Ra)(Rb), and at least one of R2 and R3 is —N(Ra)(Rb).

Ra and Rb are the same as or different from each other, and are each independently a substituted or unsubstituted C6 to C60 aryl group; or a substituted or unsubstituted C2 to C60 heteroaryl group,
R4 to R5 are the same as or different from each other, and are each independently hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group,
a is an integer from 0 to 3, b is an integer from 0 to 5, and when a and b are each an integer of 2 or higher, substituents in the parenthesis are the same as or different from each other, and
p, q and r are each an integer from 0 to 4, and when p, q and r are each an integer of 2 or higher, substituents in the parenthesis are the same as or different from each other.

Further, in an exemplary embodiment of the present application, provided is an organic light emitting device including: a first electrode; a second electrode provided to face the first electrode; and an organic material layer having one or more layers provided between the first electrode and the second electrode, in which the one or more layers of the organic material layer include the heterocyclic compound represented by Chemical Formula 1.

### Advantageous Effects

The heterocyclic compound described in the present specification may be used as a material for the organic material layer of the organic light emitting device. That is, the heterocyclic compound can serve as a light emitting material, a hole injection material, a hole transport material, an electron transport material, an electron injection material and the like in the organic light emitting device. In particular, the heterocyclic compound can be used as a material for a light emitting layer of an organic light emitting device.

Specifically, one or two or more of the heterocyclic compounds represented by Chemical Formula 1 can be used, and the heterocyclic compounds represented by Chemical Formula 1 can be used as materials for the light emitting layer. In particular, the heterocyclic compound can be used as a host material for the light emitting layer of an organic light emitting device by introducing various substituents and changing the binding position of the substituent to adjust the bandgap.

By having a disubstituted structure in which an arylamine group is introduced into a core skeleton and an aryl group or a heteroaryl group is additionally introduced into the core skeleton, the heterocyclic compound of the present invention has an advantage in that structural stability is better than a monosubstituted structure and hole mobility becomes faster than the monosubstituted structure.

In particular, when the heterocyclic compound of the present invention is used in the hole transport layer, electron blocking layer or light emitting layer of an organic light emitting device, it is possible to obtain an effect in which the driving voltage of the device is lowered, the efficiency of the device is also increased, and the service life of the device is extended.

### DESCRIPTION OF DRAWINGS

FIGS. 1 to 4 are views each schematically illustrating a stacking structure of an organic light emitting device according to an exemplary embodiment of the present application.

### EXPLANATION OF REFERENCE NUMERALS AND SYMBOLS

100: Substrate
200: Positive electrode
300: Organic material layer
301: Hole injection layer
302: Hole transport layer
303: Light emitting layer
304: Hole blocking layer
305: Electron transport layer
306: Electron injection layer
307: Electron blocking layer
400: Negative electrode

### MODE FOR INVENTION

Hereinafter, the present specification will be described in more detail.

When one part “includes” one constituent element in the present specification, unless otherwise specifically described, this does not mean that another constituent element is excluded, but means that another constituent element may be further included.

In the present specification,

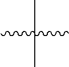

of a chemical formula means a position to which a constituent element is bonded.

The term “substitution” means that a hydrogen atom bonded to a carbon atom of a compound is changed into another substituent, and a position to be substituted is not limited as long as the position is a position at which the hydrogen atom is substituted, that is, a position at which the substituent may be substituted, and when two or more are substituted, the two or more substituents may be the same as or different from each other.

In the present specification, “substituted or unsubstituted” means being unsubstituted or substituted with one or more substituents selected from the group consisting of deuterium; a halogen group; —CN; a C1 to C60 alkyl group; a C2 to C60 alkenyl group; a C2 to C60 alkynyl group; a C1 to C60 haloalkyl group; a C1 to C60 alkoxy group; a C6 to C60 aryloxy group; a C1 to C60 alkylthioxy group; a C6 to C60 arylthioxy group; a C1 to C60 alkylsulfoxy group; a C6 to C60 arylsulfoxy group; a C3 to C60 cycloalkyl group; a C2 to C60 heterocycloalkyl group; a C6 to C60 aryl group; a C2 to C60 heteroaryl group; —SiRR′R″; —P(═O)RR′; and —NRR′, or a substituent to which two or more substituents selected among the exemplified substituents are linked, and R, R′ and R″ are each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group.

In the present specification, “when a substituent is not indicated in the structure of a chemical formula or compound” means that a hydrogen atom is bonded to a carbon atom. However, since deuterium (²H) is an isotope of hydrogen, some hydrogen atoms may be deuterium.

In an exemplary embodiment of the present application, “when a substituent is not indicated in the structure of a chemical formula or compound” may mean that all the positions that may be reached by the substituent are hydrogen or deuterium. That is, deuterium is an isotope of hydrogen, and some hydrogen atoms may be deuterium which is an isotope, and in this case, the content of deuterium may be 0% to 100%.

In an exemplary embodiment of the present application, in “the case where a substituent is not indicated in the structure of a chemical formula or compound”, when the content of deuterium is 0%, the content of hydrogen is 100%, and all the substituents do not explicitly exclude deuterium, such as hydrogen, hydrogen and deuterium may be mixed and used in the compound.

In an exemplary embodiment of the present application, deuterium is one of the isotopes of hydrogen, is an element that has a deuteron composed of one proton and one neutron as a nucleus, and may be represented by hydrogen-2, and the element symbol may also be expressed as D or ²H.

In an exemplary embodiment of the present application, the isotope means an atom with the same atomic number (Z), but different mass numbers (A), and the isotope may also be interpreted as an element which has the same number of protons, but different number of neutrons.

In an exemplary embodiment of the present application, when the total number of substituents of a basic compound is defined as T1 and the number of specific substituents among the substituents is defined as T2, the content T % of the specific substituent may be defined as T2/T1×100=T %.

That is, in an example, a deuterium content of 20% in a phenyl group represented by

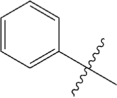 [SMILES: *c1ccccc1]

may be represented by 20% when the total number of substituents that the phenyl group can have is 5 (T1 in the formula) and the number of deuteriums among the substituents is 1 (T2 in the formula). That is, a deuterium content of 20% in the phenyl group may be represented by the following structural formula.

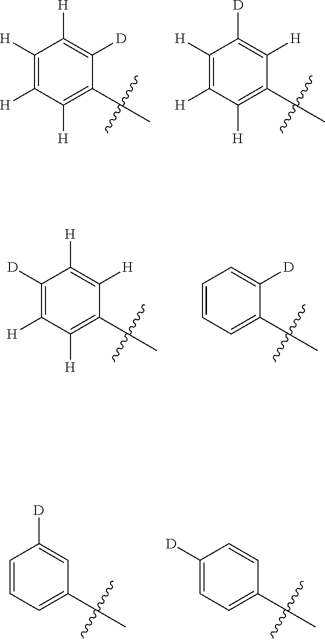 [SMILES: *c1ccccc1[2H] ; *c1cccc([2H])c1 ; *c1ccc([2H])cc1 ; *c1ccccc1[2H] ; *c1cccc([2H])c1 ; *c1ccc([2H])cc1]

Further, in an exemplary embodiment of the present application, “a phenyl group having a deuterium content of 0%” may mean a phenyl group that does not include a deuterium atom, that is, has five hydrogen atoms.

In the present specification, the halogen may be fluorine, chlorine, bromine or iodine.

In the present specification, an alkyl group includes a straight-chain or branched-chain having 1 to 60 carbon atoms, and may be additionally substituted with another substituent. The number of carbon atoms of the alkyl group may be 1 to 60, specifically 1 to 40, and more specifically 1 to 20. Specific examples thereof include a methyl group, an ethyl group, a propyl group, an n-propyl group, an isopropyl a butyl group, an n-butyl group, an isobutyl group, a tert-butyl group, a sec-butyl group, a 1-methyl-butyl group, a 1-ethyl-butyl group, a pentyl group, an n-pentyl group, an isopentyl group, a neopentyl group, a tert-pentyl group, a hexyl group, an n-hexyl group, a 1-methyl-pentyl group, a 2-methylpentyl group, a 4-methyl-2-pentyl group, a 3,3-dimethylbutyl group, a 2-ethylbutyl group, a heptyl group, an n-heptyl group, a 1-methylhexyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethyl-propyl group, a 1,1-dimethyl-propyl group, an isohexyl group, a 2-methylpentyl group, a 4-methylhexyl group, a 5-methylhexyl group, and the like, but are not limited thereto.

In the present specification, an alkenyl group includes a straight-chain or branched-chain having 2 to 60 carbon atoms, and may be additionally substituted with another substituent. The number of carbon atoms of the alkenyl group may be 2 to 60, specifically 2 to 40, and more specifically 2 to 20. Specific examples thereof include a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl)vinyl-1-yl group, a 2,2-bis(diphenyl-1-yl)vinyl-1-yl group, a stilbenyl group, a styrenyl group, and the like, but are not limited thereto.

In the present specification, an alkynyl group includes a straight-chain or branched-chain having 2 to 60 carbon atoms, and may be additionally substituted with another substituent. The number of carbon atoms of the alkynyl group may be 2 to 60, specifically 2 to 40, and more specifically 2 to 20.

In the present specification, a haloalkyl group means an alkyl group substituted with a halogen group, and specific examples thereof include —CF₃, —CF₂CF₃, and the like, but are not limited thereto.

In the present specification, an alkoxy group is represented by —O(R101), and the above-described examples of the alkyl group may be applied to R101.

In the present specification, an aryloxy group is represented by —O(R102), and the above-described examples of the aryl group may be applied to R102.

In the present specification, an alkylthioxy group is represented by —S(R103), and the above-described examples of the alkyl group may be applied to R103.

In the present specification, an arylthioxy group is represented by —S(R104), and the above-described examples of the aryl group may be applied to R104.

In the present specification, an alkylsulfoxy group is represented by —S(═O)₂(R105), and the above-described examples of the alkyl group may be applied to R105.

In the present specification, an arylsulfoxy group is represented by —S(═O)₂(R106), and the above-described examples of the aryl group may be applied to R106.

In the present specification, a cycloalkyl group includes a monocycle or polycycle having 3 to 60 carbon atoms, and may be additionally substituted with another substituent. Here, the polycycle means a group in which a cycloalkyl group is directly linked to or fused with another cyclic group. Here, another cyclic group may also be a cycloalkyl group, but may also be another kind of cyclic group, for example, a heterocycloalkyl group, an aryl group, a heteroaryl group, and the like. The number of carbon atoms of the cycloalkyl group may be 3 to 60, specifically 3 to 40, and more specifically 5 to 20. Specific examples thereof include a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, a cyclooctyl group, and the like, but are not limited thereto.

In the present specification, a heterocycloalkyl group includes O, S, Se, N, or Si as a heteroatom, includes a monocycle or polycycle having 2 to 60 carbon atoms, and may be additionally substituted with another substituent. Here, the polycycle means a group in which a heterocycloalkyl group is directly linked to or fused with another cyclic group. Here, another cyclic group may also be a heterocycloalkyl group, but may also be another kind of cyclic group, for example, a cycloalkyl group, an aryl group, a heteroaryl group, and the like. The number of carbon atoms of the heterocycloalkyl group may be 2 to 60, specifically 2 to 40, and more specifically 3 to 20.

In the present specification, an aryl group includes a monocycle or polycycle having 6 to 60 carbon atoms, and may be additionally substituted with another substituent. Here, the polycycle means a group in which an aryl group is directly linked to or fused with another cyclic group. Here, another cyclic group may also be an aryl group, but may also be another kind of cyclic group, for example, a cycloalkyl group, a heterocycloalkyl group, a heteroaryl group, and the like. The aryl group includes a spiro group. The number of carbon atoms of the aryl group may be 6 to 60, specifically 6 to 40, and more specifically 6 to 25. Specific examples of the aryl group include a phenyl group, a biphenyl group, a terphenyl group, a naphthyl group, an anthryl group, a chrysenyl group, a phenanthrenyl group, a perylenyl group, a fluoranthenyl group, a triphenylenyl group, a phenalenyl group, a pyrenyl group, a tetracenyl group, a pentacenyl group, a fluorenyl group, an indenyl group, an acenaphthylenyl group, a benzofluorenyl group, a spirobifluorenyl group, a 2,3-dihydro-1H-indenyl group, a fused cyclic group thereof, and the like, but are not limited thereto.

In the present specification, the terphenyl group may be selected from the following structures.

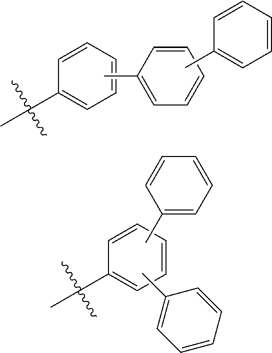

In the present specification, the fluorenyl group may be substituted, and adjacent substituents may be bonded to each other to form a ring.

When the fluorenyl group is substituted, the substituent may be

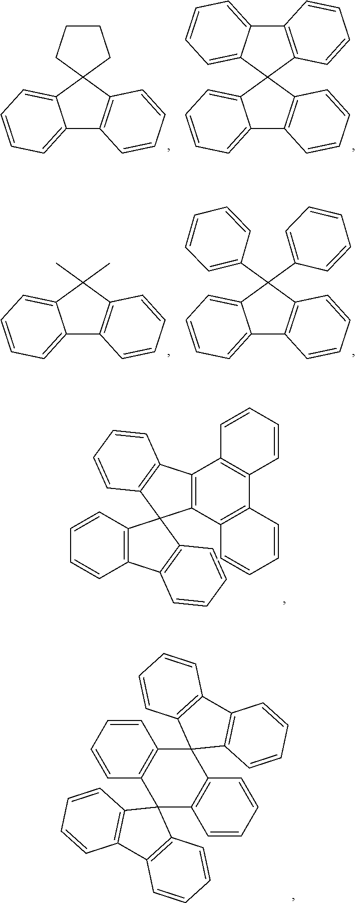

and the like, but is not limited thereto.

In the present specification, a heteroaryl group includes S, O, Se, N, or Si as a heteroatom, includes a monocycle or polycycle having 2 to 60 carbon atoms, and may be additionally substituted with another substituent. Here, the polycycle means a group in which a heteroaryl group is directly linked to or fused with another cyclic group. Here, another cyclic group may also be a heteroaryl group, but may also be another kind of cyclic group, for example, a cycloalkyl group, a heterocycloalkyl group, an aryl group, and the like. The number of carbon atoms of the heteroaryl group may be 2 to 60, specifically 2 to 40, and more specifically 3 to 25. Specific examples of the heteroaryl group include a pyridine group, a pyrrole group, a pyrimidine group, a pyridazine group, a furan group, a thiophene group, an imidazole group, a pyrazole group, an oxazole group, an isoxazole group, a thiazole group, an isothiazole group, a triazole group, a furazan group, an oxadiazole group, a thiadiazole group, a dithiazole group, a tetrazolyl group, a pyran group, a thiopyran group, a diazine group, an oxazine group, a thiazine group, a dioxin group, a triazine group, a tetrazine group, a quinoline group, isoquinoline group, a quinazoline group, an isoquinazoline group, a quinozoline group, a naphthyridine group, an acridine group, an imidazopyridine group, a diazanaphthalene group, a triazaindene group, an indole group, an indolizine group, a benzothiazole group, a benzoxazole group, a benzimidazole group, a benzothiophene group, a benzofuran group, a dibenzothiophene group, a dibenzofuran group, a carbazole group, a benzocarbazole group, a dibenzocarbazole group, a phenazine group, a dibenzosilole group, spirobi (dibenzosilole) group, a dihydrophenazine group, a phenoxazine group, a phenanthridine group, a thienyl group, an indolo[2,3-a]carbazole group, an indolo[2,3-b]carbazole group, an indoline group, a 10,11-dihydro-dibenzo[b,f]azepine group, a 9,10-dihydroacridine group, a phenanthrazine group, a phenothiazine group, a phthalazine group, a phenanthroline group, a naphthobenzofuran group, a naphthobenzothiophene group, a benzo[c][1,2,5]thiadiazole group, a 2,3-dihydrobenzo[b]thiophene group, a 2,3-dihydrobenzofuran group, a 5,10-dihydrodibenzo[b,e][1,4]azasiline group, a pyrazolo[1,5-c]quinazoline group, a pyrido[1,2-b]indazole group, a pyrido[1,2-a]imidazo[1,2-e]indoline group, a 5,11-dihydroindeno[1,2-b]carbazole group, and the like, but are not limited thereto.

In the present specification, when the substituent is a carbazole group, it means being bonded to nitrogen or carbon of carbazole.

In the present specification, when a carbazole group is substituted, an additional substituent may be substituted with the nitrogen or carbon of the carbazole.

In the present specification, a benzocarbazole group may be any one of the following structures.

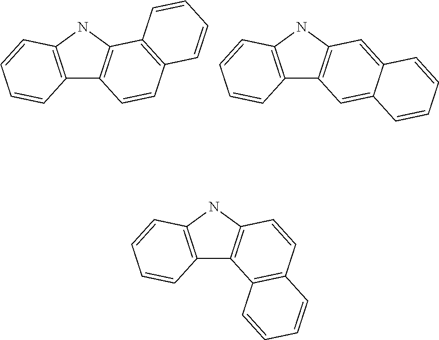

In the present specification, a dibenzocarbazole group may be any one of the following structures.

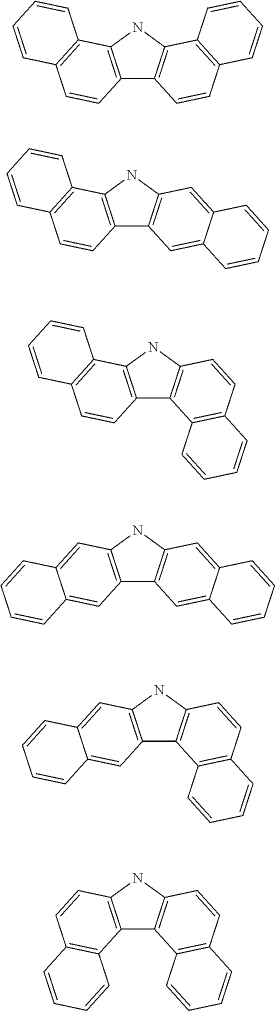 [SMILES: c1ccc2c(c1)ccc1c3ccc4ccccc4c3[nH]c21 ; c1ccc2cc3c(cc2c1)[nH]c1c2ccccc2ccc31 ; c1ccc2c(c1)ccc1c2[nH]c2ccc3ccccc3c21 ; c1ccc2cc3c(cc2c1)[nH]c1cc2ccccc2cc13 ; c1ccc2cc3c(cc2c1)[nH]c1ccc2ccccc2c13 ; c1ccc2c(c1)ccc1[nH]c3ccc4ccccc4c3c12]

In the present specification, a naphthobenzofuran group may be any one of the following structures.

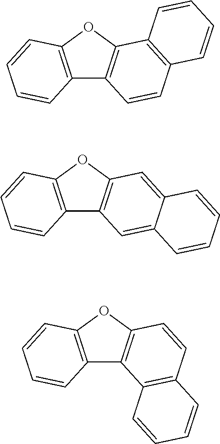 [SMILES: c1ccc2c(c1)ccc1c3ccccc3oc21 ; c1ccc2cc3c(cc2c1)oc1ccccc13 ; c1ccc2c(c1)ccc1oc3ccccc3c12]

In the present specification, a naphthobenzothiophene group may be any one of the following structures.

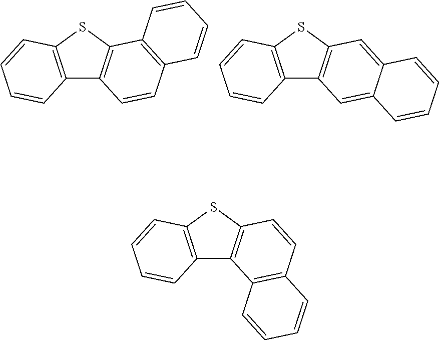

In the present specification, a silyl group includes Si and is a substituent to which the Si atom is directly linked as a radical, and is represented by —Si(R107)(R108)(R109), and R107 to R109 are the same as or different from each other, and may be each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group. Specific examples of the silyl group include

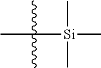 [SMILES: *[Si](C)(C)C]

(a trimethylsilyl group),

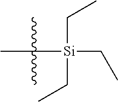 [SMILES: *[Si](CC)(CC)CC]

(a triethylsilyl group),

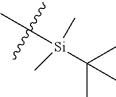 [SMILES: *[Si](C)(C)C(C)(C)C]

(a t-butyldimethylsilyl group),

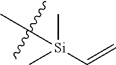 [SMILES: *[Si](C)(C)C=C]

(a vinyldimethylsilyl group),

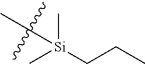 [SMILES: *[Si](C)(C)CCC]

(a propyldimethylsilyl group),

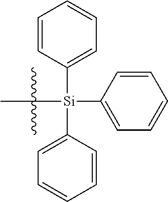 [SMILES: *[Si](c1ccccc1)(c1ccccc1)c1ccccc1]

(a triphenylsilyl group),

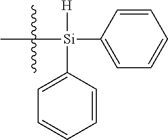 [SMILES: *[SiH](c1ccccc1)c1ccccc1]

(a diphenylsilyl group),

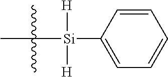 [SMILES: *[SiH2]c1ccccc1]

(a phenylsilyl group) and the like, but are not limited thereto.

In the present specification, a phosphine oxide group is represented by —P(═O)(R110)(R111), and R110 and R111 are the same as or different from each other, and may be each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group. Specifically, the phosphine oxide group may be substituted with an alkyl group or an aryl group, and the above-described example may be applied to the alkyl group and the aryl group. Examples of the phosphine oxide group include a dimethylphosphine oxide group, a diphenylphosphine oxide group, dinaphthylphosphine oxide group, and the like, but are not limited thereto.

In the present specification, an amine group is represented by —N(R112)(R113), and R112 and R113 are the same as or different from each other, and may be each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group. The amine group may be selected from the group consisting of —NH₂; a monoalkylamine group; a monoarylamine group; a monoheteroarylamine group; a dialkylamine group; a diarylamine group; a diheteroarylamine group; an alkylarylamine group; an alkylheteroarylamine group; and an arylheteroarylamine group, and the number of carbon atoms thereof is not particularly limited, but is preferably 1 to 30. Specific examples O amine group include a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group, and the like, but are not limited thereto.

In the present specification, the above-described examples of the aryl group may be applied to an arylene group except for a divalent arylene group.

In the present specification, the above-described examples of the heteroaryl group may be applied to a heteroarylene group except for a divalent heteroarylene group.

In the present specification, the “adjacent” group may mean a substituent substituted with an atom directly linked to an atom in which the corresponding substituent is substituted, a substituent disposed to be sterically closest to the corresponding substituent, or another substituent substituted with an atom in which the corresponding substituent is substituted. For example, two substituents substituted at the ortho position in a benzene ring and two substituents substituted at the same carbon in an aliphatic ring may be interpreted as groups which are “adjacent” to each other.

Hydrocarbon rings and hetero rings that adjacent groups may form include an aliphatic hydrocarbon ring, an aromatic hydrocarbon ring, an aliphatic hetero ring and an aromatic hetero ring, and structures exemplified by the above-described cycloalkyl group, aryl group, heterocycloalkyl group and heteroaryl group may be applied to the rings, except for those that are not monovalent groups.

In an exemplary embodiment of the present application, provided is the heterocyclic compound represented by Chemical Formula 1.

In an exemplary embodiment of the present application, a group not represented by a substituent; or a group represented by hydrogen may mean being all substitutable with deuterium. That is, it may be shown that hydrogen; or deuterium can be substituted with each other.

In general, compounds bonded with hydrogen and compounds substituted with deuterium exhibit a difference in thermodynamic behavior. The reason for this is that deuterium is characterized by having even lower vibration energy due to the mass of a deuterium atom is 2-fold higher than that of hydrogen.

Further, the single bond dissociation energy of carbon and deuterium is higher than the single bond dissociation energy of carbon and hydrogen. Accordingly, the deuterium-substituted structure has an effect of increasing the thermal stability of the molecule and improving the service life of the device using the increased thermal stability.

When a compound is deposited on a silicon wafer, a material including deuterium tends to be packed so that the intermolecular distance is reduced. Further, when the surface of a thin film is observed using an atomic force microscope (AFM), it can be confirmed that the thin film made of a compound including deuterium is deposited with a more uniform surface without any aggregated portion.

The deuterium content of the heterocyclic compound of Chemical Formula 1 of the present application may be more than 0% and 100% or less. The deuterium-substituted compound is characterized in that the energy in the ground state is further lower than that of the hydrogen-substituted compound, shorter the bond length between carbon and deuterium is, the smaller the molecular hardcore volume is. Accordingly, the electrical polarizability may be reduced and the intermolecular interaction can be weakened, so that when a device is manufactured, the device has a stabler stacking structure.

These characteristics induce an effect of lowering the crystallinity by creating the amorphous state of a thin film. That is, the heterocyclic compound represented by Chemical Formula 1 may be effective in improving the heat resistance of an OLED device, thereby improving the service life and driving characteristics.

In the present specification, the deuterium content means the content of deuterium with respect to the total hydrogen and deuterium included in Chemical Formula 1.

In an exemplary embodiment of the present application, the deuterium content of the heterocyclic compound of Chemical Formula 1 may be 0%, or 30% to 100%.

In an exemplary embodiment of the present application, the deuterium content of the heterocyclic compound of Chemical Formula 1 may be 0%, or 50% to 100%.

In an exemplary embodiment of the present application, the deuterium content of the heterocyclic compound of Chemical Formula 1 may be 0%, or 70% to 100%.

In an exemplary embodiment of the present application, the deuterium content of the heterocyclic compound of Chemical Formula 1 may be 0%, or 90% to 100%.

In an exemplary embodiment of the present application, the deuterium content of the heterocyclic compound of Chemical Formula 1 may be 0%.

In an exemplary embodiment of the present application, the deuterium content of the heterocyclic compound of Chemical Formula 1 may be 30% to 100%.

In an exemplary embodiment of the present application, the deuterium content of the heterocyclic compound of Chemical Formula 1 may be 50% to 100%.

In an exemplary embodiment of the present application, the deuterium content of the heterocyclic compound of Chemical Formula 1 may be 70% to 100%.

In an exemplary embodiment of the present application, the deuterium content of the heterocyclic compound of Chemical Formula 1 may be 90% to 100%.

In an exemplary embodiment of the present application, L1 to L3 are the same as or different from each other, and may be each independently a direct bond; a substituted or unsubstituted C6 to C60 arylene group; or a substituted or unsubstituted C2 to C60 heteroarylene group.

In another exemplary embodiment, L1 to L3 are the same as or different from each other, and may be each independently a direct bond; a substituted or unsubstituted C6 to C40 arylene group; or a substituted or unsubstituted C2 to C40 heteroarylene group.

In still another exemplary embodiment, L1 to L3 are the same as or different from each other, and may be each independently a direct bond; a substituted or unsubstituted C6 to C20 arylene group; or a substituted or unsubstituted C2 to C20 heteroarylene group.

In yet another exemplary embodiment, L1 to L3 are the same as or different from each other, and may be each independently a direct bond; or a substituted or unsubstituted C6 to C20 arylene group.

In still yet another exemplary embodiment, L1 to L3 are the same as or different from each other, and may be each independently a direct bond; or a substituted or unsubstituted phenylene group.

In a further exemplary embodiment, L1 to L3 are the same as or different from each other, and may be each independently a direct bond; or a phenylene group which is unsubstituted or substituted with deuterium.

In an exemplary embodiment of the present application, R1 may be a substituted or unsubstituted C6 to C60 aryl group; or a substituted or unsubstituted C2 to C60 heteroaryl group.

In another exemplary embodiment, R1 may be a substituted or unsubstituted C6 to C40 aryl group; or a substituted or unsubstituted C2 to C40 heteroaryl group.

In still another exemplary embodiment, R1 may be a substituted or unsubstituted C6 to C20 aryl group; or a substituted or unsubstituted C2 to C20 heteroaryl group.

In yet another exemplary embodiment, R1 may be a substituted or unsubstituted phenyl group; a substituted or unsubstituted biphenyl group; a substituted or unsubstituted naphthyl group; a substituted or unsubstituted phenanthrenyl group; a substituted or unsubstituted triphenylenyl group; a substituted or unsubstituted fluorenyl group; a substituted or unsubstituted dibenzofuran group; or a substituted or unsubstituted dibenzothiophene group.

In still yet another exemplary embodiment, R1 may be a phenyl group which is unsubstituted or substituted with deuterium; biphenyl group; a naphthyl group which is unsubstituted or substituted with deuterium; a phenanthrenyl a triphenylenyl group; a fluorenyl group which is substituted with a methyl group; a dibenzofuran group; or a dibenzothiophene group.

In an exemplary embodiment of the present application, R2 and R3 are the same as or different from each other, and are each independently a substituted or unsubstituted C6 to C60 aryl group; a substituted or unsubstituted C2 to C60 heteroaryl group; or —N(Ra)(Rb), and at least one of R2 and R3 may be —N(Ra)(Rb).

In another exemplary embodiment, R2 and R3 are the same as or different from each other, and are each independently a substituted or unsubstituted C6 to C40 aryl group; a substituted or unsubstituted C2 to C40 heteroaryl group; or —N(Ra)(Rb), and at least one of R2 and R3 may be —N(Ra)(Rb).

In still another exemplary embodiment, R2 and R3 are the same as or different from each other, and are each independently a substituted or unsubstituted C6 to C20 aryl group; a substituted or unsubstituted C2 to C20 heteroaryl group; or —N(Ra)(Rb), and at least one of R2 and R3 may be —N(Ra)(Rb),

In yet another exemplary embodiment, R2 and R3 are the same as or different from each other, and are each independently a substituted or unsubstituted phenyl group; a substituted or unsubstituted biphenyl group; a substituted or unsubstituted naphthyl group; a substituted or unsubstituted phenanthrenyl group; a substituted or unsubstituted triphenylenyl group; a substituted or unsubstituted fluorenyl group; a substituted or unsubstituted dibenzofuran group; a substituted or unsubstituted dibenzothiophene group; or —N(Ra)(Rb), and any one of R2 and R3 may be —N(Ra)(Rb).

In still yet another exemplary embodiment, R2 and R3 are the same as or different from each other, and are each independently a substituted or unsubstituted phenyl group; a substituted or unsubstituted biphenyl group; a substituted or unsubstituted naphthyl group; a substituted or unsubstituted phenanthrenyl group; a substituted or unsubstituted triphenylenyl group; a substituted or unsubstituted dibenzofuran group; a substituted or unsubstituted dibenzothiophene group; or —N(Ra)(Rb), and any one of R2 and R3 may be —N(Ra)(Rb).

In a further exemplary embodiment, R2 and R3 are the same as or different from each other, and are each independently a phenyl group which is unsubstituted or substituted with deuterium; a biphenyl group; a naphthyl group; a phenanthrenyl group; a triphenylenyl group; a dibenzofuran group; a dibenzothiophene group; or —N(Ra)(Rb), and at least one of R2 and R3 may be —N(Ra)(Rb).

In an exemplary embodiment of the present application, R2 is a substituted or unsubstituted C6 to C60 aryl group; or a substituted or unsubstituted C2 to C60 heteroaryl group, and R3 may be —N(Ra)(Rb).

In an exemplary embodiment of the present application, R3 is a substituted or unsubstituted C6 to C60 aryl group; or a substituted or unsubstituted C2 to C60 heteroaryl group, and R2 may be —N(Ra)(Rb).

In an exemplary embodiment of the present application, Ra and Rb are the same as or different from each other, and may be each independently a substituted or unsubstituted C6 to C60 aryl group; or a substituted or unsubstituted C2 to C60 heteroaryl group.

In another exemplary embodiment, Ra and Rb are the same as or different from each other, and may be each independently a substituted or unsubstituted C6 to C40 aryl group; or a substituted or unsubstituted C2 to C40 heteroaryl group.

In still another exemplary embodiment, Ra and Rb are the same as or different from each other, and may be each independently a substituted or unsubstituted C6 to C30 aryl group; or a substituted or unsubstituted C2 to C20 heteroaryl group.

In yet another exemplary embodiment, Ra and Rb are the same as or different from each other, and may be each independently a substituted or unsubstituted C6 to C20 aryl group; or substituted or unsubstituted C2 to C20 heteroaryl group.

In still yet another exemplary embodiment, Ra and Rb are the same as or different from each other, and may be each independently a substituted or unsubstituted phenyl group; a substituted or unsubstituted biphenyl group; a substituted or unsubstituted naphthyl group; a substituted or unsubstituted phenanthrenyl group; a substituted or unsubstituted triphenylenyl group; a substituted or unsubstituted fluorenyl a substituted or group; unsubstituted dibenzofuran group; or a substituted or unsubstituted dibenzothiophene group.

In a further exemplary embodiment, Ra and Rb are the same as or different from each other, and may be each independently a phenyl group which is unsubstituted or substituted with deuterium or a phenanthrenyl group unsubstituted or substituted with deuterium; a biphenyl group which is unsubstituted or substituted with deuterium; a naphthyl group which is unsubstituted or substituted with deuterium; a phenanthrenyl group; a triphenylenyl group; a fluorenyl group which is unsubstituted or substituted with deuterium, a phenyl or a methyl group; group a spirobifluorenyl group; a dibenzofuran or group; a dibenzothiophene group.

In another further exemplary embodiment, Ra and Rb are the same as or different from each other, and may be each independently a phenyl group which is unsubstituted or substituted with deuterium or a phenanthrenyl group unsubstituted or substituted with deuterium; a biphenyl group which is unsubstituted or substituted with deuterium; a naphthyl group which is unsubstituted or substituted with deuterium; a phenanthrenyl group; a triphenylenyl group; a fluorenyl group which is unsubstituted or substituted with deuterium, a phenyl group or a methyl group; a dibenzofuran group; or a dibenzothiophene group.

In an exemplary embodiment of the present application, R4 and R5 are the same as or different from each other, and may be each independently hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group.

In another exemplary embodiment, R4 and R5 are the same as or different from each other, and may be each independently hydrogen; deuterium; a halogen group; a cyano group; a substituted or unsubstituted C1 to C40 alkyl group; a substituted or unsubstituted C3 to C40 cycloalkyl group; a substituted or unsubstituted C2 to C40 heterocycloalkyl group; a substituted or unsubstituted C6 to C40 aryl group; or a substituted or unsubstituted C2 to C40 heteroaryl group.

In still another exemplary embodiment, R4 and R5 are the same as or different from each other, and may be each independently hydrogen; deuterium; a halogen group; a cyano group; a substituted or unsubstituted C1 to C20 alkyl group; a substituted or unsubstituted C3 to C20 cycloalkyl group; a substituted or unsubstituted C2 to C20 heterocycloalkyl group; a substituted or unsubstituted C6 to C20 aryl group; or a substituted or unsubstituted C2 to C20 heteroaryl group.

In yet another exemplary embodiment, R4 and R5 are the same as or different from each other, and may be each independently hydrogen; deuterium; a substituted or unsubstituted C1 to C20 alkyl group; a substituted or unsubstituted C6 to C20 aryl group; or a substituted or unsubstituted C2 to C20 heteroaryl group.

In still yet another exemplary embodiment, R4 and R5 are the same as or different from each other, and may be each independently hydrogen; deuterium; or a substituted or unsubstituted C6 to C20 aryl group.

In a further exemplary embodiment, R4 and R5 are the same as or different from each other, and may be each independently hydrogen; or deuterium.

In an exemplary embodiment of the present application, a is an integer from 0 to 3, b is an integer from 0 to 5, and when a and b are each an integer of 2 or higher, substituents in the parenthesis are the same as or different from each other.

In another exemplary embodiment, a is an integer from 1 to 3, b is an integer from 1 to 5, and when a and b are each an integer of 2 or higher, substituents in the parenthesis are the same as or different from each other.

In still another exemplary embodiment, a is an integer from 2 to 3, b is an integer from 3 to 5, and substituents in the parenthesis are the same as or different from each other.

In an exemplary embodiment of the present application, p, q and r are each an integer from 0 to 4, and when p, q and r are each an integer of 2 or higher, substituents in the parenthesis are the same as or different from each other.

In another exemplary embodiment, p, q and r are each an integer from 0 to 3, and when p, q and r are each an integer of 2 or higher, substituents in the parenthesis are the same as or different from each other.

In still another exemplary embodiment, p, q and r are each an integer from 0 to 2, and when p, q and r are each 2, substituents in the parenthesis are the same as or different from each other.

In yet another exemplary embodiment, p, q and r are each 0 or 1.

In still yet another exemplary embodiment, p, q and r is 1.

In a further exemplary embodiment, p, q and r is 0.

In an exemplary embodiment of the present application, Chemical Formula 1 may be represented by any one of the following Chemical Formulae 1-1 to 1-5.

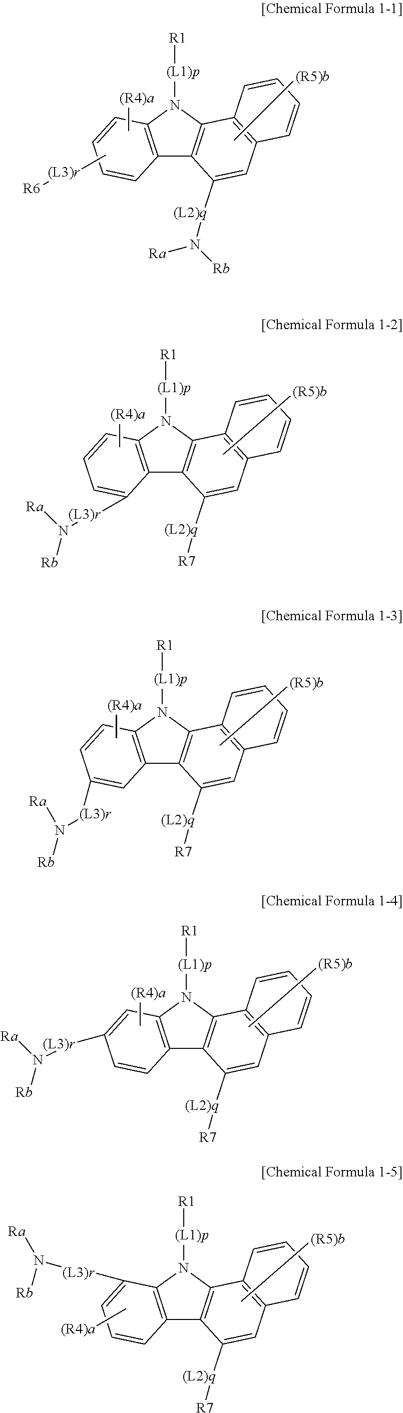

In Chemical Formulae 1-1 to 1-5,
the definitions of L1 to L3, R1, R4, R5, Ra, Rb, a, b, p, q and r are the same as the definitions in Chemical Formula 1, and
R6 and R7 are the same as or different from each other, and are each independently a substituted or unsubstituted C6 to C60 aryl group; or a substituted or unsubstituted C2 to C60 heteroaryl group.

In another exemplary embodiment, R6 and R7 are the same as or different from each other, and may be each independently a substituted or unsubstituted C6 to C40 aryl group; or a substituted or unsubstituted C2 to C40 heteroaryl group.

In still another exemplary embodiment, R6 and R7 are the same as or different from each other, and may be each independently a substituted or unsubstituted C6 to C20 aryl group; or a substituted or unsubstituted C2 to C20 heteroaryl group.

In yet another exemplary embodiment, R6 and R7 are the same as or different from each other, and may be each independently a substituted or unsubstituted phenyl group; a substituted or unsubstituted biphenyl group; a substituted or unsubstituted naphthyl group; a substituted or unsubstituted phenanthrenyl group; a substituted or unsubstituted triphenylenyl a substituted or group; unsubstituted fluorenyl group; a substituted or unsubstituted dibenzofuran group; or a substituted or unsubstituted dibenzothiophene group.

In still yet another exemplary embodiment, R6 and R7 are the same as or different from each other, and may be each independently a phenyl group which is unsubstituted or substituted with deuterium; a biphenyl group; a naphthyl group; a phenanthrenyl group; a triphenylenyl group; a dibenzofuran group; or dibenzothiophene group.

In an exemplary embodiment of the present application, Chemical Formula 1 may be represented by any one of the following Chemical Formulae 1-10 to 1-15.

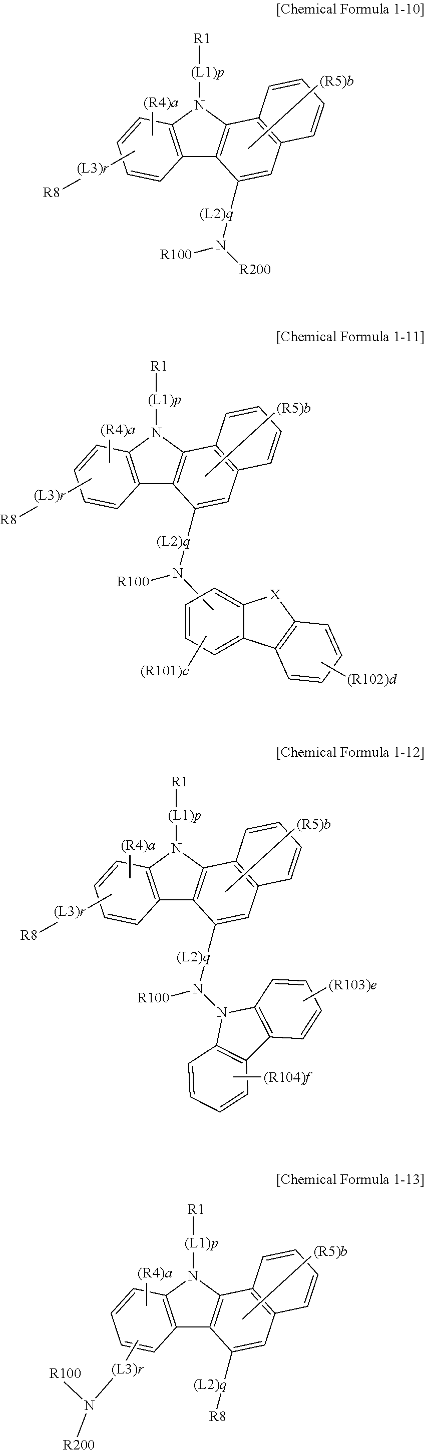

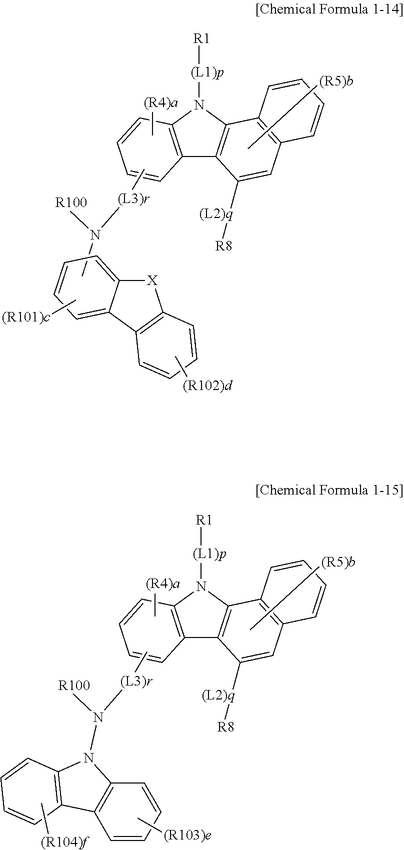

In Chemical Formulae 1-10 to 1-15,
the definitions of L1 to L3, R1, R4, R5, a, b, p, q and r are the same as the definitions in Chemical Formula 1,
R8 is a substituted or unsubstituted C6 to C60 aryl group; or a substituted or unsubstituted C2 to C60 heteroaryl group,
R100 and R200 are the same as or different from each other, and are each independently a substituted or unsubstituted C6 to C60 aryl group,
X is O, S, N or C(Rc)(Rd),
R101 to R104 are the same as or different from each other, and are each independently hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group,
Rc and Rd are the same as or different from each other, and are each independently a substituted or unsubstituted C1 to C60 alkyl group or a substituted or unsubstituted C6 to C60 aryl group, or are bonded to each other to form a substituted or unsubstituted ring, and
c is an integer from 0 to 3, d, e and f are each an integer from 0 to 4, and when c, d, e and f are each an integer of 2 or higher, substituents in the parenthesis may be the same as or different from each other.

In another exemplary embodiment, R8 may be a substituted or unsubstituted C6 to C40 aryl group; or a substituted or unsubstituted C2 to C40 heteroaryl group.

In still another exemplary embodiment, R8 may be a substituted or unsubstituted C6 to C20 aryl group; or a substituted or unsubstituted C2 to C20 heteroaryl group.

In yet another exemplary embodiment, R8 may be a substituted or unsubstituted phenyl group; a substituted or unsubstituted biphenyl group; a substituted or unsubstituted naphthyl group; a substituted or unsubstituted phenanthrenyl group; a substituted or unsubstituted triphenylenyl group; a substituted or unsubstituted fluorenyl group; a substituted or unsubstituted dibenzofuran group; or a substituted or unsubstituted dibenzothiophene group.

In still yet another exemplary embodiment, R8 may be a substituted or unsubstituted phenyl group; a substituted or unsubstituted biphenyl group; a substituted or unsubstituted naphthyl group; a substituted or unsubstituted phenanthrenyl group; a substituted or unsubstituted triphenylenyl group; a substituted or unsubstituted dibenzofuran group; or a substituted or unsubstituted dibenzothiophene group.

In a further exemplary embodiment, R8 may be a phenyl group which is unsubstituted or substituted with deuterium; a biphenyl group; a naphthyl group; a phenanthrenyl group; a triphenylenyl group; a dibenzofuran group; or a dibenzothiophene group.

In another further exemplary embodiment, R100 and R200 are the same as or different from each other, and may be each independently a substituted or unsubstituted C6 to C40 aryl group.

In still another further exemplary embodiment, R100 and R200 are the same as or different from each other, and may be each independently a substituted or unsubstituted C6 to C20 aryl group.

In yet another further exemplary embodiment, R100 and R200 are the same as or different from each other, and may be each independently a substituted or unsubstituted phenyl group; a substituted or unsubstituted biphenyl group; a substituted or unsubstituted naphthyl group; a substituted or unsubstituted phenanthrenyl group; a substituted or unsubstituted triphenylenyl group; or a substituted or unsubstituted fluorenyl group.

In still yet another further exemplary embodiment, R100 and R200 are the same as or different from each other, and may be each independently a phenyl group which is unsubstituted or substituted with deuterium, a fluorenyl group substituted with a phenyl group, or a phenanthrenyl group which is unsubstituted or substituted with deuterium; a biphenyl group which is unsubstituted or substituted with deuterium; a naphthyl group which is unsubstituted or substituted with deuterium; a phenanthrenyl group; a triphenylenyl group; or a fluorenyl group which is unsubstituted or substituted with deuterium, a phenyl group or a methyl group.

In a still further exemplary embodiment, R100 and R200 are the same as or different from each other, and may be each independently a phenyl group which is unsubstituted or substituted with deuterium or a phenanthrenyl group unsubstituted or substituted with deuterium; biphenyl group which is unsubstituted or substituted with deuterium; a naphthyl group which is unsubstituted or substituted with deuterium; a phenanthrenyl group; a triphenylenyl group; or a fluorenyl group which is unsubstituted or substituted with deuterium, a phenyl group or a methyl group.

In yet still further exemplary embodiment, R100 is a substituted or unsubstituted phenyl group; or a substituted or unsubstituted biphenyl group, and R200 may a be substituted or unsubstituted phenyl group; a substituted or unsubstituted biphenyl group; a substituted or unsubstituted naphthyl group; a substituted or unsubstituted phenanthrenyl group; a substituted or unsubstituted triphenylenyl group; or a substituted or unsubstituted fluorenyl group.

In another exemplary embodiment, R101 to R104 are the same as or different from each other, and may be each independently hydrogen; deuterium; a halogen group; a cyano group; a substituted or unsubstituted C1 to C40 alkyl group; a substituted or unsubstituted C3 to C40 cycloalkyl group; a substituted or unsubstituted C2 to C40 heterocycloalkyl group; a substituted or unsubstituted C6 to C40 aryl group; or a substituted or unsubstituted C2 to C40 heteroaryl group.

In still another exemplary embodiment, R101 to R104 are the same as or different from each other, and may be each independently hydrogen; deuterium; a halogen group; a cyano group; a substituted or unsubstituted C1 to C20 alkyl group; a substituted or unsubstituted C3 to C20 cycloalkyl group; a substituted or unsubstituted C2 to C20 heterocycloalkyl group; a substituted or unsubstituted C6 to C20 aryl group; or a substituted or unsubstituted C2 to C20 heteroaryl group.

In yet another exemplary embodiment, R101 to R104 are the same as or different from each other, and may be each independently hydrogen; deuterium; a substituted or unsubstituted C1 to C20 alkyl group; a substituted or unsubstituted C6 to C20 aryl group; or a substituted or unsubstituted C2 to C20 heteroaryl group.

In still yet another exemplary embodiment, R101 to R104 are the same as or different from each other, and may be each independently hydrogen; deuterium; or a substituted or unsubstituted C6 to C20 aryl group.

In a further exemplary embodiment, R101 to R104 are the same as or different from each other, and may be each independently hydrogen; or deuterium.

In another further exemplary embodiment, Rc and Rd are the same as or different from each other, and are each independently a substituted or unsubstituted C1 to C40 alkyl group; or a substituted or unsubstituted C6 to C40 aryl group, or may be bonded to each other to form a substituted or unsubstituted C6 to C40 ring.

In still another further exemplary embodiment, Rc and Rd are the same as or different from each other, and are each independently a substituted or unsubstituted C1 to C20 alkyl group; or a substituted or unsubstituted C6 to C20 aryl group, or may be bonded to each other to form a substituted or unsubstituted C6 to C20 ring.

In yet another further exemplary embodiment, Rc and Rd are the same as or different from each other, and are each independently a C1 to C20 alkyl group or a C6 to C20 aryl group, or may be bonded to each other to form a fluorene ring.

In still yet another further exemplary embodiment, Rc and Rd are the same as or different from each other, and are each independently a methyl group or a phenyl group, or may be bonded to each other to form a fluorene ring.

In a still further exemplary embodiment, Rc and Rd are the same as or different from each other, and may be each independently a substituted or unsubstituted C1 to C40 alkyl group or a substituted or unsubstituted C6 to C40 aryl group.

In a yet still further exemplary embodiment, Rc and Rd are the same as or different from each other, and may be each independently a substituted or unsubstituted C1 to C20 alkyl group or a substituted or unsubstituted C6 to C20 aryl group.

In another exemplary embodiment, Rc and Rd are the same as or different from each other, and may be each independently a C1 to C20 alkyl group or a substituted or unsubstituted C6 to C20 aryl group.

In still another exemplary embodiment, Rc and Rd are the same as or different from each other, and may be each independently a methyl group, a phenyl group or a fluorenyl group.

In an exemplary embodiment of the present application, provided is a heterocyclic compound in which Chemical Formula 1 is represented by any one of the following compounds.

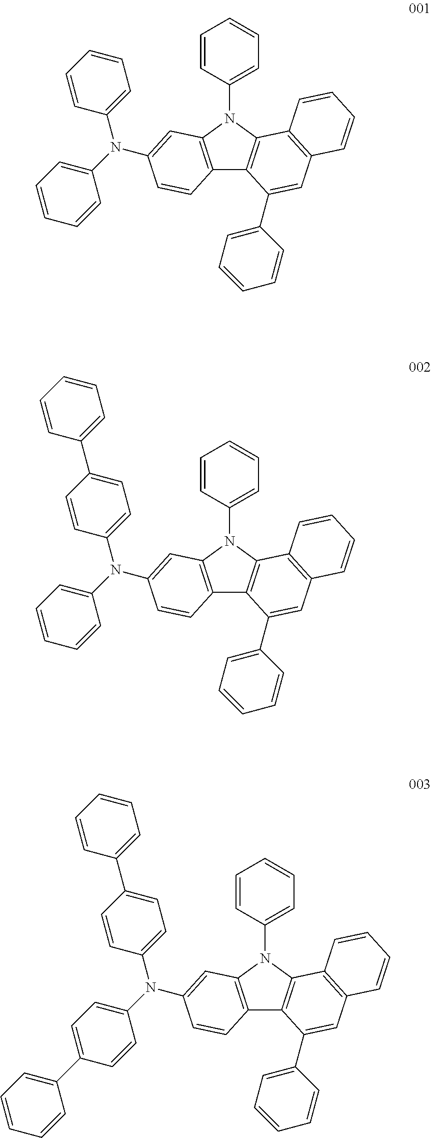 [SMILES: c1ccc(-c2cc3ccccc3c3c2c2ccc(N(c4ccccc4)c4ccccc4)cc2n3-c2ccccc2)cc1 ; c1ccc(-c2ccc(N(c3ccccc3)c3ccc4c5c(-c6ccccc6)cc6ccccc6c5n(-c5ccccc5)c4c3)cc2)cc1 ; c1ccc(-c2ccc(N(c3ccc(-c4ccccc4)cc3)c3ccc4c5c(-c6ccccc6)cc6ccccc6c5n(-c5ccccc5)c4c3)cc2)cc1]

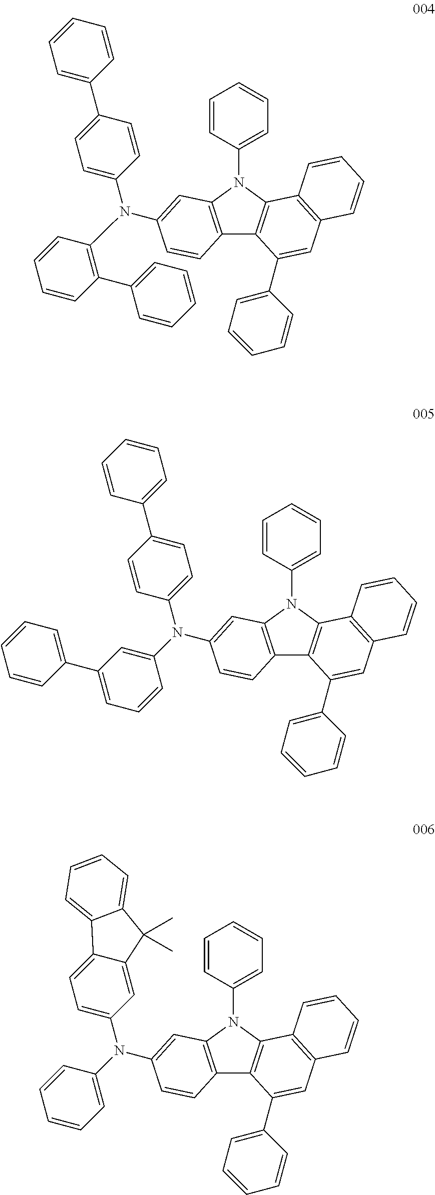 [SMILES: c1ccc(-c2ccc(N(c3ccc4c5c(-c6ccccc6)cc6ccccc6c5n(-c5ccccc5)c4c3)c3ccccc3-c3ccccc3)cc2)cc1 ; c1ccc(-c2ccc(N(c3cccc(-c4ccccc4)c3)c3ccc4c5c(-c6ccccc6)cc6ccccc6c5n(-c5ccccc5)c4c3)cc2)cc1 ; CC1(C)c2ccccc2-c2ccc(N(c3ccccc3)c3ccc4c5c(-c6ccccc6)cc6ccccc6c5n(-c5ccccc5)c4c3)cc21]

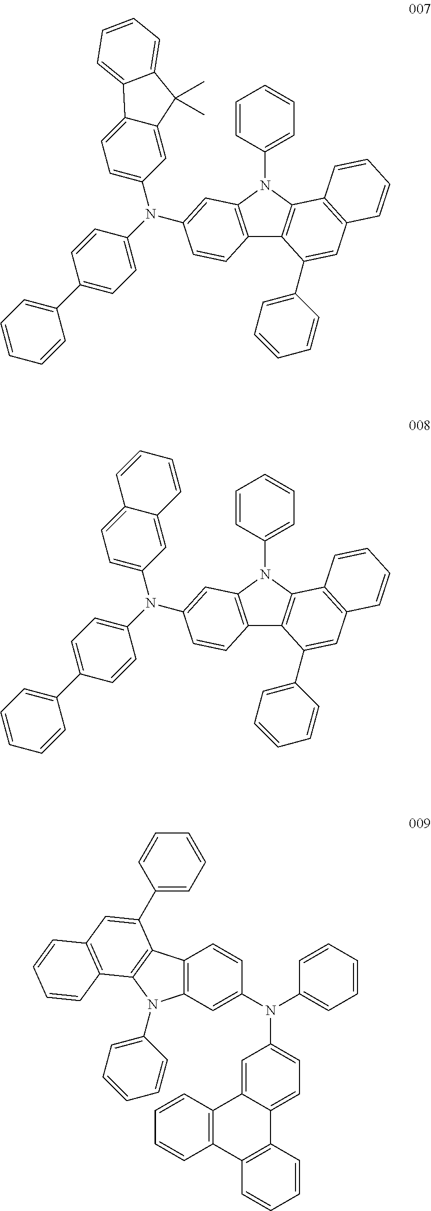 [SMILES: CC1(C)c2ccccc2-c2ccc(N(c3ccc(-c4ccccc4)cc3)c3ccc4c5c(-c6ccccc6)cc6ccccc6c5n(-c5ccccc5)c4c3)cc21 ; c1ccc(-c2ccc(N(c3ccc4ccccc4c3)c3ccc4c5c(-c6ccccc6)cc6ccccc6c5n(-c5ccccc5)c4c3)cc2)cc1 ; c1ccc(-c2cc3ccccc3c3c2c2ccc(N(c4ccccc4)c4ccc5c6ccccc6c6ccccc6c5c4)cc2n3-c2ccccc2)cc1]

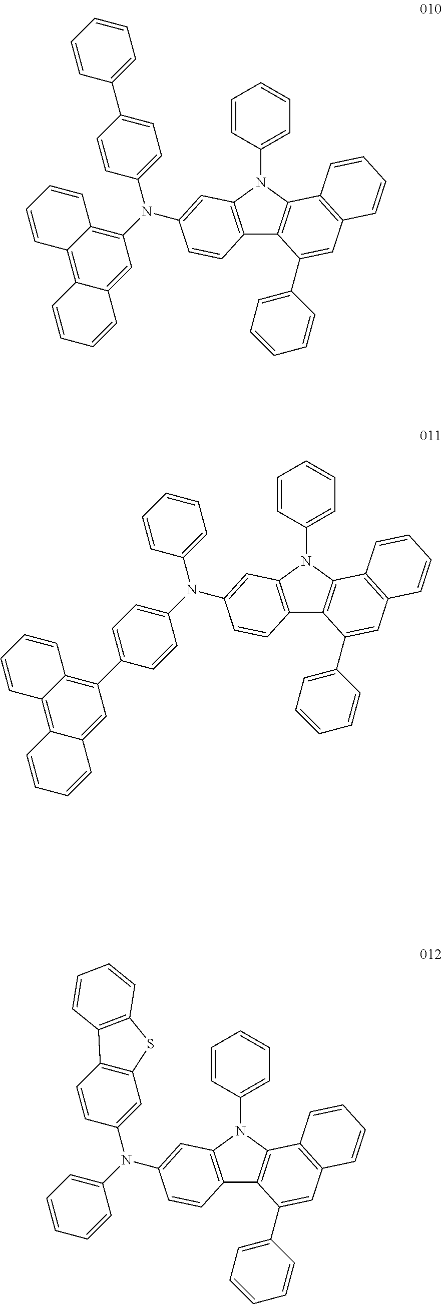 [SMILES: c1ccc(-c2ccc(N(c3ccc4c5c(-c6ccccc6)cc6ccccc6c5n(-c5ccccc5)c4c3)c3cc4ccccc4c4ccccc34)cc2)cc1 ; c1ccc(-c2cc3ccccc3c3c2c2ccc(N(c4ccccc4)c4ccc(-c5cc6ccccc6c6ccccc56)cc4)cc2n3-c2ccccc2)cc1 ; c1ccc(-c2cc3ccccc3c3c2c2ccc(N(c4ccccc4)c4ccc5c(c4)sc4ccccc45)cc2n3-c2ccccc2)cc1]

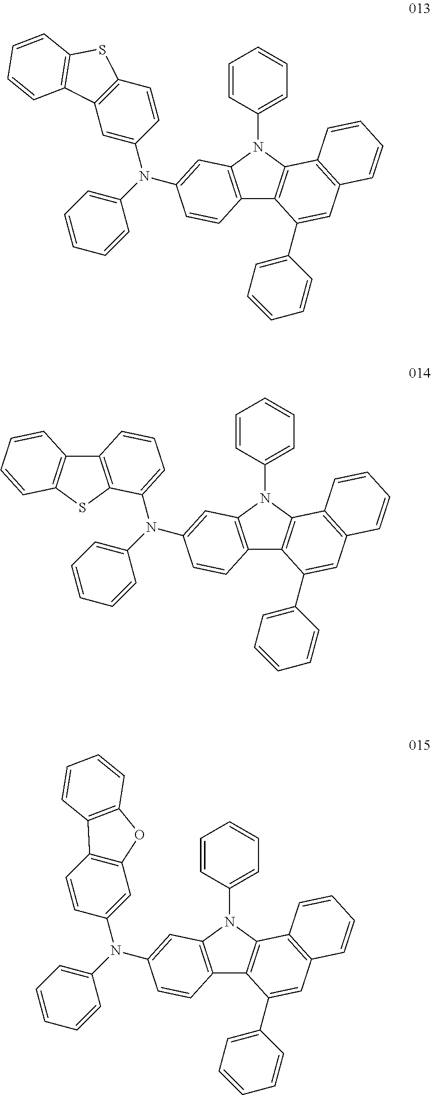 [SMILES: c1ccc(-c2cc3ccccc3c3c2c2ccc(N(c4ccccc4)c4ccc5sc6ccccc6c5c4)cc2n3-c2ccccc2)cc1 ; c1ccc(-c2cc3ccccc3c3c2c2ccc(N(c4ccccc4)c4cccc5c4sc4ccccc45)cc2n3-c2ccccc2)cc1 ; c1ccc(-c2cc3ccccc3c3c2c2ccc(N(c4ccccc4)c4ccc5c(c4)oc4ccccc45)cc2n3-c2ccccc2)cc1]

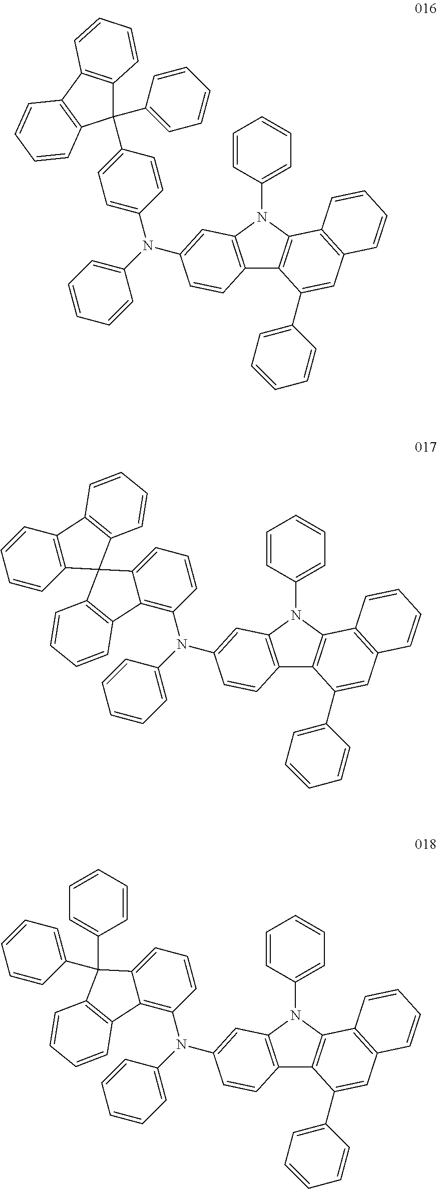 [SMILES: c1ccc(-c2cc3ccccc3c3c2c2ccc(N(c4ccccc4)c4ccc(C5(c6ccccc6)c6ccccc6-c6ccccc65)cc4)cc2n3-c2ccccc2)cc1 ; c1ccc(-c2cc3ccccc3c3c2c2ccc(N(c4ccccc4)c4cccc5c4-c4ccccc4C54c5ccccc5-c5ccccc54)cc2n3-c2ccccc2)cc1 ; c1ccc(-c2cc3ccccc3c3c2c2ccc(N(c4ccccc4)c4cccc5c4-c4ccccc4C5(c4ccccc4)c4ccccc4)cc2n3-c2ccccc2)cc1]

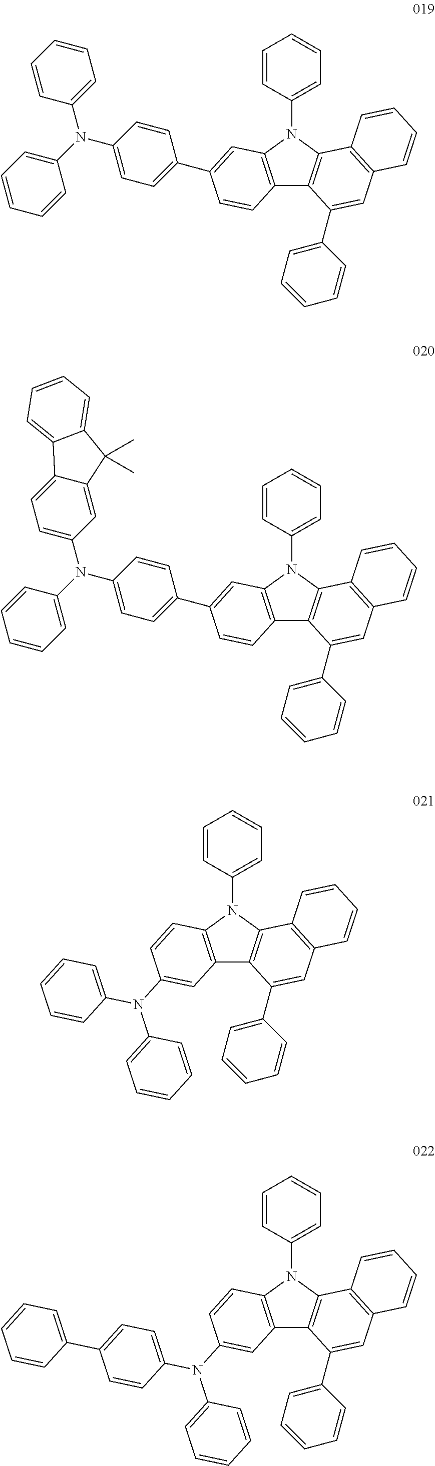 [SMILES: c1ccc(-c2cc3ccccc3c3c2c2ccc(-c4ccc(N(c5ccccc5)c5ccccc5)cc4)cc2n3-c2ccccc2)cc1 ; CC1(C)c2ccccc2-c2ccc(N(c3ccccc3)c3ccc(-c4ccc5c6c(-c7ccccc7)cc7ccccc7c6n(-c6ccccc6)c5c4)cc3)cc21 ; c1ccc(-c2cc3ccccc3c3c2c2cc(N(c4ccccc4)c4ccccc4)ccc2n3-c2ccccc2)cc1 ; c1ccc(-c2ccc(N(c3ccccc3)c3ccc4c(c3)c3c(-c5ccccc5)cc5ccccc5c3n4-c3ccccc3)cc2)cc1]

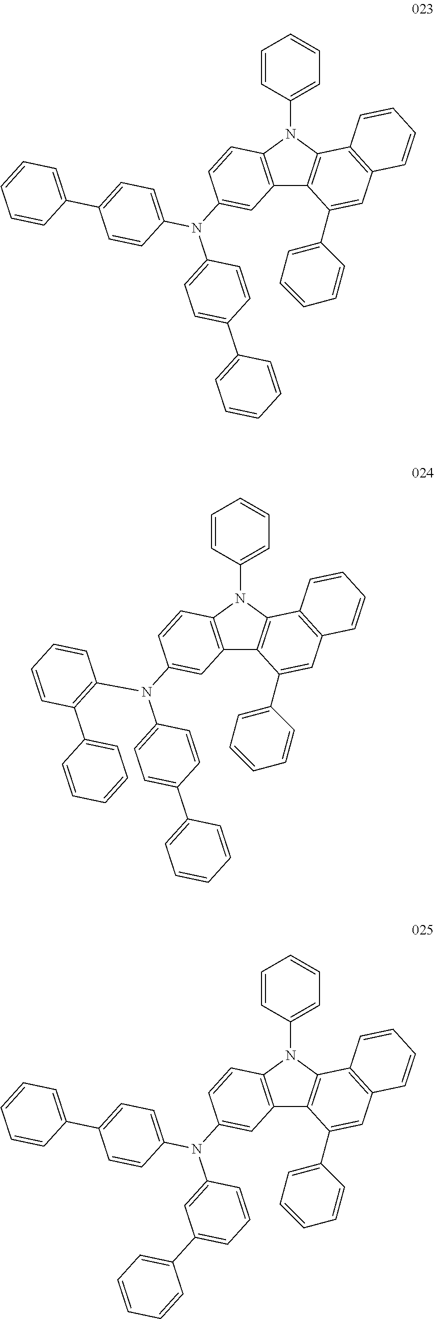 [SMILES: c1ccc(-c2ccc(N(c3ccc(-c4ccccc4)cc3)c3ccc4c(c3)c3c(-c5ccccc5)cc5ccccc5c3n4-c3ccccc3)cc2)cc1 ; c1ccc(-c2ccc(N(c3ccc4c(c3)c3c(-c5ccccc5)cc5ccccc5c3n4-c3ccccc3)c3ccccc3-c3ccccc3)cc2)cc1 ; c1ccc(-c2ccc(N(c3cccc(-c4ccccc4)c3)c3ccc4c(c3)c3c(-c5ccccc5)cc5ccccc5c3n4-c3ccccc3)cc2)cc1]

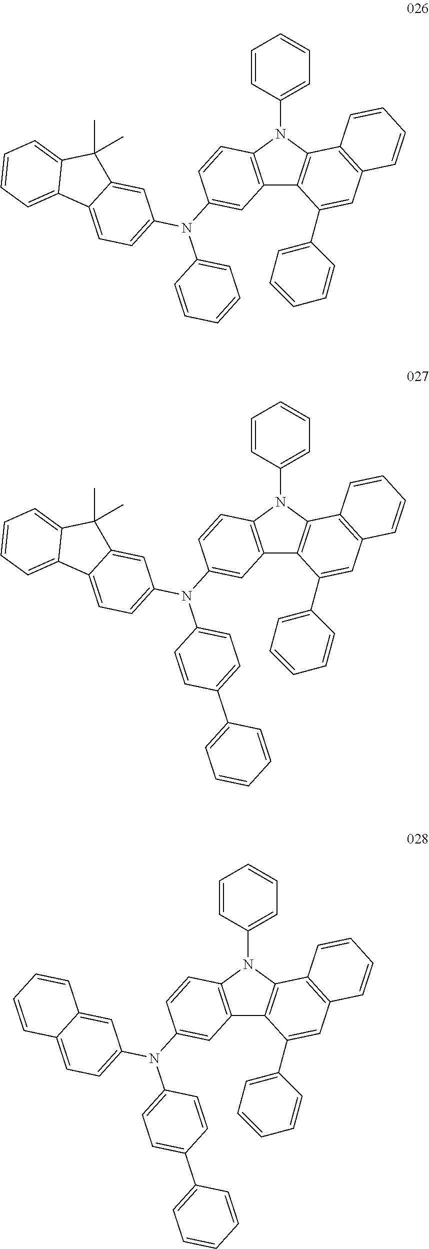 [SMILES: CC1(C)c2ccccc2-c2ccc(N(c3ccccc3)c3ccc4c(c3)c3c(-c5ccccc5)cc5ccccc5c3n4-c3ccccc3)cc21 ; CC1(C)c2ccccc2-c2ccc(N(c3ccc(-c4ccccc4)cc3)c3ccc4c(c3)c3c(-c5ccccc5)cc5ccccc5c3n4-c3ccccc3)cc21 ; c1ccc(-c2ccc(N(c3ccc4ccccc4c3)c3ccc4c(c3)c3c(-c5ccccc5)cc5ccccc5c3n4-c3ccccc3)cc2)cc1]

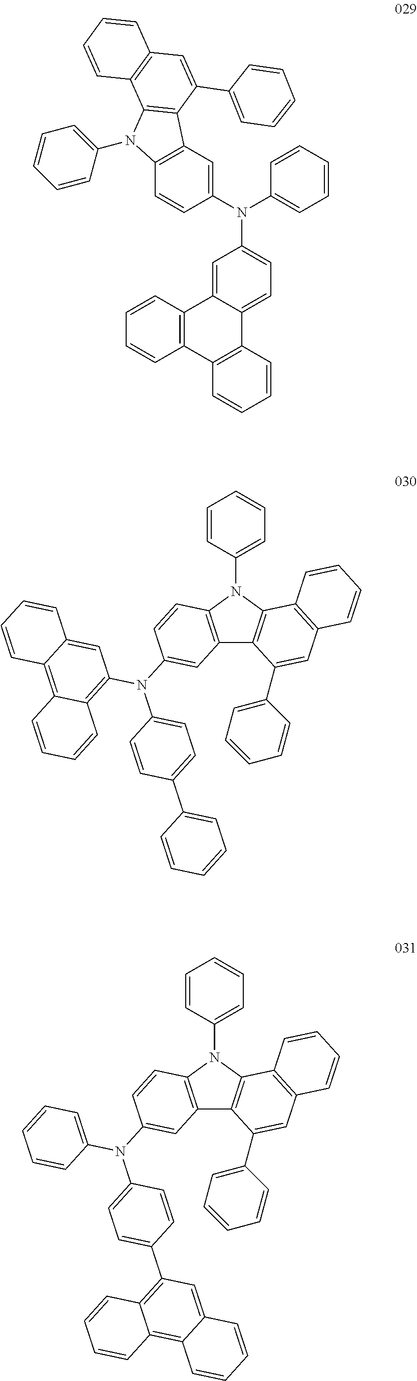 [SMILES: c1ccc(-c2cc3ccccc3c3c2c2cc(N(c4ccccc4)c4ccc5c6ccccc6c6ccccc6c5c4)ccc2n3-c2ccccc2)cc1 ; c1ccc(-c2ccc(N(c3ccc4c(c3)c3c(-c5ccccc5)cc5ccccc5c3n4-c3ccccc3)c3cc4ccccc4c4ccccc34)cc2)cc1 ; c1ccc(-c2cc3ccccc3c3c2c2cc(N(c4ccccc4)c4ccc(-c5cc6ccccc6c6ccccc56)cc4)ccc2n3-c2ccccc2)cc1]

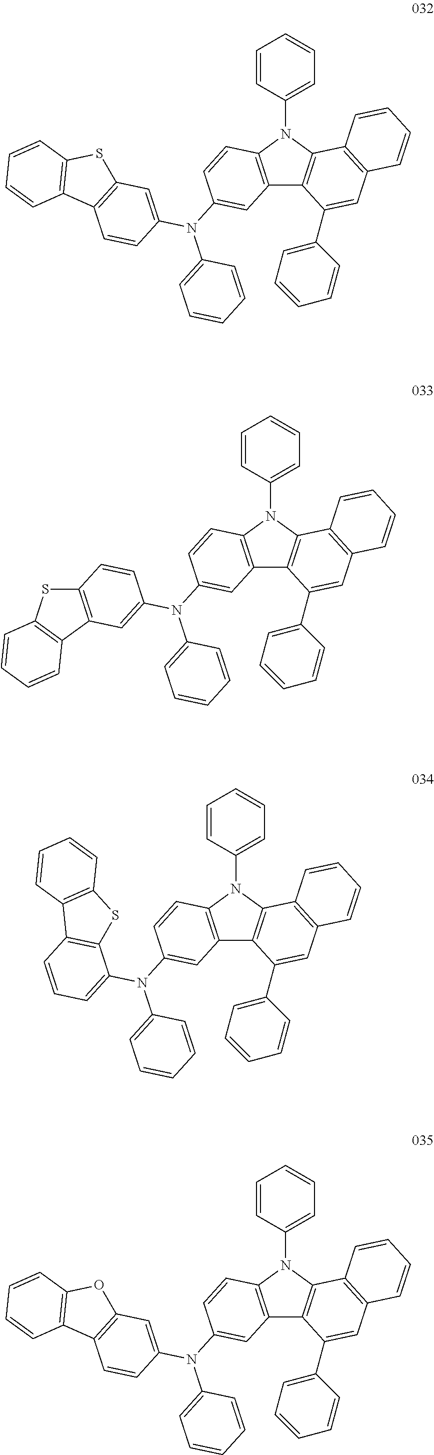 [SMILES: c1ccc(-c2cc3ccccc3c3c2c2cc(N(c4ccccc4)c4ccc5c(c4)sc4ccccc45)ccc2n3-c2ccccc2)cc1 ; c1ccc(-c2cc3ccccc3c3c2c2cc(N(c4ccccc4)c4ccc5sc6ccccc6c5c4)ccc2n3-c2ccccc2)cc1 ; c1ccc(-c2cc3ccccc3c3c2c2cc(N(c4ccccc4)c4cccc5c4sc4ccccc45)ccc2n3-c2ccccc2)cc1 ; c1ccc(-c2cc3ccccc3c3c2c2cc(N(c4ccccc4)c4ccc5c(c4)oc4ccccc45)ccc2n3-c2ccccc2)cc1]

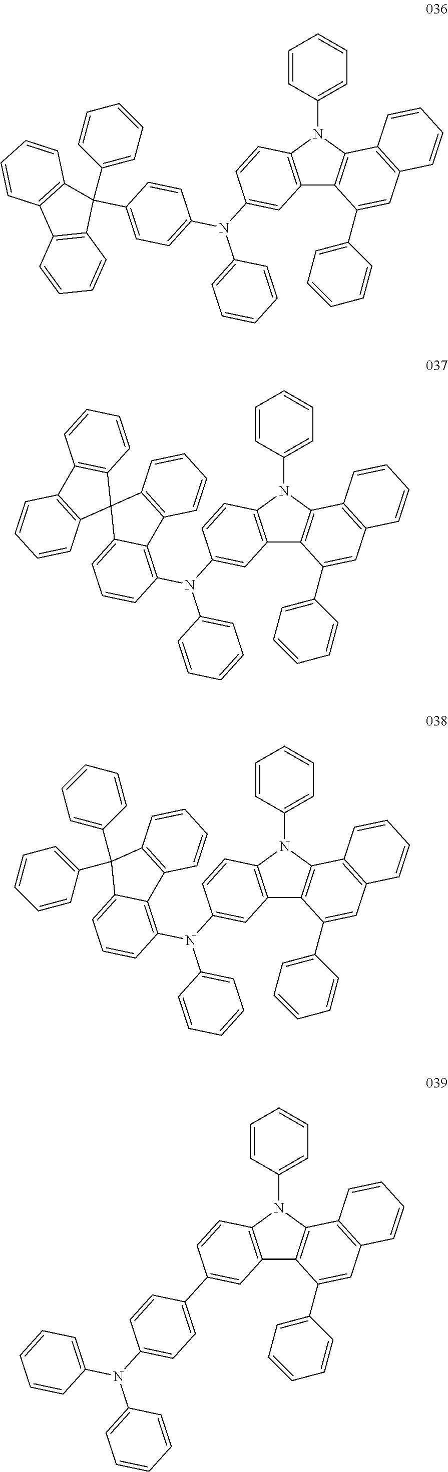 [SMILES: c1ccc(-c2cc3ccccc3c3c2c2cc(N(c4ccccc4)c4ccc(C5(c6ccccc6)c6ccccc6-c6ccccc65)cc4)ccc2n3-c2ccccc2)cc1 ; c1ccc(-c2cc3ccccc3c3c2c2cc(N(c4ccccc4)c4cccc5c4-c4ccccc4C54c5ccccc5-c5ccccc54)ccc2n3-c2ccccc2)cc1 ; c1ccc(-c2cc3ccccc3c3c2c2cc(N(c4ccccc4)c4cccc5c4-c4ccccc4C5(c4ccccc4)c4ccccc4)ccc2n3-c2ccccc2)cc1 ; c1ccc(-c2cc3ccccc3c3c2c2cc(-c4ccc(N(c5ccccc5)c5ccccc5)cc4)ccc2n3-c2ccccc2)cc1]

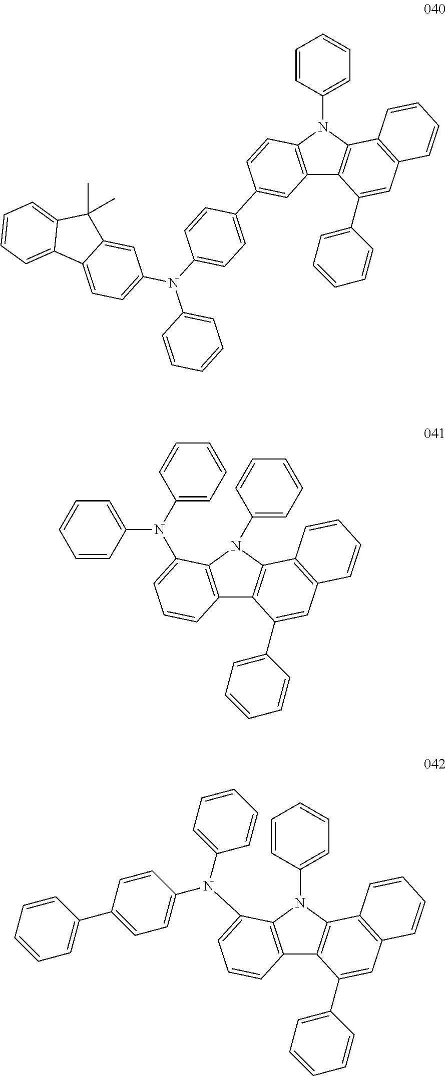 [SMILES: CC1(C)c2ccccc2-c2ccc(N(c3ccccc3)c3ccc(-c4ccc5c(c4)c4c(-c6ccccc6)cc6ccccc6c4n5-c4ccccc4)cc3)cc21 ; c1ccc(-c2cc3ccccc3c3c2c2cccc(N(c4ccccc4)c4ccccc4)c2n3-c2ccccc2)cc1 ; c1ccc(-c2ccc(N(c3ccccc3)c3cccc4c5c(-c6ccccc6)cc6ccccc6c5n(-c5ccccc5)c34)cc2)cc1]

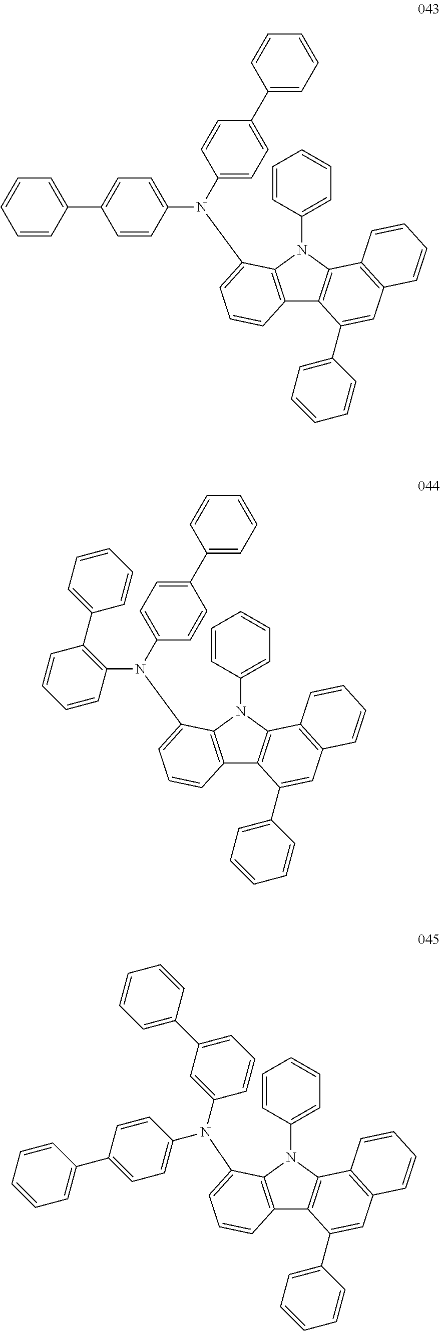 [SMILES: c1ccc(-c2ccc(N(c3ccc(-c4ccccc4)cc3)c3cccc4c5c(-c6ccccc6)cc6ccccc6c5n(-c5ccccc5)c34)cc2)cc1 ; c1ccc(-c2ccc(N(c3ccccc3-c3ccccc3)c3cccc4c5c(-c6ccccc6)cc6ccccc6c5n(-c5ccccc5)c34)cc2)cc1 ; c1ccc(-c2ccc(N(c3cccc(-c4ccccc4)c3)c3cccc4c5c(-c6ccccc6)cc6ccccc6c5n(-c5ccccc5)c34)cc2)cc1]

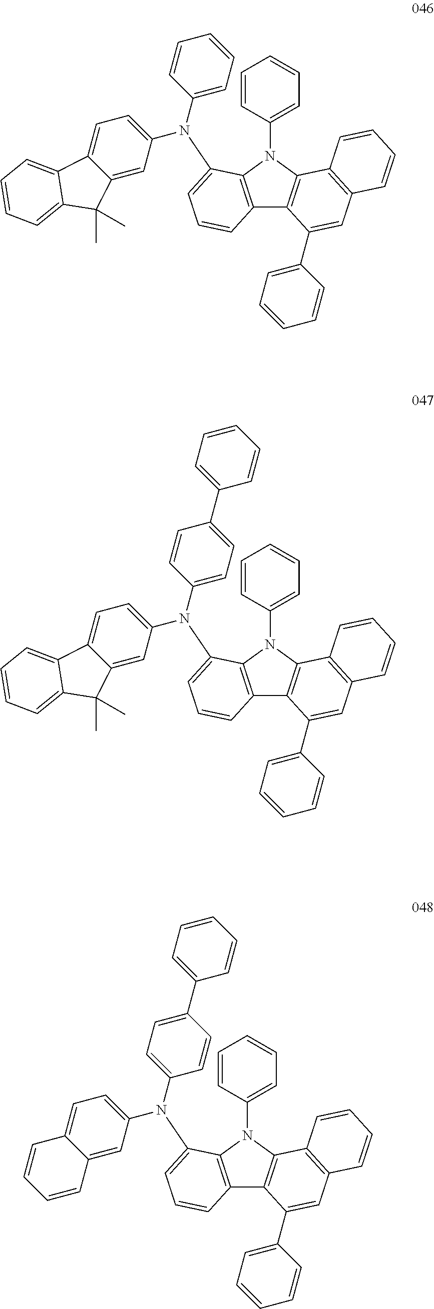 [SMILES: CC1(C)c2ccccc2-c2ccc(N(c3ccccc3)c3cccc4c5c(-c6ccccc6)cc6ccccc6c5n(-c5ccccc5)c34)cc21 ; CC1(C)c2ccccc2-c2ccc(N(c3ccc(-c4ccccc4)cc3)c3cccc4c5c(-c6ccccc6)cc6ccccc6c5n(-c5ccccc5)c34)cc21 ; c1ccc(-c2ccc(N(c3ccc4ccccc4c3)c3cccc4c5c(-c6ccccc6)cc6ccccc6c5n(-c5ccccc5)c34)cc2)cc1]

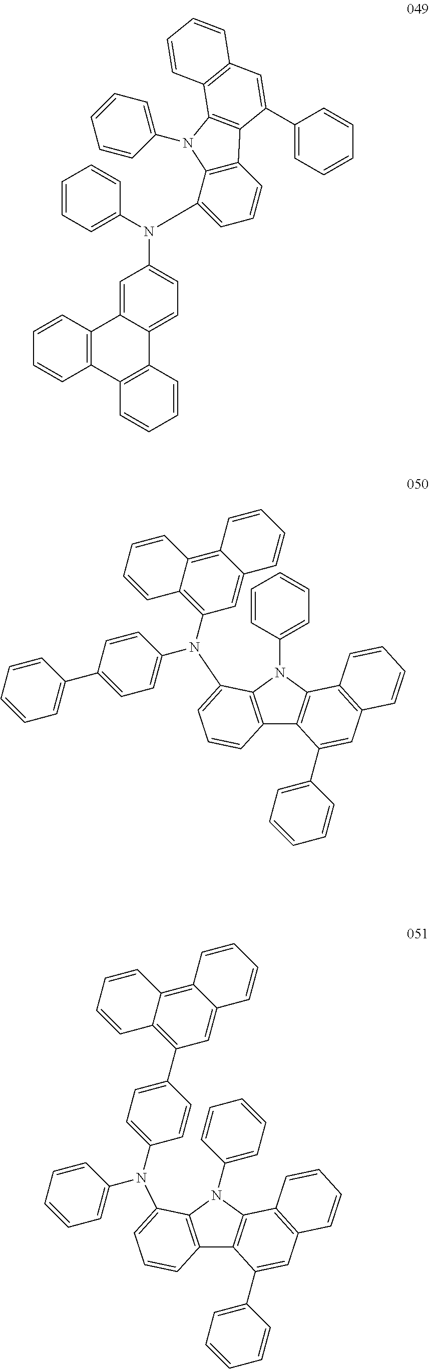 [SMILES: c1ccc(-c2cc3ccccc3c3c2c2cccc(N(c4ccccc4)c4ccc5c6ccccc6c6ccccc6c5c4)c2n3-c2ccccc2)cc1 ; c1ccc(-c2ccc(N(c3cc4ccccc4c4ccccc34)c3cccc4c5c(-c6ccccc6)cc6ccccc6c5n(-c5ccccc5)c34)cc2)cc1 ; c1ccc(-c2cc3ccccc3c3c2c2cccc(N(c4ccccc4)c4ccc(-c5cc6ccccc6c6ccccc56)cc4)c2n3-c2ccccc2)cc1]

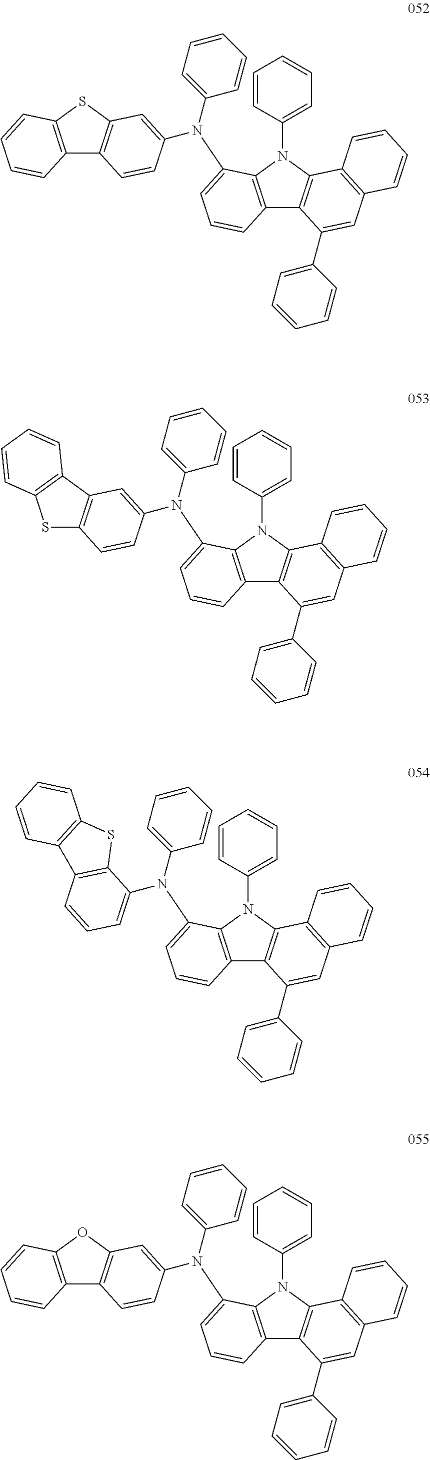 [SMILES: c1ccc(-c2cc3ccccc3c3c2c2cccc(N(c4ccccc4)c4ccc5c(c4)sc4ccccc45)c2n3-c2ccccc2)cc1 ; c1ccc(-c2cc3ccccc3c3c2c2cccc(N(c4ccccc4)c4ccc5sc6ccccc6c5c4)c2n3-c2ccccc2)cc1 ; c1ccc(-c2cc3ccccc3c3c2c2cccc(N(c4ccccc4)c4cccc5c4sc4ccccc45)c2n3-c2ccccc2)cc1 ; c1ccc(-c2cc3ccccc3c3c2c2cccc(N(c4ccccc4)c4ccc5c(c4)oc4ccccc45)c2n3-c2ccccc2)cc1]

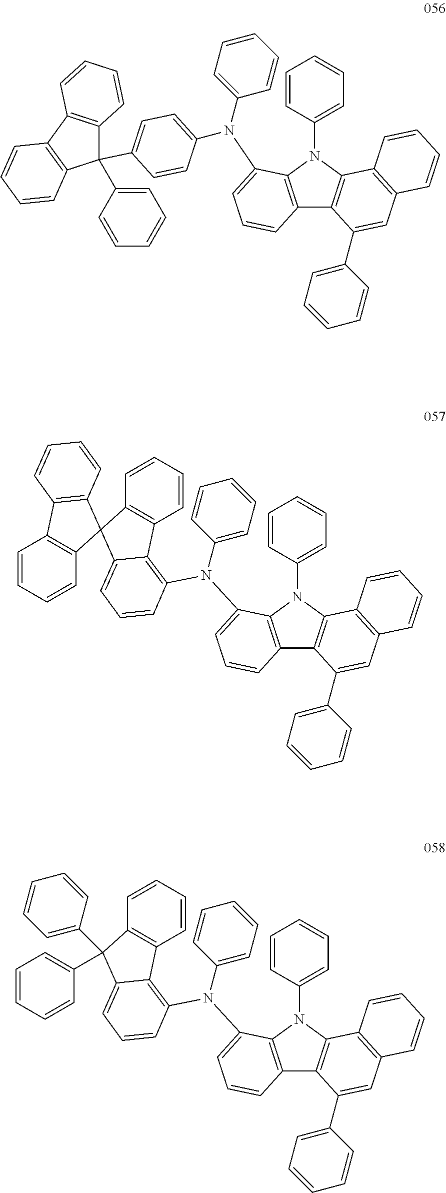 [SMILES: c1ccc(-c2cc3ccccc3c3c2c2cccc(N(c4ccccc4)c4ccc(C5(c6ccccc6)c6ccccc6-c6ccccc65)cc4)c2n3-c2ccccc2)cc1 ; c1ccc(-c2cc3ccccc3c3c2c2cccc(N(c4ccccc4)c4cccc5c4-c4ccccc4C54c5ccccc5-c5ccccc54)c2n3-c2ccccc2)cc1 ; c1ccc(-c2cc3ccccc3c3c2c2cccc(N(c4ccccc4)c4cccc5c4-c4ccccc4C5(c4ccccc4)c4ccccc4)c2n3-c2ccccc2)cc1]

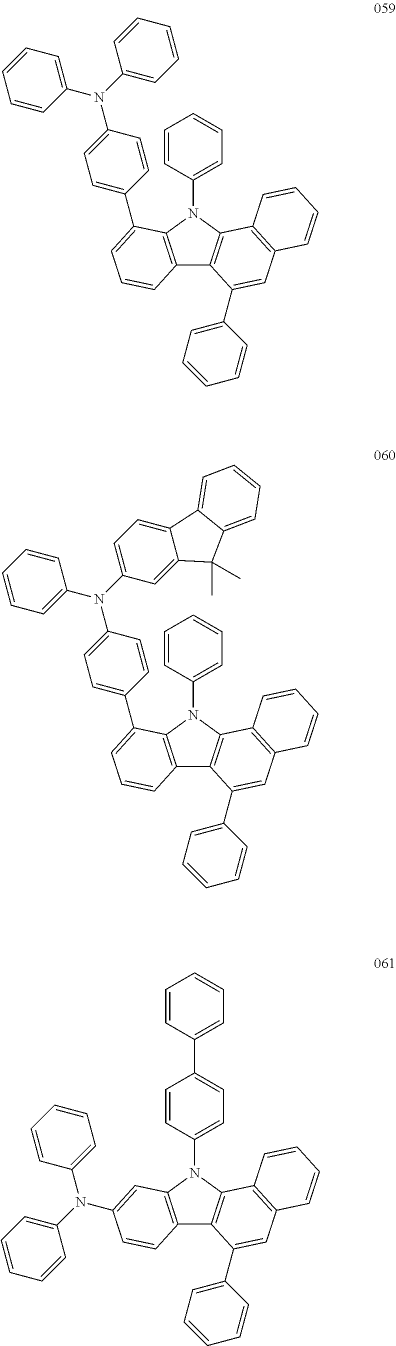 [SMILES: c1ccc(-c2cc3ccccc3c3c2c2cccc(-c4ccc(N(c5ccccc5)c5ccccc5)cc4)c2n3-c2ccccc2)cc1 ; CC1(C)c2ccccc2-c2ccc(N(c3ccccc3)c3ccc(-c4cccc5c6c(-c7ccccc7)cc7ccccc7c6n(-c6ccccc6)c45)cc3)cc21 ; c1ccc(-c2ccc(-n3c4cc(N(c5ccccc5)c5ccccc5)ccc4c4c(-c5ccccc5)cc5ccccc5c43)cc2)cc1]

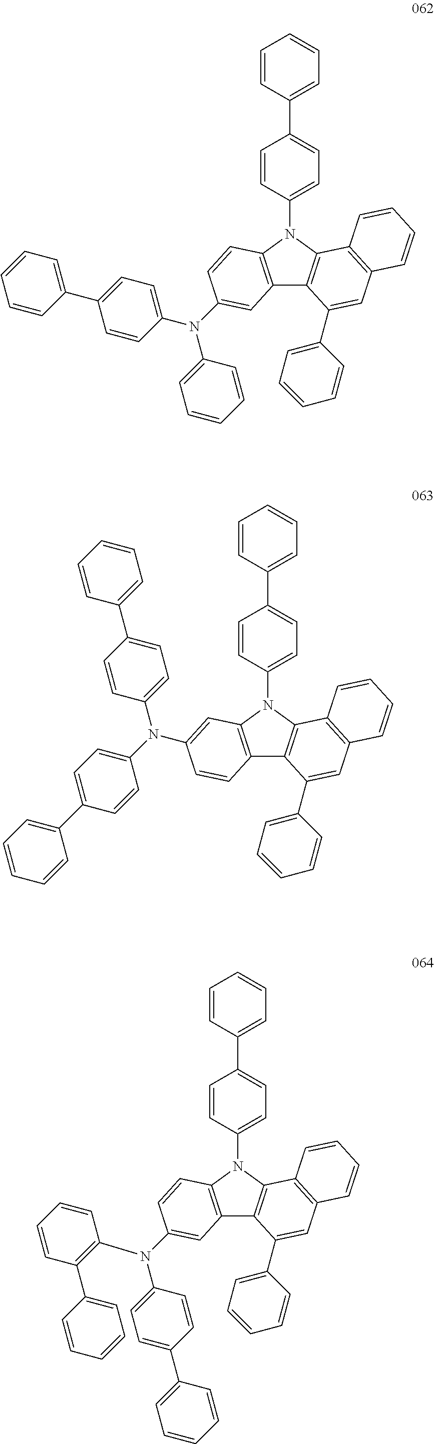 [SMILES: c1ccc(-c2ccc(N(c3ccccc3)c3ccc4c(c3)c3c(-c5ccccc5)cc5ccccc5c3n4-c3ccc(-c4ccccc4)cc3)cc2)cc1 ; c1ccc(-c2ccc(N(c3ccc(-c4ccccc4)cc3)c3ccc4c5c(-c6ccccc6)cc6ccccc6c5n(-c5ccc(-c6ccccc6)cc5)c4c3)cc2)cc1 ; c1ccc(-c2ccc(N(c3ccc4c(c3)c3c(-c5ccccc5)cc5ccccc5c3n4-c3ccc(-c4ccccc4)cc3)c3ccccc3-c3ccccc3)cc2)cc1]

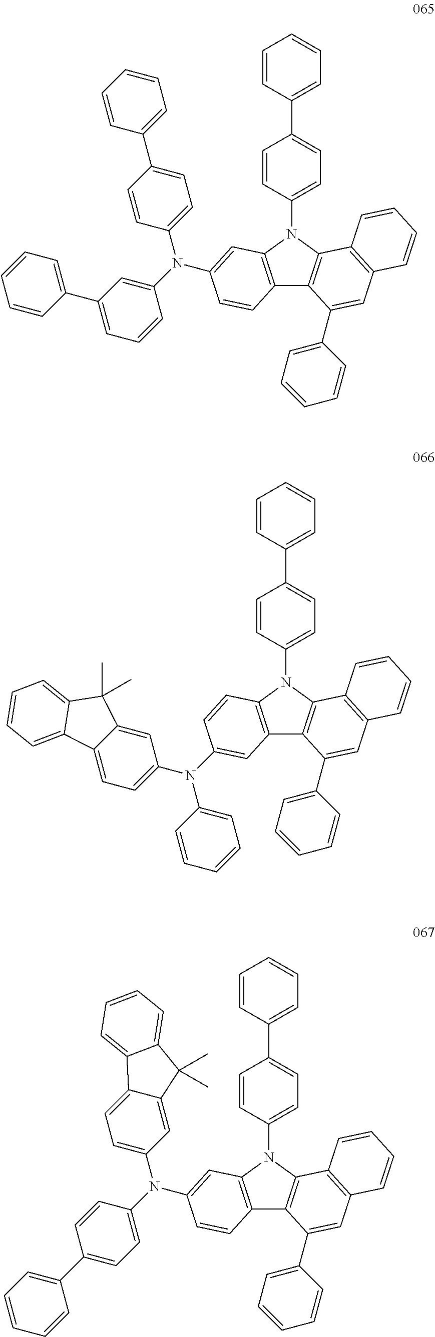 [SMILES: c1ccc(-c2ccc(N(c3cccc(-c4ccccc4)c3)c3ccc4c5c(-c6ccccc6)cc6ccccc6c5n(-c5ccc(-c6ccccc6)cc5)c4c3)cc2)cc1 ; CC1(C)c2ccccc2-c2ccc(N(c3ccccc3)c3ccc4c(c3)c3c(-c5ccccc5)cc5ccccc5c3n4-c3ccc(-c4ccccc4)cc3)cc21 ; CC1(C)c2ccccc2-c2ccc(N(c3ccc(-c4ccccc4)cc3)c3ccc4c5c(-c6ccccc6)cc6ccccc6c5n(-c5ccc(-c6ccccc6)cc5)c4c3)cc21]

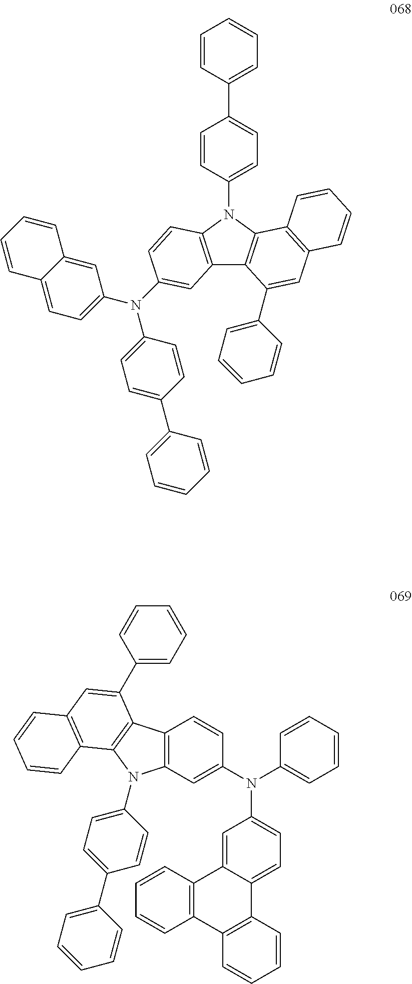 [SMILES: c1ccc(-c2ccc(N(c3ccc4ccccc4c3)c3ccc4c(c3)c3c(-c5ccccc5)cc5ccccc5c3n4-c3ccc(-c4ccccc4)cc3)cc2)cc1 ; c1ccc(-c2ccc(-n3c4cc(N(c5ccccc5)c5ccc6c7ccccc7c7ccccc7c6c5)ccc4c4c(-c5ccccc5)cc5ccccc5c43)cc2)cc1]

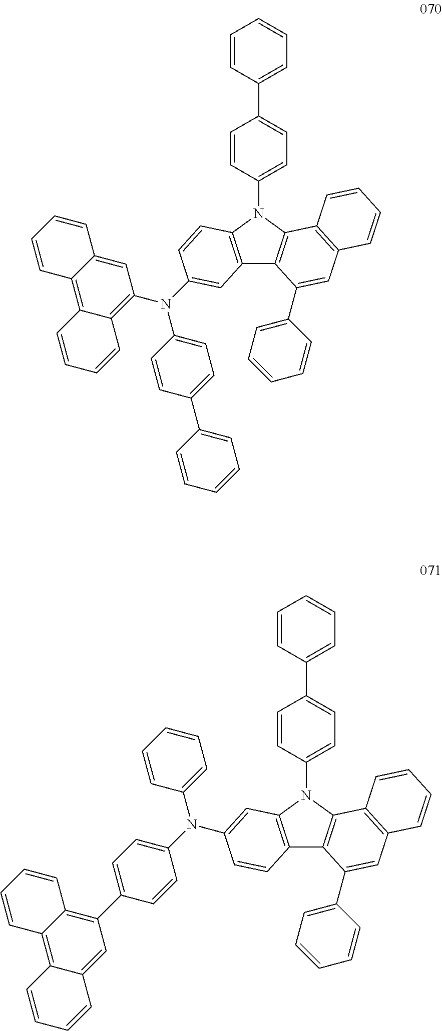 [SMILES: c1ccc(-c2ccc(N(c3ccc4c(c3)c3c(-c5ccccc5)cc5ccccc5c3n4-c3ccc(-c4ccccc4)cc3)c3cc4ccccc4c4ccccc34)cc2)cc1 ; c1ccc(-c2ccc(-n3c4cc(N(c5ccccc5)c5ccc(-c6cc7ccccc7c7ccccc67)cc5)ccc4c4c(-c5ccccc5)cc5ccccc5c43)cc2)cc1]

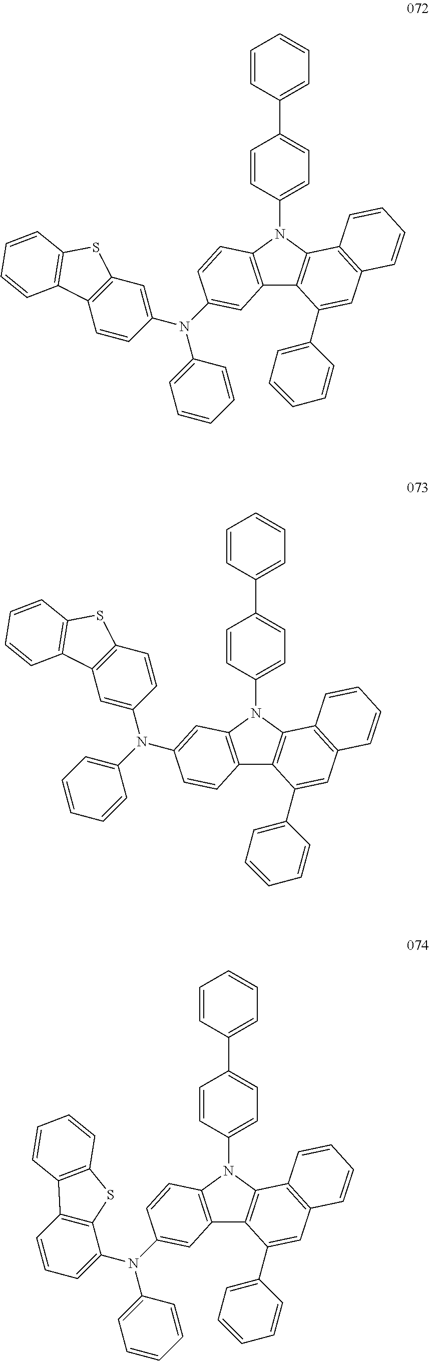 [SMILES: c1ccc(-c2ccc(-n3c4ccc(N(c5ccccc5)c5ccc6c(c5)sc5ccccc56)cc4c4c(-c5ccccc5)cc5ccccc5c43)cc2)cc1 ; c1ccc(-c2ccc(-n3c4cc(N(c5ccccc5)c5ccc6sc7ccccc7c6c5)ccc4c4c(-c5ccccc5)cc5ccccc5c43)cc2)cc1 ; c1ccc(-c2ccc(-n3c4ccc(N(c5ccccc5)c5cccc6c5sc5ccccc56)cc4c4c(-c5ccccc5)cc5ccccc5c43)cc2)cc1]

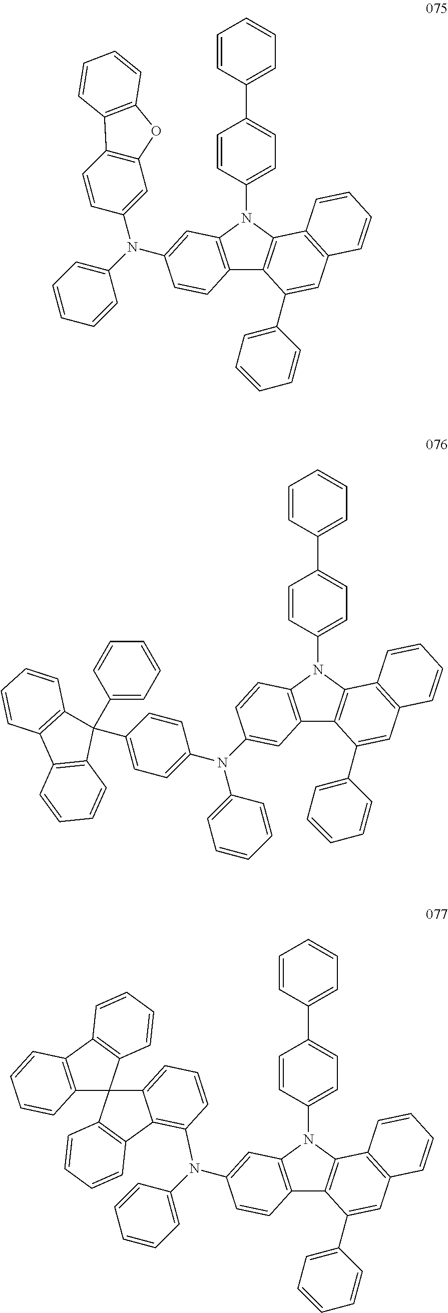 [SMILES: c1ccc(-c2ccc(-n3c4cc(N(c5ccccc5)c5ccc6c(c5)oc5ccccc56)ccc4c4c(-c5ccccc5)cc5ccccc5c43)cc2)cc1 ; c1ccc(-c2ccc(-n3c4ccc(N(c5ccccc5)c5ccc(C6(c7ccccc7)c7ccccc7-c7ccccc76)cc5)cc4c4c(-c5ccccc5)cc5ccccc5c43)cc2)cc1 ; c1ccc(-c2ccc(-n3c4cc(N(c5ccccc5)c5cccc6c5-c5ccccc5C65c6ccccc6-c6ccccc65)ccc4c4c(-c5ccccc5)cc5ccccc5c43)cc2)cc1]

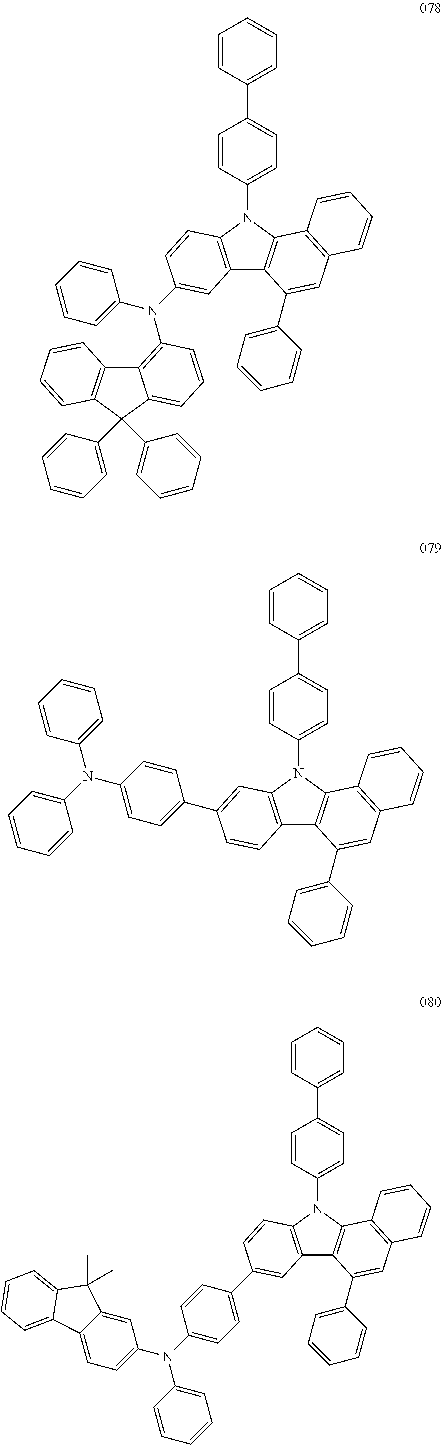 [SMILES: c1ccc(-c2ccc(-n3c4ccc(N(c5ccccc5)c5cccc6c5-c5ccccc5C6(c5ccccc5)c5ccccc5)cc4c4c(-c5ccccc5)cc5ccccc5c43)cc2)cc1 ; c1ccc(-c2ccc(-n3c4cc(-c5ccc(N(c6ccccc6)c6ccccc6)cc5)ccc4c4c(-c5ccccc5)cc5ccccc5c43)cc2)cc1 ; CC1(C)c2ccccc2-c2ccc(N(c3ccccc3)c3ccc(-c4ccc5c(c4)c4c(-c6ccccc6)cc6ccccc6c4n5-c4ccc(-c5ccccc5)cc4)cc3)cc21]

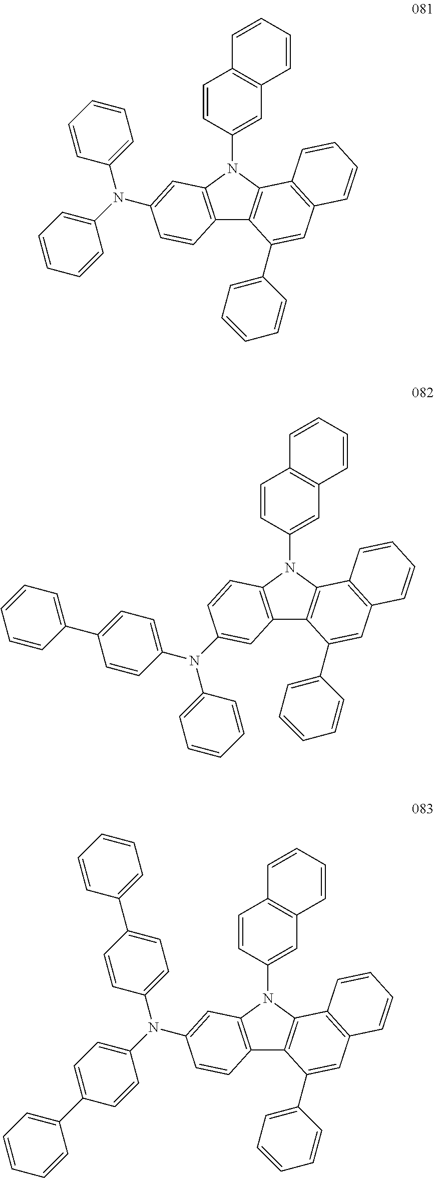 [SMILES: c1ccc(-c2cc3ccccc3c3c2c2ccc(N(c4ccccc4)c4ccccc4)cc2n3-c2ccc3ccccc3c2)cc1 ; c1ccc(-c2ccc(N(c3ccccc3)c3ccc4c(c3)c3c(-c5ccccc5)cc5ccccc5c3n4-c3ccc4ccccc4c3)cc2)cc1 ; c1ccc(-c2ccc(N(c3ccc(-c4ccccc4)cc3)c3ccc4c5c(-c6ccccc6)cc6ccccc6c5n(-c5ccc6ccccc6c5)c4c3)cc2)cc1]

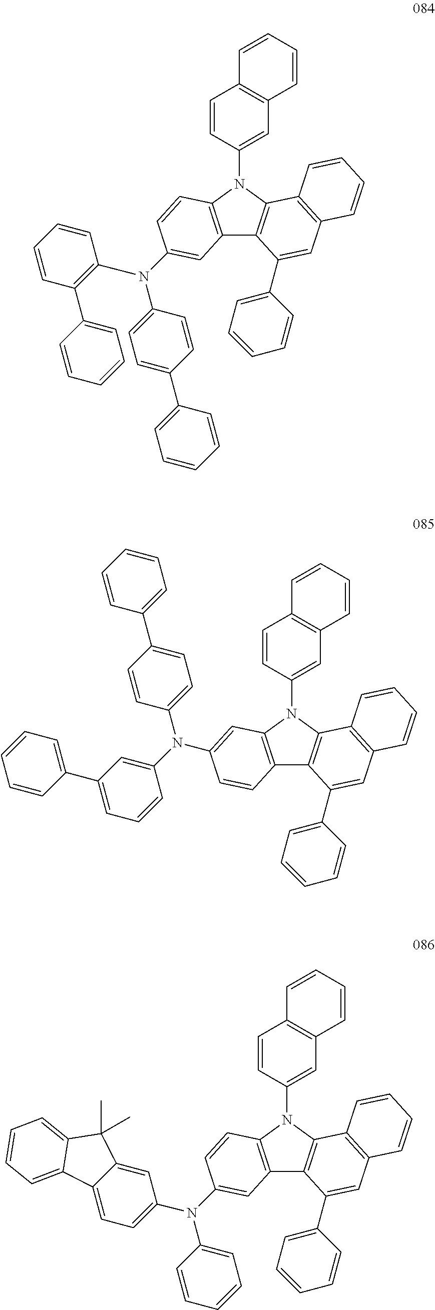 [SMILES: c1ccc(-c2ccc(N(c3ccc4c(c3)c3c(-c5ccccc5)cc5ccccc5c3n4-c3ccc4ccccc4c3)c3ccccc3-c3ccccc3)cc2)cc1 ; c1ccc(-c2ccc(N(c3cccc(-c4ccccc4)c3)c3ccc4c5c(-c6ccccc6)cc6ccccc6c5n(-c5ccc6ccccc6c5)c4c3)cc2)cc1 ; CC1(C)c2ccccc2-c2ccc(N(c3ccccc3)c3ccc4c(c3)c3c(-c5ccccc5)cc5ccccc5c3n4-c3ccc4ccccc4c3)cc21]

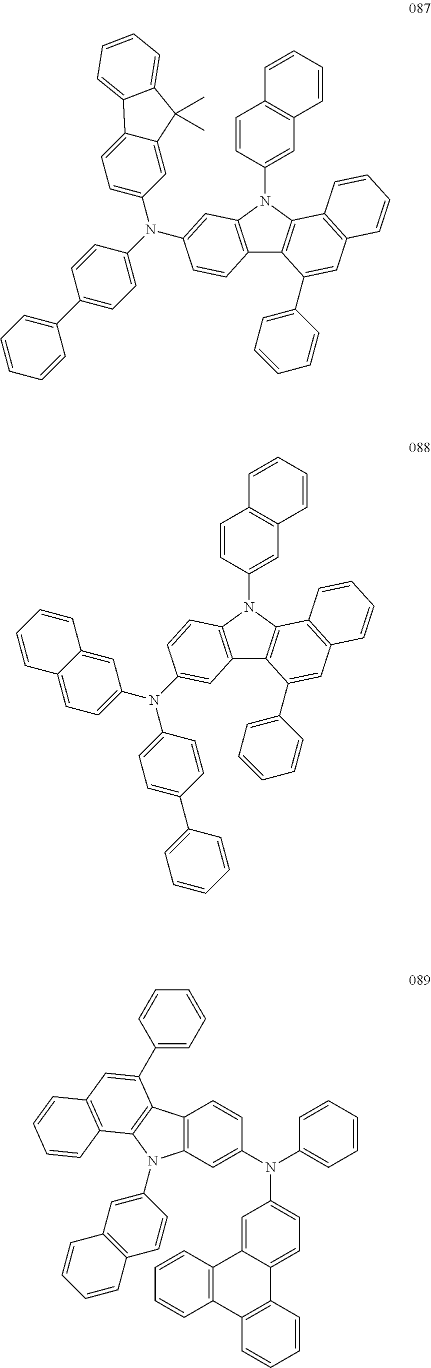 [SMILES: CC1(C)c2ccccc2-c2ccc(N(c3ccc(-c4ccccc4)cc3)c3ccc4c5c(-c6ccccc6)cc6ccccc6c5n(-c5ccc6ccccc6c5)c4c3)cc21 ; c1ccc(-c2ccc(N(c3ccc4ccccc4c3)c3ccc4c(c3)c3c(-c5ccccc5)cc5ccccc5c3n4-c3ccc4ccccc4c3)cc2)cc1 ; c1ccc(-c2cc3ccccc3c3c2c2ccc(N(c4ccccc4)c4ccc5c6ccccc6c6ccccc6c5c4)cc2n3-c2ccc3ccccc3c2)cc1]

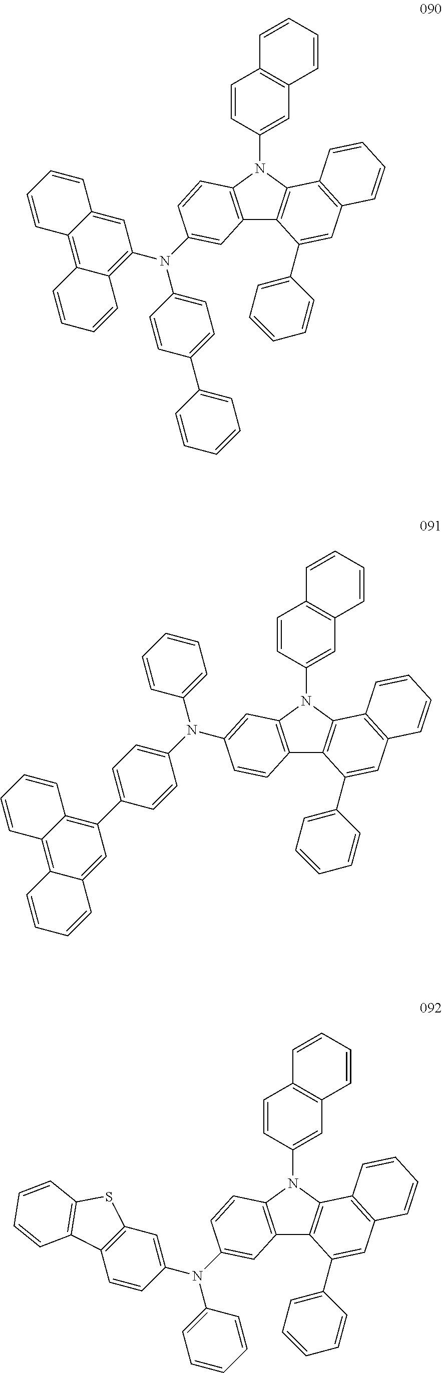 [SMILES: c1ccc(-c2ccc(N(c3ccc4c(c3)c3c(-c5ccccc5)cc5ccccc5c3n4-c3ccc4ccccc4c3)c3cc4ccccc4c4ccccc34)cc2)cc1 ; c1ccc(-c2cc3ccccc3c3c2c2ccc(N(c4ccccc4)c4ccc(-c5cc6ccccc6c6ccccc56)cc4)cc2n3-c2ccc3ccccc3c2)cc1 ; c1ccc(-c2cc3ccccc3c3c2c2cc(N(c4ccccc4)c4ccc5c(c4)sc4ccccc45)ccc2n3-c2ccc3ccccc3c2)cc1]

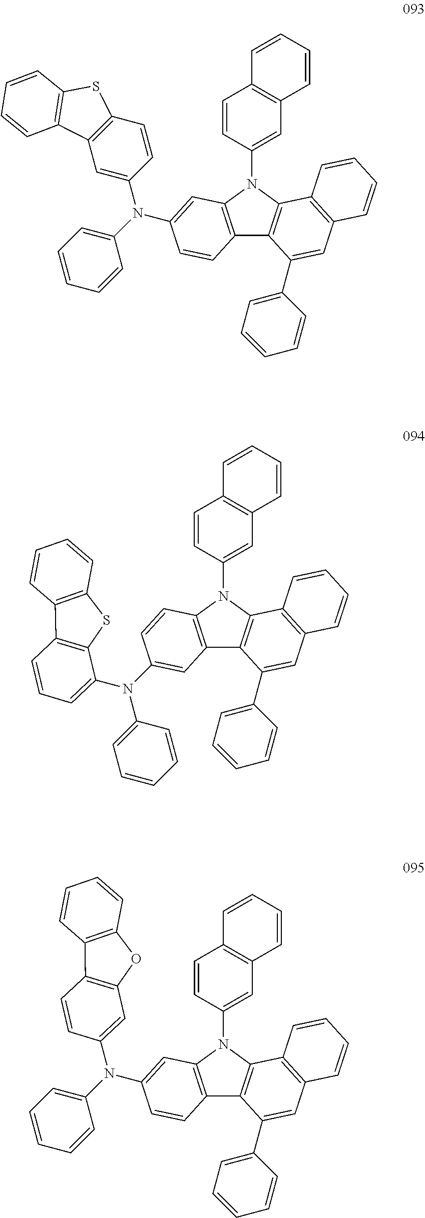 [SMILES: c1ccc(-c2cc3ccccc3c3c2c2ccc(N(c4ccccc4)c4ccc5sc6ccccc6c5c4)cc2n3-c2ccc3ccccc3c2)cc1 ; c1ccc(-c2cc3ccccc3c3c2c2cc(N(c4ccccc4)c4cccc5c4sc4ccccc45)ccc2n3-c2ccc3ccccc3c2)cc1 ; c1ccc(-c2cc3ccccc3c3c2c2ccc(N(c4ccccc4)c4ccc5c(c4)oc4ccccc45)cc2n3-c2ccc3ccccc3c2)cc1]

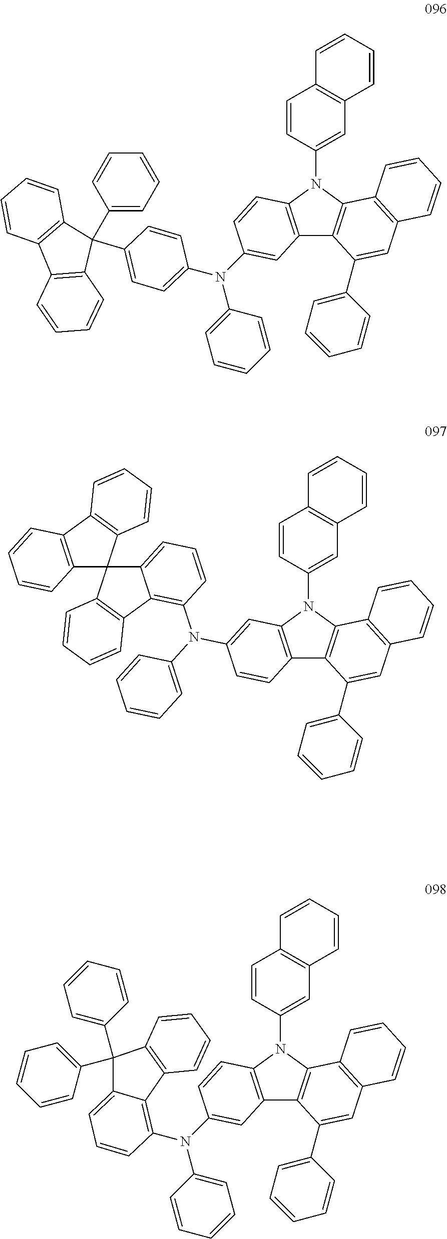 [SMILES: c1ccc(-c2cc3ccccc3c3c2c2cc(N(c4ccccc4)c4ccc(C5(c6ccccc6)c6ccccc6-c6ccccc65)cc4)ccc2n3-c2ccc3ccccc3c2)cc1 ; c1ccc(-c2cc3ccccc3c3c2c2ccc(N(c4ccccc4)c4cccc5c4-c4ccccc4C54c5ccccc5-c5ccccc54)cc2n3-c2ccc3ccccc3c2)cc1 ; c1ccc(-c2cc3ccccc3c3c2c2cc(N(c4ccccc4)c4cccc5c4-c4ccccc4C5(c4ccccc4)c4ccccc4)ccc2n3-c2ccc3ccccc3c2)cc1]

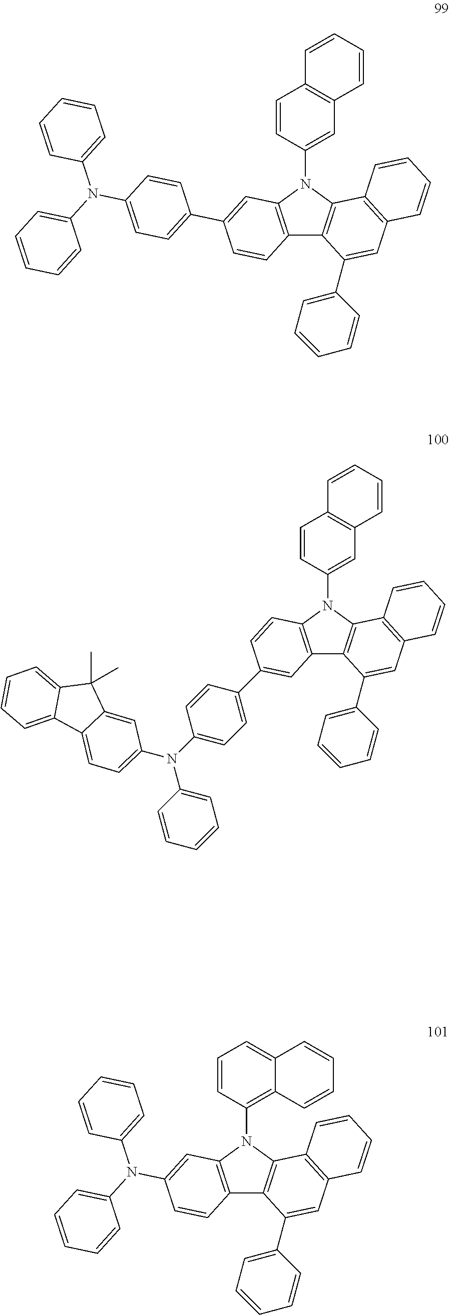 [SMILES: c1ccc(-c2cc3ccccc3c3c2c2ccc(-c4ccc(N(c5ccccc5)c5ccccc5)cc4)cc2n3-c2ccc3ccccc3c2)cc1 ; CC1(C)c2ccccc2-c2ccc(N(c3ccccc3)c3ccc(-c4ccc5c(c4)c4c(-c6ccccc6)cc6ccccc6c4n5-c4ccc5ccccc5c4)cc3)cc21 ; c1ccc(-c2cc3ccccc3c3c2c2ccc(N(c4ccccc4)c4ccccc4)cc2n3-c2cccc3ccccc23)cc1]

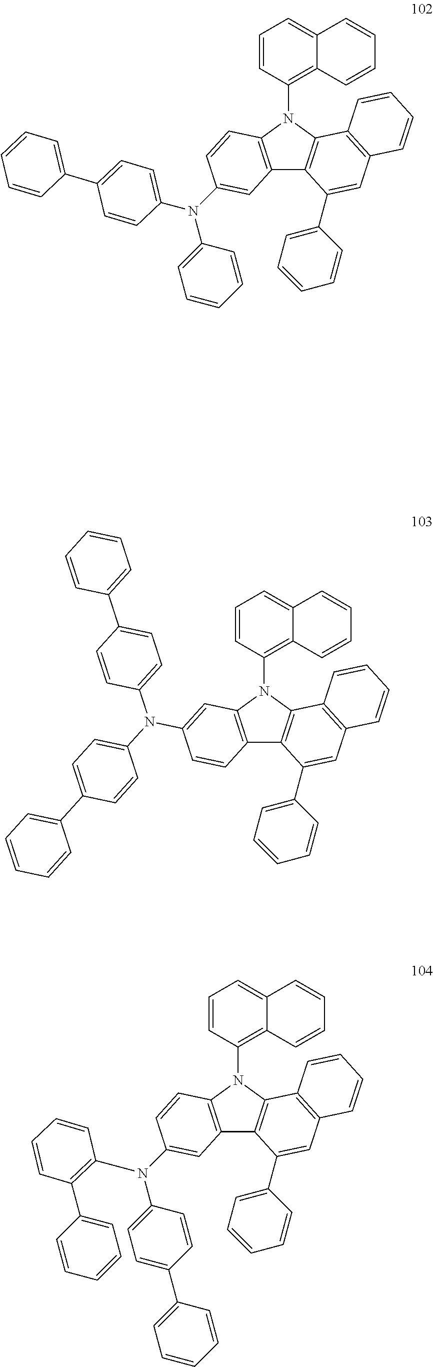 [SMILES: c1ccc(-c2ccc(N(c3ccccc3)c3ccc4c(c3)c3c(-c5ccccc5)cc5ccccc5c3n4-c3cccc4ccccc34)cc2)cc1 ; c1ccc(-c2ccc(N(c3ccc(-c4ccccc4)cc3)c3ccc4c5c(-c6ccccc6)cc6ccccc6c5n(-c5cccc6ccccc56)c4c3)cc2)cc1 ; c1ccc(-c2ccc(N(c3ccc4c(c3)c3c(-c5ccccc5)cc5ccccc5c3n4-c3cccc4ccccc34)c3ccccc3-c3ccccc3)cc2)cc1]

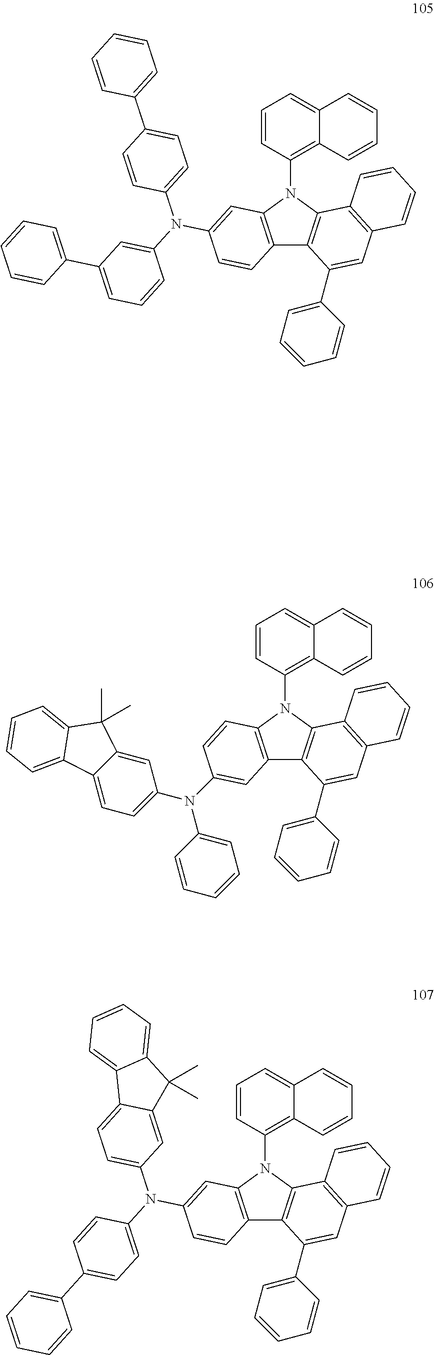 [SMILES: c1ccc(-c2ccc(N(c3cccc(-c4ccccc4)c3)c3ccc4c5c(-c6ccccc6)cc6ccccc6c5n(-c5cccc6ccccc56)c4c3)cc2)cc1 ; CC1(C)c2ccccc2-c2ccc(N(c3ccccc3)c3ccc4c(c3)c3c(-c5ccccc5)cc5ccccc5c3n4-c3cccc4ccccc34)cc21 ; CC1(C)c2ccccc2-c2ccc(N(c3ccc(-c4ccccc4)cc3)c3ccc4c5c(-c6ccccc6)cc6ccccc6c5n(-c5cccc6ccccc56)c4c3)cc21]

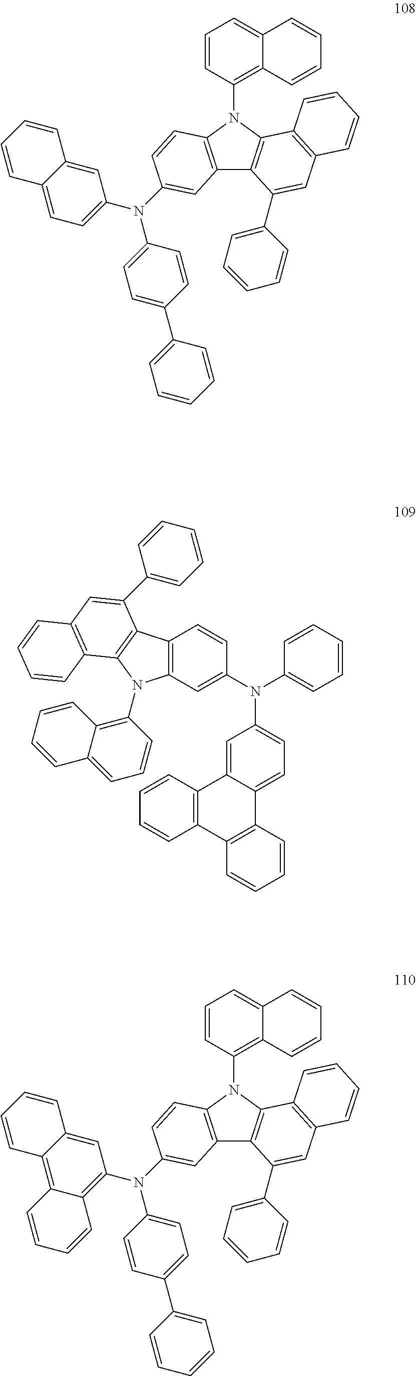 [SMILES: c1ccc(-c2ccc(N(c3ccc4ccccc4c3)c3ccc4c(c3)c3c(-c5ccccc5)cc5ccccc5c3n4-c3cccc4ccccc34)cc2)cc1 ; c1ccc(-c2cc3ccccc3c3c2c2ccc(N(c4ccccc4)c4ccc5c6ccccc6c6ccccc6c5c4)cc2n3-c2cccc3ccccc23)cc1 ; c1ccc(-c2ccc(N(c3ccc4c(c3)c3c(-c5ccccc5)cc5ccccc5c3n4-c3cccc4ccccc34)c3cc4ccccc4c4ccccc34)cc2)cc1]

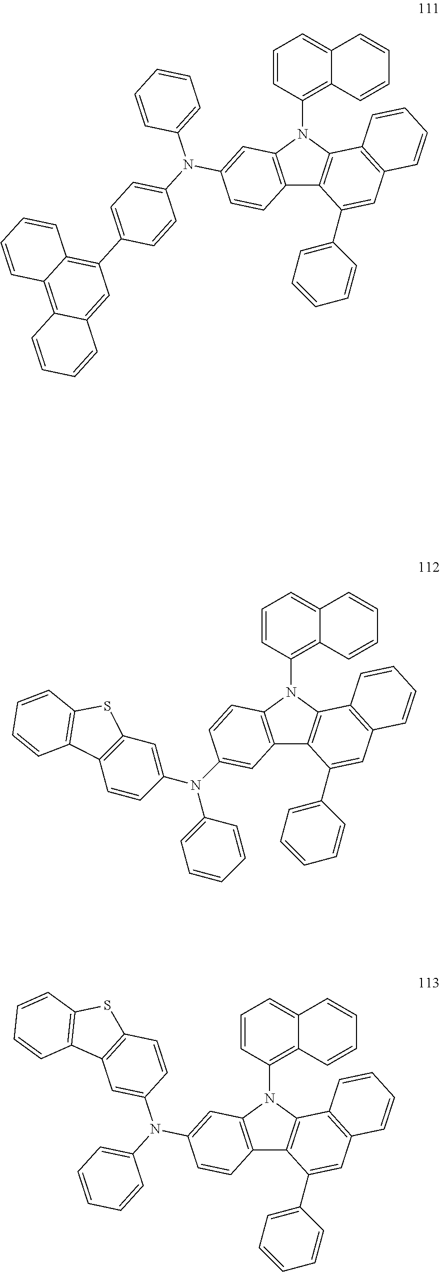 [SMILES: c1ccc(-c2cc3ccccc3c3c2c2ccc(N(c4ccccc4)c4ccc(-c5cc6ccccc6c6ccccc56)cc4)cc2n3-c2cccc3ccccc23)cc1 ; c1ccc(-c2cc3ccccc3c3c2c2cc(N(c4ccccc4)c4ccc5c(c4)sc4ccccc45)ccc2n3-c2cccc3ccccc23)cc1 ; c1ccc(-c2cc3ccccc3c3c2c2ccc(N(c4ccccc4)c4ccc5sc6ccccc6c5c4)cc2n3-c2cccc3ccccc23)cc1]

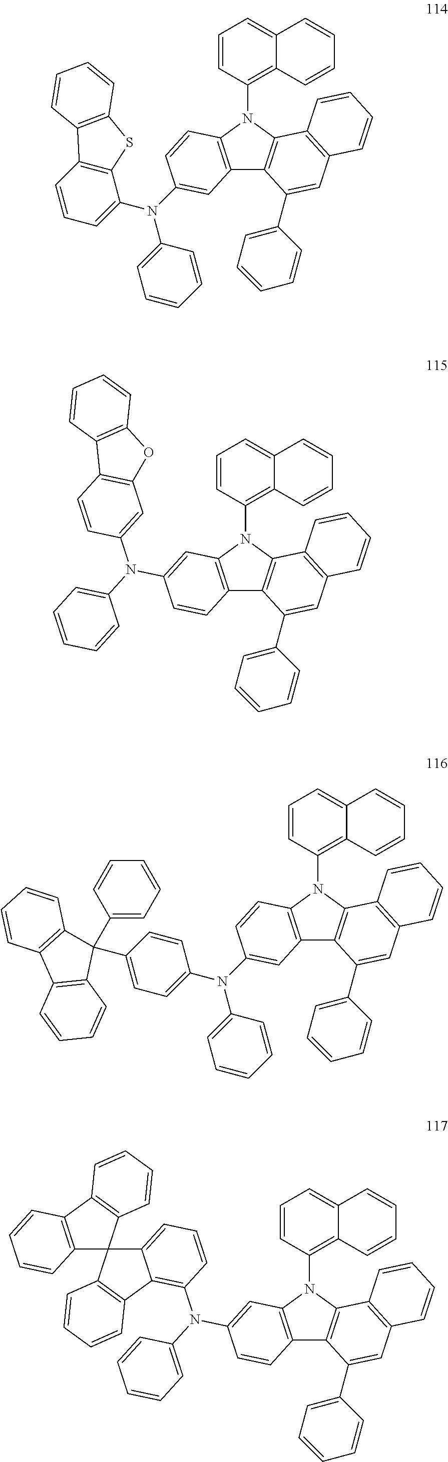 [SMILES: c1ccc(-c2cc3ccccc3c3c2c2cc(N(c4ccccc4)c4cccc5c4sc4ccccc45)ccc2n3-c2cccc3ccccc23)cc1 ; c1ccc(-c2cc3ccccc3c3c2c2ccc(N(c4ccccc4)c4ccc5c(c4)oc4ccccc45)cc2n3-c2cccc3ccccc23)cc1 ; c1ccc(-c2cc3ccccc3c3c2c2cc(N(c4ccccc4)c4ccc(C5(c6ccccc6)c6ccccc6-c6ccccc65)cc4)ccc2n3-c2cccc3ccccc23)cc1 ; c1ccc(-c2cc3ccccc3c3c2c2ccc(N(c4ccccc4)c4cccc5c4-c4ccccc4C54c5ccccc5-c5ccccc54)cc2n3-c2cccc3ccccc23)cc1]

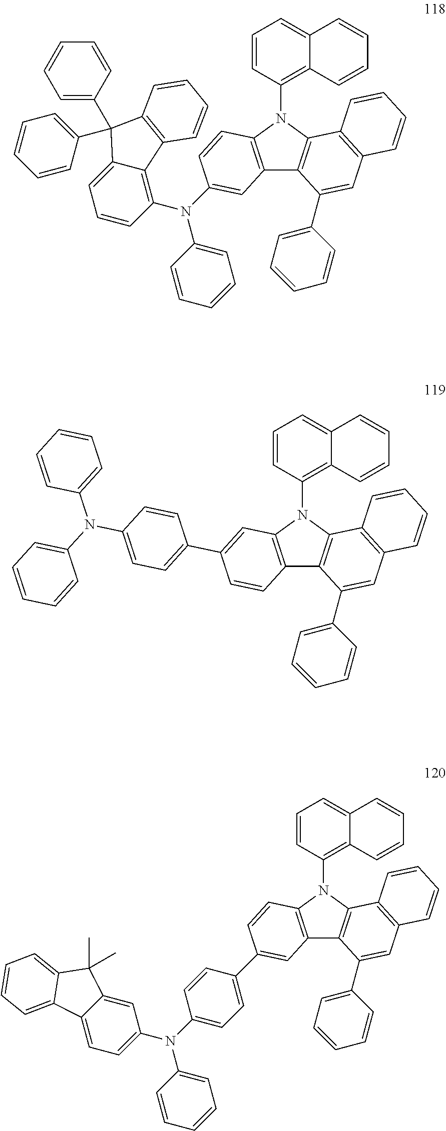 [SMILES: c1ccc(-c2cc3ccccc3c3c2c2cc(N(c4ccccc4)c4cccc5c4-c4ccccc4C5(c4ccccc4)c4ccccc4)ccc2n3-c2cccc3ccccc23)cc1 ; c1ccc(-c2cc3ccccc3c3c2c2ccc(-c4ccc(N(c5ccccc5)c5ccccc5)cc4)cc2n3-c2cccc3ccccc23)cc1 ; CC1(C)c2ccccc2-c2ccc(N(c3ccccc3)c3ccc(-c4ccc5c(c4)c4c(-c6ccccc6)cc6ccccc6c4n5-c4cccc5ccccc45)cc3)cc21]

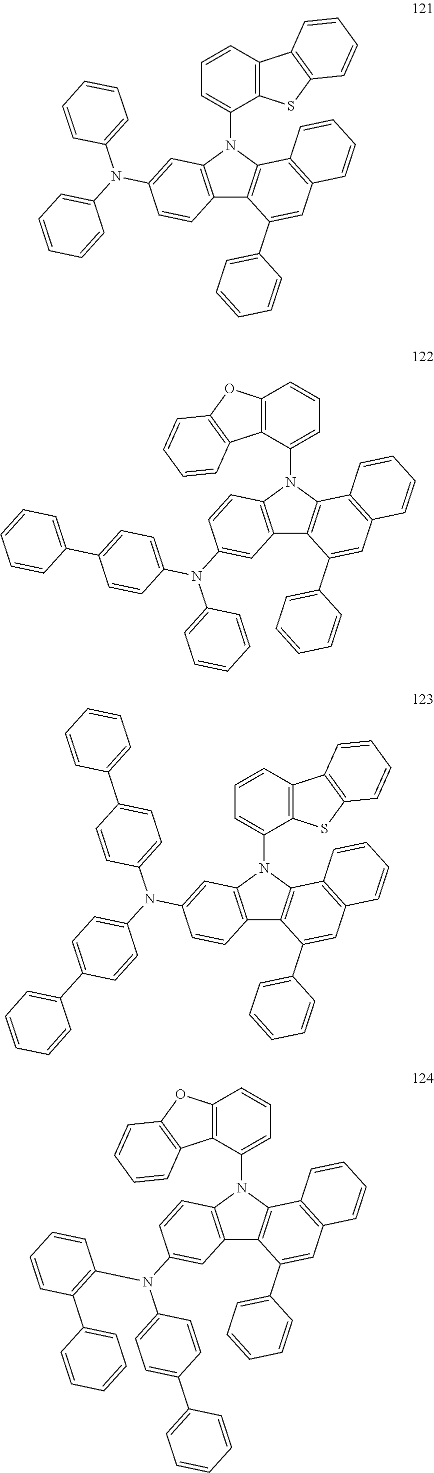 [SMILES: c1ccc(-c2cc3ccccc3c3c2c2ccc(N(c4ccccc4)c4ccccc4)cc2n3-c2cccc3c2sc2ccccc23)cc1 ; c1ccc(-c2ccc(N(c3ccccc3)c3ccc4c(c3)c3c(-c5ccccc5)cc5ccccc5c3n4-c3cccc4oc5ccccc5c34)cc2)cc1 ; c1ccc(-c2ccc(N(c3ccc(-c4ccccc4)cc3)c3ccc4c5c(-c6ccccc6)cc6ccccc6c5n(-c5cccc6c5sc5ccccc56)c4c3)cc2)cc1 ; c1ccc(-c2ccc(N(c3ccc4c(c3)c3c(-c5ccccc5)cc5ccccc5c3n4-c3cccc4oc5ccccc5c34)c3ccccc3-c3ccccc3)cc2)cc1]

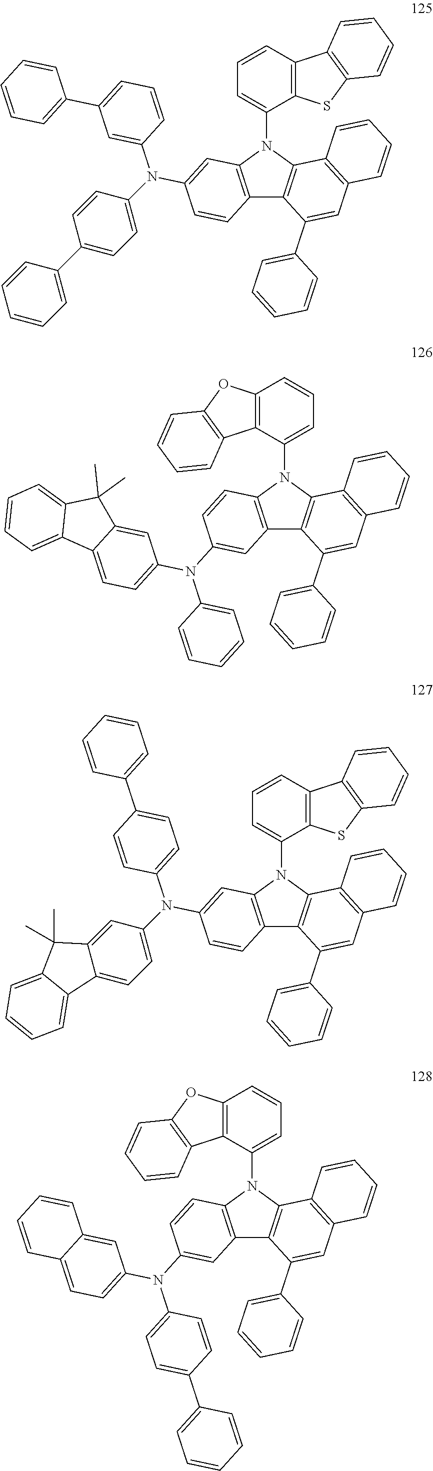 [SMILES: c1ccc(-c2ccc(N(c3cccc(-c4ccccc4)c3)c3ccc4c5c(-c6ccccc6)cc6ccccc6c5n(-c5cccc6c5sc5ccccc56)c4c3)cc2)cc1 ; CC1(C)c2ccccc2-c2ccc(N(c3ccccc3)c3ccc4c(c3)c3c(-c5ccccc5)cc5ccccc5c3n4-c3cccc4oc5ccccc5c34)cc21 ; CC1(C)c2ccccc2-c2ccc(N(c3ccc(-c4ccccc4)cc3)c3ccc4c5c(-c6ccccc6)cc6ccccc6c5n(-c5cccc6c5sc5ccccc56)c4c3)cc21 ; c1ccc(-c2ccc(N(c3ccc4ccccc4c3)c3ccc4c(c3)c3c(-c5ccccc5)cc5ccccc5c3n4-c3cccc4oc5ccccc5c34)cc2)cc1]

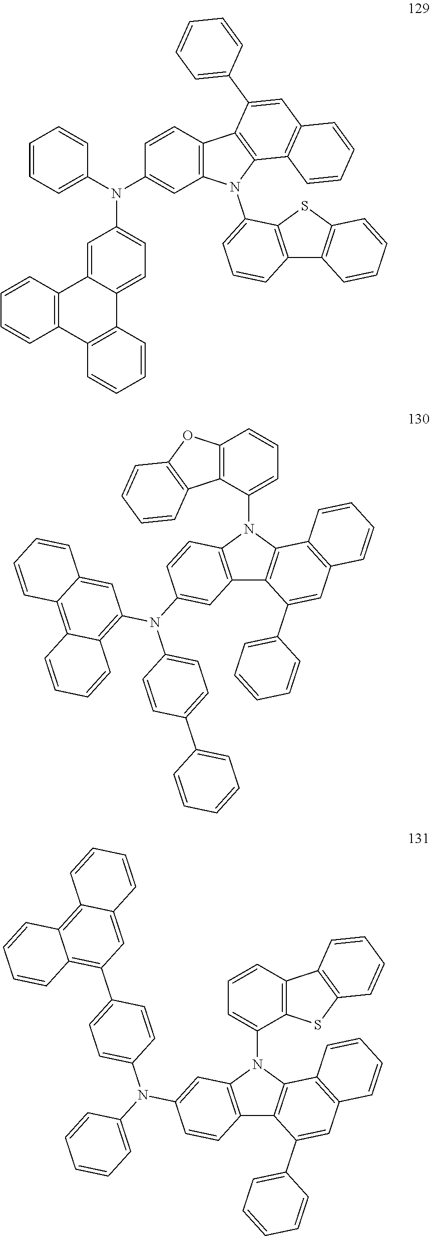 [SMILES: c1ccc(-c2cc3ccccc3c3c2c2ccc(N(c4ccccc4)c4ccc5c6ccccc6c6ccccc6c5c4)cc2n3-c2cccc3c2sc2ccccc23)cc1 ; c1ccc(-c2ccc(N(c3ccc4c(c3)c3c(-c5ccccc5)cc5ccccc5c3n4-c3cccc4oc5ccccc5c34)c3cc4ccccc4c4ccccc34)cc2)cc1 ; c1ccc(-c2cc3ccccc3c3c2c2ccc(N(c4ccccc4)c4ccc(-c5cc6ccccc6c6ccccc56)cc4)cc2n3-c2cccc3c2sc2ccccc23)cc1]

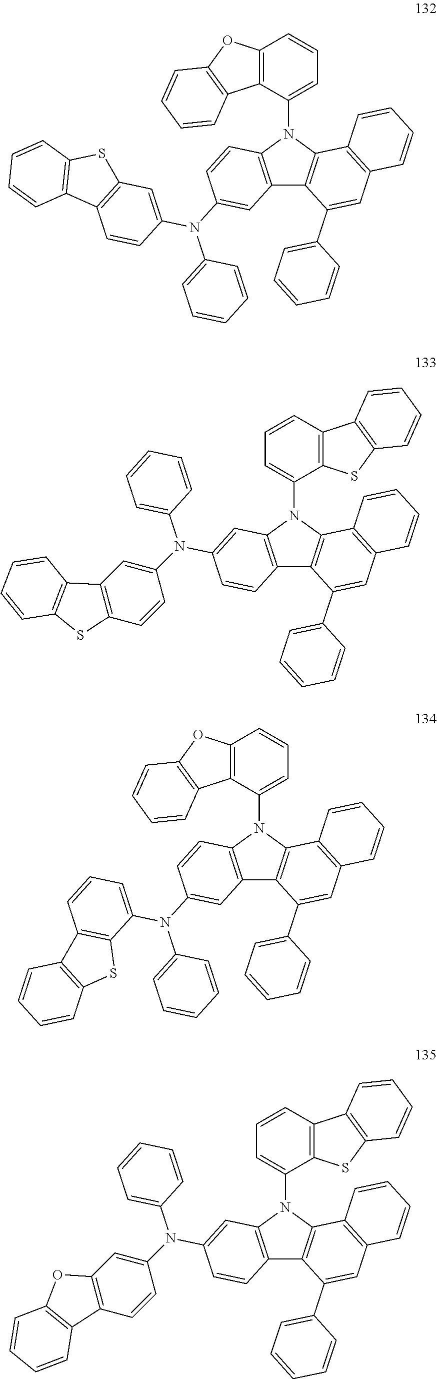 [SMILES: c1ccc(-c2cc3ccccc3c3c2c2cc(N(c4ccccc4)c4ccc5c(c4)sc4ccccc45)ccc2n3-c2cccc3oc4ccccc4c23)cc1 ; c1ccc(-c2cc3ccccc3c3c2c2ccc(N(c4ccccc4)c4ccc5sc6ccccc6c5c4)cc2n3-c2cccc3c2sc2ccccc23)cc1 ; c1ccc(-c2cc3ccccc3c3c2c2cc(N(c4ccccc4)c4cccc5c4sc4ccccc45)ccc2n3-c2cccc3oc4ccccc4c23)cc1 ; c1ccc(-c2cc3ccccc3c3c2c2ccc(N(c4ccccc4)c4ccc5c(c4)oc4ccccc45)cc2n3-c2cccc3c2sc2ccccc23)cc1]

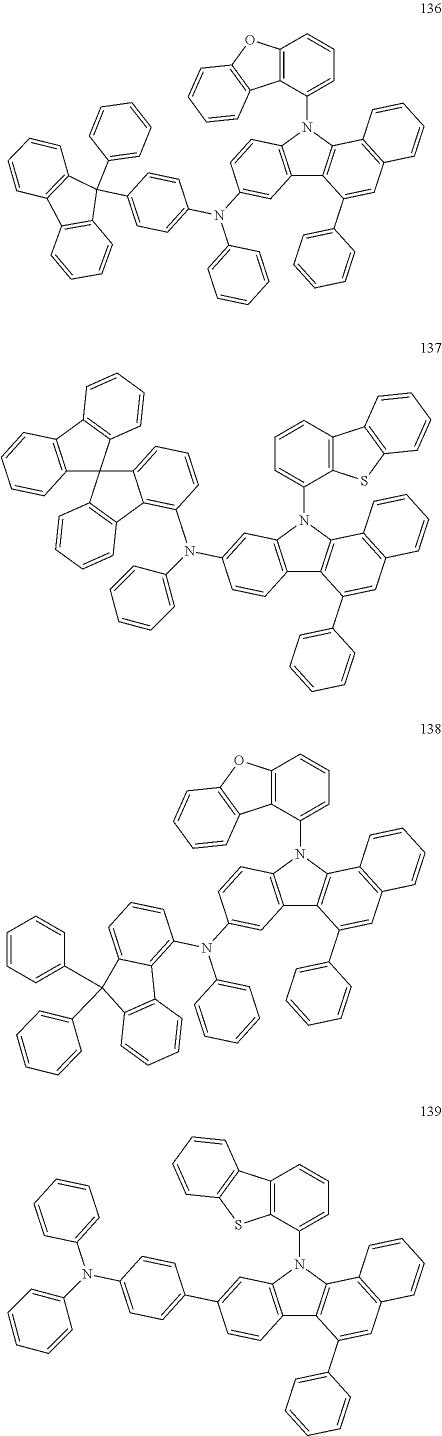 [SMILES: c1ccc(-c2cc3ccccc3c3c2c2cc(N(c4ccccc4)c4ccc(C5(c6ccccc6)c6ccccc6-c6ccccc65)cc4)ccc2n3-c2cccc3oc4ccccc4c23)cc1 ; c1ccc(-c2cc3ccccc3c3c2c2ccc(N(c4ccccc4)c4cccc5c4-c4ccccc4C54c5ccccc5-c5ccccc54)cc2n3-c2cccc3c2sc2ccccc23)cc1 ; c1ccc(-c2cc3ccccc3c3c2c2cc(N(c4ccccc4)c4cccc5c4-c4ccccc4C5(c4ccccc4)c4ccccc4)ccc2n3-c2cccc3oc4ccccc4c23)cc1 ; c1ccc(-c2cc3ccccc3c3c2c2ccc(-c4ccc(N(c5ccccc5)c5ccccc5)cc4)cc2n3-c2cccc3c2sc2ccccc23)cc1]

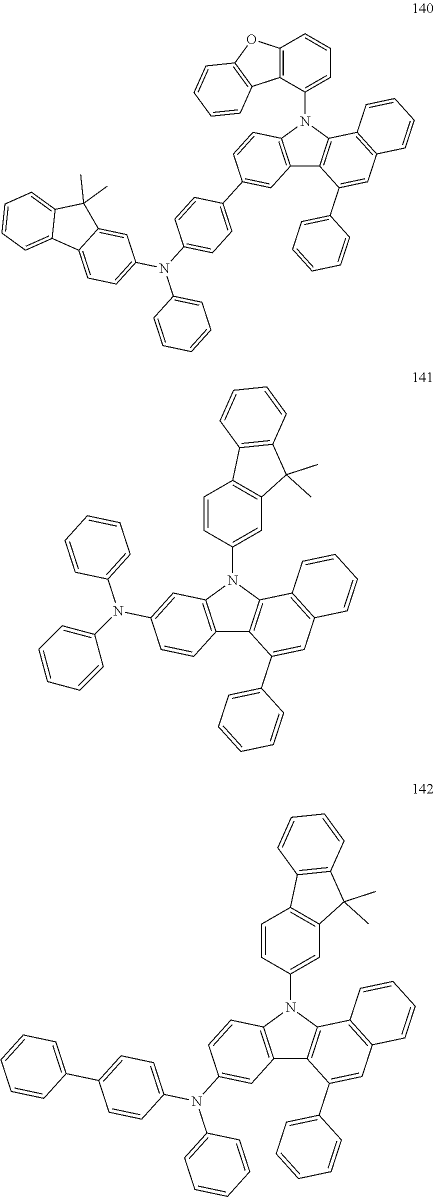 [SMILES: CC1(C)c2ccccc2-c2ccc(N(c3ccccc3)c3ccc(-c4ccc5c(c4)c4c(-c6ccccc6)cc6ccccc6c4n5-c4cccc5oc6ccccc6c45)cc3)cc21 ; CC1(C)c2ccccc2-c2ccc(-n3c4cc(N(c5ccccc5)c5ccccc5)ccc4c4c(-c5ccccc5)cc5ccccc5c43)cc21 ; CC1(C)c2ccccc2-c2ccc(-n3c4ccc(N(c5ccccc5)c5ccc(-c6ccccc6)cc5)cc4c4c(-c5ccccc5)cc5ccccc5c43)cc21]

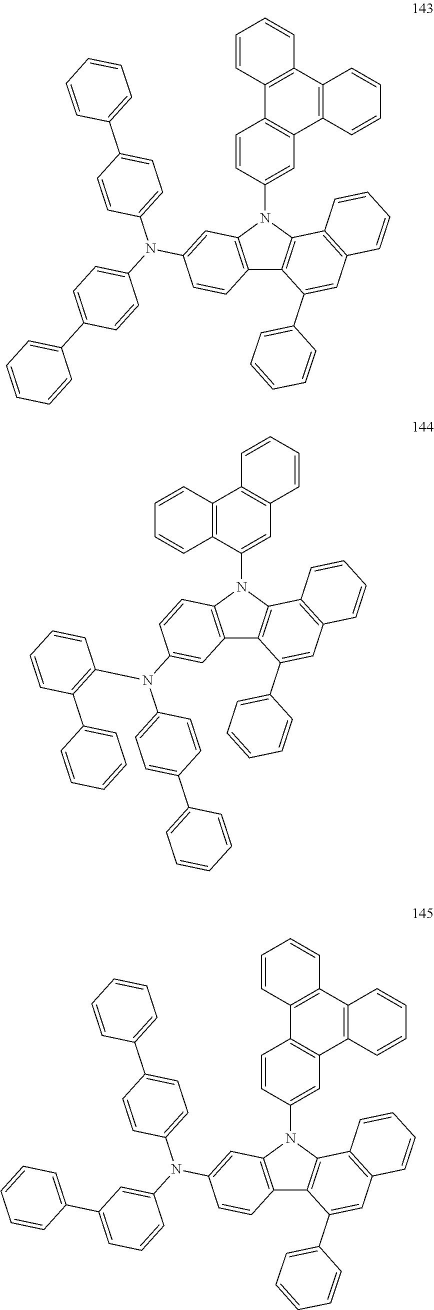 [SMILES: c1ccc(-c2ccc(N(c3ccc(-c4ccccc4)cc3)c3ccc4c5c(-c6ccccc6)cc6ccccc6c5n(-c5ccc6c7ccccc7c7ccccc7c6c5)c4c3)cc2)cc1 ; c1ccc(-c2ccc(N(c3ccc4c(c3)c3c(-c5ccccc5)cc5ccccc5c3n4-c3cc4ccccc4c4ccccc34)c3ccccc3-c3ccccc3)cc2)cc1 ; c1ccc(-c2ccc(N(c3cccc(-c4ccccc4)c3)c3ccc4c5c(-c6ccccc6)cc6ccccc6c5n(-c5ccc6c7ccccc7c7ccccc7c6c5)c4c3)cc2)cc1]

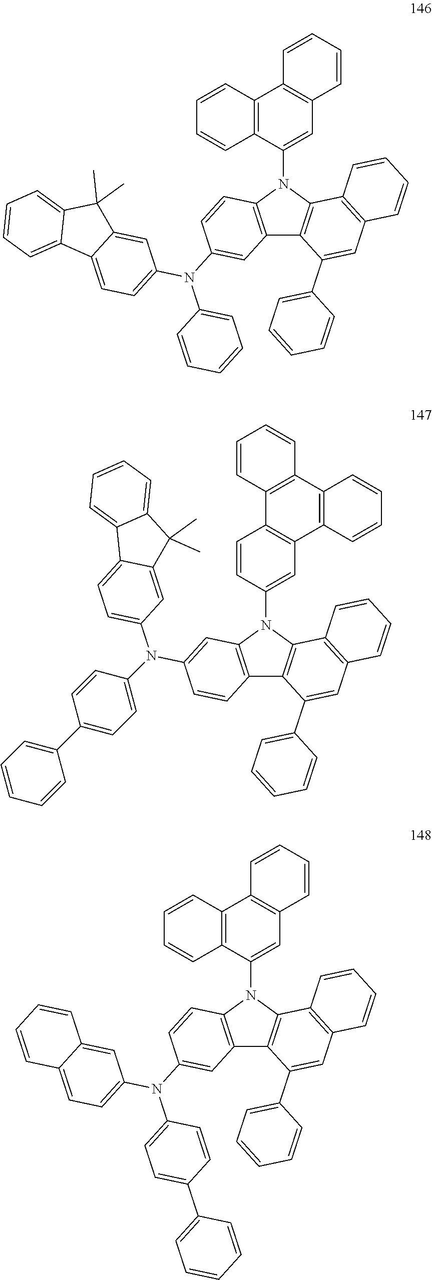 [SMILES: CC1(C)c2ccccc2-c2ccc(N(c3ccccc3)c3ccc4c(c3)c3c(-c5ccccc5)cc5ccccc5c3n4-c3cc4ccccc4c4ccccc34)cc21 ; CC1(C)c2ccccc2-c2ccc(N(c3ccc(-c4ccccc4)cc3)c3ccc4c5c(-c6ccccc6)cc6ccccc6c5n(-c5ccc6c7ccccc7c7ccccc7c6c5)c4c3)cc21 ; c1ccc(-c2ccc(N(c3ccc4ccccc4c3)c3ccc4c(c3)c3c(-c5ccccc5)cc5ccccc5c3n4-c3cc4ccccc4c4ccccc34)cc2)cc1]

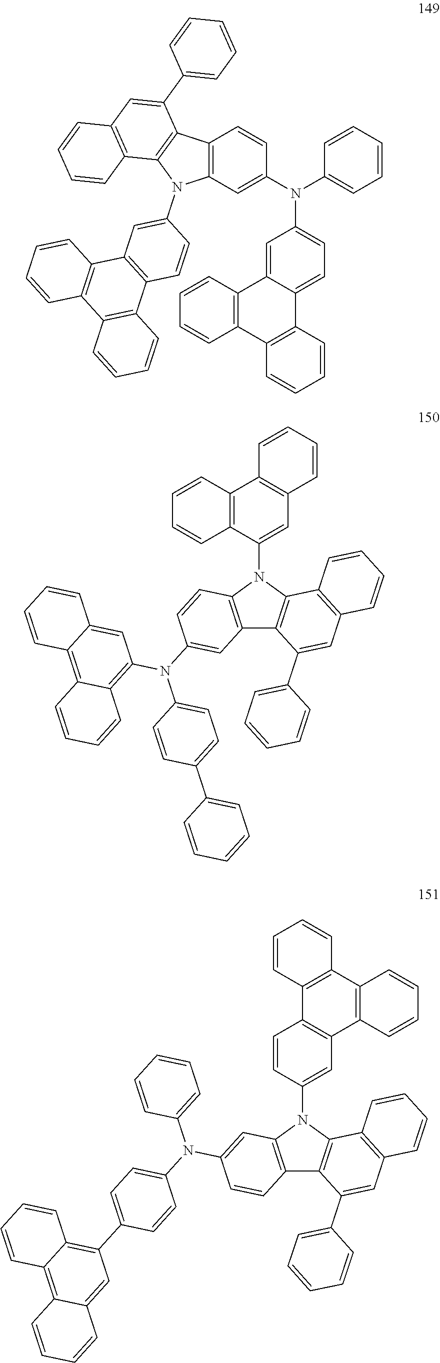

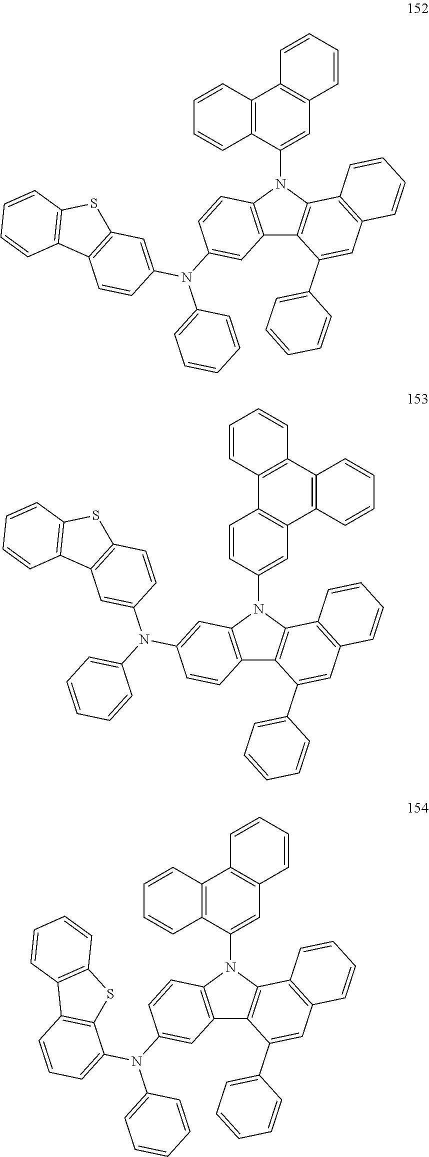

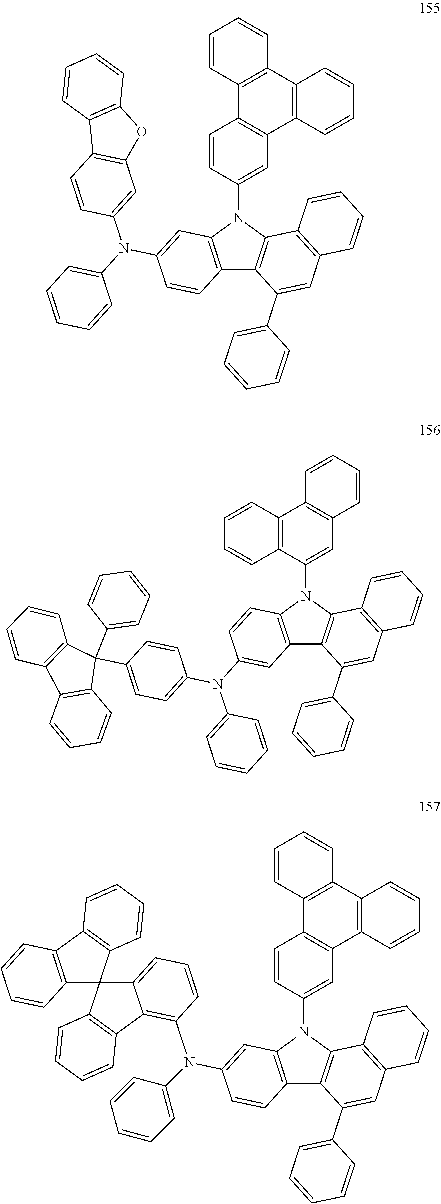 [SMILES: c1ccc(-c2cc3ccccc3c3c2c2ccc(N(c4ccccc4)c4ccc5c(c4)oc4ccccc45)cc2n3-c2ccc3c4ccccc4c4ccccc4c3c2)cc1 ; c1ccc(-c2cc3ccccc3c3c2c2cc(N(c4ccccc4)c4ccc(C5(c6ccccc6)c6ccccc6-c6ccccc65)cc4)ccc2n3-c2cc3ccccc3c3ccccc23)cc1 ; c1ccc(-c2cc3ccccc3c3c2c2ccc(N(c4ccccc4)c4cccc5c4-c4ccccc4C54c5ccccc5-c5ccccc54)cc2n3-c2ccc3c4ccccc4c4ccccc4c3c2)cc1]

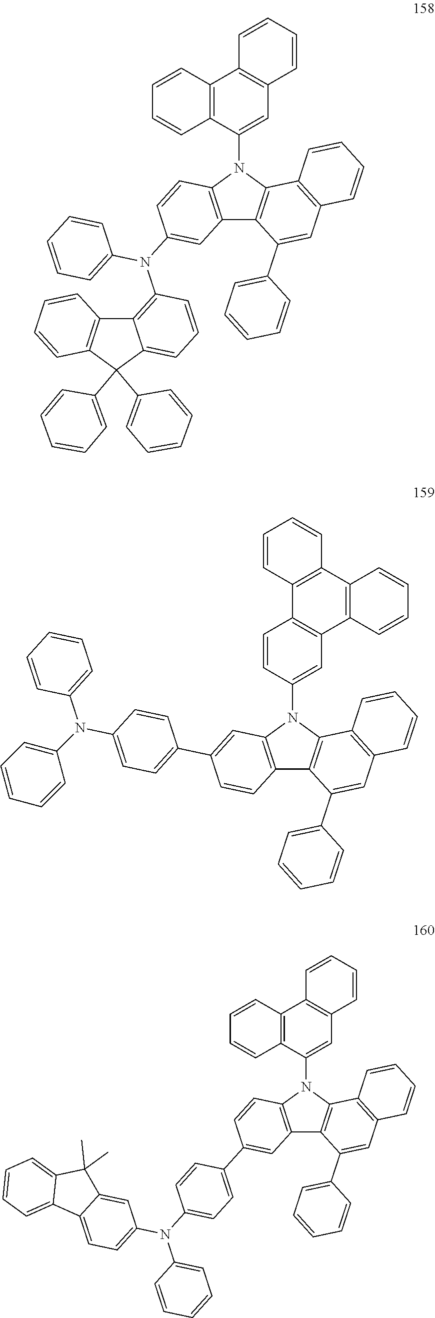 [SMILES: c1ccc(-c2cc3ccccc3c3c2c2cc(N(c4ccccc4)c4cccc5c4-c4ccccc4C5(c4ccccc4)c4ccccc4)ccc2n3-c2cc3ccccc3c3ccccc23)cc1 ; c1ccc(-c2cc3ccccc3c3c2c2ccc(-c4ccc(N(c5ccccc5)c5ccccc5)cc4)cc2n3-c2ccc3c4ccccc4c4ccccc4c3c2)cc1 ; CC1(C)c2ccccc2-c2ccc(N(c3ccccc3)c3ccc(-c4ccc5c(c4)c4c(-c6ccccc6)cc6ccccc6c4n5-c4cc5ccccc5c5ccccc45)cc3)cc21]

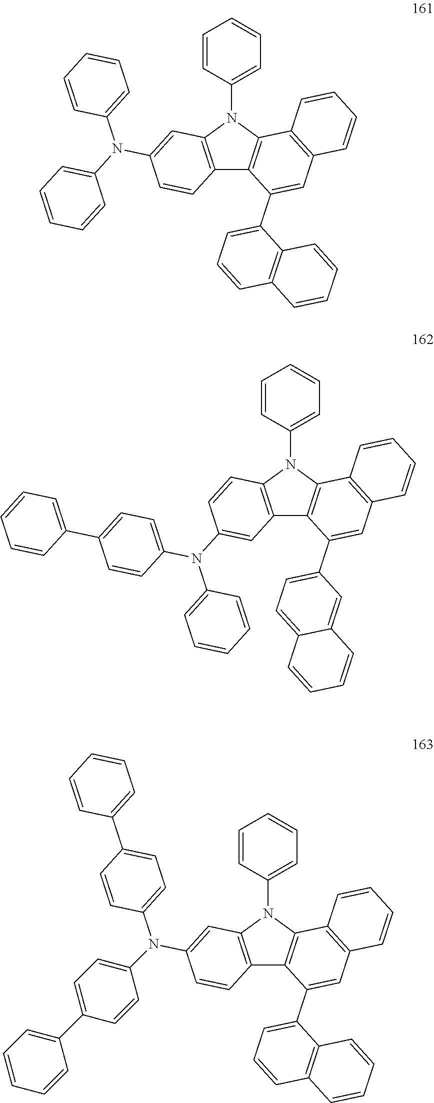 [SMILES: c1ccc(N(c2ccccc2)c2ccc3c4c(-c5cccc6ccccc56)cc5ccccc5c4n(-c4ccccc4)c3c2)cc1 ; c1ccc(-c2ccc(N(c3ccccc3)c3ccc4c(c3)c3c(-c5ccc6ccccc6c5)cc5ccccc5c3n4-c3ccccc3)cc2)cc1 ; c1ccc(-c2ccc(N(c3ccc(-c4ccccc4)cc3)c3ccc4c5c(-c6cccc7ccccc67)cc6ccccc6c5n(-c5ccccc5)c4c3)cc2)cc1]

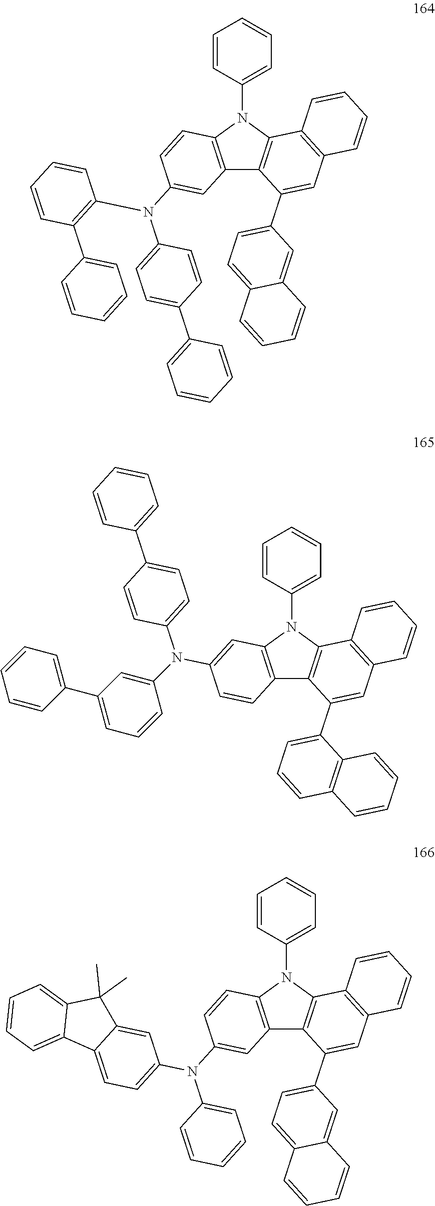 [SMILES: c1ccc(-c2ccc(N(c3ccc4c(c3)c3c(-c5ccc6ccccc6c5)cc5ccccc5c3n4-c3ccccc3)c3ccccc3-c3ccccc3)cc2)cc1 ; c1ccc(-c2ccc(N(c3cccc(-c4ccccc4)c3)c3ccc4c5c(-c6cccc7ccccc67)cc6ccccc6c5n(-c5ccccc5)c4c3)cc2)cc1 ; CC1(C)c2ccccc2-c2ccc(N(c3ccccc3)c3ccc4c(c3)c3c(-c5ccc6ccccc6c5)cc5ccccc5c3n4-c3ccccc3)cc21]

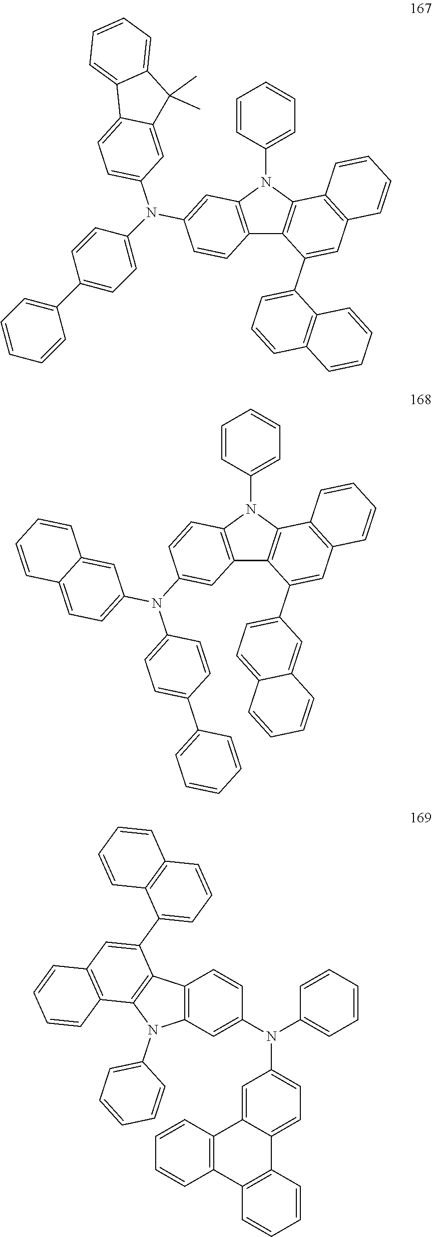 [SMILES: CC1(C)c2ccccc2-c2ccc(N(c3ccc(-c4ccccc4)cc3)c3ccc4c5c(-c6cccc7ccccc67)cc6ccccc6c5n(-c5ccccc5)c4c3)cc21 ; c1ccc(-c2ccc(N(c3ccc4ccccc4c3)c3ccc4c(c3)c3c(-c5ccc6ccccc6c5)cc5ccccc5c3n4-c3ccccc3)cc2)cc1 ; c1ccc(N(c2ccc3c4ccccc4c4ccccc4c3c2)c2ccc3c4c(-c5cccc6ccccc56)cc5ccccc5c4n(-c4ccccc4)c3c2)cc1]

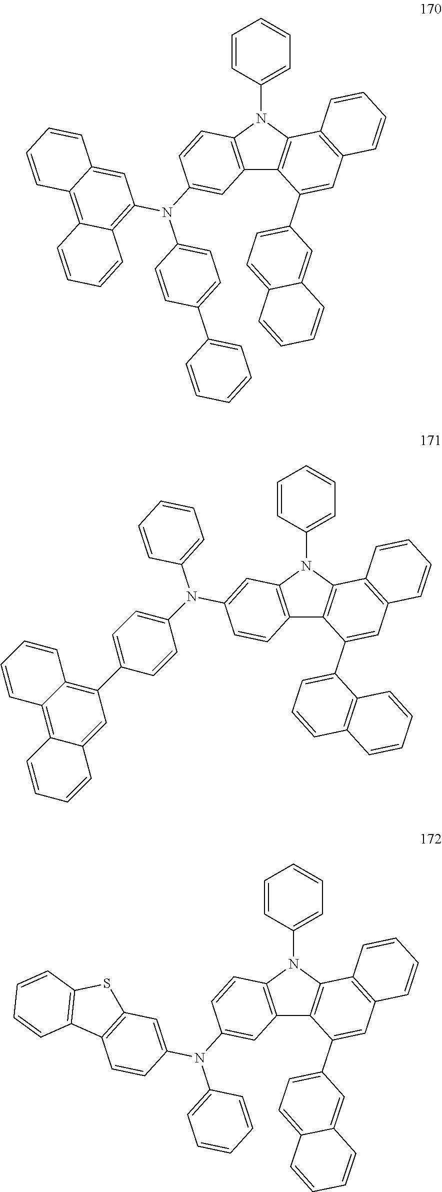 [SMILES: c1ccc(-c2ccc(N(c3ccc4c(c3)c3c(-c5ccc6ccccc6c5)cc5ccccc5c3n4-c3ccccc3)c3cc4ccccc4c4ccccc34)cc2)cc1 ; c1ccc(N(c2ccc(-c3cc4ccccc4c4ccccc34)cc2)c2ccc3c4c(-c5cccc6ccccc56)cc5ccccc5c4n(-c4ccccc4)c3c2)cc1 ; c1ccc(N(c2ccc3c(c2)sc2ccccc23)c2ccc3c(c2)c2c(-c4ccc5ccccc5c4)cc4ccccc4c2n3-c2ccccc2)cc1]

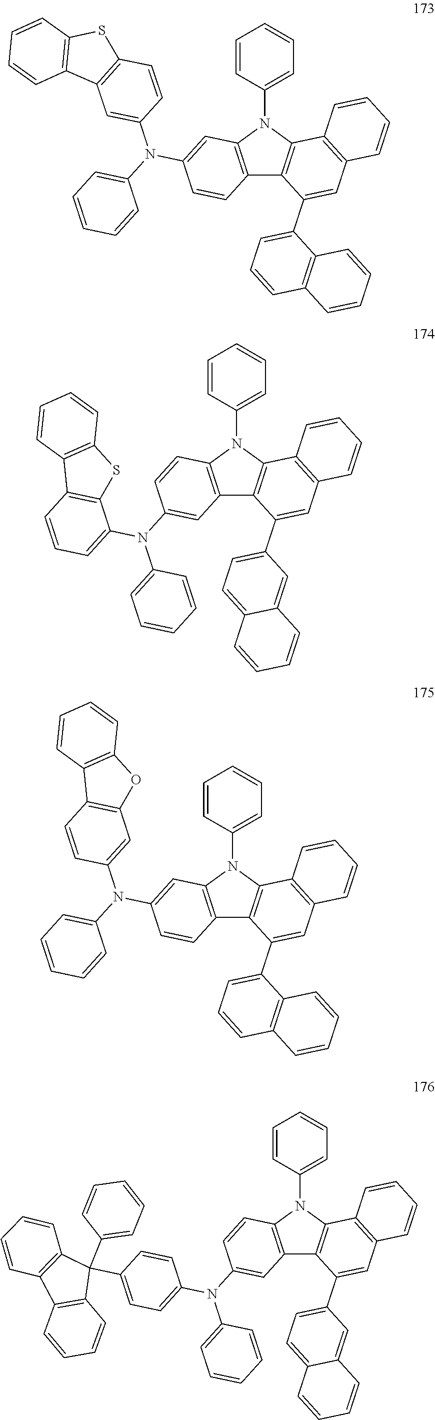 [SMILES: c1ccc(N(c2ccc3sc4ccccc4c3c2)c2ccc3c4c(-c5cccc6ccccc56)cc5ccccc5c4n(-c4ccccc4)c3c2)cc1 ; c1ccc(N(c2ccc3c(c2)c2c(-c4ccc5ccccc5c4)cc4ccccc4c2n3-c2ccccc2)c2cccc3c2sc2ccccc23)cc1 ; c1ccc(N(c2ccc3c(c2)oc2ccccc23)c2ccc3c4c(-c5cccc6ccccc56)cc5ccccc5c4n(-c4ccccc4)c3c2)cc1 ; c1ccc(N(c2ccc(C3(c4ccccc4)c4ccccc4-c4ccccc43)cc2)c2ccc3c(c2)c2c(-c4ccc5ccccc5c4)cc4ccccc4c2n3-c2ccccc2)cc1]

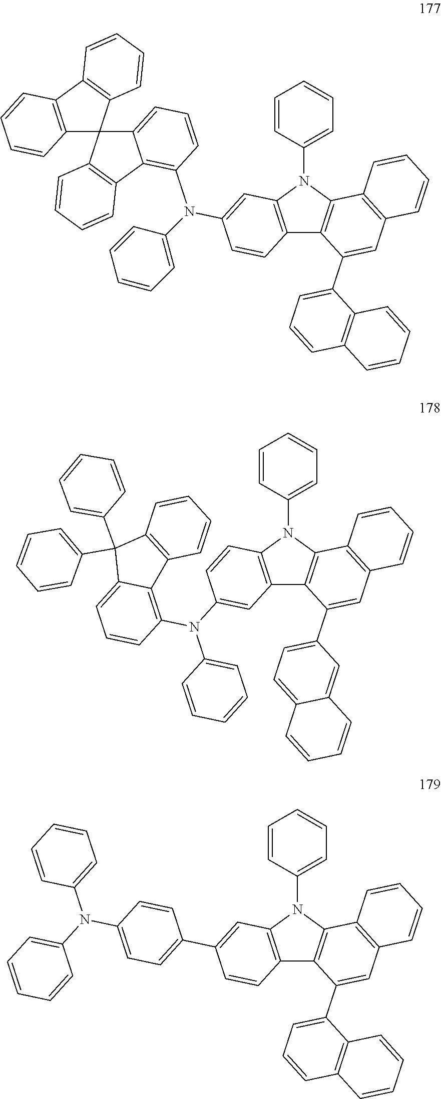 [SMILES: c1ccc(N(c2ccc3c4c(-c5cccc6ccccc56)cc5ccccc5c4n(-c4ccccc4)c3c2)c2cccc3c2-c2ccccc2C32c3ccccc3-c3ccccc32)cc1 ; c1ccc(N(c2ccc3c(c2)c2c(-c4ccc5ccccc5c4)cc4ccccc4c2n3-c2ccccc2)c2cccc3c2-c2ccccc2C3(c2ccccc2)c2ccccc2)cc1 ; c1ccc(N(c2ccccc2)c2ccc(-c3ccc4c5c(-c6cccc7ccccc67)cc6ccccc6c5n(-c5ccccc5)c4c3)cc2)cc1]

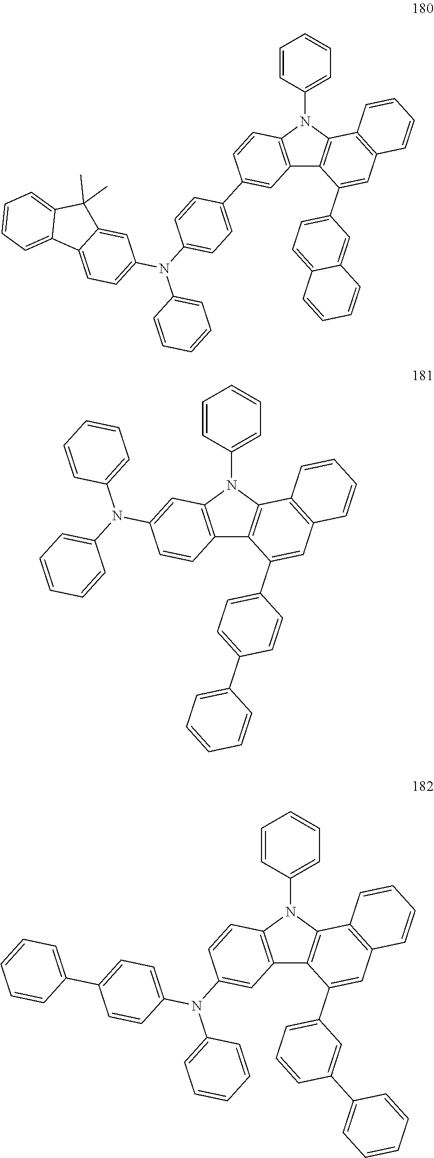 [SMILES: CC1(C)c2ccccc2-c2ccc(N(c3ccccc3)c3ccc(-c4ccc5c(c4)c4c(-c6ccc7ccccc7c6)cc6ccccc6c4n5-c4ccccc4)cc3)cc21 ; c1ccc(-c2ccc(-c3cc4ccccc4c4c3c3ccc(N(c5ccccc5)c5ccccc5)cc3n4-c3ccccc3)cc2)cc1 ; c1ccc(-c2ccc(N(c3ccccc3)c3ccc4c(c3)c3c(-c5cccc(-c6ccccc6)c5)cc5ccccc5c3n4-c3ccccc3)cc2)cc1]

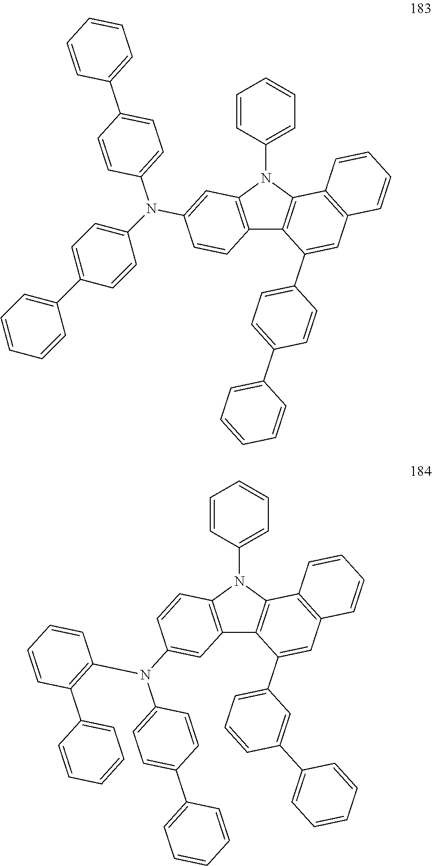

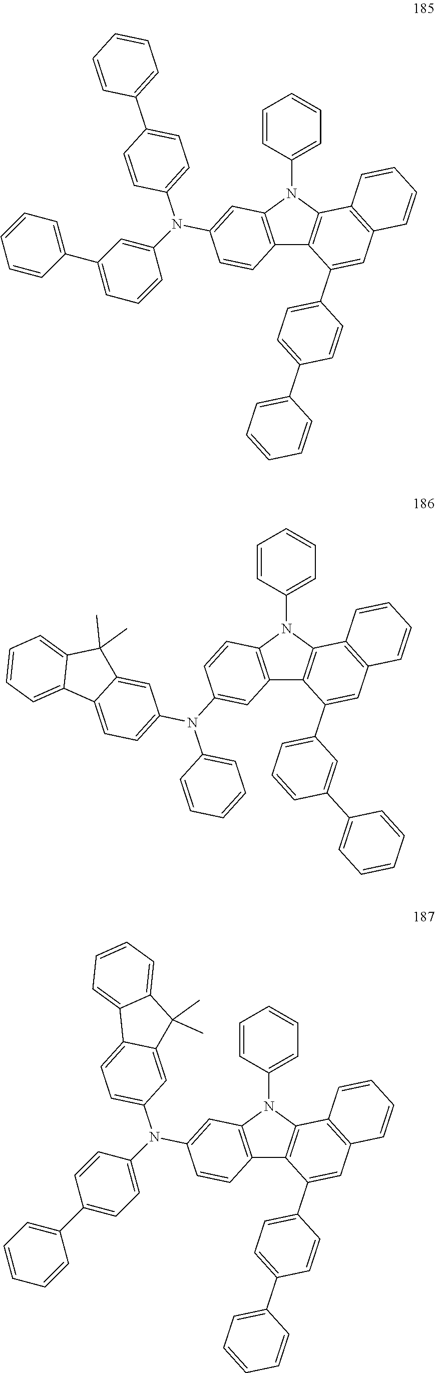 [SMILES: c1ccc(-c2ccc(-c3cc4ccccc4c4c3c3ccc(N(c5ccc(-c6ccccc6)cc5)c5cccc(-c6ccccc6)c5)cc3n4-c3ccccc3)cc2)cc1 ; CC1(C)c2ccccc2-c2ccc(N(c3ccccc3)c3ccc4c(c3)c3c(-c5cccc(-c6ccccc6)c5)cc5ccccc5c3n4-c3ccccc3)cc21 ; CC1(C)c2ccccc2-c2ccc(N(c3ccc(-c4ccccc4)cc3)c3ccc4c5c(-c6ccc(-c7ccccc7)cc6)cc6ccccc6c5n(-c5ccccc5)c4c3)cc21]

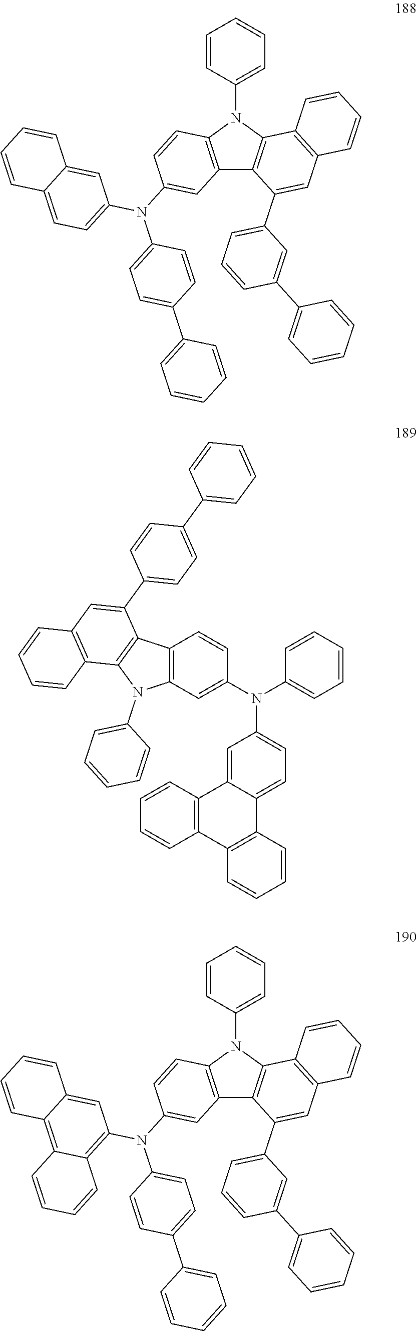

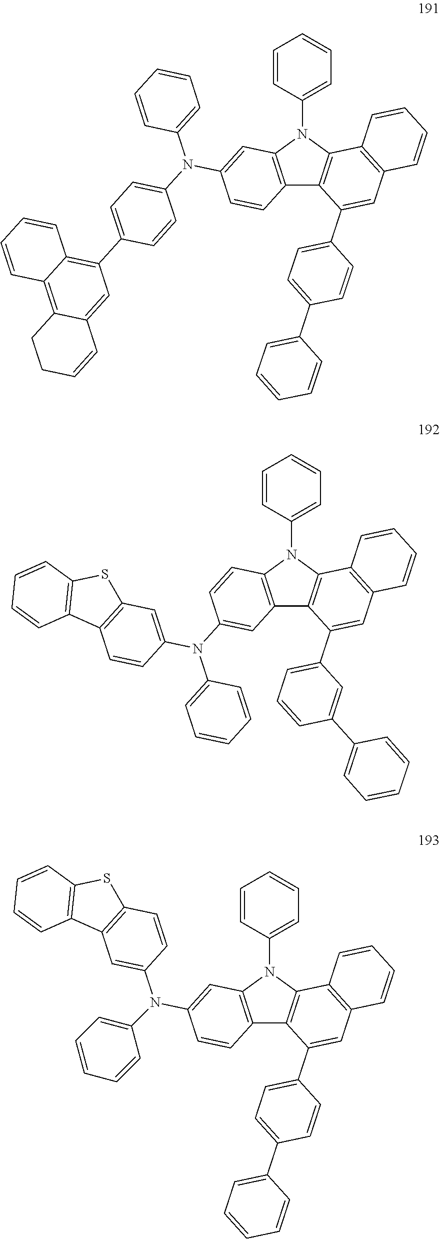 [SMILES: C1=Cc2cc(-c3ccc(N(c4ccccc4)c4ccc5c6c(-c7ccc(-c8ccccc8)cc7)cc7ccccc7c6n(-c6ccccc6)c5c4)cc3)c3ccccc3c2CC1 ; c1ccc(-c2cccc(-c3cc4ccccc4c4c3c3cc(N(c5ccccc5)c5ccc6c(c5)sc5ccccc56)ccc3n4-c3ccccc3)c2)cc1 ; c1ccc(-c2ccc(-c3cc4ccccc4c4c3c3ccc(N(c5ccccc5)c5ccc6sc7ccccc7c6c5)cc3n4-c3ccccc3)cc2)cc1]

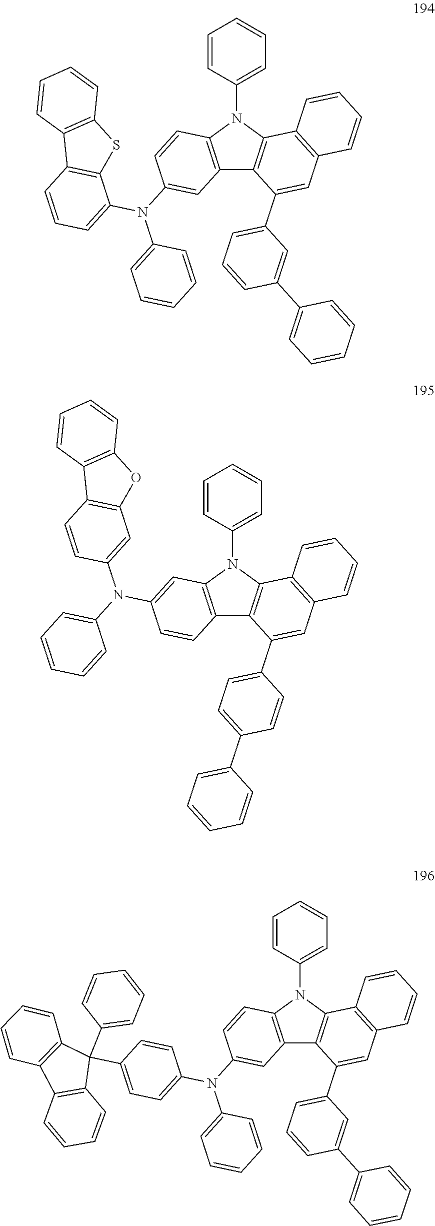 [SMILES: c1ccc(-c2cccc(-c3cc4ccccc4c4c3c3cc(N(c5ccccc5)c5cccc6c5sc5ccccc56)ccc3n4-c3ccccc3)c2)cc1 ; c1ccc(-c2ccc(-c3cc4ccccc4c4c3c3ccc(N(c5ccccc5)c5ccc6c(c5)oc5ccccc56)cc3n4-c3ccccc3)cc2)cc1 ; c1ccc(-c2cccc(-c3cc4ccccc4c4c3c3cc(N(c5ccccc5)c5ccc(C6(c7ccccc7)c7ccccc7-c7ccccc76)cc5)ccc3n4-c3ccccc3)c2)cc1]

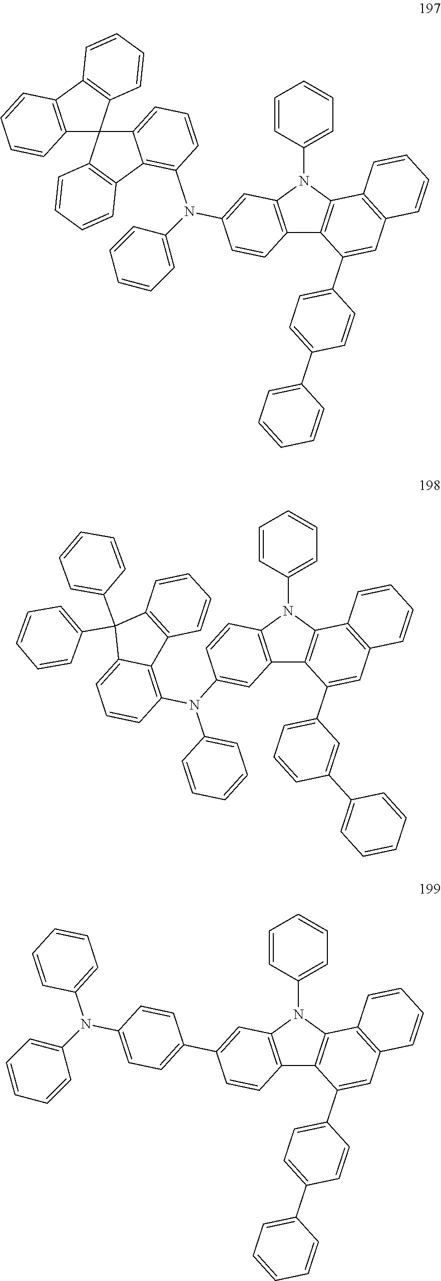 [SMILES: c1ccc(-c2ccc(-c3cc4ccccc4c4c3c3ccc(N(c5ccccc5)c5cccc6c5-c5ccccc5C65c6ccccc6-c6ccccc65)cc3n4-c3ccccc3)cc2)cc1 ; c1ccc(-c2cccc(-c3cc4ccccc4c4c3c3cc(N(c5ccccc5)c5cccc6c5-c5ccccc5C6(c5ccccc5)c5ccccc5)ccc3n4-c3ccccc3)c2)cc1 ; c1ccc(-c2ccc(-c3cc4ccccc4c4c3c3ccc(-c5ccc(N(c6ccccc6)c6ccccc6)cc5)cc3n4-c3ccccc3)cc2)cc1]

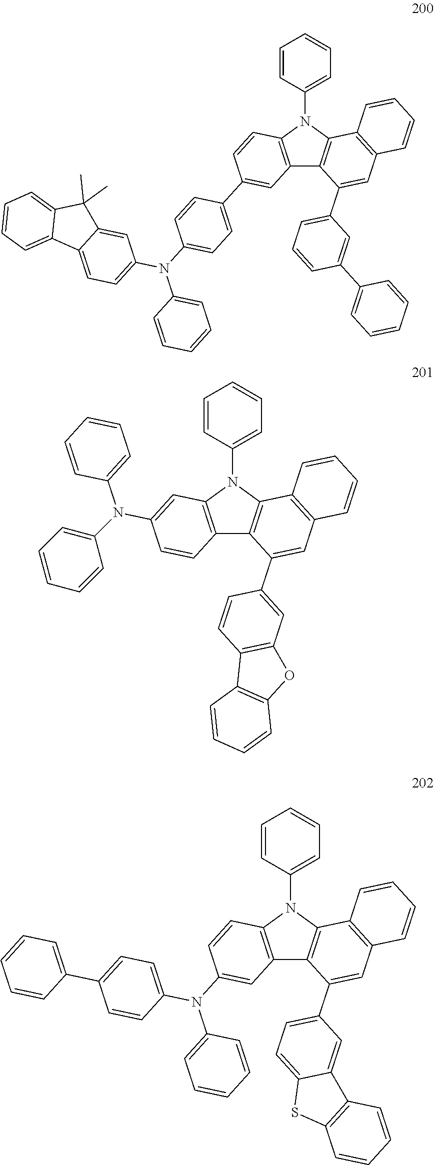 [SMILES: CC1(C)c2ccccc2-c2ccc(N(c3ccccc3)c3ccc(-c4ccc5c(c4)c4c(-c6cccc(-c7ccccc7)c6)cc6ccccc6c4n5-c4ccccc4)cc3)cc21 ; c1ccc(N(c2ccccc2)c2ccc3c4c(-c5ccc6c(c5)oc5ccccc56)cc5ccccc5c4n(-c4ccccc4)c3c2)cc1 ; c1ccc(-c2ccc(N(c3ccccc3)c3ccc4c(c3)c3c(-c5ccc6sc7ccccc7c6c5)cc5ccccc5c3n4-c3ccccc3)cc2)cc1]

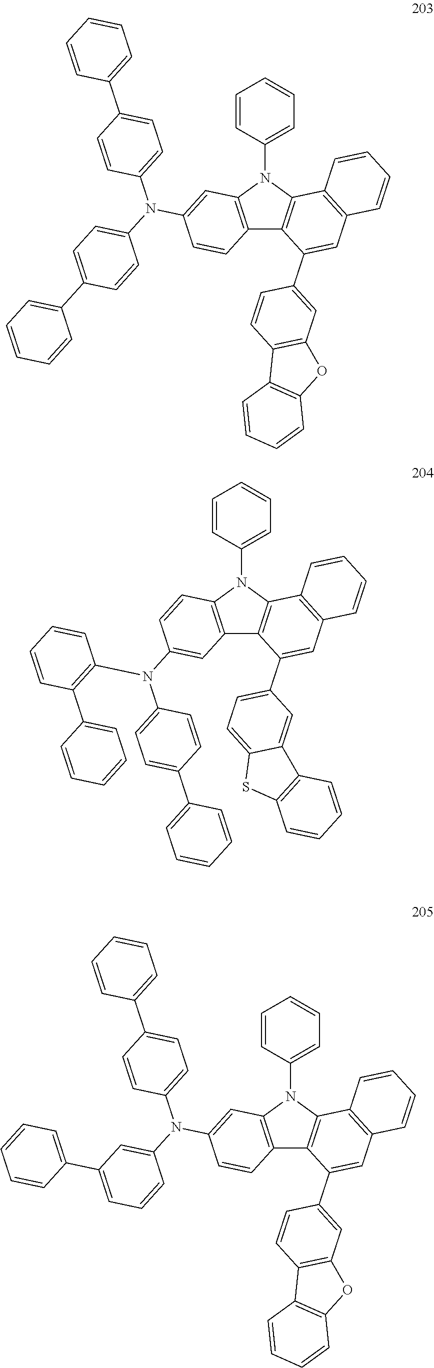 [SMILES: c1ccc(-c2ccc(N(c3ccc(-c4ccccc4)cc3)c3ccc4c5c(-c6ccc7c(c6)oc6ccccc67)cc6ccccc6c5n(-c5ccccc5)c4c3)cc2)cc1 ; c1ccc(-c2ccc(N(c3ccc4c(c3)c3c(-c5ccc6sc7ccccc7c6c5)cc5ccccc5c3n4-c3ccccc3)c3ccccc3-c3ccccc3)cc2)cc1 ; c1ccc(-c2ccc(N(c3cccc(-c4ccccc4)c3)c3ccc4c5c(-c6ccc7c(c6)oc6ccccc67)cc6ccccc6c5n(-c5ccccc5)c4c3)cc2)cc1]

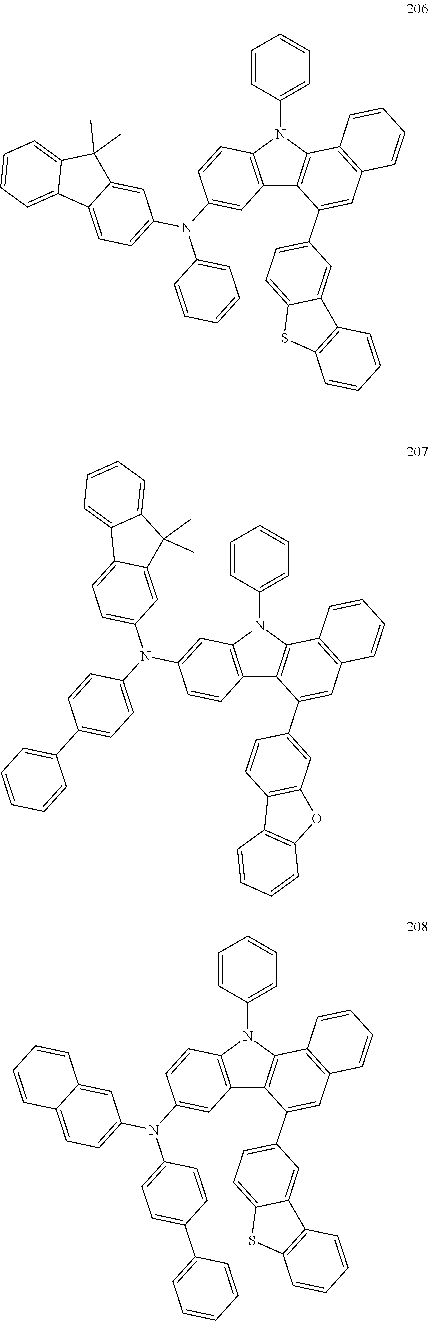

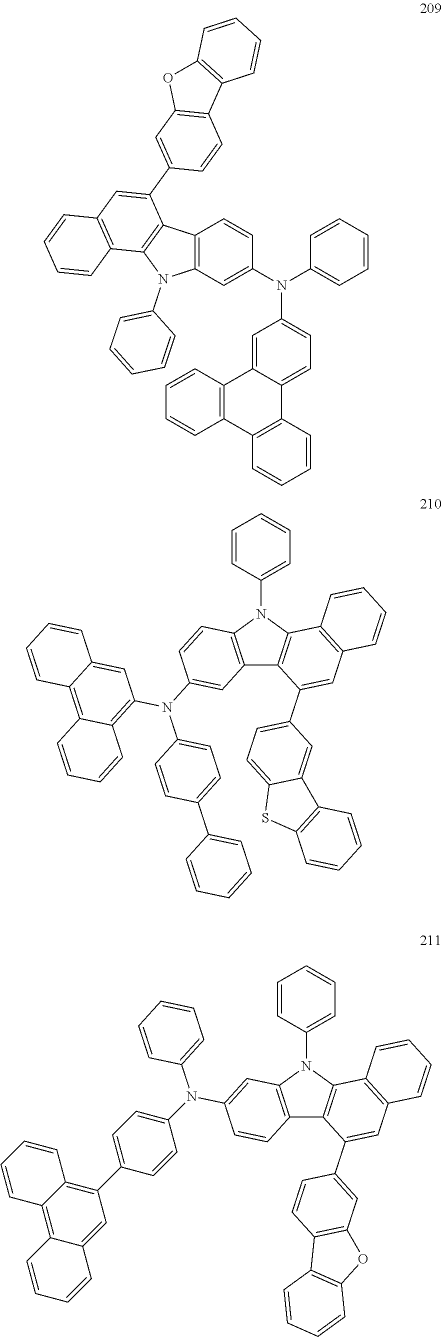

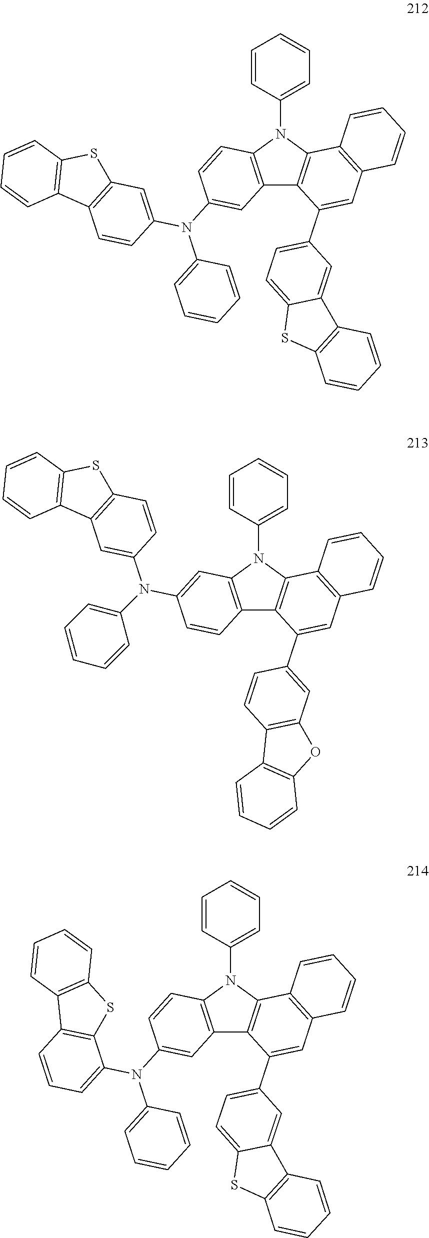 [SMILES: c1ccc(N(c2ccc3c(c2)sc2ccccc23)c2ccc3c(c2)c2c(-c4ccc5sc6ccccc6c5c4)cc4ccccc4c2n3-c2ccccc2)cc1 ; c1ccc(N(c2ccc3sc4ccccc4c3c2)c2ccc3c4c(-c5ccc6c(c5)oc5ccccc56)cc5ccccc5c4n(-c4ccccc4)c3c2)cc1 ; c1ccc(N(c2ccc3c(c2)c2c(-c4ccc5sc6ccccc6c5c4)cc4ccccc4c2n3-c2ccccc2)c2cccc3c2sc2ccccc23)cc1]

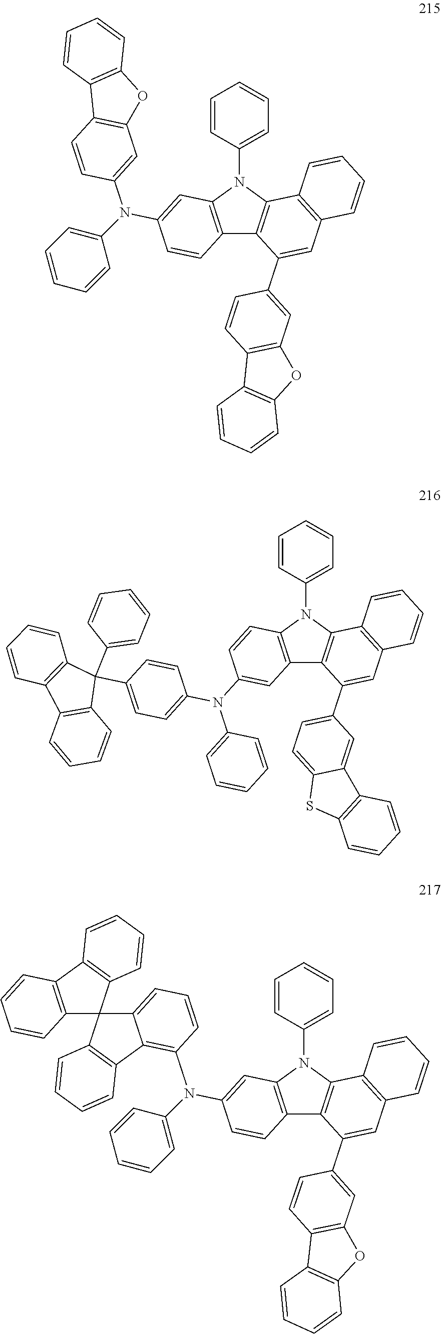 [SMILES: c1ccc(N(c2ccc3c(c2)oc2ccccc23)c2ccc3c4c(-c5ccc6c(c5)oc5ccccc56)cc5ccccc5c4n(-c4ccccc4)c3c2)cc1 ; c1ccc(N(c2ccc(C3(c4ccccc4)c4ccccc4-c4ccccc43)cc2)c2ccc3c(c2)c2c(-c4ccc5sc6ccccc6c5c4)cc4ccccc4c2n3-c2ccccc2)cc1 ; c1ccc(N(c2ccc3c4c(-c5ccc6c(c5)oc5ccccc56)cc5ccccc5c4n(-c4ccccc4)c3c2)c2cccc3c2-c2ccccc2C32c3ccccc3-c3ccccc32)cc1]

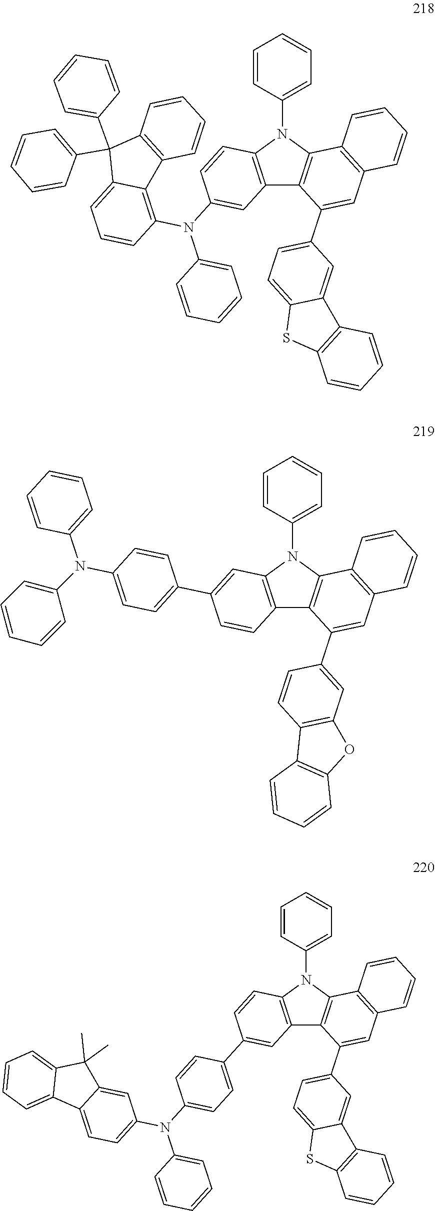 [SMILES: c1ccc(N(c2ccc3c(c2)c2c(-c4ccc5sc6ccccc6c5c4)cc4ccccc4c2n3-c2ccccc2)c2cccc3c2-c2ccccc2C3(c2ccccc2)c2ccccc2)cc1 ; c1ccc(N(c2ccccc2)c2ccc(-c3ccc4c5c(-c6ccc7c(c6)oc6ccccc67)cc6ccccc6c5n(-c5ccccc5)c4c3)cc2)cc1 ; CC1(C)c2ccccc2-c2ccc(N(c3ccccc3)c3ccc(-c4ccc5c(c4)c4c(-c6ccc7sc8ccccc8c7c6)cc6ccccc6c4n5-c4ccccc4)cc3)cc21]

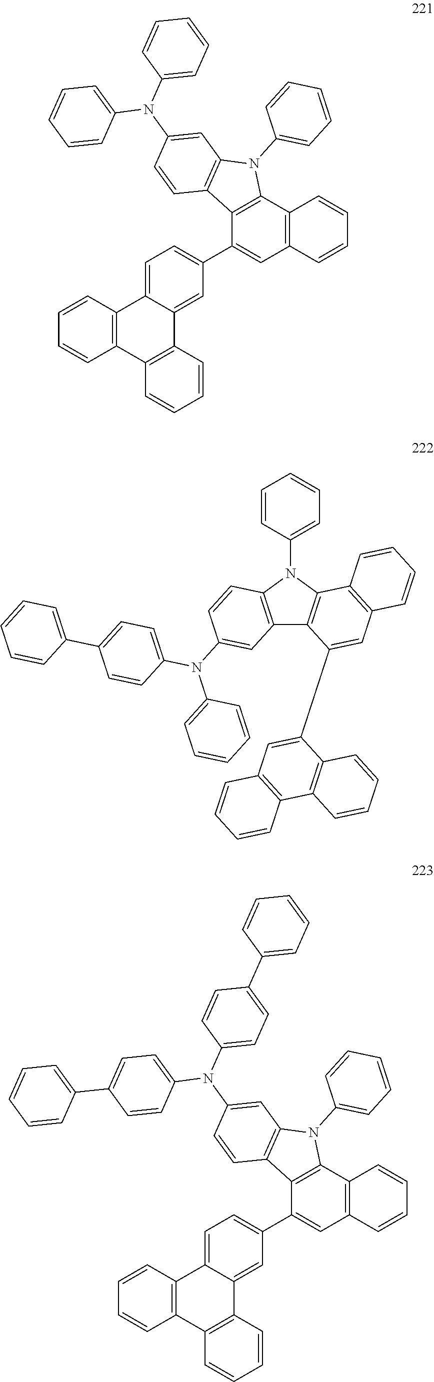 [SMILES: c1ccc(N(c2ccccc2)c2ccc3c4c(-c5ccc6c7ccccc7c7ccccc7c6c5)cc5ccccc5c4n(-c4ccccc4)c3c2)cc1 ; c1ccc(-c2ccc(N(c3ccccc3)c3ccc4c(c3)c3c(-c5cc6ccccc6c6ccccc56)cc5ccccc5c3n4-c3ccccc3)cc2)cc1 ; c1ccc(-c2ccc(N(c3ccc(-c4ccccc4)cc3)c3ccc4c5c(-c6ccc7c8ccccc8c8ccccc8c7c6)cc6ccccc6c5n(-c5ccccc5)c4c3)cc2)cc1]

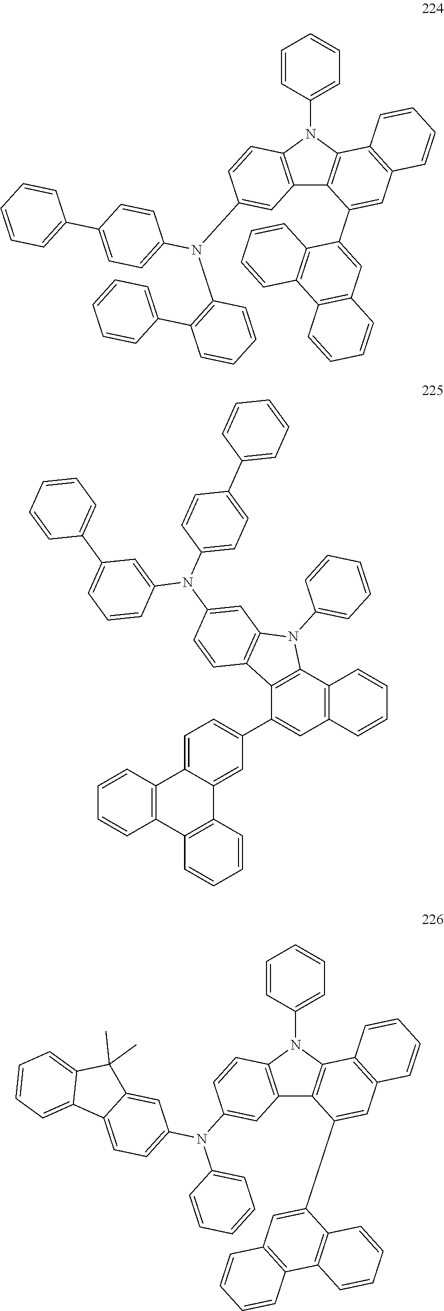

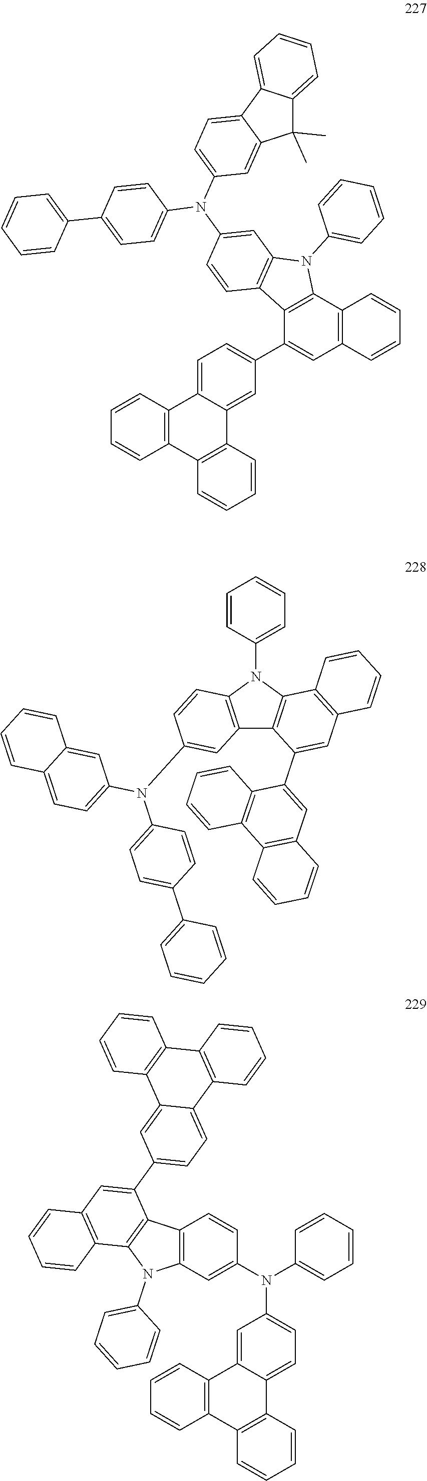

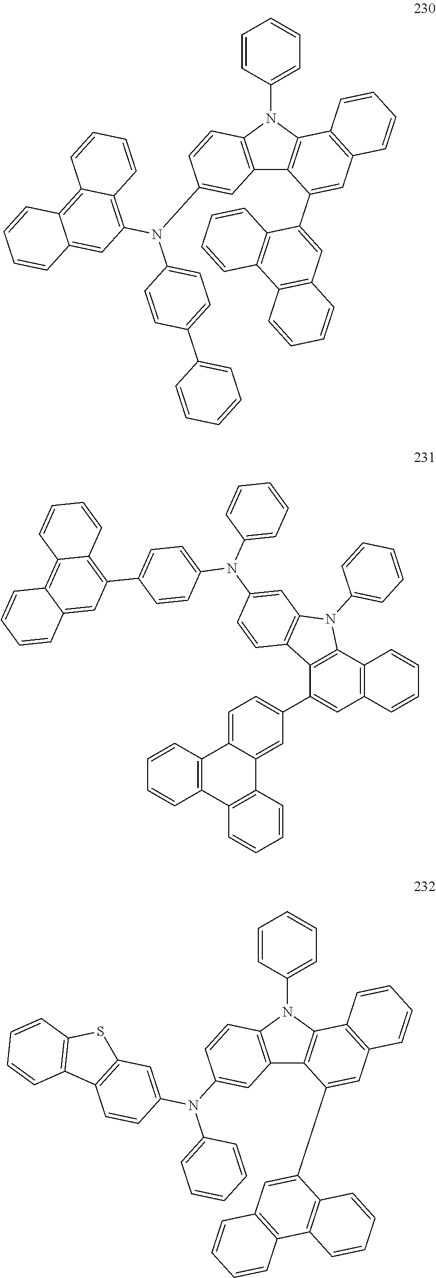 [SMILES: c1ccc(-c2ccc(N(c3ccc4c(c3)c3c(-c5cc6ccccc6c6ccccc56)cc5ccccc5c3n4-c3ccccc3)c3cc4ccccc4c4ccccc34)cc2)cc1 ; c1ccc(N(c2ccc(-c3cc4ccccc4c4ccccc34)cc2)c2ccc3c4c(-c5ccc6c7ccccc7c7ccccc7c6c5)cc5ccccc5c4n(-c4ccccc4)c3c2)cc1 ; c1ccc(N(c2ccc3c(c2)sc2ccccc23)c2ccc3c(c2)c2c(-c4cc5ccccc5c5ccccc45)cc4ccccc4c2n3-c2ccccc2)cc1]

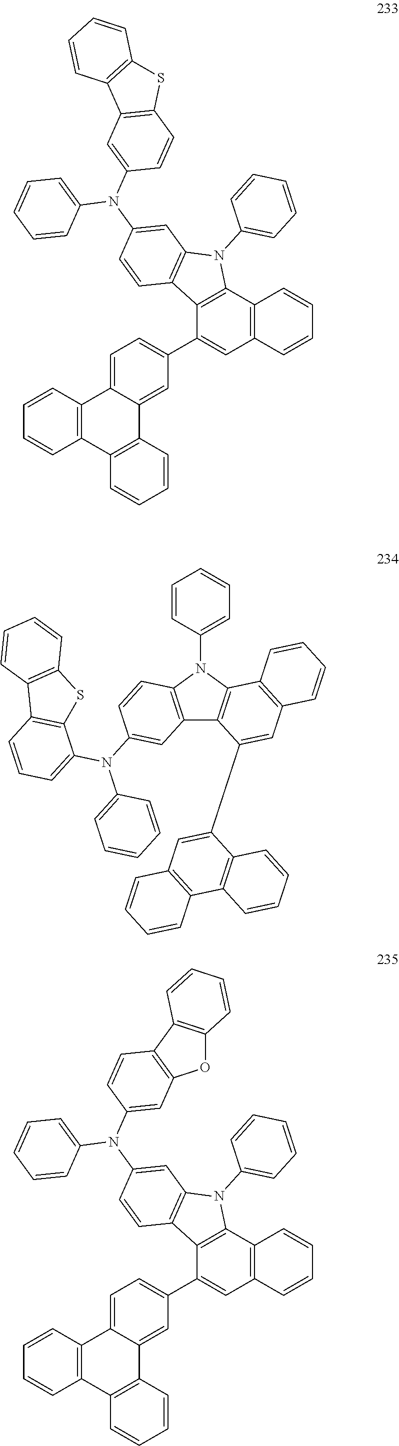

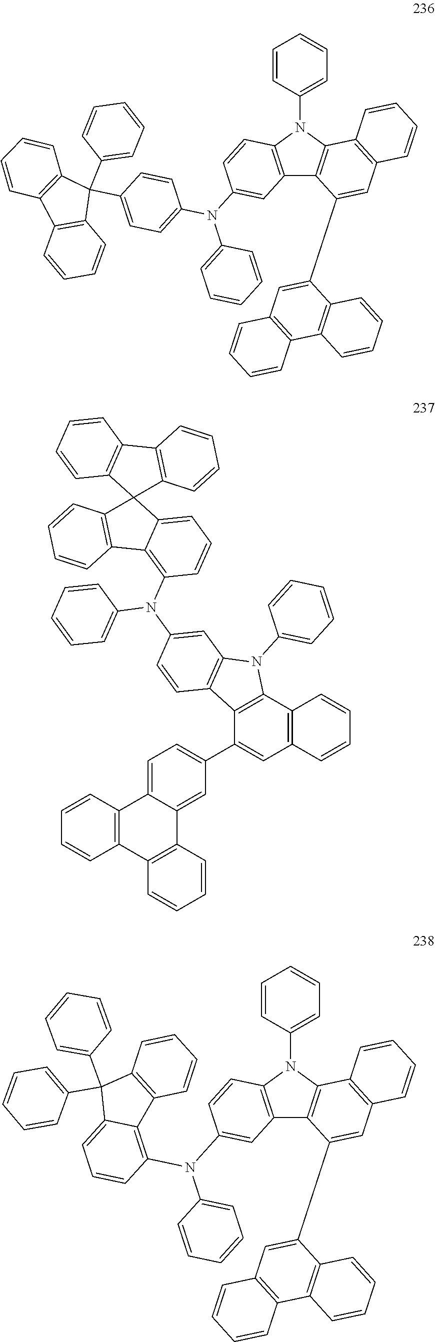 [SMILES: c1ccc(N(c2ccc(C3(c4ccccc4)c4ccccc4-c4ccccc43)cc2)c2ccc3c(c2)c2c(-c4cc5ccccc5c5ccccc45)cc4ccccc4c2n3-c2ccccc2)cc1 ; c1ccc(N(c2ccc3c4c(-c5ccc6c7ccccc7c7ccccc7c6c5)cc5ccccc5c4n(-c4ccccc4)c3c2)c2cccc3c2-c2ccccc2C32c3ccccc3-c3ccccc32)cc1 ; c1ccc(N(c2ccc3c(c2)c2c(-c4cc5ccccc5c5ccccc45)cc4ccccc4c2n3-c2ccccc2)c2cccc3c2-c2ccccc2C3(c2ccccc2)c2ccccc2)cc1]

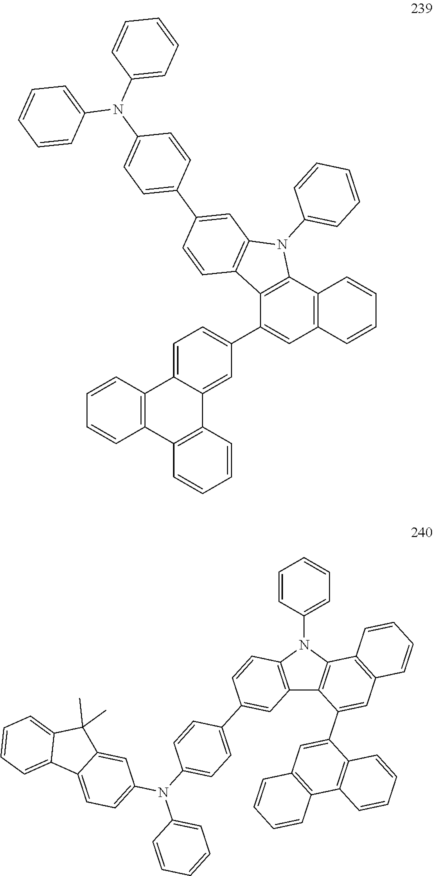 [SMILES: c1ccc(N(c2ccccc2)c2ccc(-c3ccc4c5c(-c6ccc7c8ccccc8c8ccccc8c7c6)cc6ccccc6c5n(-c5ccccc5)c4c3)cc2)cc1 ; CC1(C)c2ccccc2-c2ccc(N(c3ccccc3)c3ccc(-c4ccc5c(c4)c4c(-c6cc7ccccc7c7ccccc67)cc6ccccc6c4n5-c4ccccc4)cc3)cc21]

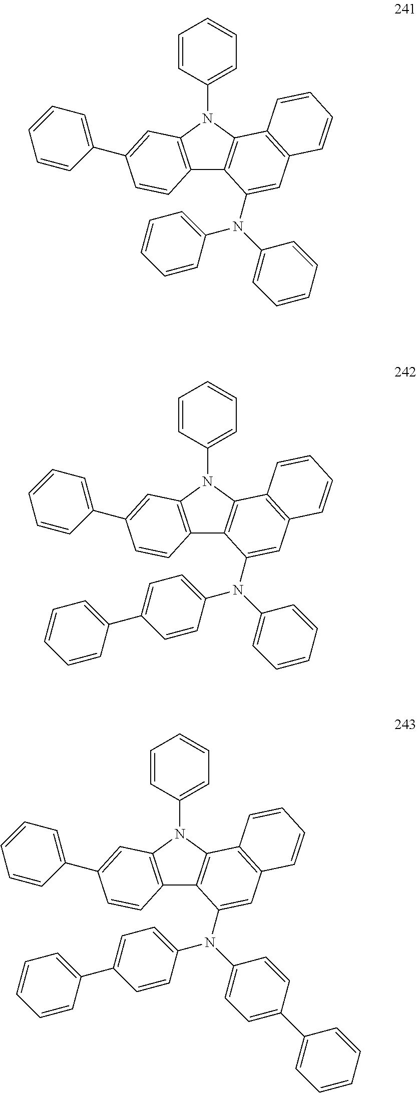 [SMILES: c1ccc(-c2ccc3c4c(N(c5ccccc5)c5ccccc5)cc5ccccc5c4n(-c4ccccc4)c3c2)cc1 ; c1ccc(-c2ccc(N(c3ccccc3)c3cc4ccccc4c4c3c3ccc(-c5ccccc5)cc3n4-c3ccccc3)cc2)cc1 ; c1ccc(-c2ccc(N(c3ccc(-c4ccccc4)cc3)c3cc4ccccc4c4c3c3ccc(-c5ccccc5)cc3n4-c3ccccc3)cc2)cc1]

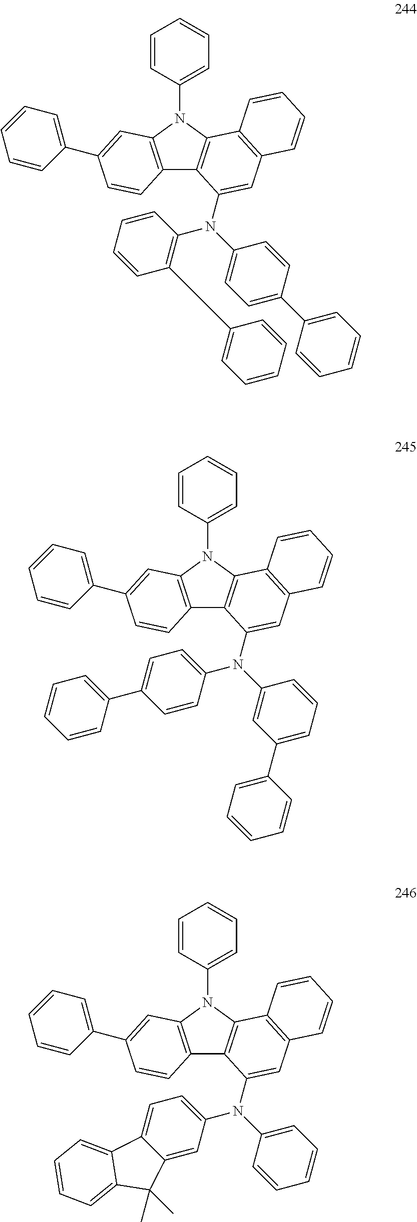 [SMILES: c1ccc(-c2ccc(N(c3ccccc3-c3ccccc3)c3cc4ccccc4c4c3c3ccc(-c5ccccc5)cc3n4-c3ccccc3)cc2)cc1 ; c1ccc(-c2ccc(N(c3cccc(-c4ccccc4)c3)c3cc4ccccc4c4c3c3ccc(-c5ccccc5)cc3n4-c3ccccc3)cc2)cc1 ; CC1(C)c2ccccc2-c2ccc(N(c3ccccc3)c3cc4ccccc4c4c3c3ccc(-c5ccccc5)cc3n4-c3ccccc3)cc21]

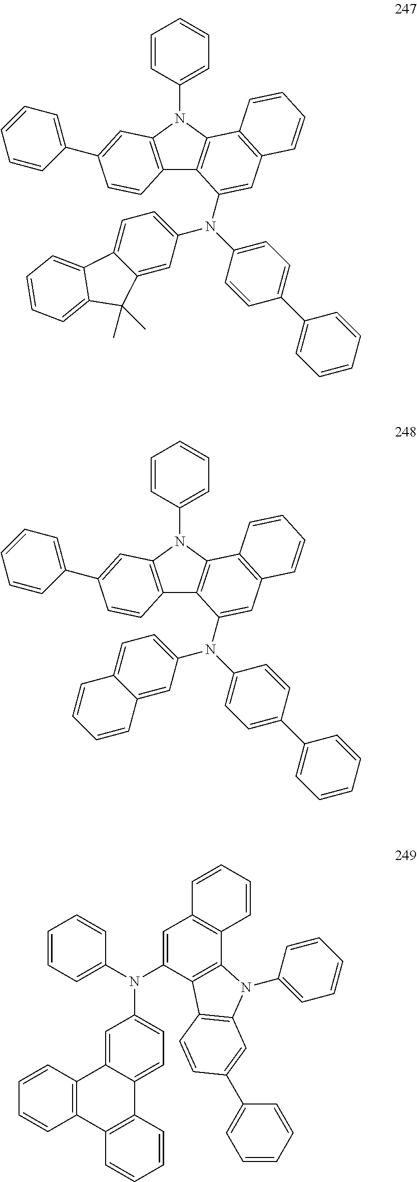 [SMILES: CC1(C)c2ccccc2-c2ccc(N(c3ccc(-c4ccccc4)cc3)c3cc4ccccc4c4c3c3ccc(-c5ccccc5)cc3n4-c3ccccc3)cc21 ; c1ccc(-c2ccc(N(c3ccc4ccccc4c3)c3cc4ccccc4c4c3c3ccc(-c5ccccc5)cc3n4-c3ccccc3)cc2)cc1 ; c1ccc(-c2ccc3c4c(N(c5ccccc5)c5ccc6c7ccccc7c7ccccc7c6c5)cc5ccccc5c4n(-c4ccccc4)c3c2)cc1]

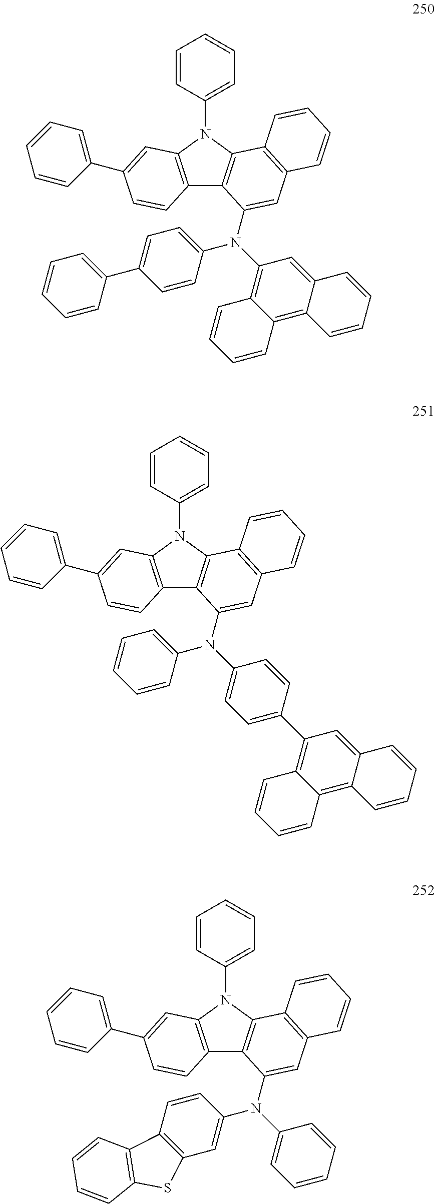 [SMILES: c1ccc(-c2ccc(N(c3cc4ccccc4c4ccccc34)c3cc4ccccc4c4c3c3ccc(-c5ccccc5)cc3n4-c3ccccc3)cc2)cc1 ; c1ccc(-c2ccc3c4c(N(c5ccccc5)c5ccc(-c6cc7ccccc7c7ccccc67)cc5)cc5ccccc5c4n(-c4ccccc4)c3c2)cc1 ; c1ccc(-c2ccc3c4c(N(c5ccccc5)c5ccc6c(c5)sc5ccccc56)cc5ccccc5c4n(-c4ccccc4)c3c2)cc1]

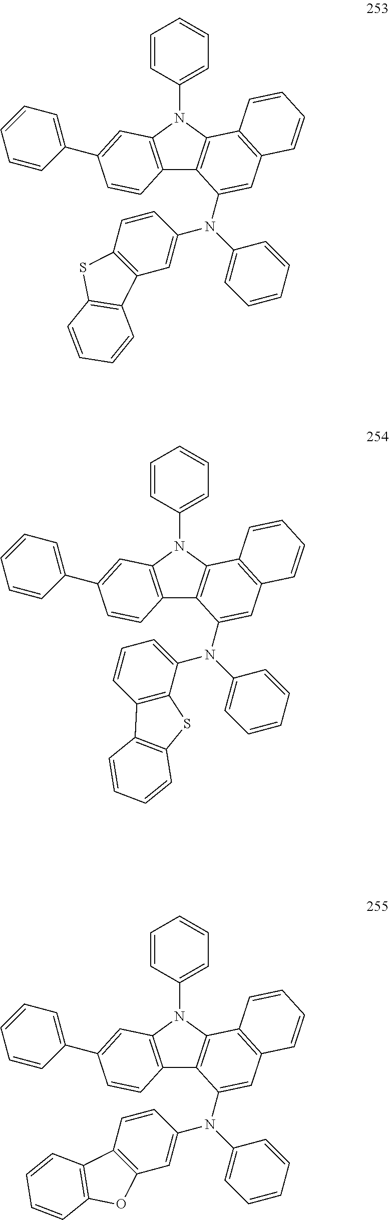 [SMILES: c1ccc(-c2ccc3c4c(N(c5ccccc5)c5ccc6sc7ccccc7c6c5)cc5ccccc5c4n(-c4ccccc4)c3c2)cc1 ; c1ccc(-c2ccc3c4c(N(c5ccccc5)c5cccc6c5sc5ccccc56)cc5ccccc5c4n(-c4ccccc4)c3c2)cc1 ; c1ccc(-c2ccc3c4c(N(c5ccccc5)c5ccc6c(c5)oc5ccccc56)cc5ccccc5c4n(-c4ccccc4)c3c2)cc1]

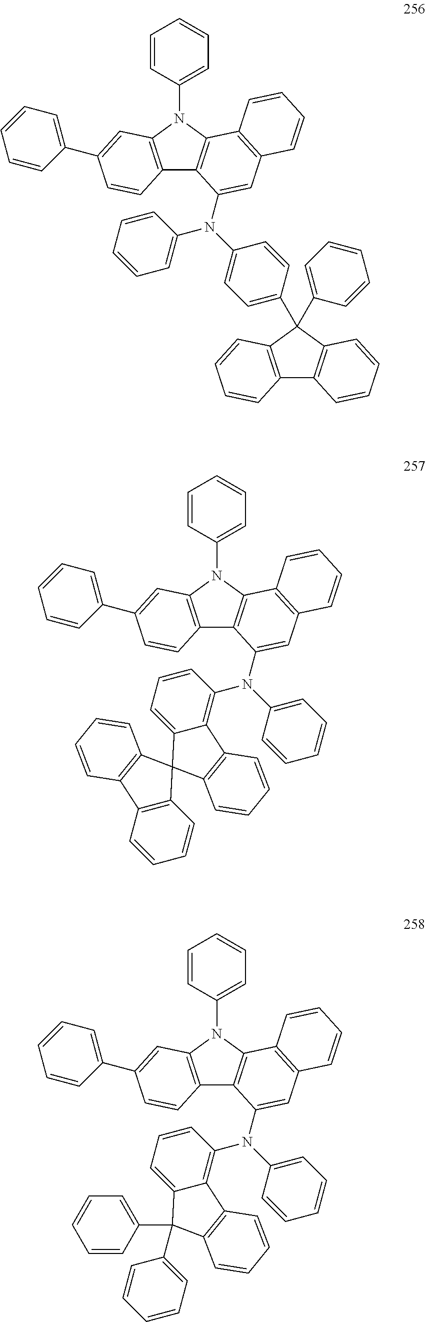 [SMILES: c1ccc(-c2ccc3c4c(N(c5ccccc5)c5ccc(C6(c7ccccc7)c7ccccc7-c7ccccc76)cc5)cc5ccccc5c4n(-c4ccccc4)c3c2)cc1 ; c1ccc(-c2ccc3c4c(N(c5ccccc5)c5cccc6c5-c5ccccc5C65c6ccccc6-c6ccccc65)cc5ccccc5c4n(-c4ccccc4)c3c2)cc1 ; c1ccc(-c2ccc3c4c(N(c5ccccc5)c5cccc6c5-c5ccccc5C6(c5ccccc5)c5ccccc5)cc5ccccc5c4n(-c4ccccc4)c3c2)cc1]

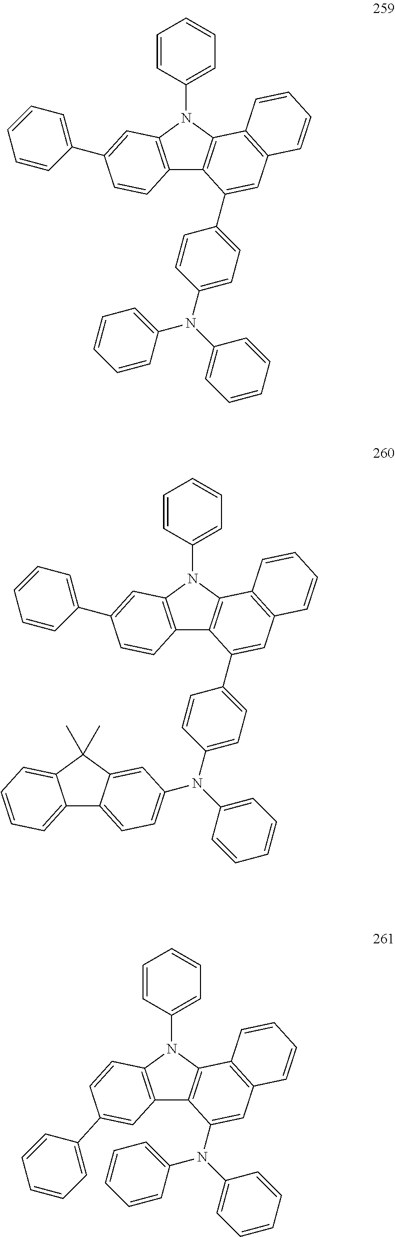 [SMILES: c1ccc(-c2ccc3c4c(-c5ccc(N(c6ccccc6)c6ccccc6)cc5)cc5ccccc5c4n(-c4ccccc4)c3c2)cc1 ; CC1(C)c2ccccc2-c2ccc(N(c3ccccc3)c3ccc(-c4cc5ccccc5c5c4c4ccc(-c6ccccc6)cc4n5-c4ccccc4)cc3)cc21 ; c1ccc(-c2ccc3c(c2)c2c(N(c4ccccc4)c4ccccc4)cc4ccccc4c2n3-c2ccccc2)cc1]

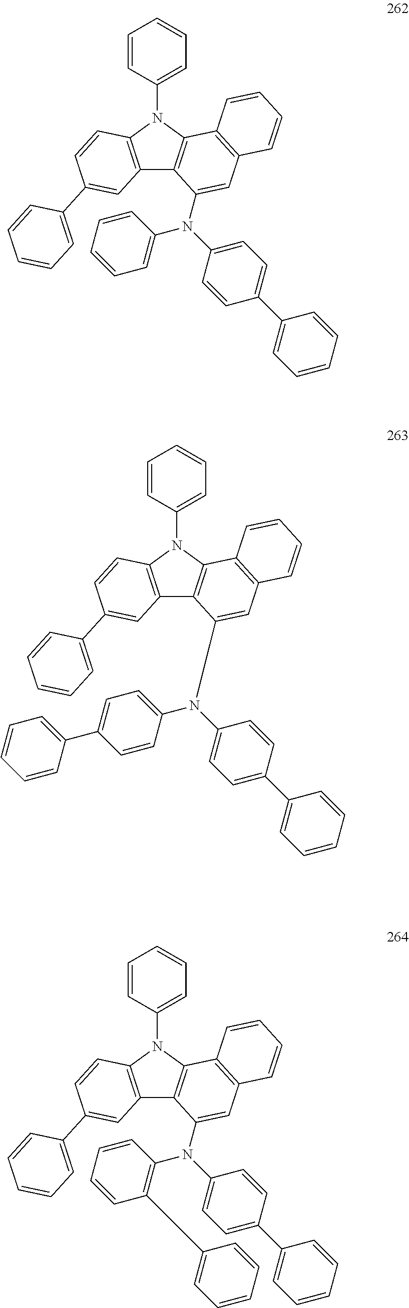 [SMILES: c1ccc(-c2ccc(N(c3ccccc3)c3cc4ccccc4c4c3c3cc(-c5ccccc5)ccc3n4-c3ccccc3)cc2)cc1 ; c1ccc(-c2ccc(N(c3ccc(-c4ccccc4)cc3)c3cc4ccccc4c4c3c3cc(-c5ccccc5)ccc3n4-c3ccccc3)cc2)cc1 ; c1ccc(-c2ccc(N(c3ccccc3-c3ccccc3)c3cc4ccccc4c4c3c3cc(-c5ccccc5)ccc3n4-c3ccccc3)cc2)cc1]

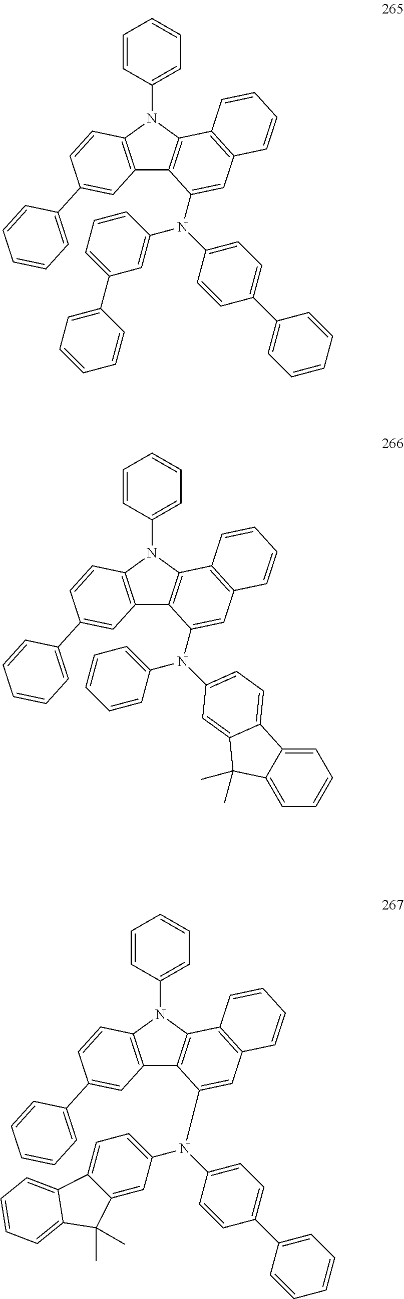 [SMILES: c1ccc(-c2ccc(N(c3cccc(-c4ccccc4)c3)c3cc4ccccc4c4c3c3cc(-c5ccccc5)ccc3n4-c3ccccc3)cc2)cc1 ; CC1(C)c2ccccc2-c2ccc(N(c3ccccc3)c3cc4ccccc4c4c3c3cc(-c5ccccc5)ccc3n4-c3ccccc3)cc21 ; CC1(C)c2ccccc2-c2ccc(N(c3ccc(-c4ccccc4)cc3)c3cc4ccccc4c4c3c3cc(-c5ccccc5)ccc3n4-c3ccccc3)cc21]

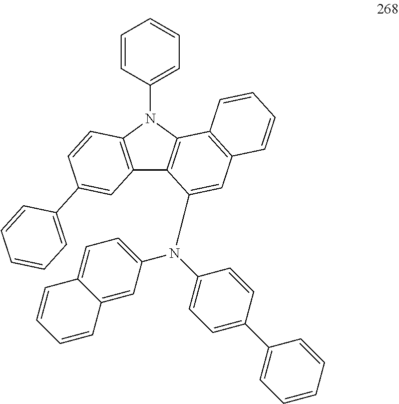 [SMILES: c1ccc(-c2ccc(N(c3ccc4ccccc4c3)c3cc4ccccc4c4c3c3cc(-c5ccccc5)ccc3n4-c3ccccc3)cc2)cc1]

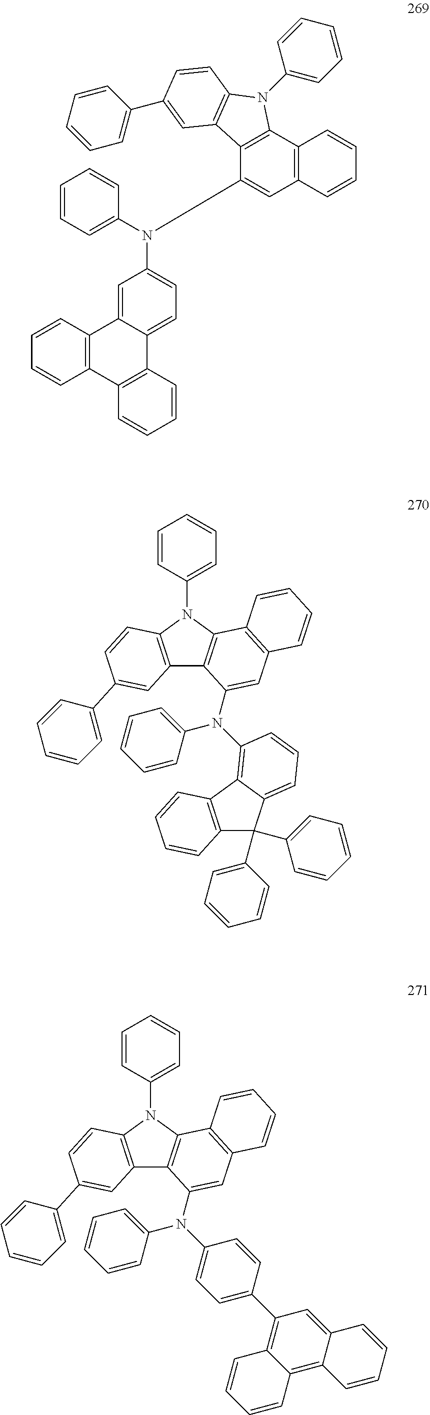 [SMILES: c1ccc(-c2ccc3c(c2)c2c(N(c4ccccc4)c4ccc5c6ccccc6c6ccccc6c5c4)cc4ccccc4c2n3-c2ccccc2)cc1 ; c1ccc(-c2ccc3c(c2)c2c(N(c4ccccc4)c4cccc5c4-c4ccccc4C5(c4ccccc4)c4ccccc4)cc4ccccc4c2n3-c2ccccc2)cc1 ; c1ccc(-c2ccc3c(c2)c2c(N(c4ccccc4)c4ccc(-c5cc6ccccc6c6ccccc56)cc4)cc4ccccc4c2n3-c2ccccc2)cc1]

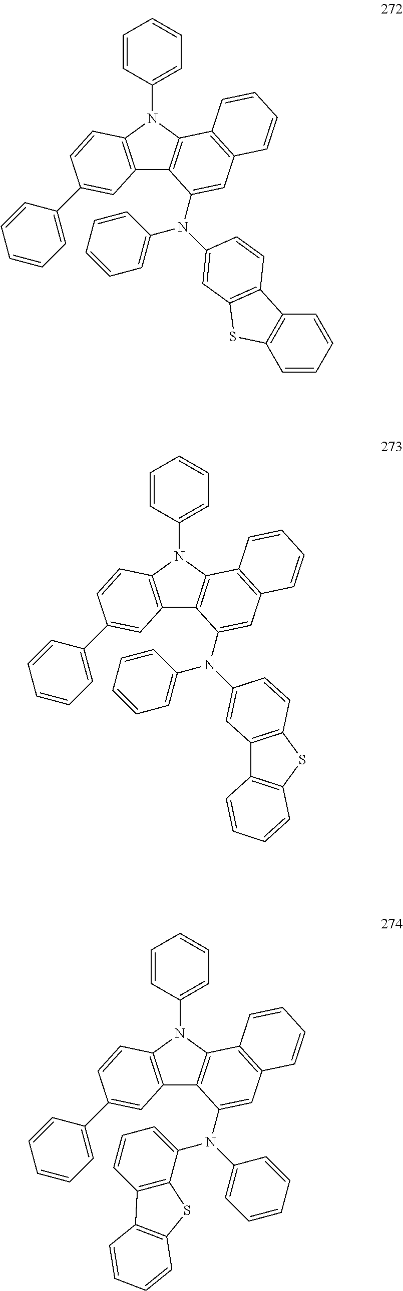 [SMILES: c1ccc(-c2ccc3c(c2)c2c(N(c4ccccc4)c4ccc5c(c4)sc4ccccc45)cc4ccccc4c2n3-c2ccccc2)cc1 ; c1ccc(-c2ccc3c(c2)c2c(N(c4ccccc4)c4ccc5sc6ccccc6c5c4)cc4ccccc4c2n3-c2ccccc2)cc1 ; c1ccc(-c2ccc3c(c2)c2c(N(c4ccccc4)c4cccc5c4sc4ccccc45)cc4ccccc4c2n3-c2ccccc2)cc1]

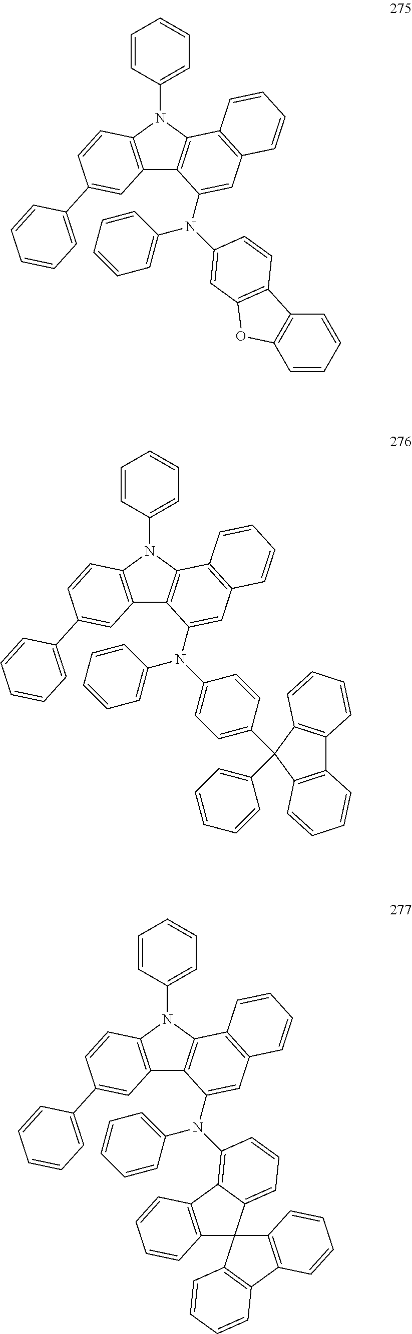 [SMILES: c1ccc(-c2ccc3c(c2)c2c(N(c4ccccc4)c4ccc5c(c4)oc4ccccc45)cc4ccccc4c2n3-c2ccccc2)cc1 ; c1ccc(-c2ccc3c(c2)c2c(N(c4ccccc4)c4ccc(C5(c6ccccc6)c6ccccc6-c6ccccc65)cc4)cc4ccccc4c2n3-c2ccccc2)cc1 ; c1ccc(-c2ccc3c(c2)c2c(N(c4ccccc4)c4cccc5c4-c4ccccc4C54c5ccccc5-c5ccccc54)cc4ccccc4c2n3-c2ccccc2)cc1]

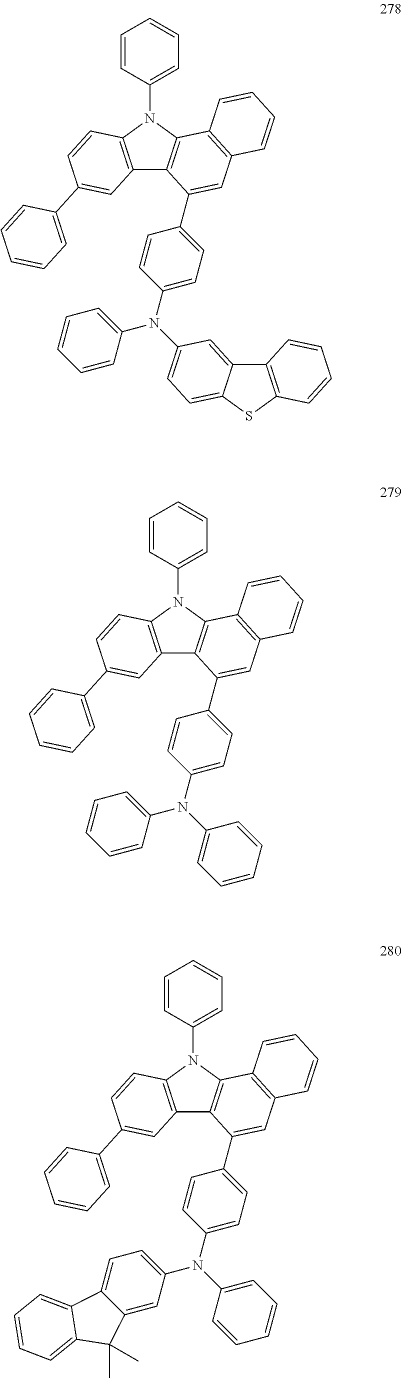 [SMILES: c1ccc(-c2ccc3c(c2)c2c(-c4ccc(N(c5ccccc5)c5ccc6sc7ccccc7c6c5)cc4)cc4ccccc4c2n3-c2ccccc2)cc1 ; c1ccc(-c2ccc3c(c2)c2c(-c4ccc(N(c5ccccc5)c5ccccc5)cc4)cc4ccccc4c2n3-c2ccccc2)cc1 ; CC1(C)c2ccccc2-c2ccc(N(c3ccccc3)c3ccc(-c4cc5ccccc5c5c4c4cc(-c6ccccc6)ccc4n5-c4ccccc4)cc3)cc21]

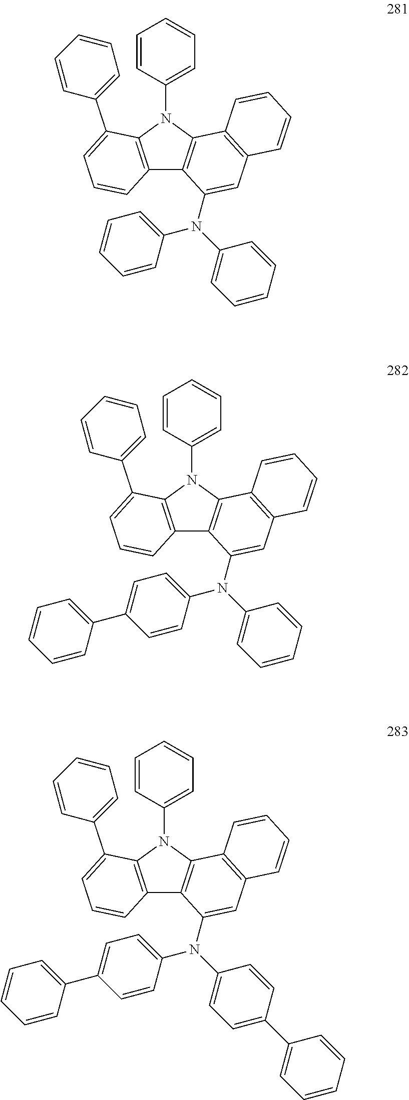 [SMILES: c1ccc(-c2cccc3c4c(N(c5ccccc5)c5ccccc5)cc5ccccc5c4n(-c4ccccc4)c23)cc1 ; c1ccc(-c2ccc(N(c3ccccc3)c3cc4ccccc4c4c3c3cccc(-c5ccccc5)c3n4-c3ccccc3)cc2)cc1 ; c1ccc(-c2ccc(N(c3ccc(-c4ccccc4)cc3)c3cc4ccccc4c4c3c3cccc(-c5ccccc5)c3n4-c3ccccc3)cc2)cc1]

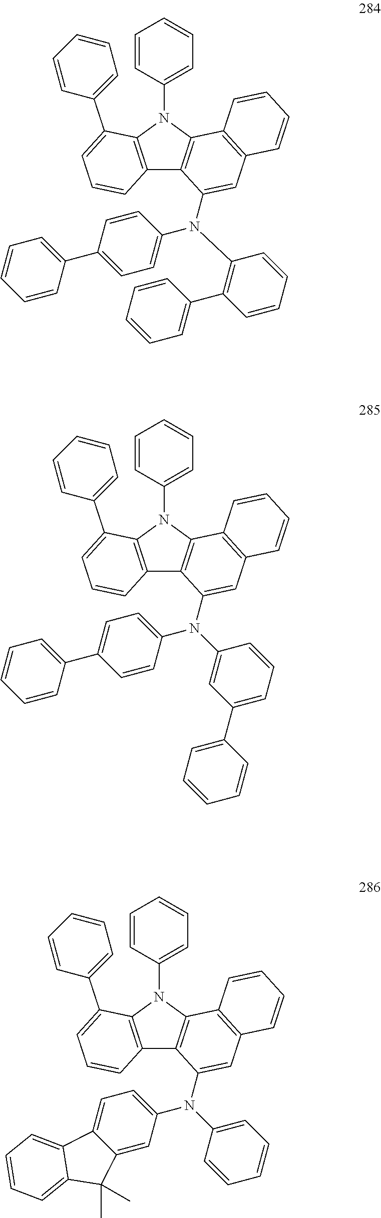 [SMILES: c1ccc(-c2ccc(N(c3ccccc3-c3ccccc3)c3cc4ccccc4c4c3c3cccc(-c5ccccc5)c3n4-c3ccccc3)cc2)cc1 ; c1ccc(-c2ccc(N(c3cccc(-c4ccccc4)c3)c3cc4ccccc4c4c3c3cccc(-c5ccccc5)c3n4-c3ccccc3)cc2)cc1 ; CC1(C)c2ccccc2-c2ccc(N(c3ccccc3)c3cc4ccccc4c4c3c3cccc(-c5ccccc5)c3n4-c3ccccc3)cc21]

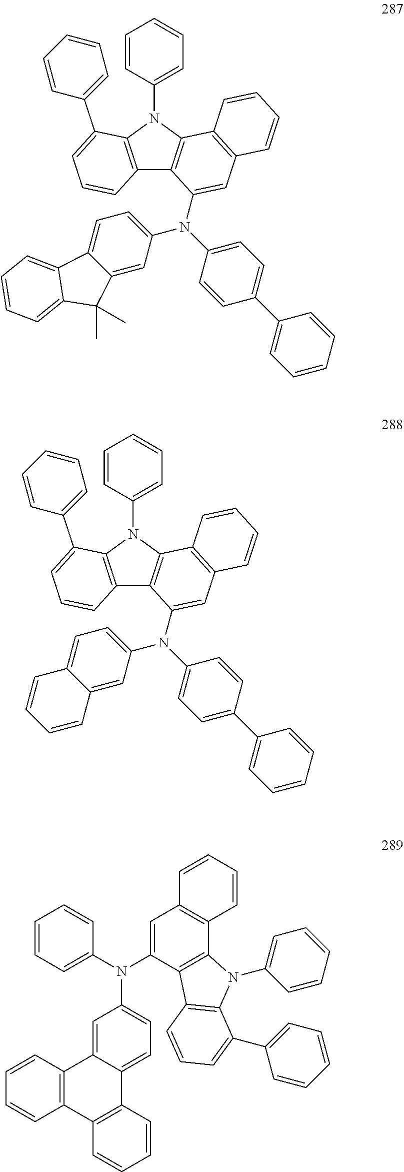 [SMILES: CC1(C)c2ccccc2-c2ccc(N(c3ccc(-c4ccccc4)cc3)c3cc4ccccc4c4c3c3cccc(-c5ccccc5)c3n4-c3ccccc3)cc21 ; c1ccc(-c2ccc(N(c3ccc4ccccc4c3)c3cc4ccccc4c4c3c3cccc(-c5ccccc5)c3n4-c3ccccc3)cc2)cc1 ; c1ccc(-c2cccc3c4c(N(c5ccccc5)c5ccc6c7ccccc7c7ccccc7c6c5)cc5ccccc5c4n(-c4ccccc4)c23)cc1]

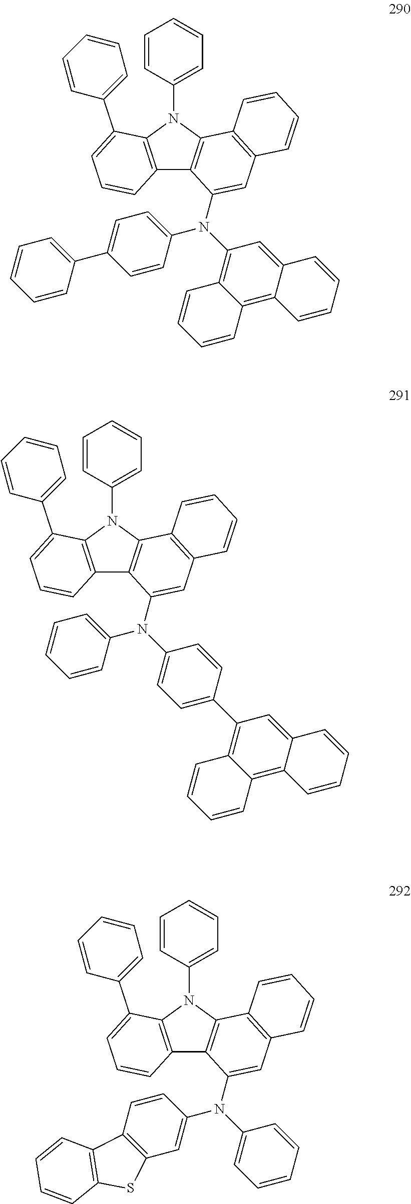 [SMILES: c1ccc(-c2ccc(N(c3cc4ccccc4c4ccccc34)c3cc4ccccc4c4c3c3cccc(-c5ccccc5)c3n4-c3ccccc3)cc2)cc1 ; c1ccc(-c2cccc3c4c(N(c5ccccc5)c5ccc(-c6cc7ccccc7c7ccccc67)cc5)cc5ccccc5c4n(-c4ccccc4)c23)cc1 ; c1ccc(-c2cccc3c4c(N(c5ccccc5)c5ccc6c(c5)sc5ccccc56)cc5ccccc5c4n(-c4ccccc4)c23)cc1]

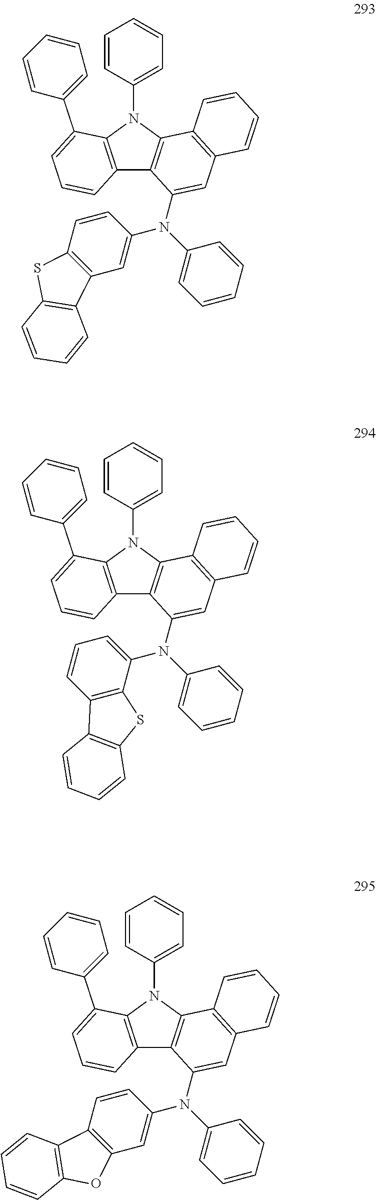 [SMILES: c1ccc(-c2cccc3c4c(N(c5ccccc5)c5ccc6sc7ccccc7c6c5)cc5ccccc5c4n(-c4ccccc4)c23)cc1 ; c1ccc(-c2cccc3c4c(N(c5ccccc5)c5cccc6c5sc5ccccc56)cc5ccccc5c4n(-c4ccccc4)c23)cc1 ; c1ccc(-c2cccc3c4c(N(c5ccccc5)c5ccc6c(c5)oc5ccccc56)cc5ccccc5c4n(-c4ccccc4)c23)cc1]

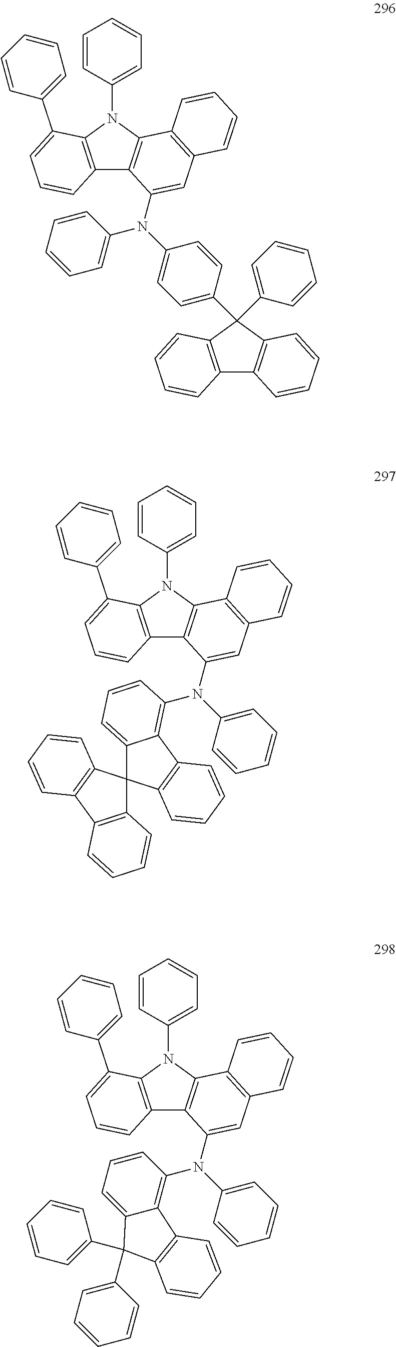 [SMILES: c1ccc(-c2cccc3c4c(N(c5ccccc5)c5ccc(C6(c7ccccc7)c7ccccc7-c7ccccc76)cc5)cc5ccccc5c4n(-c4ccccc4)c23)cc1 ; c1ccc(-c2cccc3c4c(N(c5ccccc5)c5cccc6c5-c5ccccc5C65c6ccccc6-c6ccccc65)cc5ccccc5c4n(-c4ccccc4)c23)cc1 ; c1ccc(-c2cccc3c4c(N(c5ccccc5)c5cccc6c5-c5ccccc5C6(c5ccccc5)c5ccccc5)cc5ccccc5c4n(-c4ccccc4)c23)cc1]

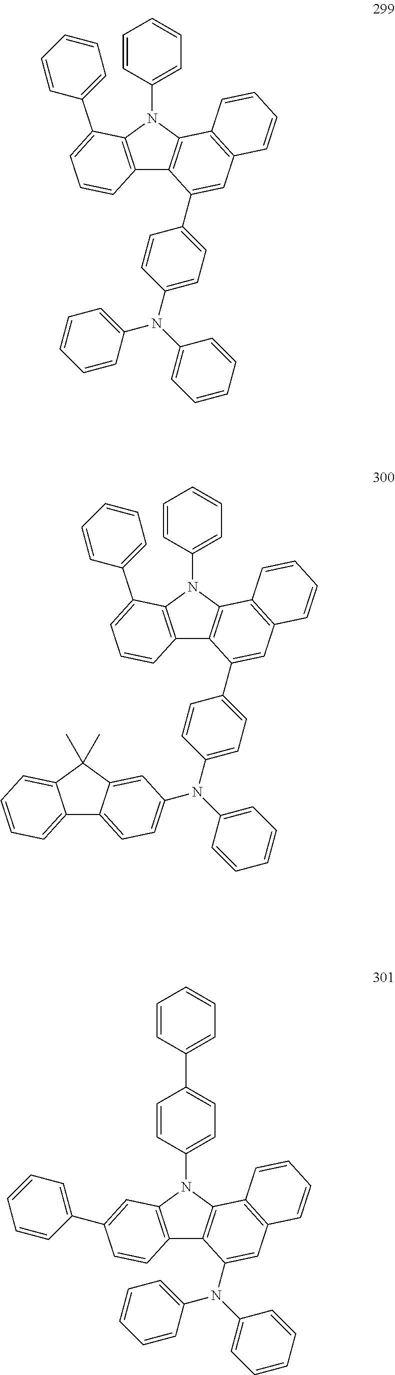 [SMILES: c1ccc(-c2cccc3c4c(-c5ccc(N(c6ccccc6)c6ccccc6)cc5)cc5ccccc5c4n(-c4ccccc4)c23)cc1 ; CC1(C)c2ccccc2-c2ccc(N(c3ccccc3)c3ccc(-c4cc5ccccc5c5c4c4cccc(-c6ccccc6)c4n5-c4ccccc4)cc3)cc21 ; c1ccc(-c2ccc(-n3c4cc(-c5ccccc5)ccc4c4c(N(c5ccccc5)c5ccccc5)cc5ccccc5c43)cc2)cc1]

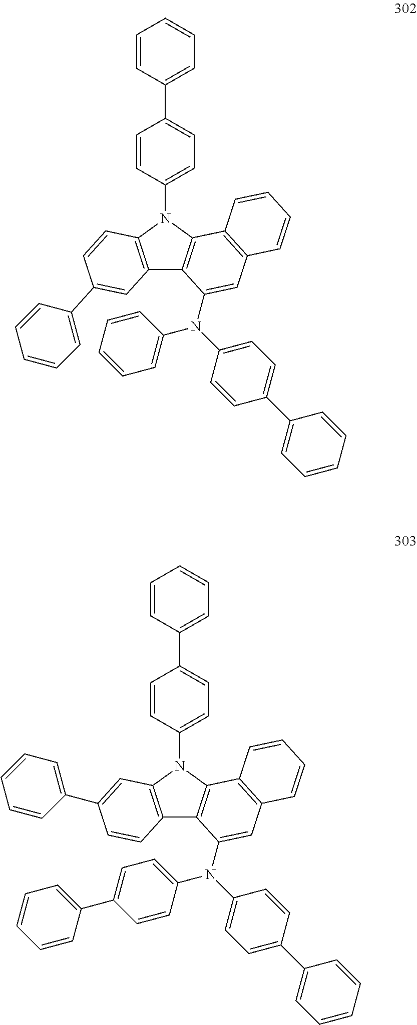 [SMILES: c1ccc(-c2ccc(N(c3ccccc3)c3cc4ccccc4c4c3c3cc(-c5ccccc5)ccc3n4-c3ccc(-c4ccccc4)cc3)cc2)cc1 ; c1ccc(-c2ccc(N(c3ccc(-c4ccccc4)cc3)c3cc4ccccc4c4c3c3ccc(-c5ccccc5)cc3n4-c3ccc(-c4ccccc4)cc3)cc2)cc1]

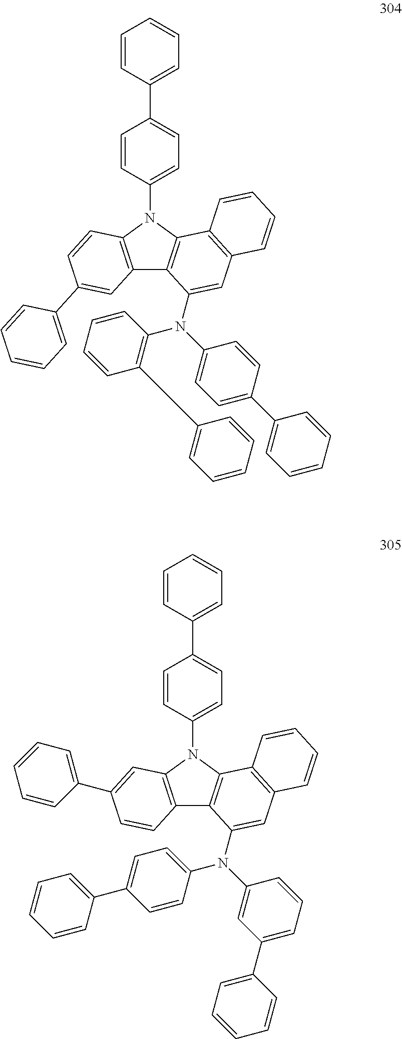 [SMILES: c1ccc(-c2ccc(N(c3ccccc3-c3ccccc3)c3cc4ccccc4c4c3c3cc(-c5ccccc5)ccc3n4-c3ccc(-c4ccccc4)cc3)cc2)cc1 ; c1ccc(-c2ccc(N(c3cccc(-c4ccccc4)c3)c3cc4ccccc4c4c3c3ccc(-c5ccccc5)cc3n4-c3ccc(-c4ccccc4)cc3)cc2)cc1]

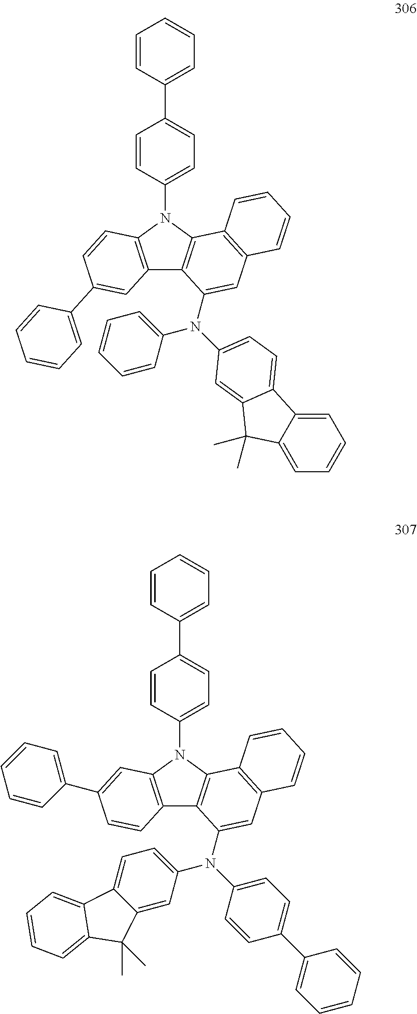 [SMILES: CC1(C)c2ccccc2-c2ccc(N(c3ccccc3)c3cc4ccccc4c4c3c3cc(-c5ccccc5)ccc3n4-c3ccc(-c4ccccc4)cc3)cc21 ; CC1(C)c2ccccc2-c2ccc(N(c3ccc(-c4ccccc4)cc3)c3cc4ccccc4c4c3c3ccc(-c5ccccc5)cc3n4-c3ccc(-c4ccccc4)cc3)cc21]

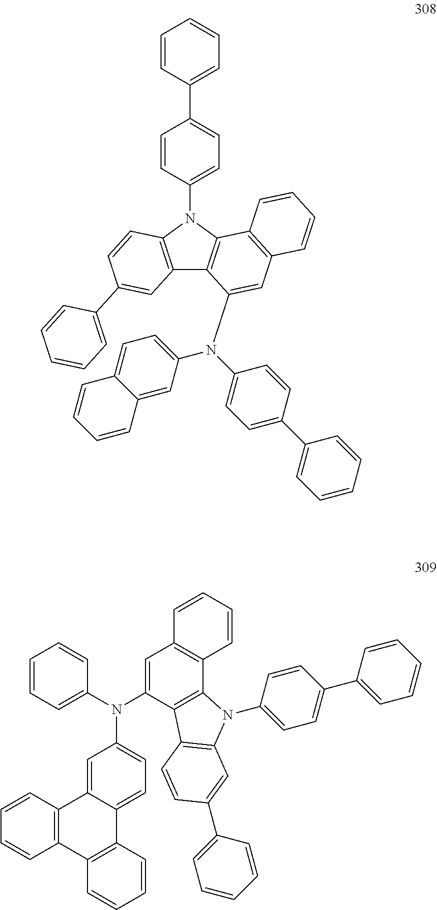 [SMILES: c1ccc(-c2ccc(N(c3ccc4ccccc4c3)c3cc4ccccc4c4c3c3cc(-c5ccccc5)ccc3n4-c3ccc(-c4ccccc4)cc3)cc2)cc1 ; c1ccc(-c2ccc(-n3c4cc(-c5ccccc5)ccc4c4c(N(c5ccccc5)c5ccc6c7ccccc7c7ccccc7c6c5)cc5ccccc5c43)cc2)cc1]

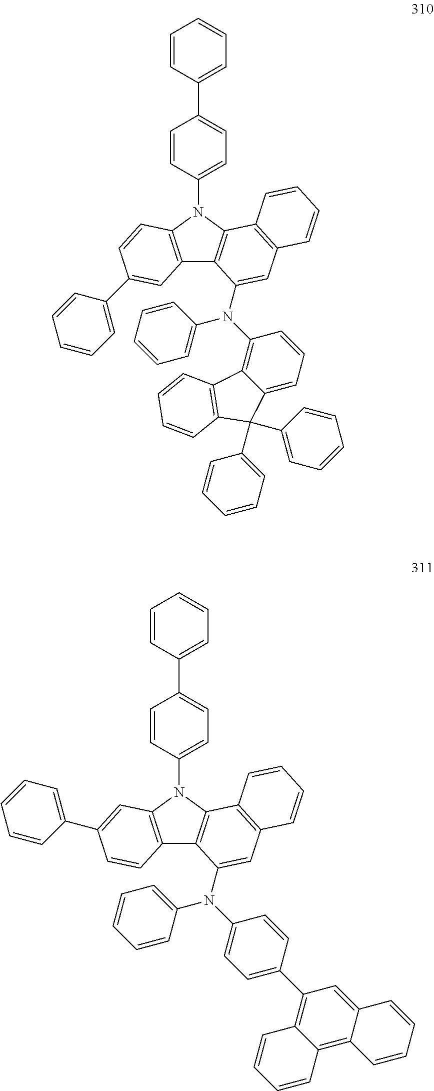 [SMILES: c1ccc(-c2ccc(-n3c4ccc(-c5ccccc5)cc4c4c(N(c5ccccc5)c5cccc6c5-c5ccccc5C6(c5ccccc5)c5ccccc5)cc5ccccc5c43)cc2)cc1 ; c1ccc(-c2ccc(-n3c4cc(-c5ccccc5)ccc4c4c(N(c5ccccc5)c5ccc(-c6cc7ccccc7c7ccccc67)cc5)cc5ccccc5c43)cc2)cc1]

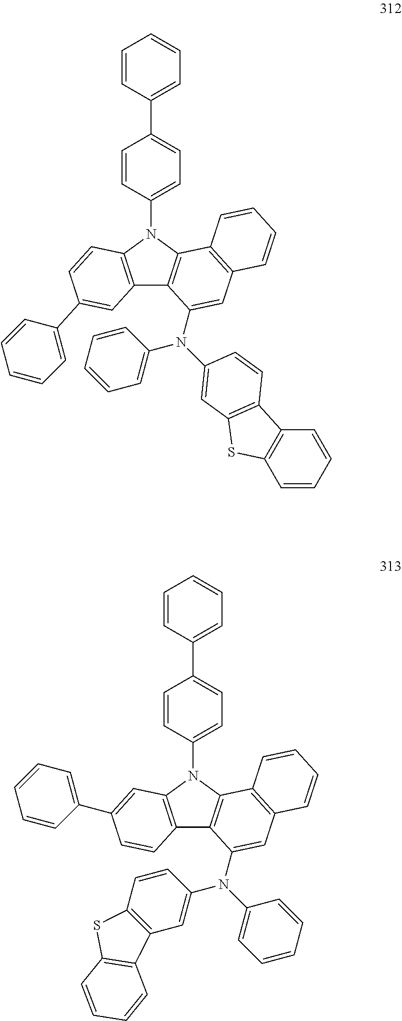 [SMILES: c1ccc(-c2ccc(-n3c4ccc(-c5ccccc5)cc4c4c(N(c5ccccc5)c5ccc6c(c5)sc5ccccc56)cc5ccccc5c43)cc2)cc1 ; c1ccc(-c2ccc(-n3c4cc(-c5ccccc5)ccc4c4c(N(c5ccccc5)c5ccc6sc7ccccc7c6c5)cc5ccccc5c43)cc2)cc1]

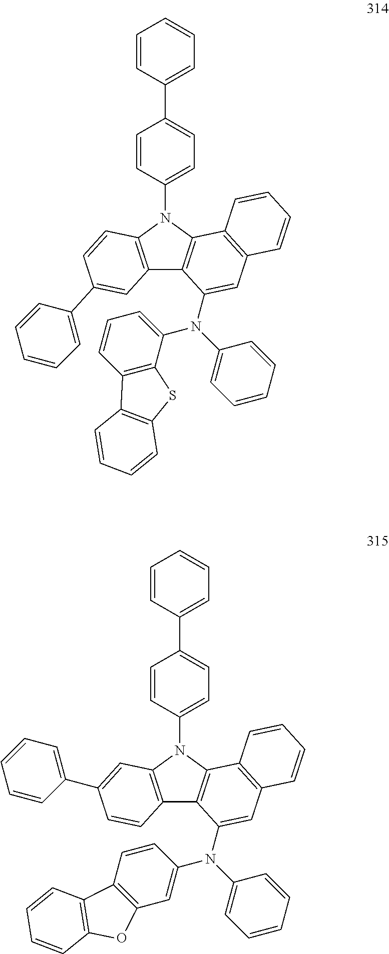 [SMILES: c1ccc(-c2ccc(-n3c4ccc(-c5ccccc5)cc4c4c(N(c5ccccc5)c5cccc6c5sc5ccccc56)cc5ccccc5c43)cc2)cc1 ; c1ccc(-c2ccc(-n3c4cc(-c5ccccc5)ccc4c4c(N(c5ccccc5)c5ccc6c(c5)oc5ccccc56)cc5ccccc5c43)cc2)cc1]

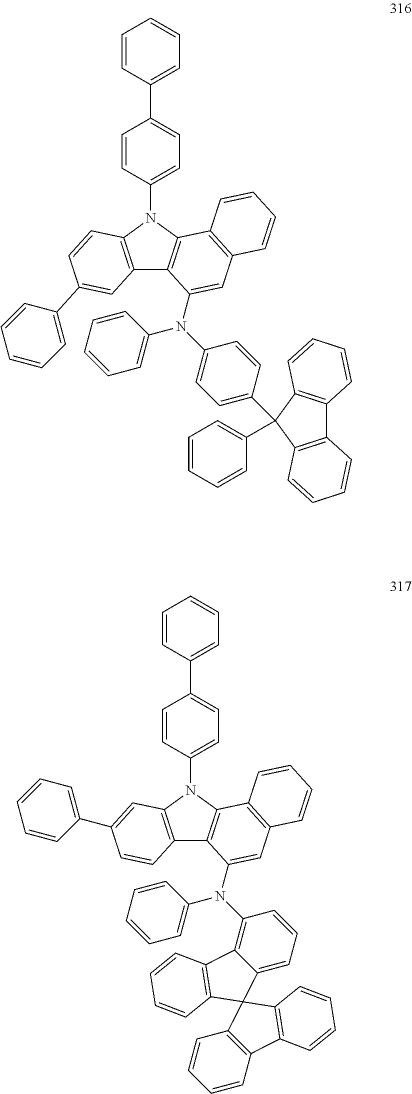 [SMILES: c1ccc(-c2ccc(-n3c4ccc(-c5ccccc5)cc4c4c(N(c5ccccc5)c5ccc(C6(c7ccccc7)c7ccccc7-c7ccccc76)cc5)cc5ccccc5c43)cc2)cc1 ; c1ccc(-c2ccc(-n3c4cc(-c5ccccc5)ccc4c4c(N(c5ccccc5)c5cccc6c5-c5ccccc5C65c6ccccc6-c6ccccc65)cc5ccccc5c43)cc2)cc1]

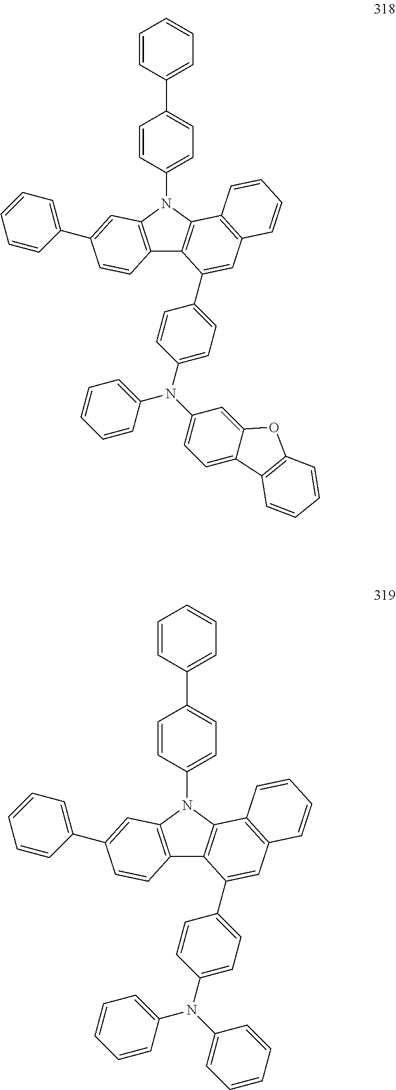 [SMILES: c1ccc(-c2ccc(-n3c4cc(-c5ccccc5)ccc4c4c(-c5ccc(N(c6ccccc6)c6ccc7c(c6)oc6ccccc67)cc5)cc5ccccc5c43)cc2)cc1 ; c1ccc(-c2ccc(-n3c4cc(-c5ccccc5)ccc4c4c(-c5ccc(N(c6ccccc6)c6ccccc6)cc5)cc5ccccc5c43)cc2)cc1]

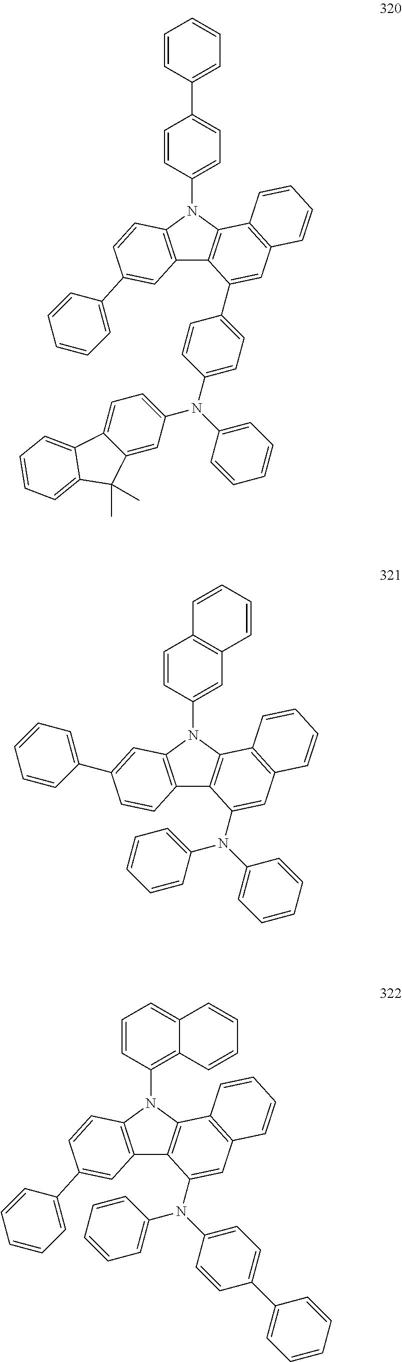 [SMILES: CC1(C)c2ccccc2-c2ccc(N(c3ccccc3)c3ccc(-c4cc5ccccc5c5c4c4cc(-c6ccccc6)ccc4n5-c4ccc(-c5ccccc5)cc4)cc3)cc21 ; c1ccc(-c2ccc3c4c(N(c5ccccc5)c5ccccc5)cc5ccccc5c4n(-c4ccc5ccccc5c4)c3c2)cc1 ; c1ccc(-c2ccc(N(c3ccccc3)c3cc4ccccc4c4c3c3cc(-c5ccccc5)ccc3n4-c3cccc4ccccc34)cc2)cc1]

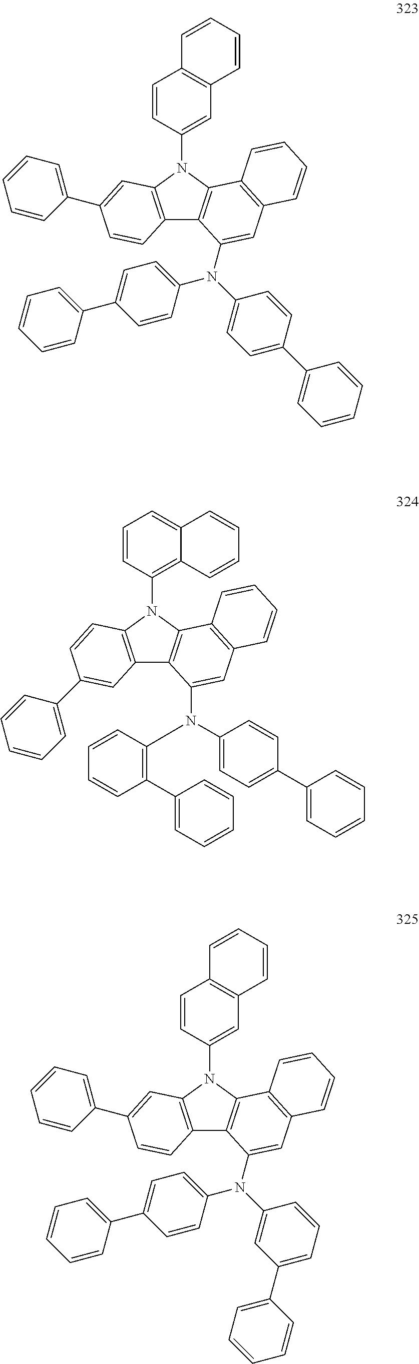 [SMILES: c1ccc(-c2ccc(N(c3ccc(-c4ccccc4)cc3)c3cc4ccccc4c4c3c3ccc(-c5ccccc5)cc3n4-c3ccc4ccccc4c3)cc2)cc1 ; c1ccc(-c2ccc(N(c3ccccc3-c3ccccc3)c3cc4ccccc4c4c3c3cc(-c5ccccc5)ccc3n4-c3cccc4ccccc34)cc2)cc1 ; c1ccc(-c2ccc(N(c3cccc(-c4ccccc4)c3)c3cc4ccccc4c4c3c3ccc(-c5ccccc5)cc3n4-c3ccc4ccccc4c3)cc2)cc1]

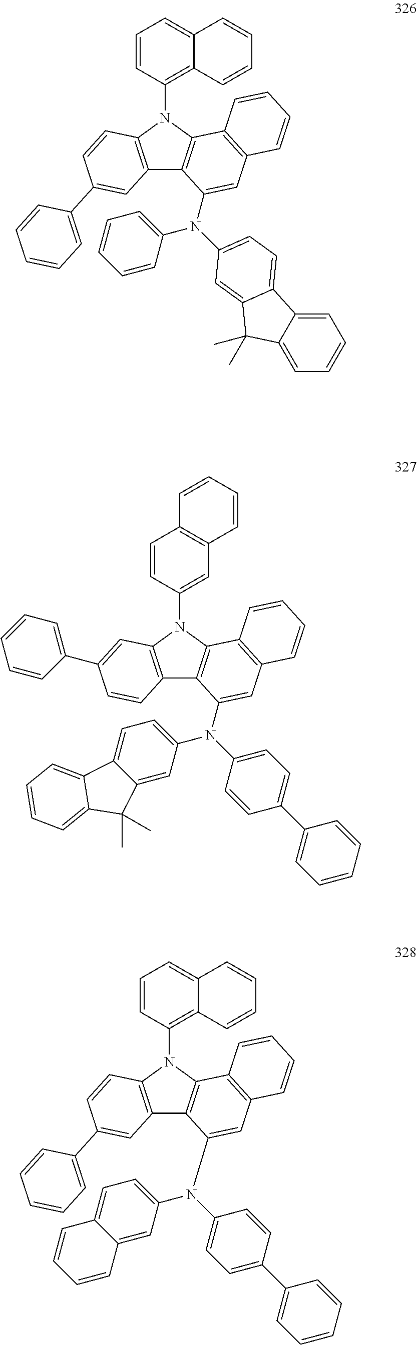 [SMILES: CC1(C)c2ccccc2-c2ccc(N(c3ccccc3)c3cc4ccccc4c4c3c3cc(-c5ccccc5)ccc3n4-c3cccc4ccccc34)cc21 ; CC1(C)c2ccccc2-c2ccc(N(c3ccc(-c4ccccc4)cc3)c3cc4ccccc4c4c3c3ccc(-c5ccccc5)cc3n4-c3ccc4ccccc4c3)cc21 ; c1ccc(-c2ccc(N(c3ccc4ccccc4c3)c3cc4ccccc4c4c3c3cc(-c5ccccc5)ccc3n4-c3cccc4ccccc34)cc2)cc1]

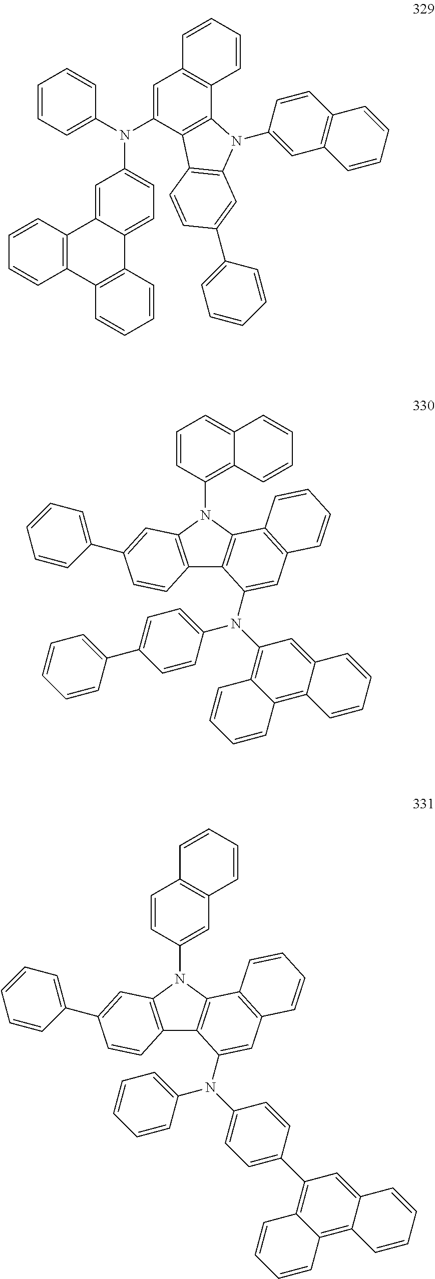 [SMILES: c1ccc(-c2ccc3c4c(N(c5ccccc5)c5ccc6c7ccccc7c7ccccc7c6c5)cc5ccccc5c4n(-c4ccc5ccccc5c4)c3c2)cc1 ; c1ccc(-c2ccc(N(c3cc4ccccc4c4ccccc34)c3cc4ccccc4c4c3c3ccc(-c5ccccc5)cc3n4-c3cccc4ccccc34)cc2)cc1 ; c1ccc(-c2ccc3c4c(N(c5ccccc5)c5ccc(-c6cc7ccccc7c7ccccc67)cc5)cc5ccccc5c4n(-c4ccc5ccccc5c4)c3c2)cc1]

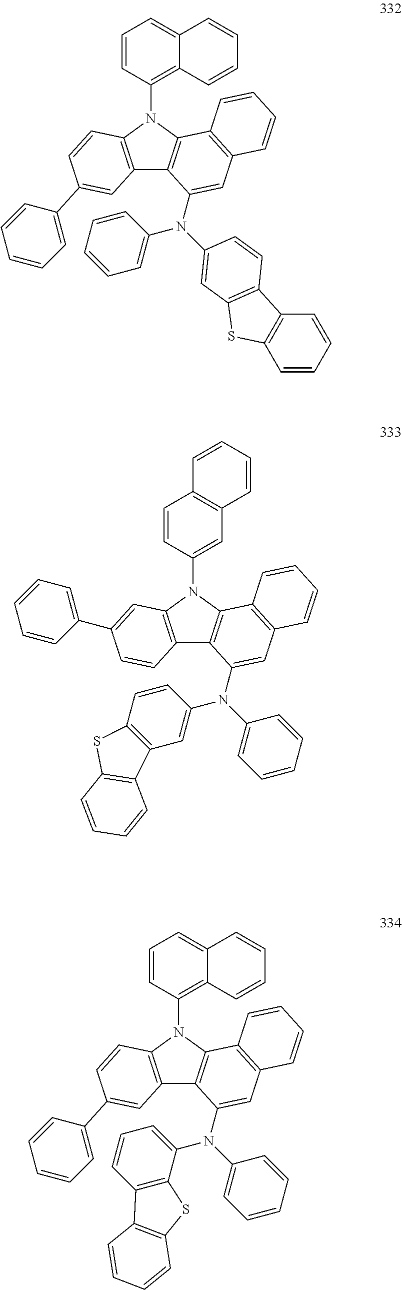 [SMILES: c1ccc(-c2ccc3c(c2)c2c(N(c4ccccc4)c4ccc5c(c4)sc4ccccc45)cc4ccccc4c2n3-c2cccc3ccccc23)cc1 ; c1ccc(-c2ccc3c4c(N(c5ccccc5)c5ccc6sc7ccccc7c6c5)cc5ccccc5c4n(-c4ccc5ccccc5c4)c3c2)cc1 ; c1ccc(-c2ccc3c(c2)c2c(N(c4ccccc4)c4cccc5c4sc4ccccc45)cc4ccccc4c2n3-c2cccc3ccccc23)cc1]

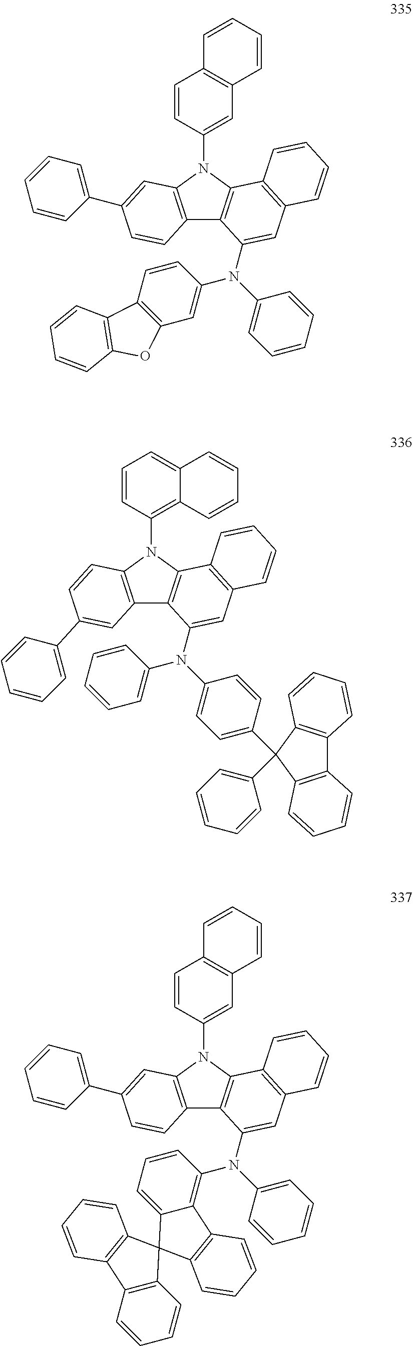 [SMILES: c1ccc(-c2ccc3c4c(N(c5ccccc5)c5ccc6c(c5)oc5ccccc56)cc5ccccc5c4n(-c4ccc5ccccc5c4)c3c2)cc1 ; c1ccc(-c2ccc3c(c2)c2c(N(c4ccccc4)c4ccc(C5(c6ccccc6)c6ccccc6-c6ccccc65)cc4)cc4ccccc4c2n3-c2cccc3ccccc23)cc1 ; c1ccc(-c2ccc3c4c(N(c5ccccc5)c5cccc6c5-c5ccccc5C65c6ccccc6-c6ccccc65)cc5ccccc5c4n(-c4ccc5ccccc5c4)c3c2)cc1]

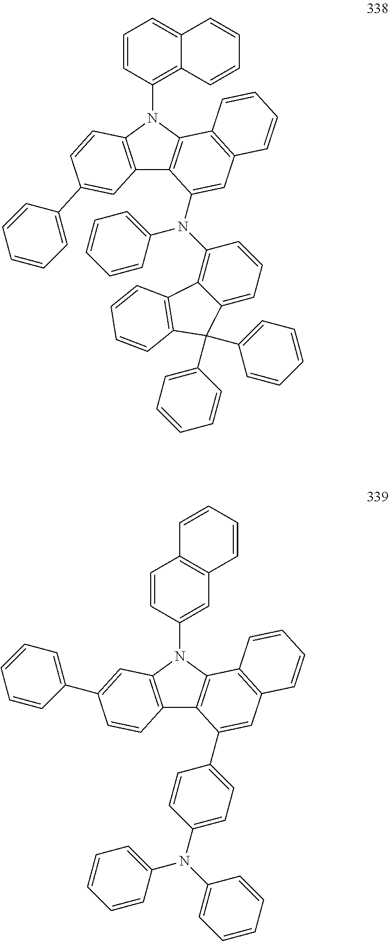 [SMILES: c1ccc(-c2ccc3c(c2)c2c(N(c4ccccc4)c4cccc5c4-c4ccccc4C5(c4ccccc4)c4ccccc4)cc4ccccc4c2n3-c2cccc3ccccc23)cc1 ; c1ccc(-c2ccc3c4c(-c5ccc(N(c6ccccc6)c6ccccc6)cc5)cc5ccccc5c4n(-c4ccc5ccccc5c4)c3c2)cc1]

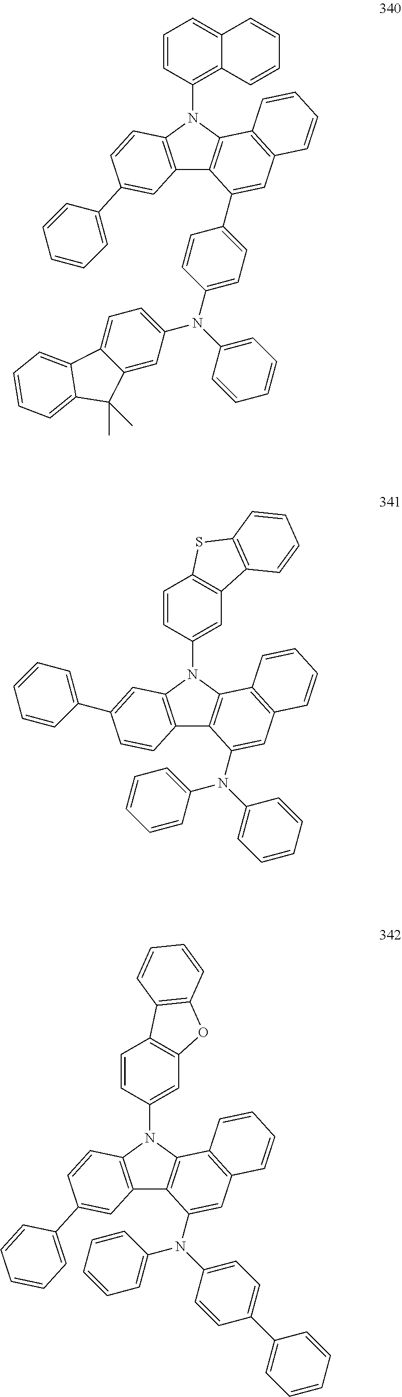 [SMILES: CC1(C)c2ccccc2-c2ccc(N(c3ccccc3)c3ccc(-c4cc5ccccc5c5c4c4cc(-c6ccccc6)ccc4n5-c4cccc5ccccc45)cc3)cc21 ; c1ccc(-c2ccc3c4c(N(c5ccccc5)c5ccccc5)cc5ccccc5c4n(-c4ccc5sc6ccccc6c5c4)c3c2)cc1 ; c1ccc(-c2ccc(N(c3ccccc3)c3cc4ccccc4c4c3c3cc(-c5ccccc5)ccc3n4-c3ccc4c(c3)oc3ccccc34)cc2)cc1]

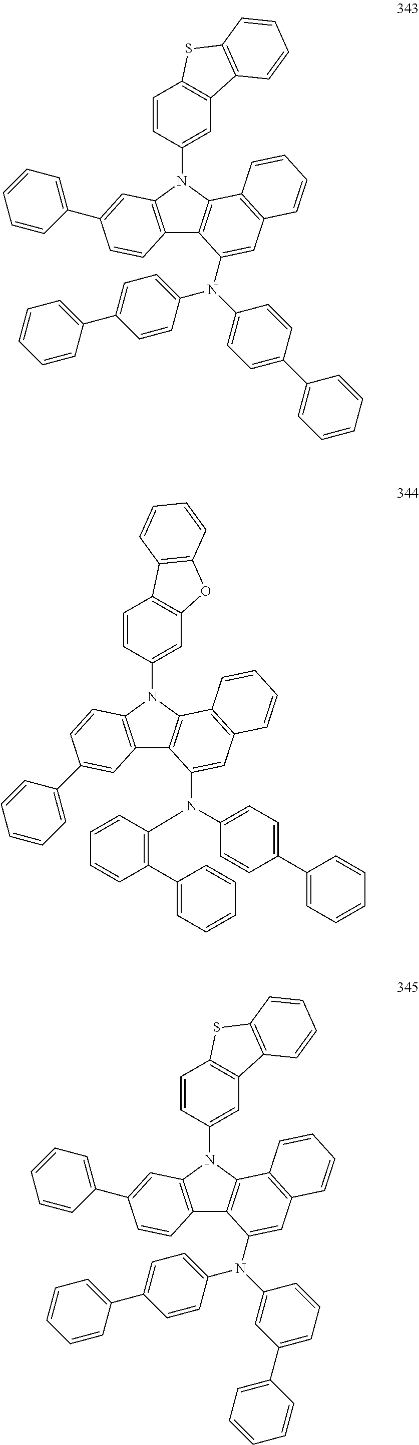 [SMILES: c1ccc(-c2ccc(N(c3ccc(-c4ccccc4)cc3)c3cc4ccccc4c4c3c3ccc(-c5ccccc5)cc3n4-c3ccc4sc5ccccc5c4c3)cc2)cc1 ; c1ccc(-c2ccc(N(c3ccccc3-c3ccccc3)c3cc4ccccc4c4c3c3cc(-c5ccccc5)ccc3n4-c3ccc4c(c3)oc3ccccc34)cc2)cc1 ; c1ccc(-c2ccc(N(c3cccc(-c4ccccc4)c3)c3cc4ccccc4c4c3c3ccc(-c5ccccc5)cc3n4-c3ccc4sc5ccccc5c4c3)cc2)cc1]

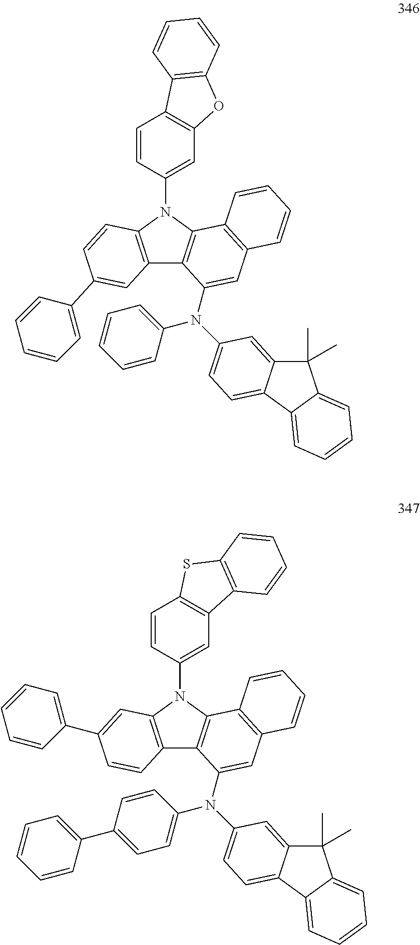 [SMILES: CC1(C)c2ccccc2-c2ccc(N(c3ccccc3)c3cc4ccccc4c4c3c3cc(-c5ccccc5)ccc3n4-c3ccc4c(c3)oc3ccccc34)cc21 ; CC1(C)c2ccccc2-c2ccc(N(c3ccc(-c4ccccc4)cc3)c3cc4ccccc4c4c3c3ccc(-c5ccccc5)cc3n4-c3ccc4sc5ccccc5c4c3)cc21]

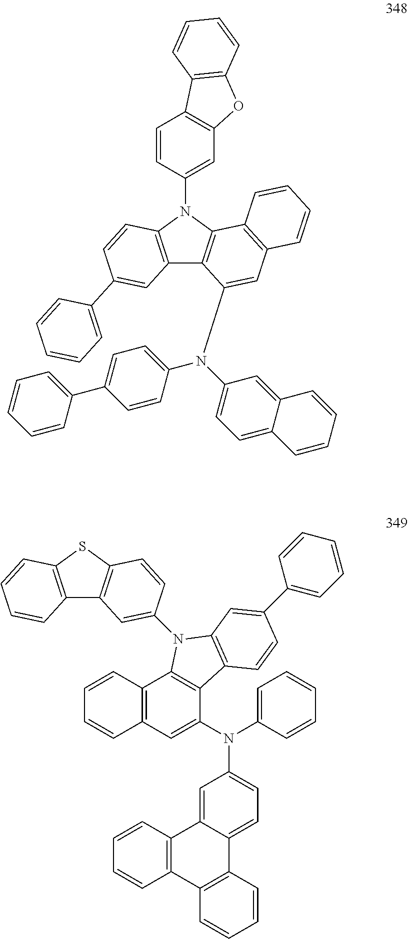 [SMILES: c1ccc(-c2ccc(N(c3ccc4ccccc4c3)c3cc4ccccc4c4c3c3cc(-c5ccccc5)ccc3n4-c3ccc4c(c3)oc3ccccc34)cc2)cc1 ; c1ccc(-c2ccc3c4c(N(c5ccccc5)c5ccc6c7ccccc7c7ccccc7c6c5)cc5ccccc5c4n(-c4ccc5sc6ccccc6c5c4)c3c2)cc1]

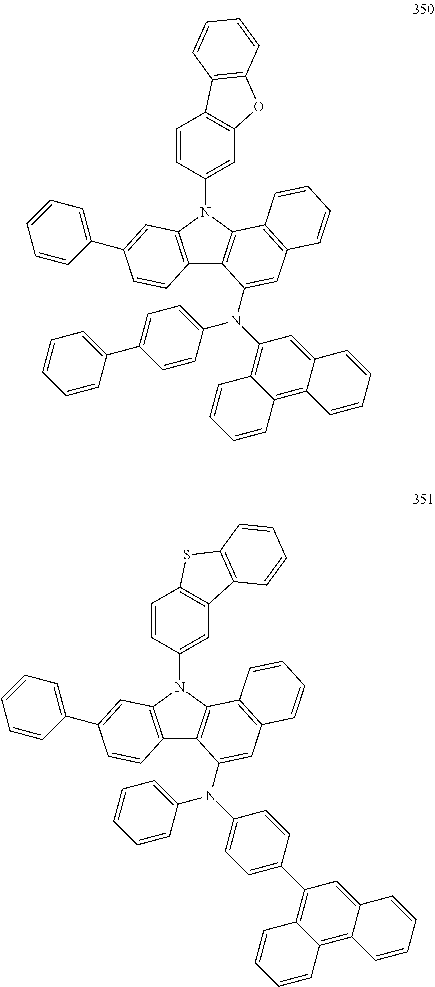 [SMILES: c1ccc(-c2ccc(N(c3cc4ccccc4c4ccccc34)c3cc4ccccc4c4c3c3ccc(-c5ccccc5)cc3n4-c3ccc4c(c3)oc3ccccc34)cc2)cc1 ; c1ccc(-c2ccc3c4c(N(c5ccccc5)c5ccc(-c6cc7ccccc7c7ccccc67)cc5)cc5ccccc5c4n(-c4ccc5sc6ccccc6c5c4)c3c2)cc1]

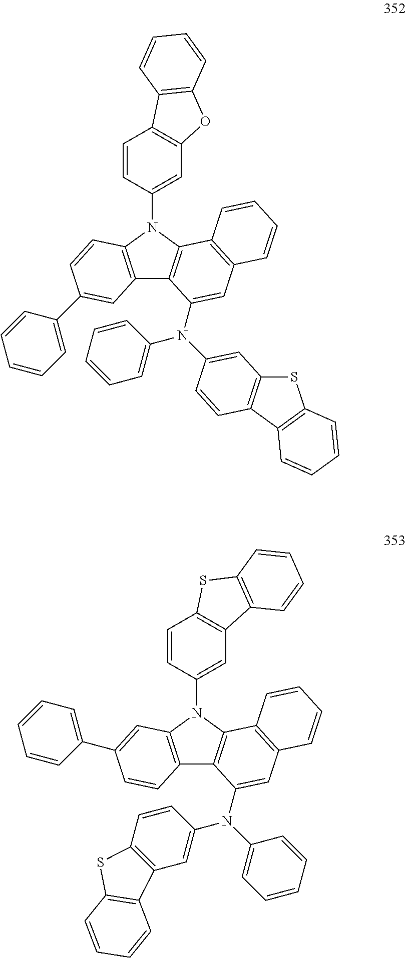 [SMILES: c1ccc(-c2ccc3c(c2)c2c(N(c4ccccc4)c4ccc5c(c4)sc4ccccc45)cc4ccccc4c2n3-c2ccc3c(c2)oc2ccccc23)cc1 ; c1ccc(-c2ccc3c4c(N(c5ccccc5)c5ccc6sc7ccccc7c6c5)cc5ccccc5c4n(-c4ccc5sc6ccccc6c5c4)c3c2)cc1]

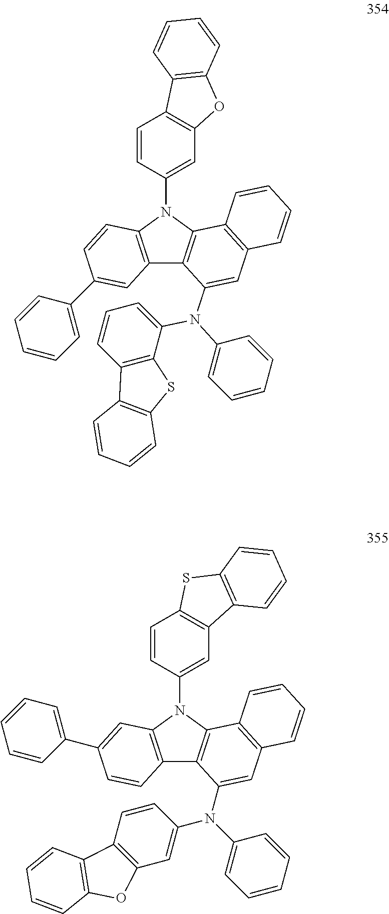 [SMILES: c1ccc(-c2ccc3c(c2)c2c(N(c4ccccc4)c4cccc5c4sc4ccccc45)cc4ccccc4c2n3-c2ccc3c(c2)oc2ccccc23)cc1 ; c1ccc(-c2ccc3c4c(N(c5ccccc5)c5ccc6c(c5)oc5ccccc56)cc5ccccc5c4n(-c4ccc5sc6ccccc6c5c4)c3c2)cc1]

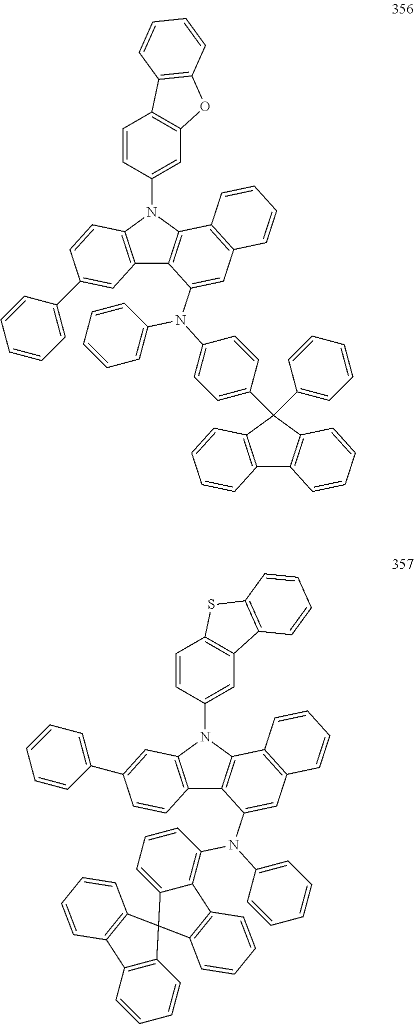 [SMILES: c1ccc(-c2ccc3c(c2)c2c(N(c4ccccc4)c4ccc(C5(c6ccccc6)c6ccccc6-c6ccccc65)cc4)cc4ccccc4c2n3-c2ccc3c(c2)oc2ccccc23)cc1 ; c1ccc(-c2ccc3c4c(N(c5ccccc5)c5cccc6c5-c5ccccc5C65c6ccccc6-c6ccccc65)cc5ccccc5c4n(-c4ccc5sc6ccccc6c5c4)c3c2)cc1]

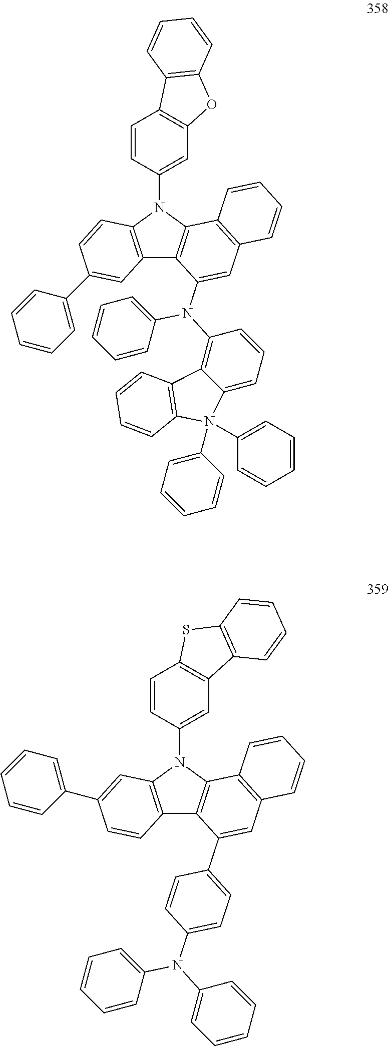

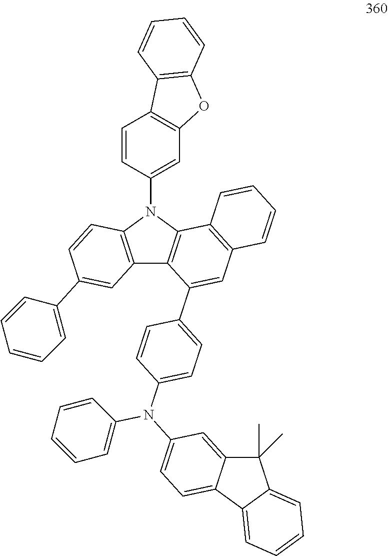 [SMILES: CC1(C)c2ccccc2-c2ccc(N(c3ccccc3)c3ccc(-c4cc5ccccc5c5c4c4cc(-c6ccccc6)ccc4n5-c4ccc5c(c4)oc4ccccc45)cc3)cc21]

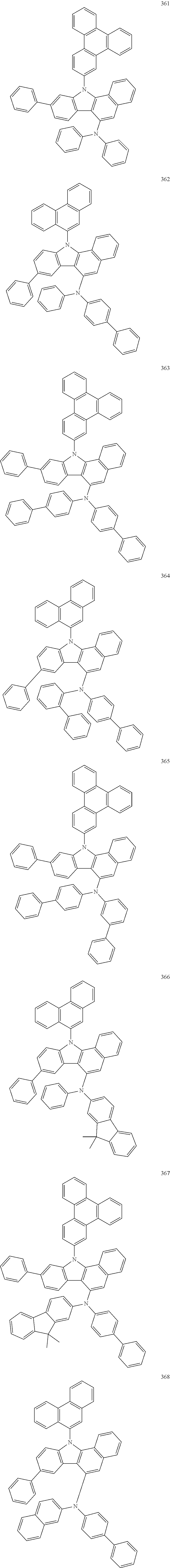

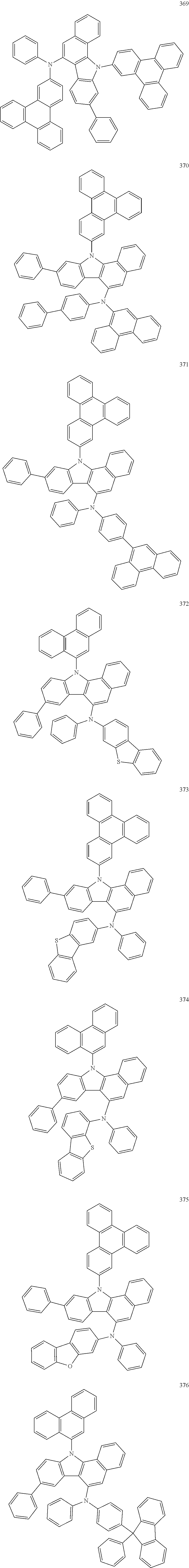

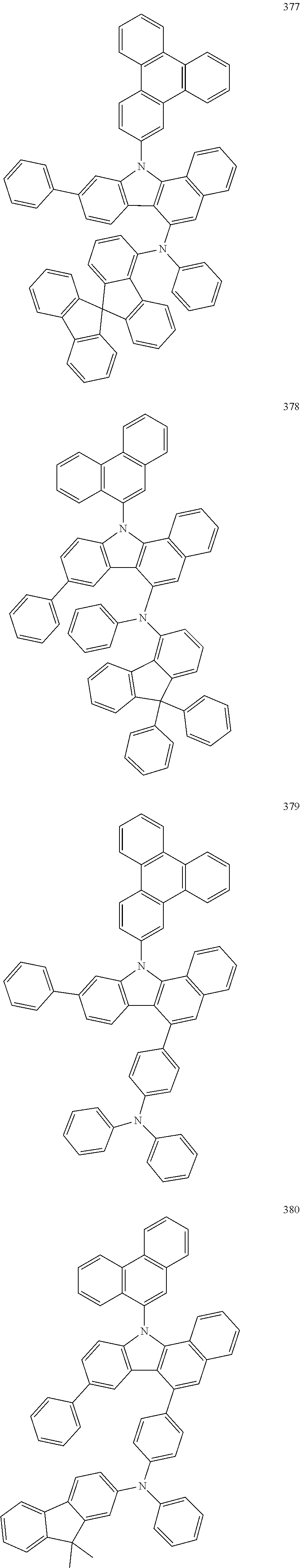

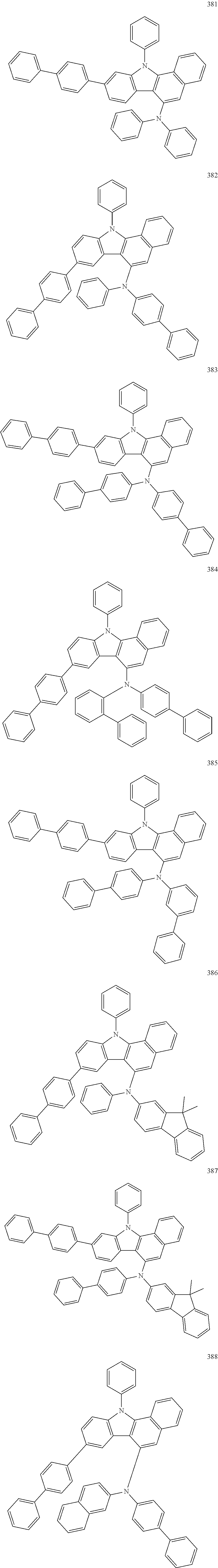

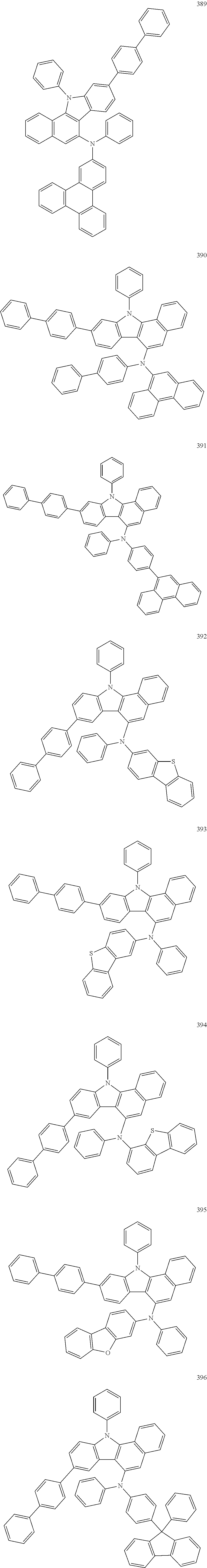

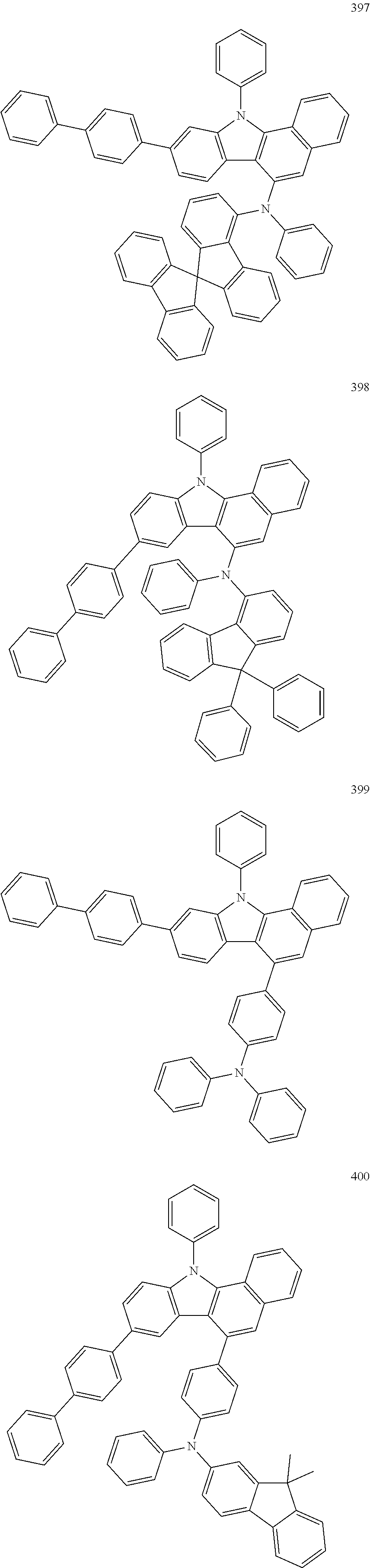 [SMILES: c1ccc(-c2ccc(-c3ccc4c5c(N(c6ccccc6)c6cccc7c6-c6ccccc6C76c7ccccc7-c7ccccc76)cc6ccccc6c5n(-c5ccccc5)c4c3)cc2)cc1 ; c1ccc(-c2ccc(-c3ccc4c(c3)c3c(N(c5ccccc5)c5cccc6c5-c5ccccc5C6(c5ccccc5)c5ccccc5)cc5ccccc5c3n4-c3ccccc3)cc2)cc1 ; c1ccc(-c2ccc(-c3ccc4c5c(-c6ccc(N(c7ccccc7)c7ccccc7)cc6)cc6ccccc6c5n(-c5ccccc5)c4c3)cc2)cc1 ; CC1(C)c2ccccc2-c2ccc(N(c3ccccc3)c3ccc(-c4cc5ccccc5c5c4c4cc(-c6ccc(-c7ccccc7)cc6)ccc4n5-c4ccccc4)cc3)cc21]

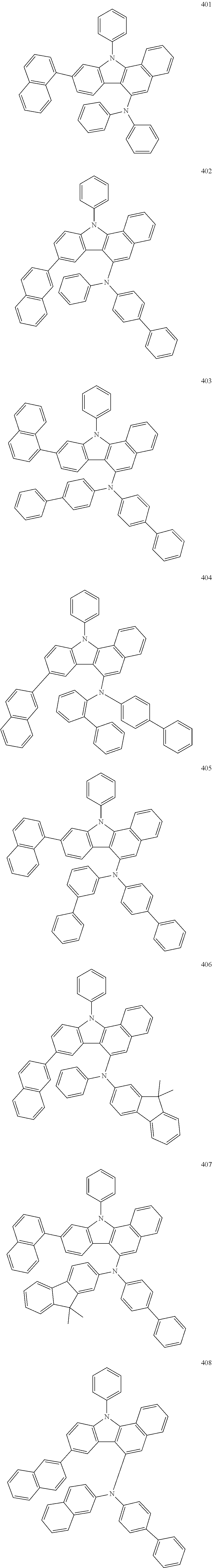

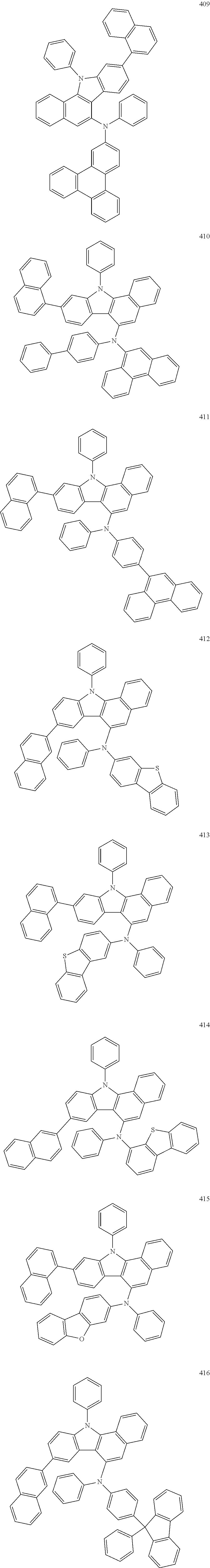

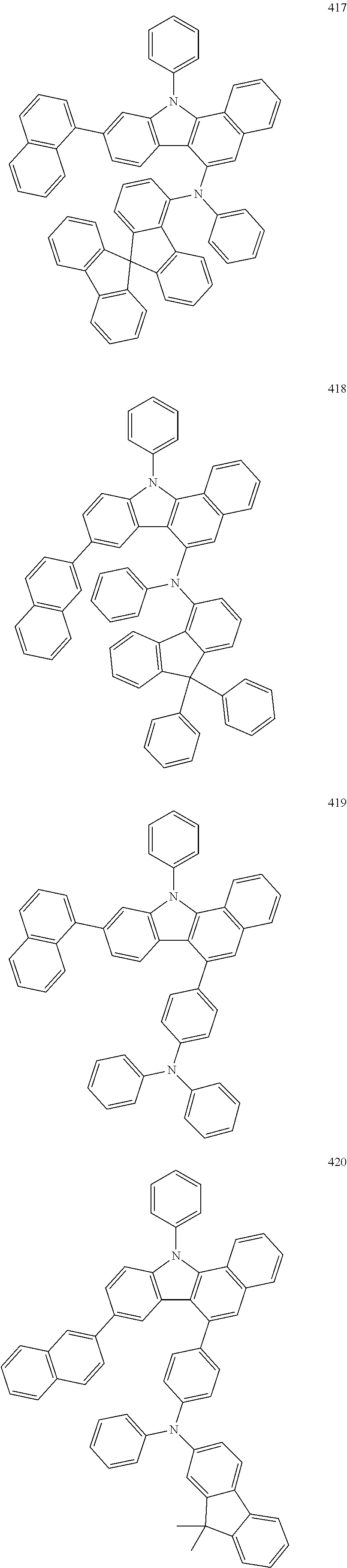 [SMILES: c1ccc(N(c2cccc3c2-c2ccccc2C32c3ccccc3-c3ccccc32)c2cc3ccccc3c3c2c2ccc(-c4cccc5ccccc45)cc2n3-c2ccccc2)cc1 ; c1ccc(N(c2cccc3c2-c2ccccc2C3(c2ccccc2)c2ccccc2)c2cc3ccccc3c3c2c2cc(-c4ccc5ccccc5c4)ccc2n3-c2ccccc2)cc1 ; c1ccc(N(c2ccccc2)c2ccc(-c3cc4ccccc4c4c3c3ccc(-c5cccc6ccccc56)cc3n4-c3ccccc3)cc2)cc1 ; CC1(C)c2ccccc2-c2ccc(N(c3ccccc3)c3ccc(-c4cc5ccccc5c5c4c4cc(-c6ccc7ccccc7c6)ccc4n5-c4ccccc4)cc3)cc21]

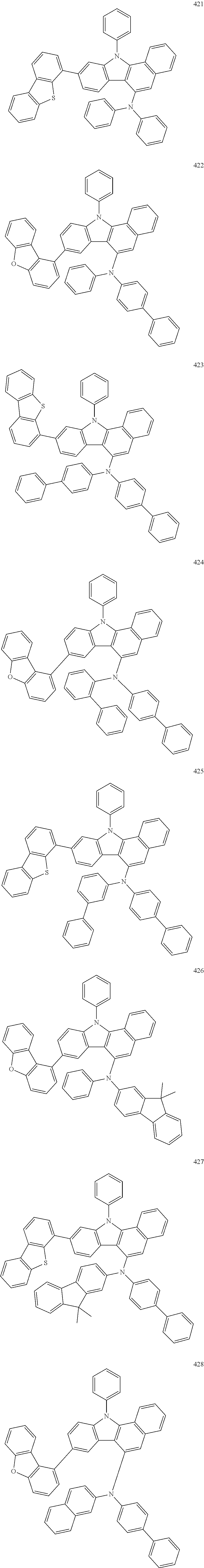

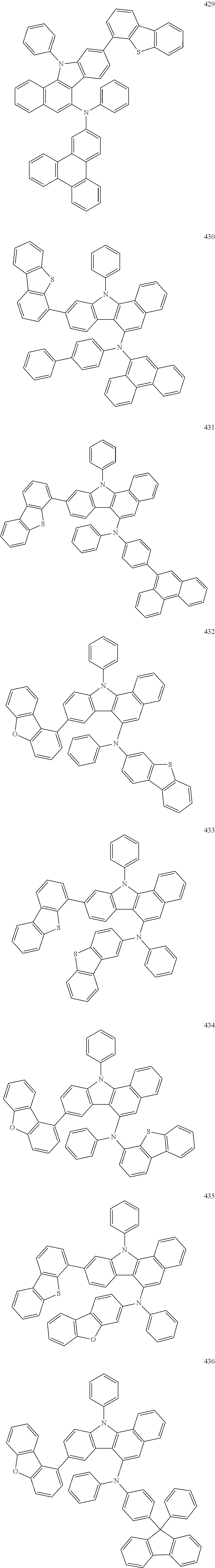

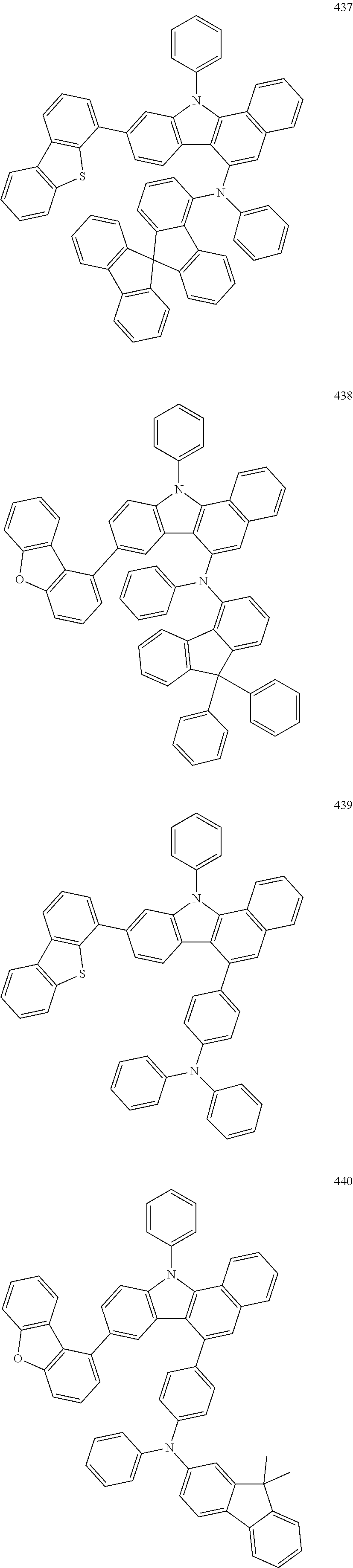 [SMILES: c1ccc(N(c2cccc3c2-c2ccccc2C32c3ccccc3-c3ccccc32)c2cc3ccccc3c3c2c2ccc(-c4cccc5c4sc4ccccc45)cc2n3-c2ccccc2)cc1 ; c1ccc(N(c2cccc3c2-c2ccccc2C3(c2ccccc2)c2ccccc2)c2cc3ccccc3c3c2c2cc(-c4cccc5oc6ccccc6c45)ccc2n3-c2ccccc2)cc1 ; c1ccc(N(c2ccccc2)c2ccc(-c3cc4ccccc4c4c3c3ccc(-c5cccc6c5sc5ccccc56)cc3n4-c3ccccc3)cc2)cc1 ; CC1(C)c2ccccc2-c2ccc(N(c3ccccc3)c3ccc(-c4cc5ccccc5c5c4c4cc(-c6cccc7oc8ccccc8c67)ccc4n5-c4ccccc4)cc3)cc21]

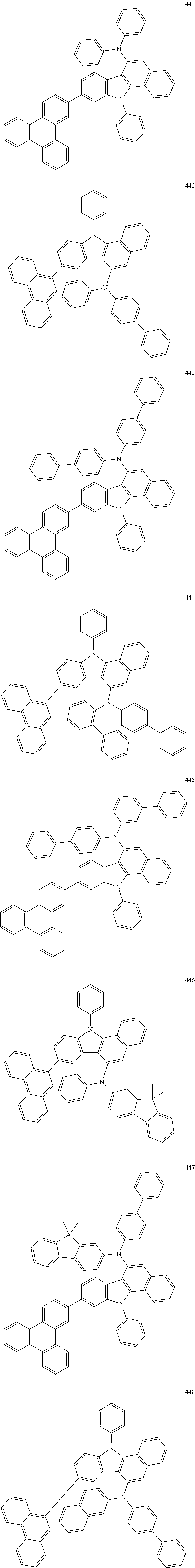

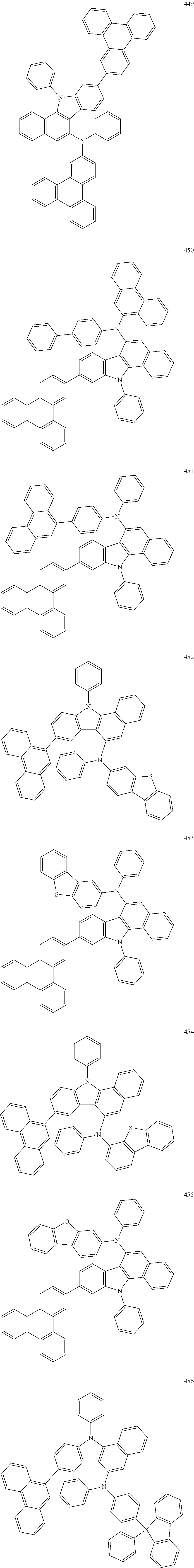

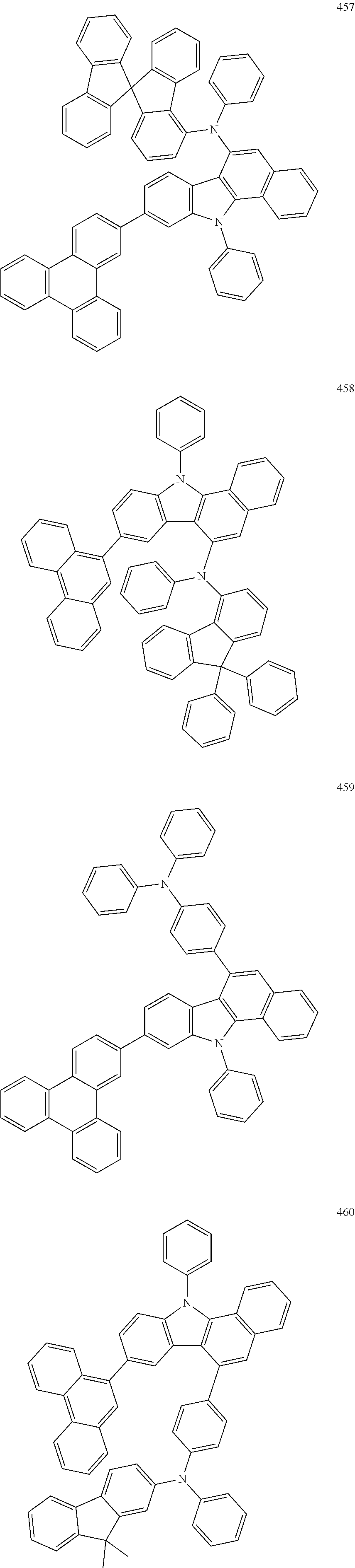 [SMILES: c1ccc(N(c2cccc3c2-c2ccccc2C32c3ccccc3-c3ccccc32)c2cc3ccccc3c3c2c2ccc(-c4ccc5c6ccccc6c6ccccc6c5c4)cc2n3-c2ccccc2)cc1 ; c1ccc(N(c2cccc3c2-c2ccccc2C3(c2ccccc2)c2ccccc2)c2cc3ccccc3c3c2c2cc(-c4cc5ccccc5c5ccccc45)ccc2n3-c2ccccc2)cc1 ; c1ccc(N(c2ccccc2)c2ccc(-c3cc4ccccc4c4c3c3ccc(-c5ccc6c7ccccc7c7ccccc7c6c5)cc3n4-c3ccccc3)cc2)cc1 ; CC1(C)c2ccccc2-c2ccc(N(c3ccccc3)c3ccc(-c4cc5ccccc5c5c4c4cc(-c6cc7ccccc7c7ccccc67)ccc4n5-c4ccccc4)cc3)cc21]

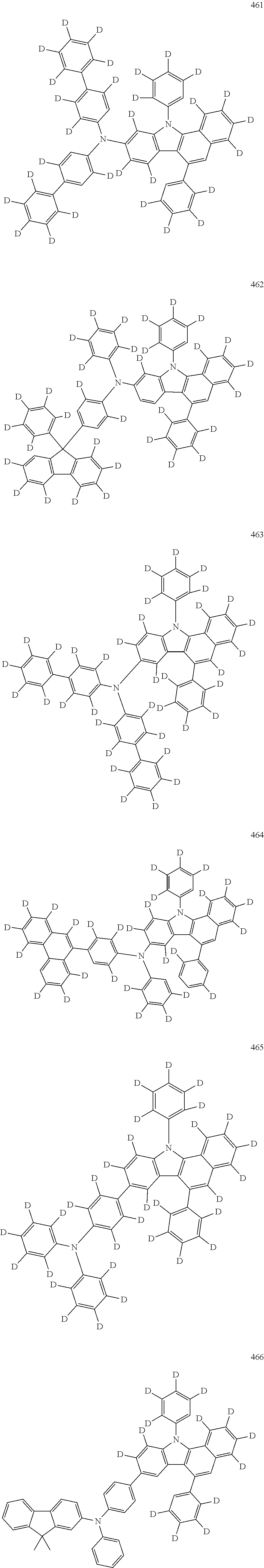

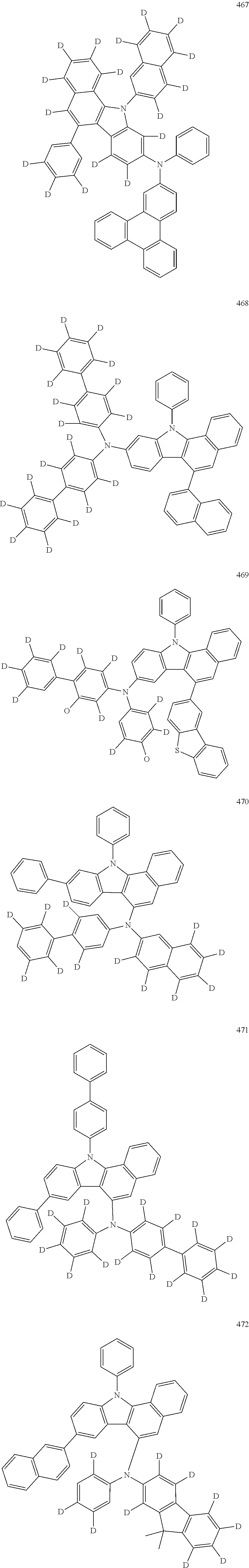

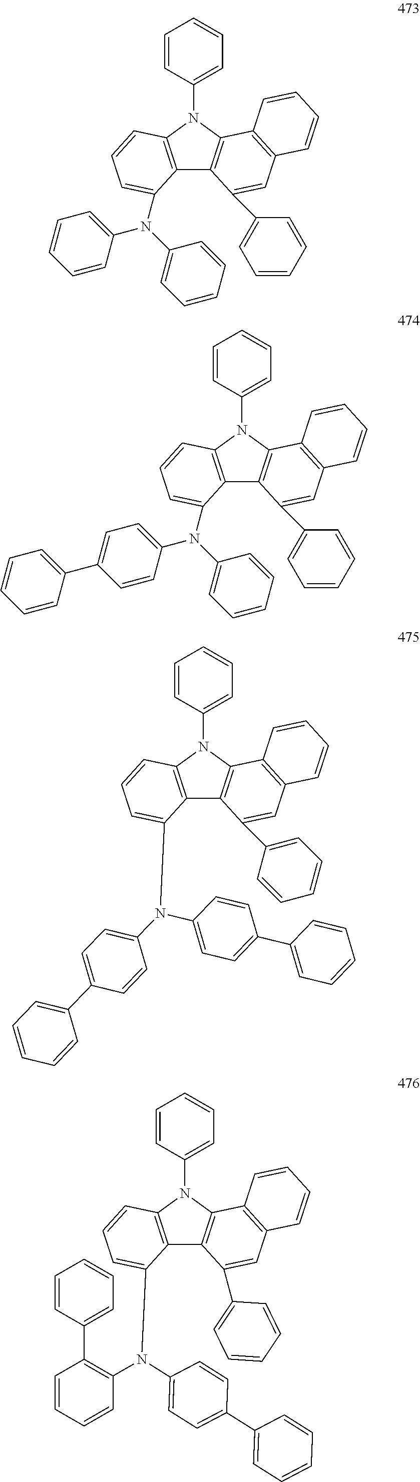

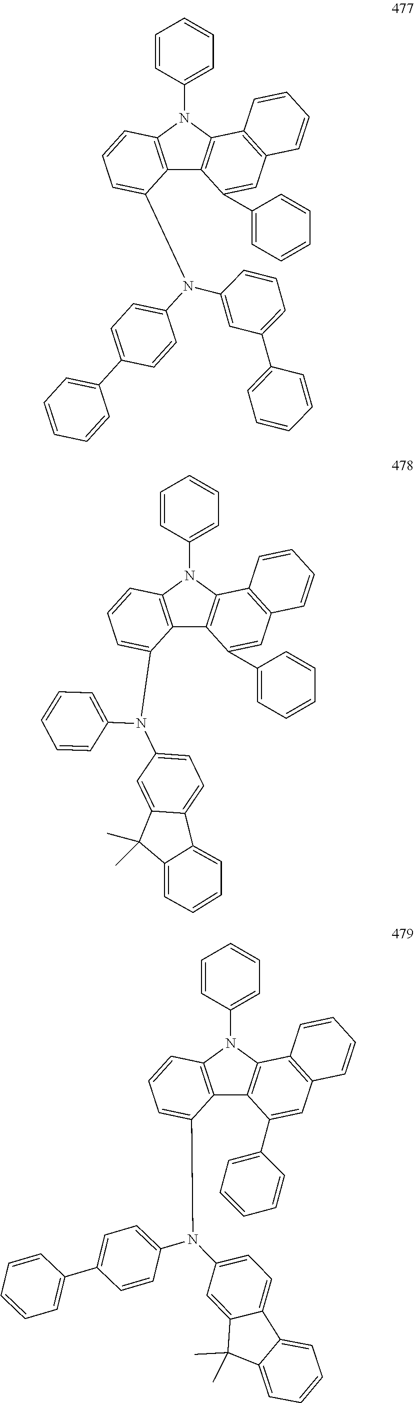 [SMILES: c1ccc(-c2ccc(N(c3cccc(-c4ccccc4)c3)c3cccc4c3c3c(-c5ccccc5)cc5ccccc5c3n4-c3ccccc3)cc2)cc1 ; CC1(C)c2ccccc2-c2ccc(N(c3ccccc3)c3cccc4c3c3c(-c5ccccc5)cc5ccccc5c3n4-c3ccccc3)cc21 ; CC1(C)c2ccccc2-c2ccc(N(c3ccc(-c4ccccc4)cc3)c3cccc4c3c3c(-c5ccccc5)cc5ccccc5c3n4-c3ccccc3)cc21]

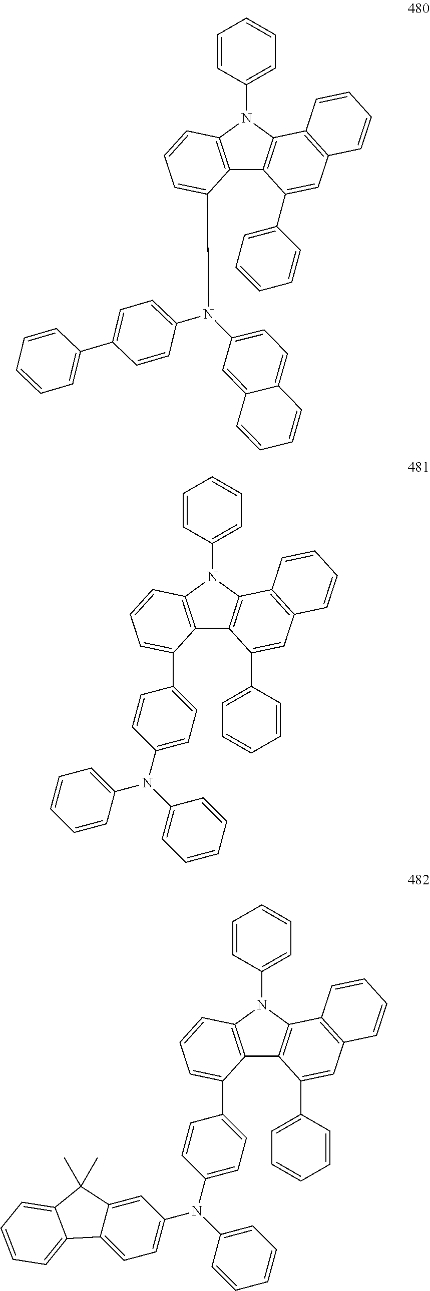 [SMILES: c1ccc(-c2ccc(N(c3ccc4ccccc4c3)c3cccc4c3c3c(-c5ccccc5)cc5ccccc5c3n4-c3ccccc3)cc2)cc1 ; c1ccc(-c2cc3ccccc3c3c2c2c(-c4ccc(N(c5ccccc5)c5ccccc5)cc4)cccc2n3-c2ccccc2)cc1 ; CC1(C)c2ccccc2-c2ccc(N(c3ccccc3)c3ccc(-c4cccc5c4c4c(-c6ccccc6)cc6ccccc6c4n5-c4ccccc4)cc3)cc21]

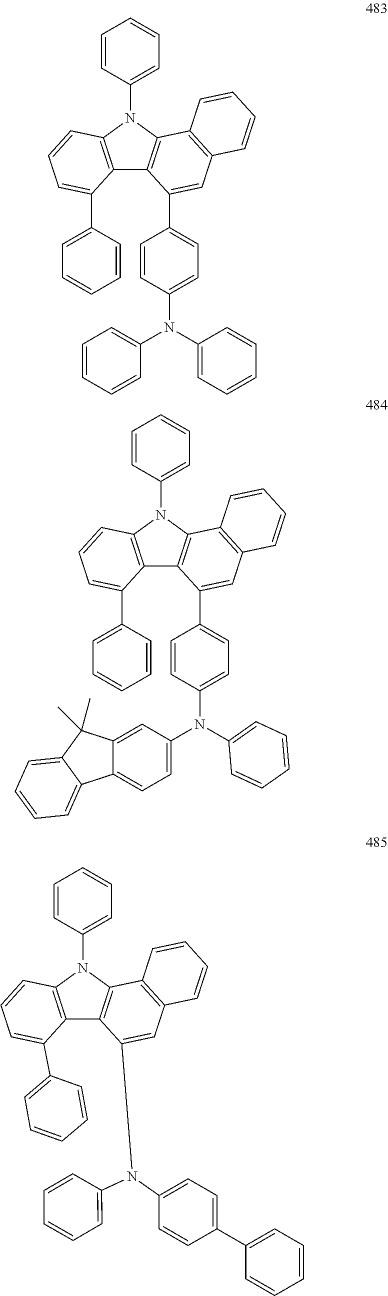

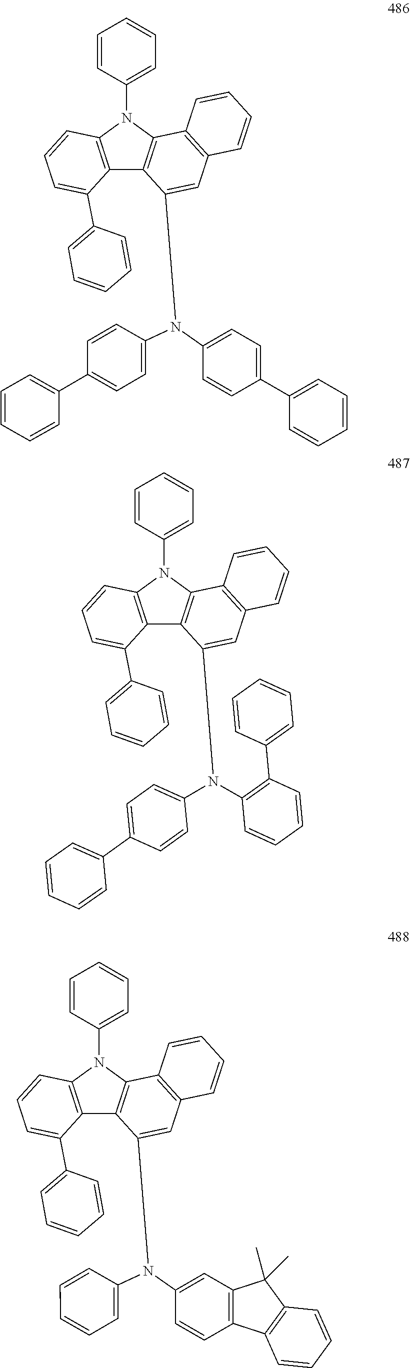 [SMILES: c1ccc(-c2ccc(N(c3ccc(-c4ccccc4)cc3)c3cc4ccccc4c4c3c3c(-c5ccccc5)cccc3n4-c3ccccc3)cc2)cc1 ; c1ccc(-c2ccc(N(c3ccccc3-c3ccccc3)c3cc4ccccc4c4c3c3c(-c5ccccc5)cccc3n4-c3ccccc3)cc2)cc1 ; CC1(C)c2ccccc2-c2ccc(N(c3ccccc3)c3cc4ccccc4c4c3c3c(-c5ccccc5)cccc3n4-c3ccccc3)cc21]

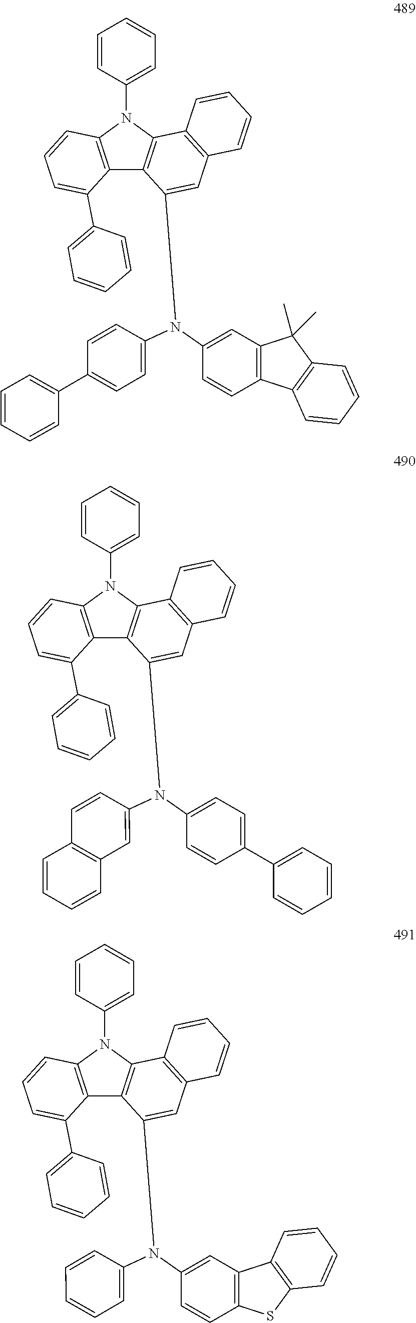 [SMILES: CC1(C)c2ccccc2-c2ccc(N(c3ccc(-c4ccccc4)cc3)c3cc4ccccc4c4c3c3c(-c5ccccc5)cccc3n4-c3ccccc3)cc21 ; c1ccc(-c2ccc(N(c3ccc4ccccc4c3)c3cc4ccccc4c4c3c3c(-c5ccccc5)cccc3n4-c3ccccc3)cc2)cc1 ; c1ccc(-c2cccc3c2c2c(N(c4ccccc4)c4ccc5sc6ccccc6c5c4)cc4ccccc4c2n3-c2ccccc2)cc1]

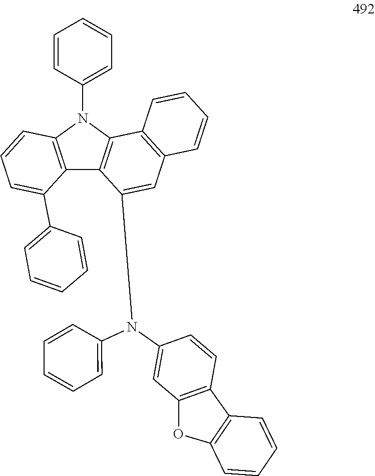 [SMILES: c1ccc(-c2cccc3c2c2c(N(c4ccccc4)c4ccc5c(c4)oc4ccccc45)cc4ccccc4c2n3-c2ccccc2)cc1]

In an exemplary embodiment of the present application, the compound is just one example and is not limited thereto, and may include other compounds included in Chemical Formula 1 which includes an additional substituent. In addition, the substitution position of deuterium of the compound may be present while a specific position is excluded and hydrogen and deuterium are mixed during the process of deuterium substitution and synthesis.

Further, various substituents may be introduced into the structure of Chemical Formula 1 to synthesize a compound having inherent characteristics of a substituent introduced. For example, a substituent usually used for a hole injection material, a hole transport material, a light emitting material, an electron transport material and an electron injection material, which are used when manufacturing an organic light emitting device, may be introduced into the core structure to synthesize a material which satisfies conditions required for each organic material layer.

In addition, by introducing various substituents into the structure of Chemical Formula 1 or changing the binding position, the bandgap may be finely adjusted, and meanwhile, the characteristics at the interface between the organic material layers may be improved.

In addition, the compound of Chemical Formula 1 has excellent thermal stability, and such thermal stability provides driving stability to the organic light emitting device and improves service life characteristics.

In an exemplary embodiment of the present application, provided is an organic light emitting device including: a first electrode; a second electrode provided to face the first electrode; and an organic material layer having one or more layers provided between the first electrode and the second electrode, in which the one or more layers of the organic material layer include the heterocyclic compound represented by Chemical Formula 1.

In another exemplary embodiment, provided is an organic light emitting device including: a first electrode; a second electrode provided to face the first electrode; and an organic material layer having one or more layers provided between the first electrode and the second electrode, in which the one or more layers of the organic material layer include one heterocyclic compound represented by Chemical Formula 1.

In still another exemplary embodiment, provided is an organic light emitting device including: a first electrode; a second electrode provided to face the first electrode; and an organic material layer having one or more layers provided between the first electrode and second electrode, in which the one or more layers of the organic material layer include two or more of the heterocyclic compound represented by Chemical Formula 1.

In yet another exemplary embodiment, the heterocyclic compound represented by Chemical Formula 1 can be used as a light emitting material for a light emitting layer of the organic light emitting device.

In still yet another exemplary embodiment, the heterocyclic compound represented by Chemical Formula 1 can be used as a host material for a light emitting layer of the organic light emitting device.

In an exemplary embodiment of the present application, the first electrode may be a positive electrode, and the second electrode may be a negative electrode.

In another exemplary embodiment, the first electrode may be a negative electrode, and the second electrode may be a positive electrode.

In an exemplary embodiment of the present application, the organic light emitting device may be a blue organic light emitting device, and the heterocyclic compound according to Chemical Formula 1 may be used as a material for the blue organic light emitting device.

In an exemplary embodiment of the present application, the organic light emitting device may be a green organic light emitting device, and the heterocyclic compound represented by Chemical Formula 1 may be used as a material for the green organic light emitting device.

In an exemplary embodiment of the present application, the organic light emitting device may be a red organic light device, and the heterocyclic compound represented by Chemical Formula 1 may be used as a material for the red organic light emitting device.

In an exemplary embodiment of the present application, the organic light emitting device may be a blue organic light emitting device, and the heterocyclic compound according to Chemical Formula 1 may be used as a material for a light emitting layer of the blue organic light emitting device.

In an exemplary embodiment of the present application, the organic light emitting device may be a green organic light emitting device, and the heterocyclic compound represented by Chemical Formula 1 may be used as a material for a light emitting layer of the green organic light emitting device.

In an exemplary embodiment of the present application, the organic light emitting device may be a red organic light emitting device, and the heterocyclic compound represented by Chemical Formula 1 may be used as a material for a light emitting layer of the red organic light emitting device.

In an exemplary embodiment of the present application, the specific content on the heterocyclic compound represented by Chemical Formula 1 is the same as that described above.

The organic light emitting device of the present invention may be manufactured using typical manufacturing methods and materials of an organic light emitting device, except that the above-described heterocyclic compound is used to form an organic material layer having one or more layers.

The heterocyclic compound may be formed as an organic material layer by not only a vacuum deposition method, but also a solution application method when an organic light emitting device is manufactured. Here, the solution application method means spin coating, dip coating, inkjet printing, screen printing, a spray method, roll coating, and the like, but is not limited thereto.

The organic material layer of the organic light emitting device of the present invention may be composed of a single-layered structure, but may be composed of a multi-layered structure in which two or more organic material layers are stacked. For example, the organic light emitting device of the present invention may have a structure including a hole injection layer, a hole transport layer, a light emitting layer, an electron transport layer, an electron injection layer, and the like as organic material layers. However, the structure of the organic light emitting device is not limited thereto, and may include a fewer number of organic material layers.

In an exemplary embodiment of the present application, as the iridium-based dopant, Ir(ppy)₃, which is a green phosphorescent dopant, may be used.

In an exemplary embodiment of the present application, as the iridium-based dopant, (piq)₂(Ir)(acac), which is a red phosphorescent dopant, may be used.

In an exemplary embodiment of the present application, provided is an organic light emitting device, in which the organic material layer of the organic light emitting device includes a light emitting layer, and the light emitting layer includes the heterocyclic compound.

In an exemplary embodiment of the present application, provided is an organic light emitting device, in which the organic material layer of the organic light emitting device includes a light emitting layer, and the light emitting layer includes a host material, and the host material includes the heterocyclic compound.

In the organic light emitting device of the present invention, the organic material layer includes an electron injection layer or an electron transport layer, and the electron injection layer or electron transport layer may include the heterocyclic compound.

In another organic light emitting device, the organic material layer includes a hole blocking layer, and the hole blocking layer may include the heterocyclic compound.

In still another organic light emitting device, the organic material layer includes an electron blocking layer, and the electron blocking layer may include the heterocyclic compound.

In yet another organic light emitting device, the organic material layer includes a hole transport layer, a light emitting layer or an electron blocking layer, and the hole transport layer, the light emitting layer or the electron blocking layer may include the heterocyclic compound.

In still yet another organic light emitting device, the organic material layer includes a hole transport layer or a hole transport auxiliary layer, and the hole transport layer or the hole transport auxiliary layer may include the heterocyclic compound.

In the organic light emitting device of the present application, as a positive electrode material, materials having a relatively high work function may be used, and a transparent conductive oxide, a metal or a conductive polymer, and the like may be used. Specific examples of the positive electrode material include: a metal such as vanadium, chromium, copper, zinc, and gold, or an alloy thereof; a metal oxide such as zinc oxide, indium oxide, indium tin oxide (ITO), and indium zinc oxide (IZO); a combination of a metal and an oxide, such as ZnO:Al or SnO₂:Sb; a conductive polymer such as poly(3-methylthiophene), poly[3,4-(ethylene-1,2-dioxy)thiophene] (PEDOT), polypyrrole, and polyaniline; and the like, but are not limited thereto.

As a negative electrode material, materials having a relatively low work function may be used, and a metal, a metal oxide, or a conductive polymer, and the like may be used. Specific examples of the negative electrode material include: a metal such as magnesium, calcium, sodium, potassium, titanium, indium, yttrium, lithium, gadolinium, aluminum, silver, tin, and lead, or an alloy thereof; a multi-layer structured material, such as LiF/Al or LiO₂/Al; and the like, but are not limited thereto.

As a hole injection material, a publicly-known hole injection material may also be used, and it is possible to use, for example, a phthalocyanine compound such as copper phthalocyanine disclosed in U.S. Pat. No. 4,356,429 or starburst-type amine derivatives described in the document [Advanced Material, 6, p. 677 (1994)], for example, tris(4-carbazoyl-9-ylphenyl)amine (TCTA), 4,4′,4″-tri[phenyl(m-tolyl)amino]triphenylamine (m-MTDATA), 1,3,5-tris[4-(3-methylphenylphenylamino)phenyl]benzene (m-MTDAPB), polyaniline/dodecylbenzenesulfonic acid or poly(3,4-ethylenedioxythiophene)/poly(4-styrenesulfonate), which is a soluble conductive polymer, polyaniline/camphor sulfonic acid or polyaniline/poly(4-styrene-sulfonate), and the like.

As a hole transport material, a pyrazoline derivative, an arylamine-based derivative, a stilbene derivative, a triphenyldiamine derivative, and the like may be used, and a low-molecular weight or polymer material may also be used.

As an electron transport material, it is possible to use an oxadiazole derivative, anthraquinodimethane and a derivative thereof, benzoquinone and a derivative thereof, naphthoquinone and a derivative thereof, anthraquinone and a derivative thereof, tetracyanoanthraquinodimethane and a derivative thereof, a fluorenone derivative, diphenyldicyanoethylene and a derivative thereof, a diphenoquinone derivative, a metal complex of 8-hydroxyquinoline and a derivative thereof, and the like, and a low-molecular weight material and a polymer material may also be used.

As an electron injection material, for example, LiF is representatively used in the art, but the present application is not limited thereto.

As a light emitting material, a red, green, or blue light emitting material may be used, and if necessary, two or more light emitting materials may be mixed and used. In this case, two or more light emitting materials are deposited or used as an individual supply source, or pre-mixed to be deposited and used as one supply source. Further, a fluorescent material may also be used as the light emitting material, but may also be used as a phosphorescent material. As the light emitting material, it is also possible to use alone a material which emits light by combining holes and electrons each injected from a positive electrode and a negative electrode, but materials in which a host material and a dopant material are involved in light emission together may also be used.

When hosts of the light emitting material are mixed and used, the same series of hosts may also be mixed and used, and different series of hosts may also be mixed and used. For example, two or more types of materials selected from n-type host materials or p-type host materials may be used as a host material for a light emitting layer.

The organic light emitting device according to an exemplary embodiment of the present application may be a top emission type, a bottom emission type, or a dual emission type according to the material to be used.

The heterocyclic compound according to an exemplary embodiment of the present application may act even in organic electronic devices including organic solar cells, organic photoconductors, organic transistors, and the like, based on the principle similar to those applied to organic light emitting devices.

The organic light emitting device of the present invention may further include one or two or more layers selected from the group consisting of a light emitting layer, a hole injection layer, a hole transport layer, a hole transport auxiliary layer, an electron blocking layer, a hole blocking layer, an electron transport layer and an electron injection layer.

The organic light emitting device of the present invention may further include one or two or more layers selected from the group consisting of a light emitting layer, a hole injection layer, a hole transport layer, an electron blocking layer, a hole blocking layer, an electron transport layer and an electron injection layer.

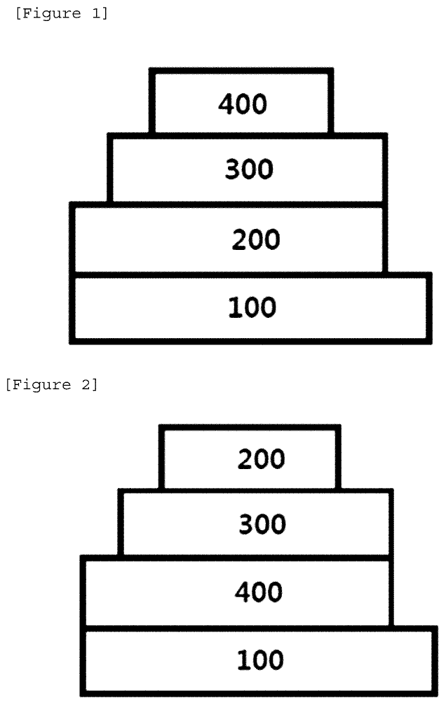

FIGS. 1 to 4 exemplify the stacking sequence of the electrodes and the organic material layer of the organic light emitting device according to an exemplary embodiment of the present application. However, the scope of the present application is not intended to be limited by these drawings, and the structure of the organic light emitting device known in the art may also be applied to the present application.

According to FIG. 1, an organic light emitting device in which a positive electrode 200, an organic material layer 300, and a negative electrode 400 are sequentially stacked on a substrate 100 is illustrated. However, the organic light emitting device is not limited only to such a structure, and as in FIG. 2, an organic light emitting device in which a negative electrode, an organic material layer, and a positive electrode are sequentially stacked on a substrate may also be implemented.

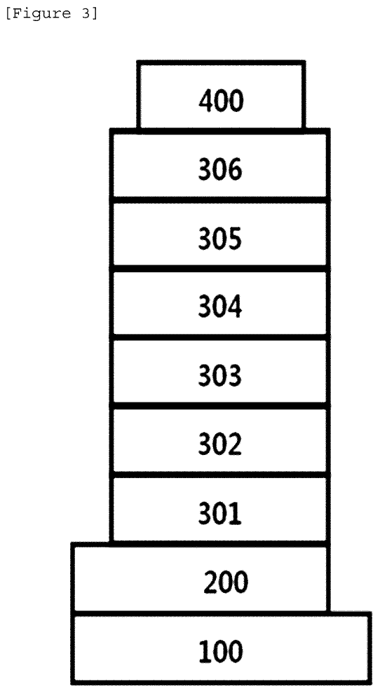

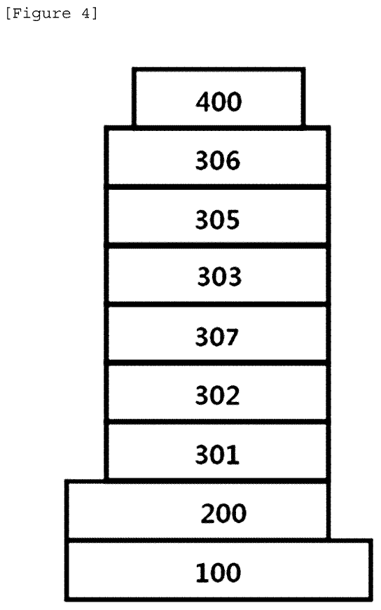

FIGS. 3 and 4 exemplify a case where an organic material layer is a multilayer.

An organic light emitting device according to FIG. 3 includes a hole injection layer 301, a hole transport layer 302, a light emitting layer 303, a hole blocking layer 304, an electron transport layer 305, and an electron injection layer 306.

An organic light emitting device according to FIG. 4 includes a hole injection layer 301, a hole transport layer 302, an electron blocking layer 307, a light emitting layer 303, an electron transport layer 305, and an electron injection layer 306. However, the scope of the present application is not limited by the stacking structure as described above, and if necessary, the other layers except for the light emitting layer may be omitted, and another necessary functional layer may be further added.

In an exemplary embodiment of the present application, provided is a method for manufacturing an organic light emitting device, the method including: preparing a substrate; forming a first electrode on the substrate; forming an organic material layer having one or more layers on the first electrode; and forming a second electrode on the organic material layer, in which the forming of the organic material layer includes forming the organic material layer having one or more layers by using the composition for an organic material layer including the heterocyclic compound according to an exemplary embodiment of the present application.

Hereinafter, the present specification will be described in more detail through Examples, but these Examples are provided only for exemplifying the present application, and are not intended to limit the scope of the present application.

### SYNTHESIS EXAMPLES

### [Preparation Example 1] Preparation of Compound 003

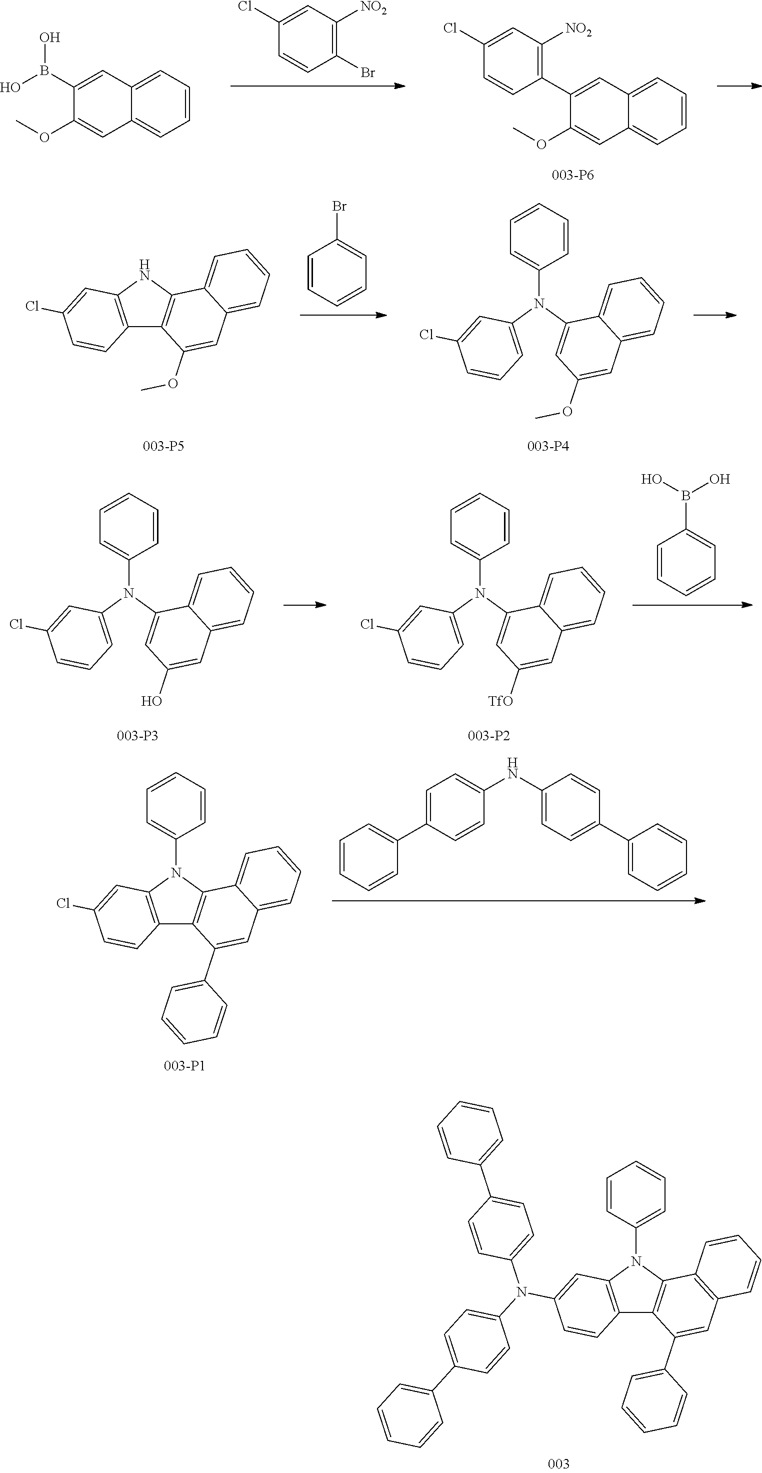

### 1) Preparation of Compound 003-P6

After (3-methoxynaphthalen-2-yl)boronic acid (50 g, 247.51 mmol) and 1-bromo-4-chloro-2-nitrobenzene (61.45 g, 259.89 mmol) were dissolved in 500 ml of 1,4-dioxane and 100 ml of distilled water, Pd(PPh₃)₄ (14.30 g, 12.38 mmol) and K₂CO₃ (85.52 g, 618.78 mmol) were added thereto, and the resulting mixture was stirred under reflux for 12 hours. After the reaction was completed, the reaction solution was extracted with dichloromethane and distilled water, the organic layer was dried over anhydrous MgSO₄, and then the solvent was removed by a rotary evaporator, and then the residue was purified by column chromatography using dichloromethane and hexane as an eluting solvent, thereby obtaining Compound 003-P6 (64 g, 82%).

### 2) Preparation of Compound 003-P5

Compound 003-P6 (64 g, 203.99 mmol) and triphenylphosphine (133.76 g, 509.98 mmol) were added to 700 ml of 1,2-dichlorobenzene, and the resulting mixture was stirred under reflux for 7 hours. After the reaction was completed, dichloromethane was added to the reaction solution for dissolution, and then the resulting solution was extracted with distilled water, the organic layer was dried over anhydrous MgSO₄, and then the solvent was removed by a rotary evaporator, and then the residue was purified by column chromatography using dichloromethane and hexane as an eluting solvent, thereby obtaining Compound 003-P5 (48 g, 86%).

### 3) Preparation of Compound 003-P4

After Compound 003-P5 (48 g 170.37 mmol) and bromobenzene (32.1 g, 204.44 mmol) were dissolved in 500 ml of toluene, Pd₂(dba)₃ (7.80 g, 8.52 mmol), Xphos (8.12 g, 17.04 mmol), and NaOtBu (32.75 g, 340.74 mmol) were added thereto and the resulting mixture was stirred under reflux for 3 hours. After the reaction was completed, the reaction was extracted with dichloromethane and distilled water, the organic layer was dried over anhydrous MgSO₄, and then the solvent was removed by a rotary evaporator, and then the residue was purified by column chromatography using dichloromethane and hexane as an eluting solvent, thereby obtaining Compound 003-P4 (45 g, 74%).

### 4) Preparation of Compound 003-P3

After Compound 003-P4 (45 g, 125.76 mmol) was dissolved in 500 ml of dichloromethane, boron tribromide (47.26 g, 188.64 mmol) was slowly added thereto at 0° C., and then the resulting mixture was stirred for 3 hours. After the reaction was completed, and then terminated by slowly adding distilled water to the reaction solution, extraction was performed using dichloromethane and distilled water, the organic layer was dried over anhydrous MgSO₄, and then the solvent was removed by a rotary evaporator, and then, the residue was purified by column chromatography using dichloromethane and hexane as an eluting solvent, thereby obtaining Compound 003-P3 (39 g, 90%).

### 5) Preparation of Compound 003-P2

After Compound 003-P3 (39 g, 74.85 mmol) was dissolved in 400 ml of dichloromethane, triethylamine (13.77 g, 136.12 mmol) was added thereto, and then triflic anhydride (38.41 g, 136.12 mmol) was slowly added thereto at 0° C., and then the resulting mixture was stirred for 1 hour. After the reaction was completed, and then terminated by slowly adding distilled water to the reaction solution, extraction was performed using dichloromethane and distilled water, the organic layer was dried over anhydrous MgSO₄, and then the solvent was removed by a rotary evaporator, and then, the residue was purified by column chromatography using dichloromethane and hexane as an eluting solvent, thereby obtaining Compound 003-P2 (48 g, 89%).

### 6) Preparation of Compound 003-P1

After Compound 003-P2 (48 g, 100.87 mmol) and phenylboronic acid (13.53 g, 110.95 mmol) were dissolved in 500 ml of 1,4-dioxane and 100 ml of distilled water, Pd(PPh₃)₄ (5.83 g, 5.04 mmol) and K₂CO₃ (34.85 g, 252.17 mmol) were added thereto, and the resulting mixture was stirred under reflux for 10 hours. After the reaction was completed, the reaction solution was extracted with dichloromethane and distilled water, the organic layer was dried over anhydrous MgSO₄, and then the solvent was removed by a rotary evaporator, and then the residue was purified by column chromatography using dichloromethane and hexane as an eluting solvent, thereby obtaining Compound 003-P1 (33 g, 81%).

### 7) Preparation of Compound 003

After Compound 003-P1 (10 g, 24.76 mmol) and di([1,1′-biphenyl]-4-yl)amine (8.36 g, 26.00 mmol) were dissolved in 100 ml of toluene, Pd₂(dba)₃ (1.13 g, 1.24 mmol), Xphos (1.18 g, 2.48 mmol), and NaOtBu (4.76 g, 49.52 mmol) were added thereto, and the resulting mixture was stirred under reflux for 2 hours. After the reaction was completed, the reaction solution was extracted with dichloromethane and distilled water, the organic layer was dried over anhydrous MgSO₄, and then the solvent was removed by a rotary evaporator, and then the residue was purified by column chromatography using dichloromethane and hexane as an eluting solvent, thereby obtaining Compound 003 (13 g, 76%).

In the following Table 1, the target compound was synthesized by performing preparation in the same manner as in Preparation Example 1, except that Compound A was used instead of 1-bromo-4-chloro-2-nitrobenzene, Compound B was used instead of bromobenzene, Compound C was used instead of phenylboronic acid, and Compound D was used instead of di([1,1′-biphenyl]-4-yl)amine.

TABLE 1

| Com- | | |
|---|---|---|
| pound | | |
| No. | Compound A | Compound B |
| 006 | 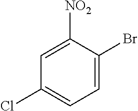 [SMILES: O=[N+]([O-])c1cc(Cl)ccc1Br] | 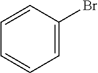 |
| 007 | 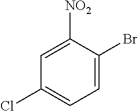 [SMILES: O=[N+]([O-])c1cc(Cl)ccc1Br] | 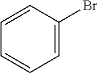 |
| 016 | 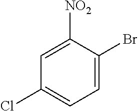 [SMILES: O=[N+]([O-])c1cc(Cl)ccc1Br] | 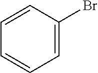 |
| 017 | 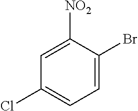 [SMILES: O=[N+]([O-])c1cc(Cl)ccc1Br] | 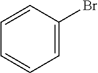 |
| 018 | 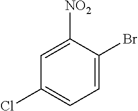 [SMILES: O=[N+]([O-])c1cc(Cl)ccc1Br] | 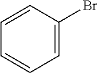 |
| 023 | 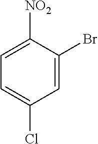 [SMILES: O=[N+]([O-])c1ccc(Cl)cc1Br] | 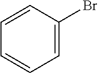 |
| 031 | 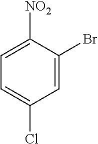 [SMILES: O=[N+]([O-])c1ccc(Cl)cc1Br] | 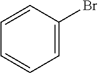 |
| 042 | 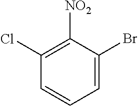 [SMILES: O=[N+]([O-])c1c(Cl)cccc1Br] | 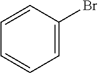 |
| 073 | 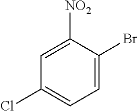 [SMILES: O=[N+]([O-])c1cc(Cl)ccc1Br] | 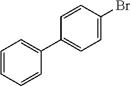 [SMILES: Brc1ccc(-c2ccccc2)cc1] |
| 076 | 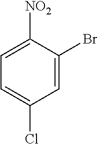 [SMILES: O=[N+]([O-])c1ccc(Cl)cc1Br] | 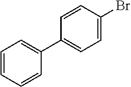 [SMILES: Brc1ccc(-c2ccccc2)cc1] |
| 089 | 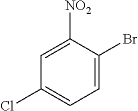 [SMILES: O=[N+]([O-])c1cc(Cl)ccc1Br] | 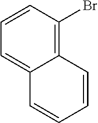 [SMILES: Brc1cccc2ccccc12] |
| 095 | 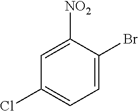 [SMILES: O=[N+]([O-])c1cc(Cl)ccc1Br] | 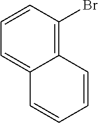 [SMILES: Brc1cccc2ccccc12] |
| 105 | 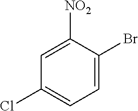 [SMILES: O=[N+]([O-])c1cc(Cl)ccc1Br] | 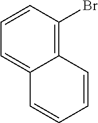 [SMILES: Brc1cccc2ccccc12] |
| 108 | 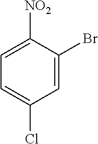 [SMILES: O=[N+]([O-])c1ccc(Cl)cc1Br] | 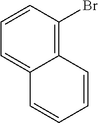 [SMILES: Brc1cccc2ccccc12] |
| 122 | 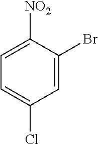 [SMILES: O=[N+]([O-])c1ccc(Cl)cc1Br] | 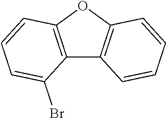 [SMILES: Brc1cccc2oc3ccccc3c12] |
| 144 | 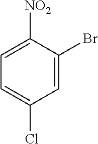 [SMILES: O=[N+]([O-])c1ccc(Cl)cc1Br] | 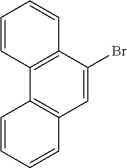 [SMILES: Brc1cc2ccccc2c2ccccc12] |
| 163 | 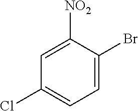 [SMILES: O=[N+]([O-])c1cc(Cl)ccc1Br] | 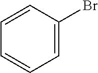 |
| 175 | 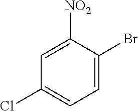 [SMILES: O=[N+]([O-])c1cc(Cl)ccc1Br] | 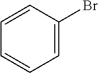 |
| 184 | 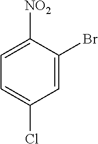 [SMILES: O=[N+]([O-])c1ccc(Cl)cc1Br] | 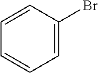 |
| 202 | 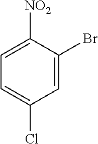 [SMILES: O=[N+]([O-])c1ccc(Cl)cc1Br] | 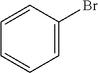 |
| 221 | 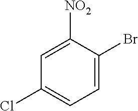 [SMILES: O=[N+]([O-])c1cc(Cl)ccc1Br] | 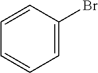 |
| 468 | 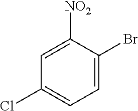 [SMILES: O=[N+]([O-])c1cc(Cl)ccc1Br] | 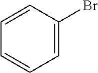 |
| 474 | 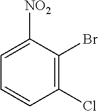 [SMILES: O=[N+]([O-])c1cccc(Cl)c1Br] | 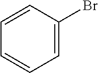 |
| Com- | | |
| pound | | |
| No. | Compound C | Compound D |
| 006 | 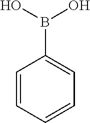 | 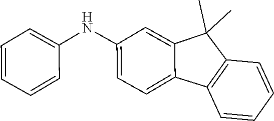 [SMILES: CC1(C)c2ccccc2-c2ccc(Nc3ccccc3)cc21] |
| 007 | 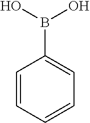 | 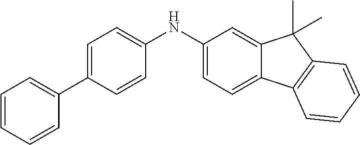 [SMILES: CC1(C)c2ccccc2-c2ccc(Nc3ccc(-c4ccccc4)cc3)cc21] |
| 016 | 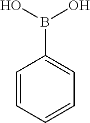 | 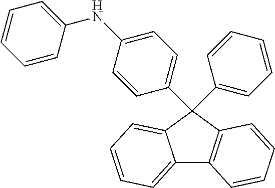 [SMILES: c1ccc(Nc2ccc(C3(c4ccccc4)c4ccccc4-c4ccccc43)cc2)cc1] |
| 017 | 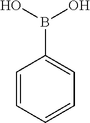 | 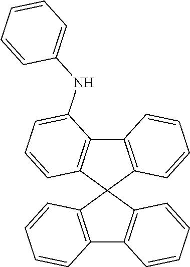 [SMILES: c1ccc(Nc2cccc3c2-c2ccccc2C32c3ccccc3-c3ccccc32)cc1] |
| 018 | 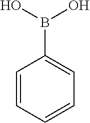 | 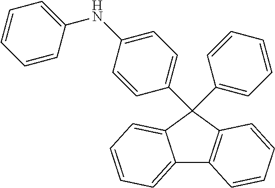 [SMILES: c1ccc(Nc2ccc(C3(c4ccccc4)c4ccccc4-c4ccccc43)cc2)cc1] |
| 023 | 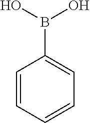 | 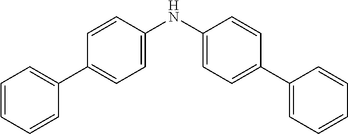 [SMILES: c1ccc(-c2ccc(Nc3ccc(-c4ccccc4)cc3)cc2)cc1] |
| 031 | 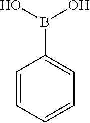 | 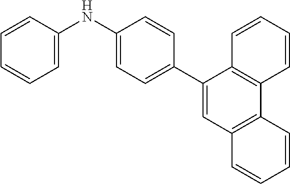 [SMILES: c1ccc(Nc2ccc(-c3cc4ccccc4c4ccccc34)cc2)cc1] |
| 042 | 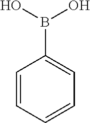 | 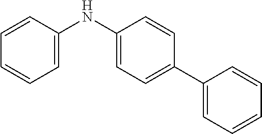 [SMILES: c1ccc(Nc2ccc(-c3ccccc3)cc2)cc1] |
| 073 | 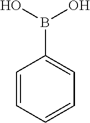 | 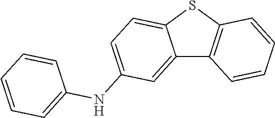 [SMILES: c1ccc(Nc2ccc3sc4ccccc4c3c2)cc1] |
| 076 | 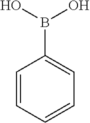 | 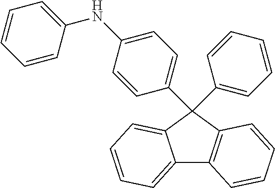 [SMILES: c1ccc(Nc2ccc(C3(c4ccccc4)c4ccccc4-c4ccccc43)cc2)cc1] |
| 089 | 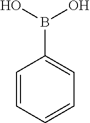 | 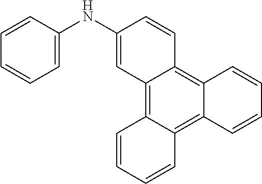 [SMILES: c1ccc(Nc2ccc3c4ccccc4c4ccccc4c3c2)cc1] |
| 095 | 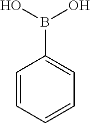 | 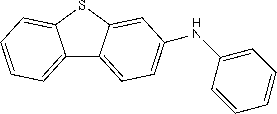 [SMILES: c1ccc(Nc2ccc3c(c2)sc2ccccc23)cc1] |
| 105 | 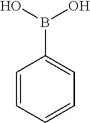 | 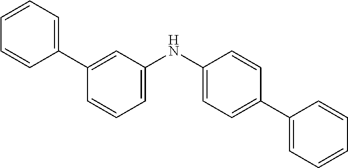 [SMILES: c1ccc(-c2ccc(Nc3cccc(-c4ccccc4)c3)cc2)cc1] |
| 108 | 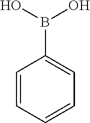 | 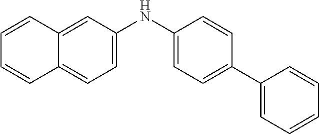 [SMILES: c1ccc(-c2ccc(Nc3ccc4ccccc4c3)cc2)cc1] |
| 122 | 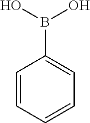 | 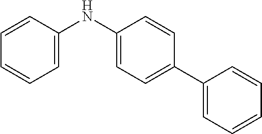 [SMILES: c1ccc(Nc2ccc(-c3ccccc3)cc2)cc1] |
| 144 | 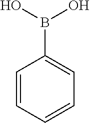 | 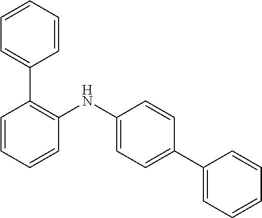 [SMILES: c1ccc(-c2ccc(Nc3ccccc3-c3ccccc3)cc2)cc1] |
| 163 | 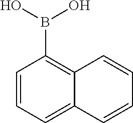 [SMILES: OB(O)c1cccc2ccccc12] | 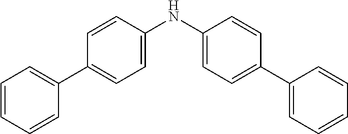 [SMILES: c1ccc(-c2ccc(Nc3ccc(-c4ccccc4)cc3)cc2)cc1] |
| 175 | 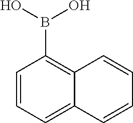 [SMILES: OB(O)c1cccc2ccccc12] | 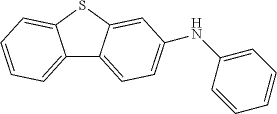 [SMILES: c1ccc(Nc2ccc3c(c2)sc2ccccc23)cc1] |
| 184 | 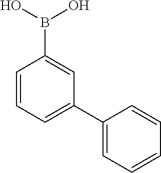 [SMILES: OB(O)c1cccc(-c2ccccc2)c1] | 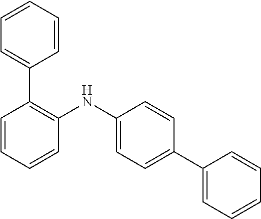 [SMILES: c1ccc(-c2ccc(Nc3ccccc3-c3ccccc3)cc2)cc1] |
| 202 | 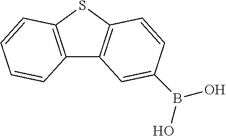 [SMILES: OB(O)c1ccc2sc3ccccc3c2c1] | 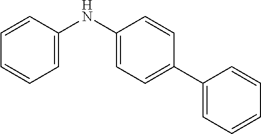 [SMILES: c1ccc(Nc2ccc(-c3ccccc3)cc2)cc1] |
| 221 | 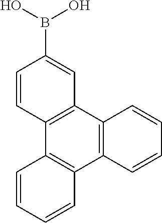 [SMILES: OB(O)c1ccc2c3ccccc3c3ccccc3c2c1] | 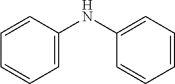 [SMILES: c1ccc(Nc2ccccc2)cc1] |
| 468 | 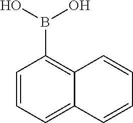 [SMILES: OB(O)c1cccc2ccccc12] | 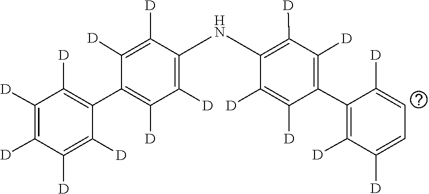 [SMILES: [2H]c1ccc([2H])c(-c2c([2H])c([2H])c(Nc3c([2H])c([2H])c(-c4c([2H])c([2H])c([2H])c([2H])c4[2H])c([2H])c3[2H])c([2H])c2[2H])c1[2H]] |
| 474 | 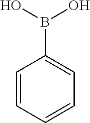 | 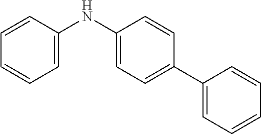 [SMILES: c1ccc(Nc2ccc(-c3ccccc3)cc2)cc1] |

| Compound No. | Target compound | yield |
|---|---|---|
| 006 | 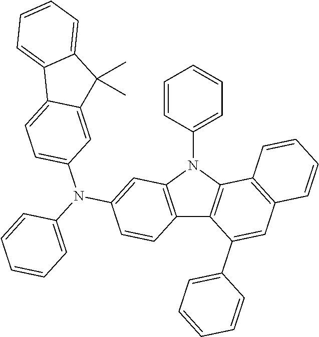 [SMILES: CC1(C)c2ccccc2-c2ccc(N(c3ccccc3)c3ccc4c5c(-c6ccccc6)cc6ccccc6c5n(-c5ccccc5)c4c3)cc21] | 72% |
| 007 | 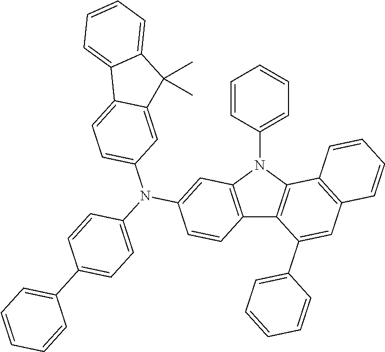 [SMILES: CC1(C)c2ccccc2-c2ccc(N(c3ccc(-c4ccccc4)cc3)c3ccc4c5c(-c6ccccc6)cc6ccccc6c5n(-c5ccccc5)c4c3)cc21] | 79% |
| 016 | 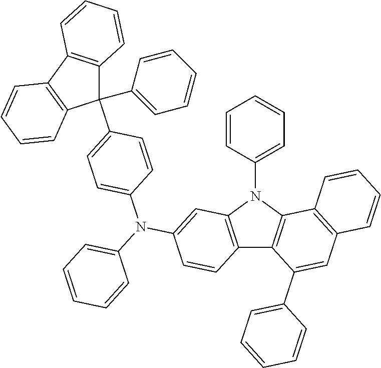 [SMILES: c1ccc(-c2cc3ccccc3c3c2c2ccc(N(c4ccccc4)c4ccc(C5(c6ccccc6)c6ccccc6-c6ccccc65)cc4)cc2n3-c2ccccc2)cc1] | 78% |
| 017 | 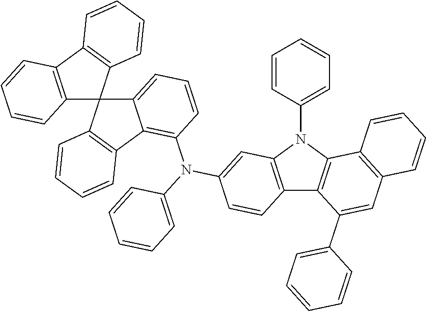 [SMILES: c1ccc(-c2cc3ccccc3c3c2c2ccc(N(c4ccccc4)c4cccc5c4-c4ccccc4C54c5ccccc5-c5ccccc54)cc2n3-c2ccccc2)cc1] | 81% |
| 018 | 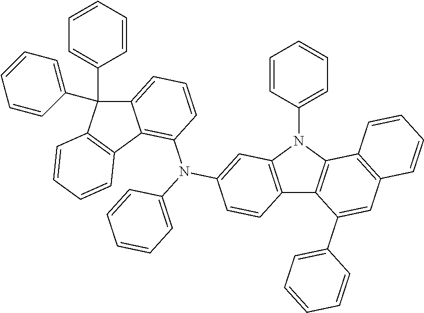 [SMILES: c1ccc(-c2cc3ccccc3c3c2c2ccc(N(c4ccccc4)c4cccc5c4-c4ccccc4C5(c4ccccc4)c4ccccc4)cc2n3-c2ccccc2)cc1] | 84% |
| 023 | 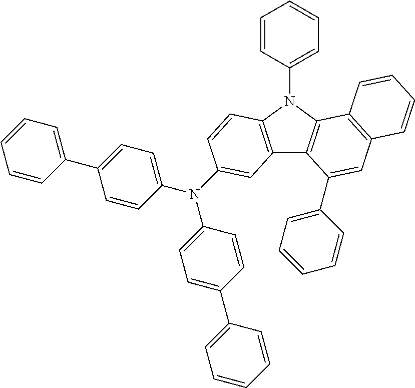 [SMILES: c1ccc(-c2ccc(N(c3ccc(-c4ccccc4)cc3)c3ccc4c(c3)c3c(-c5ccccc5)cc5ccccc5c3n4-c3ccccc3)cc2)cc1] | 76% |
| 031 | 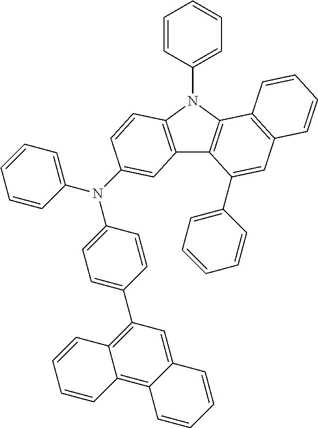 [SMILES: c1ccc(-c2cc3ccccc3c3c2c2cc(N(c4ccccc4)c4ccc(-c5cc6ccccc6c6ccccc56)cc4)ccc2n3-c2ccccc2)cc1] | 79% |
| 042 | 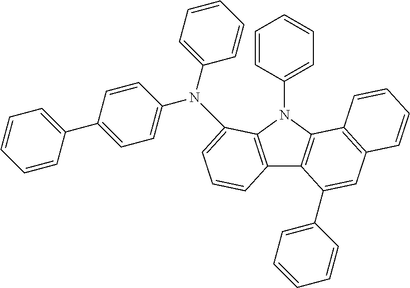 [SMILES: c1ccc(-c2ccc(N(c3ccccc3)c3cccc4c5c(-c6ccccc6)cc6ccccc6c5n(-c5ccccc5)c34)cc2)cc1] | 75% |
| 073 | 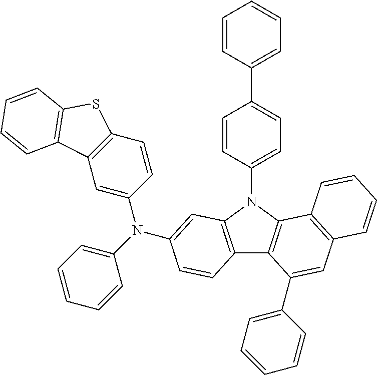 [SMILES: c1ccc(-c2ccc(-n3c4cc(N(c5ccccc5)c5ccc6sc7ccccc7c6c5)ccc4c4c(-c5ccccc5)cc5ccccc5c43)cc2)cc1] | 73% |
| 076 | 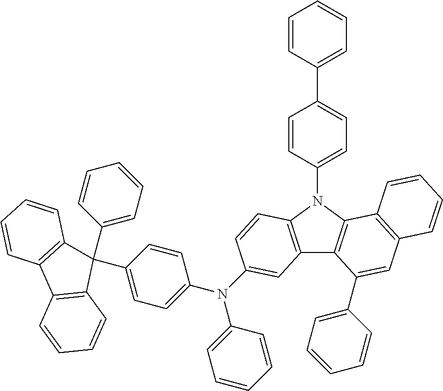 [SMILES: c1ccc(-c2ccc(-n3c4ccc(N(c5ccccc5)c5ccc(C6(c7ccccc7)c7ccccc7-c7ccccc76)cc5)cc4c4c(-c5ccccc5)cc5ccccc5c43)cc2)cc1] | 83% |
| 089 | 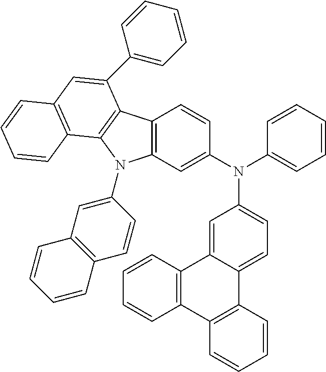 [SMILES: c1ccc(-c2cc3ccccc3c3c2c2ccc(N(c4ccccc4)c4ccc5c6ccccc6c6ccccc6c5c4)cc2n3-c2ccc3ccccc3c2)cc1] | 73% |
| 095 | 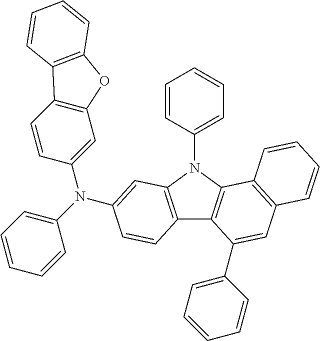 [SMILES: c1ccc(-c2cc3ccccc3c3c2c2ccc(N(c4ccccc4)c4ccc5c(c4)oc4ccccc45)cc2n3-c2ccccc2)cc1] | 80% |
| 105 | 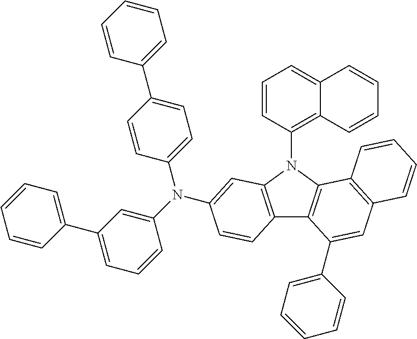 [SMILES: c1ccc(-c2ccc(N(c3cccc(-c4ccccc4)c3)c3ccc4c5c(-c6ccccc6)cc6ccccc6c5n(-c5cccc6ccccc56)c4c3)cc2)cc1] | 78% |
| 108 | 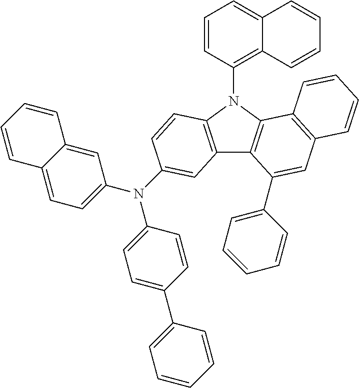 [SMILES: c1ccc(-c2ccc(N(c3ccc4ccccc4c3)c3ccc4c(c3)c3c(-c5ccccc5)cc5ccccc5c3n4-c3cccc4ccccc34)cc2)cc1] | 81% |
| 122 | 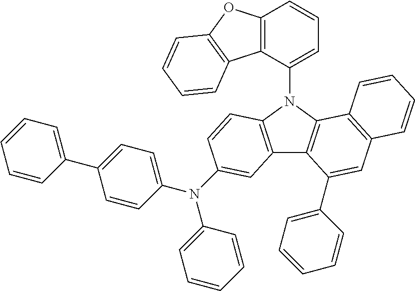 [SMILES: c1ccc(-c2ccc(N(c3ccccc3)c3ccc4c(c3)c3c(-c5ccccc5)cc5ccccc5c3n4-c3cccc4oc5ccccc5c34)cc2)cc1] | 74% |
| 144 | 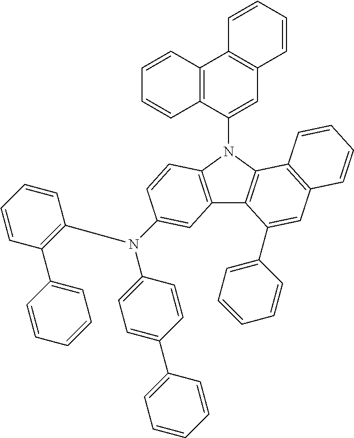 [SMILES: c1ccc(-c2ccc(N(c3ccc4c(c3)c3c(-c5ccccc5)cc5ccccc5c3n4-c3cc4ccccc4c4ccccc34)c3ccccc3-c3ccccc3)cc2)cc1] | 76% |
| 163 | 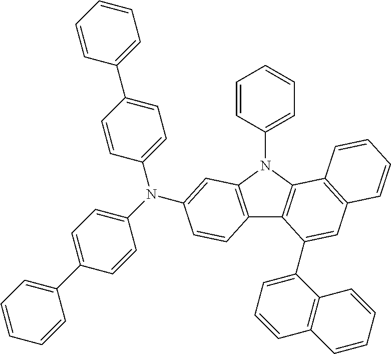 [SMILES: c1ccc(-c2ccc(N(c3ccc(-c4ccccc4)cc3)c3ccc4c5c(-c6cccc7ccccc67)cc6ccccc6c5n(-c5ccccc5)c4c3)cc2)cc1] | 73% |
| 175 | 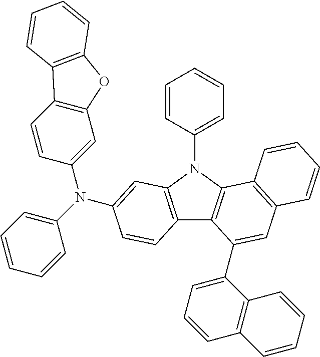 [SMILES: c1ccc(N(c2ccc3c(c2)oc2ccccc23)c2ccc3c4c(-c5cccc6ccccc56)cc5ccccc5c4n(-c4ccccc4)c3c2)cc1] | 76% |
| 184 | 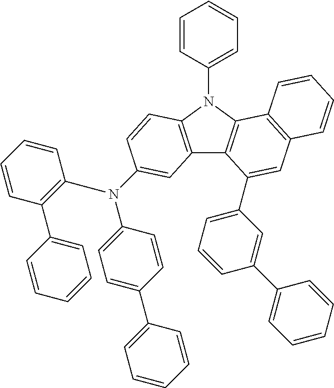 [SMILES: c1ccc(-c2ccc(N(c3ccc4c(c3)c3c(-c5cccc(-c6ccccc6)c5)cc5ccccc5c3n4-c3ccccc3)c3ccccc3-c3ccccc3)cc2)cc1] | 77% |
| 202 | 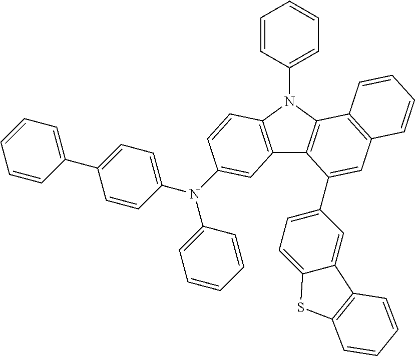 [SMILES: c1ccc(-c2ccc(N(c3ccccc3)c3ccc4c(c3)c3c(-c5ccc6sc7ccccc7c6c5)cc5ccccc5c3n4-c3ccccc3)cc2)cc1] | 78% |
| 221 | 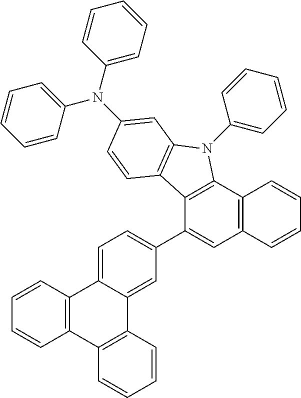 [SMILES: c1ccc(N(c2ccccc2)c2ccc3c4c(-c5ccc6c7ccccc7c7ccccc7c6c5)cc5ccccc5c4n(-c4ccccc4)c3c2)cc1] | 78% |
| 468 | 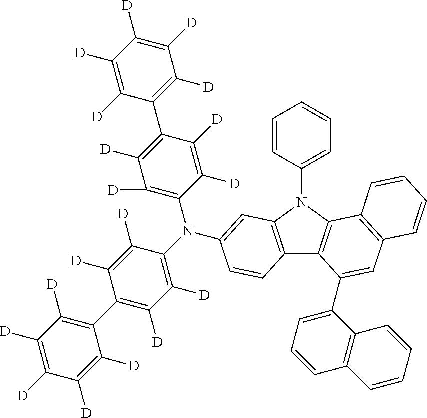 [SMILES: [2H]c1c([2H])c([2H])c(-c2c([2H])c([2H])c(N(c3ccc4c5c(-c6cccc7ccccc67)cc6ccccc6c5n(-c5ccccc5)c4c3)c3c([2H])c([2H])c(-c4c([2H])c([2H])c([2H])c([2H])c4[2H])c([2H])c3[2H])c([2H])c2[2H])c([2H])c1[2H]] | 74% |
| 474 | 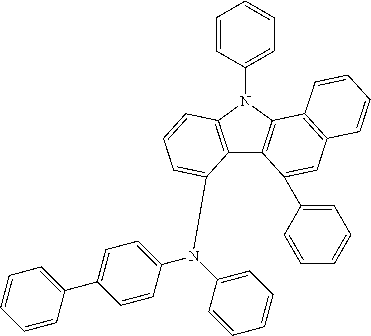 [SMILES: c1ccc(-c2ccc(N(c3ccccc3)c3cccc4c3c3c(-c5ccccc5)cc5ccccc5c3n4-c3ccccc3)cc2)cc1] | 68% |

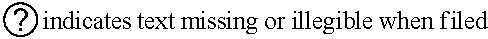

### [Preparation Example 2] Preparation of Compound 248

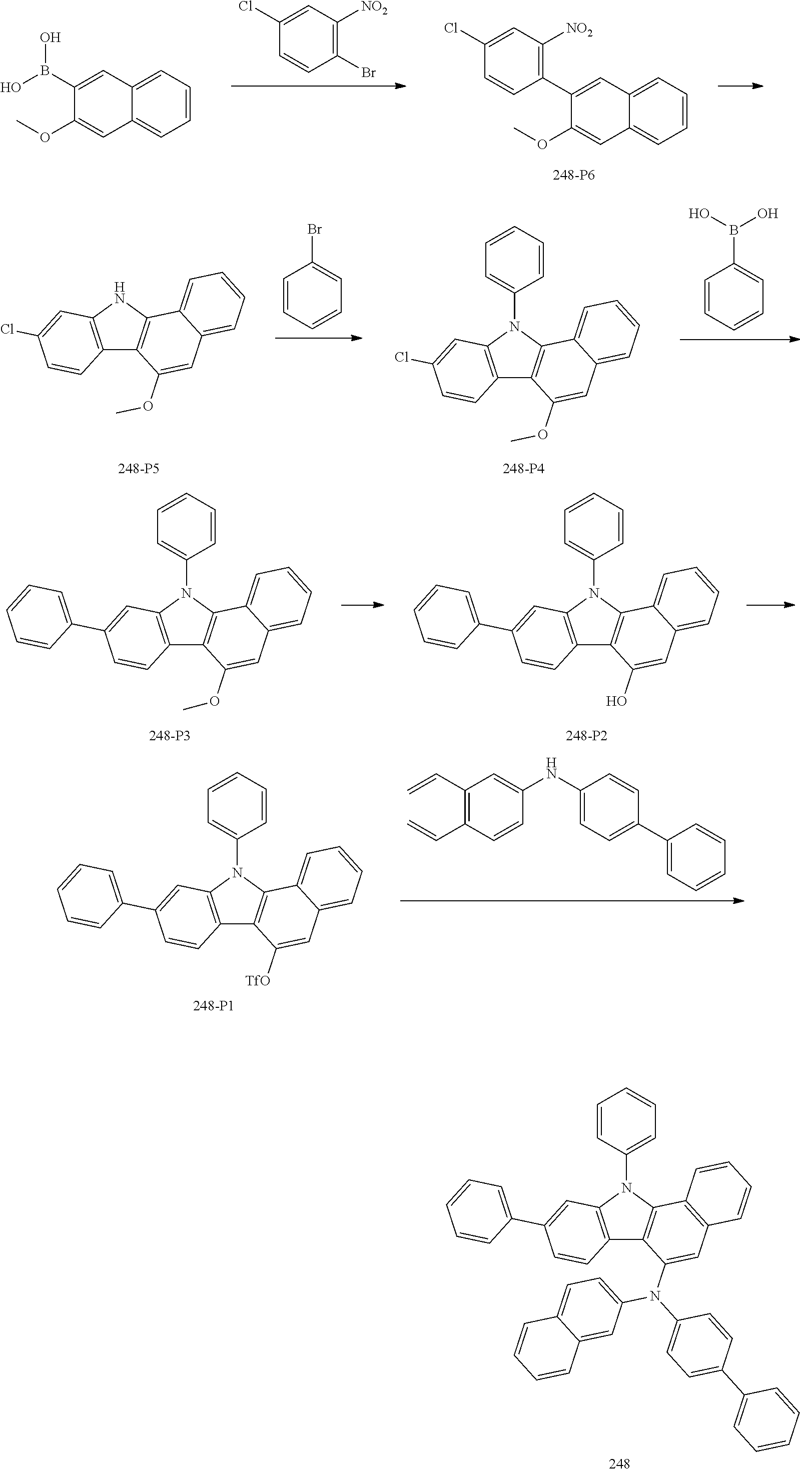

### 1) Preparation of Compound 248-P6

After (3-methoxynaphthalen-2-yl)boronic acid (50 g, 247.51 mmol) and 1-bromo-4-chloro-2-nitrobenzene (61.45 g, 259.89 mmol) were dissolved in 500 ml of 1,4-dioxane and 100 ml of distilled water, Pd(PPh₃)₄ (14.30 g, 12.38 mmol) and K₂CO₃ (85.52 g, 618.78 mmol) were added thereto, and the resulting mixture was stirred under reflux for 10 hours. After the reaction was completed, the reaction solution was extracted with dichloromethane and distilled water, the organic layer was dried over anhydrous MgSO₄, and then the solvent was removed by a rotary evaporator, and then the residue was purified by column chromatography using dichloromethane and hexane as an eluting solvent, thereby obtaining Compound 248-P6 (64 g, 82%).

### 2) Preparation of Compound 248-P5

Compound 248-P6 (64 g, 203.99 mmol) and triphenylphosphine (133.76 g, 509.98 mmol) were added to 700 ml of 1,2-dichlorobenzene, and the resulting mixture was stirred under reflux for 7 hours. After the reaction was completed, dichloromethane was added to the reaction solution for dissolution, and then the resulting solution was extracted with distilled water, the organic layer was dried over anhydrous MgSO₄, and then the solvent was removed by a rotary evaporator, and then the residue was purified by column chromatography using dichloromethane and hexane as an eluting solvent, thereby obtaining Compound 248-P5 (48 g, 86%).

### 3) Preparation of Compound 248-P4

After Compound 248-P5 (48 g, 170.37 mmol) and bromobenzene (32.1 g, 204.44 mmol) were dissolved in 500 ml of toluene, Pd₂(dba)₃ (7.80 g, 8.52 mmol), Xphos (8.12 g, 17.04 mmol), and NaOtBu (32.75 g, 340.74 mmol) were added thereto and the resulting mixture was stirred under reflux for 2 hours. After the reaction was completed, the reaction solution was extracted with dichloromethane and distilled water, the organic layer was dried over anhydrous MgSO₄, and then the solvent was removed by a rotary evaporator, and then the residue was purified by column chromatography using dichloromethane and hexane as an eluting solvent, thereby obtaining Compound 248-P4 (45 g, 74%).

### 4) Preparation of Compound 248-P3

After Compound 248-P4 (45 g, 125.76 mmol) and phenylboronic acid (16.87 g, 138.33 mmol) were dissolved in 500 ml of 1,4-dioxane and 100 ml of distilled water, Pd(dba)₂ (1.45 g, 2.52 mmol), Xphos (3 g, 6.29 mmol) and K₂CO₃ (43.45 g, 314.40 mmol) were added thereto, and the resulting mixture was stirred under reflux for 12 hours. After the reaction was completed, the reaction solution was extracted with dichloromethane and distilled water, the organic layer was dried over anhydrous MgSO₄, and then the solvent was removed by a rotary evaporator, and then the residue was purified by column chromatography using dichloromethane and hexane as an eluting solvent, thereby obtaining Compound 248-P3 (41 g, 82%).

### 5) Preparation of Compound 248-P2

After Compound 248-P3 (41 g, 102.63 mmol) was dissolved in 500 ml of dichloromethane, boron tribromide (38.57 g, 153.95 mmol) was slowly added thereto at 0° C., and then the resulting mixture was stirred for 3 hours. After the reaction was completed, and then terminated by slowly adding distilled water to the reaction solution, extraction was performed using dichloromethane and distilled water, the organic layer was dried over anhydrous MgSO₄, and then the solvent was removed by a rotary evaporator, and then, the residue was purified by column chromatography using dichloromethane and hexane as an eluting solvent, thereby obtaining Compound 248-P2 (35 g, 88%).

### 6) Preparation of Compound 248-P1

After Compound 248-P2 (35 g, 90.80 mmol) was dissolved in 400 ml of dichloromethane, triethylamine (11.03 g, 108.96 mmol) was added thereto, and then triflic anhydride (30.74 g, 108.96 mmol) was slowly added thereto at 0° C., and then the resulting mixture was stirred for 1 hour. After the reaction was completed, and then terminated by slowly adding distilled water to the reaction solution, extraction was performed using dichloromethane and distilled water, the organic layer was dried over anhydrous MgSO₄, and then the solvent was removed by a rotary evaporator, and then, the residue was purified by column chromatography using dichloromethane and hexane as an eluting solvent, thereby obtaining Compound 248-P1 (41 g, 87%).

### 7) Preparation of Compound 248

After Compound 248-P1 (10 g, 19.32 mmol) and N-([1,1′-biphenyl]-4-yl)naphthalen-2-amine (5.99 g, 20.29 mmol) were dissolved in 100 ml of toluene, Pd₂(dba)₃ (0.88 g, 0.97 mmol), Xphos (0.92 g, 1.93 mmol), and NaOtBu (3.71 g, 38.65 mmol) thereto, and the resulting mixture was stirred under reflux for 2 hours. After the reaction was completed, the reaction solution was extracted with dichloromethane and distilled water, the organic layer was dried over anhydrous MgSO₄, and then the solvent was removed by a rotary evaporator, and then the residue was purified by column chromatography using dichloromethane and hexane as an eluting solvent, thereby obtaining Compound 248 (11 g, 86%).

In the following Table 2, the target compound was synthesized by performing preparation in the same manner as in Preparation Example 2, except that Compound E was used instead of 1-bromo-4-chloro-2-nitrobenzene, Compound F was used instead of bromobenzene, Compound G was used instead of phenylboronic acid, and Compound H was used instead of N-([1,1′-biphenyl]-4-yl)naphthalen-2-amine.

TABLE 2

| Com- | | | |
|---|---|---|---|
| pound | | | |
| No. | Compound E | Compound F | Compound G |
| 266 | 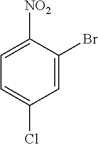 [SMILES: O=[N+]([O-])c1ccc(Cl)cc1Br] | 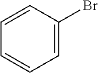 | 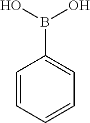 |
| 296 | 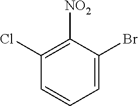 [SMILES: O=[N+]([O-])c1c(Cl)cccc1Br] | 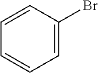 | 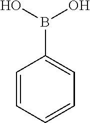 |
| 302 | 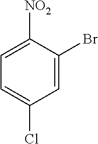 [SMILES: O=[N+]([O-])c1ccc(Cl)cc1Br] | 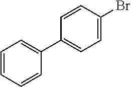 [SMILES: Brc1ccc(-c2ccccc2)cc1] | 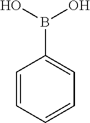 |
| 323 | 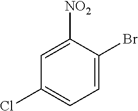 [SMILES: O=[N+]([O-])c1cc(Cl)ccc1Br] | 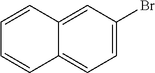 [SMILES: Brc1ccc2ccccc2c1] | 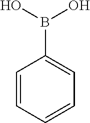 |
| 341 | 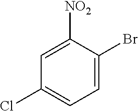 [SMILES: O=[N+]([O-])c1cc(Cl)ccc1Br] | 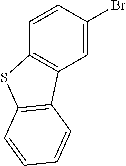 [SMILES: Brc1ccc2sc3ccccc3c2c1] | 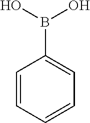 |
| 361 | 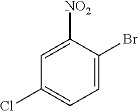 [SMILES: O=[N+]([O-])c1cc(Cl)ccc1Br] | 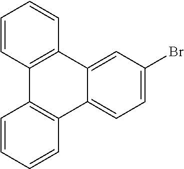 [SMILES: Brc1ccc2c3ccccc3c3ccccc3c2c1] | 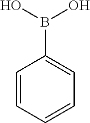 |
| 381 | 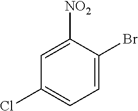 [SMILES: O=[N+]([O-])c1cc(Cl)ccc1Br] | 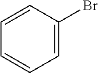 | 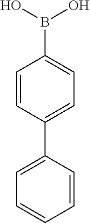 [SMILES: OB(O)c1ccc(-c2ccccc2)cc1] |
| 403 | 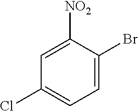 [SMILES: O=[N+]([O-])c1cc(Cl)ccc1Br] | 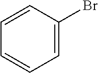 | 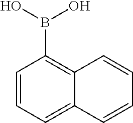 [SMILES: OB(O)c1cccc2ccccc12] |
| 405 | 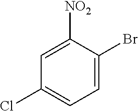 [SMILES: O=[N+]([O-])c1cc(Cl)ccc1Br] | 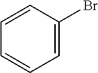 | 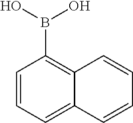 [SMILES: OB(O)c1cccc2ccccc12] |
| 435 | 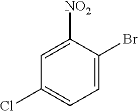 [SMILES: O=[N+]([O-])c1cc(Cl)ccc1Br] | 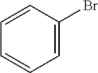 | 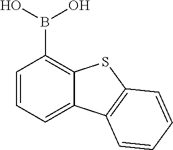 [SMILES: OB(O)c1cccc2c1sc1ccccc12] |
| 442 | 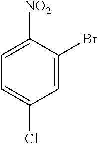 [SMILES: O=[N+]([O-])c1ccc(Cl)cc1Br] | 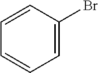 | 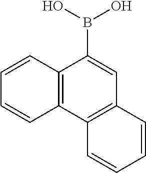 [SMILES: OB(O)c1cc2ccccc2c2ccccc12] |
| 488 | 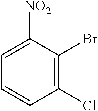 [SMILES: O=[N+]([O-])c1cccc(Cl)c1Br] | 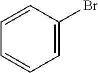 | 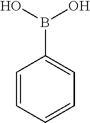 |
| Com- | | | |
| pound | | | |
| No. | Compound H | Target compound | yield |
| 266 | 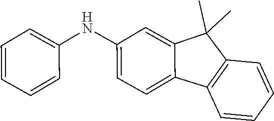 [SMILES: CC1(C)c2ccccc2-c2ccc(Nc3ccccc3)cc21] | 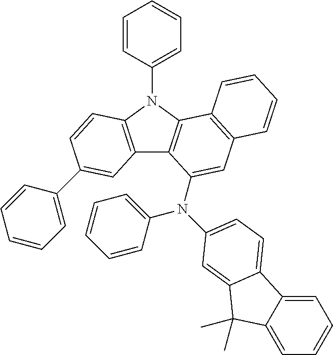 [SMILES: CC1(C)c2ccccc2-c2ccc(N(c3ccccc3)c3cc4ccccc4c4c3c3cc(-c5ccccc5)ccc3n4-c3ccccc3)cc21] | 79% |
| 296 | 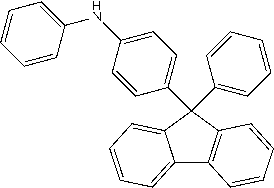 [SMILES: c1ccc(Nc2ccc(C3(c4ccccc4)c4ccccc4-c4ccccc43)cc2)cc1] | 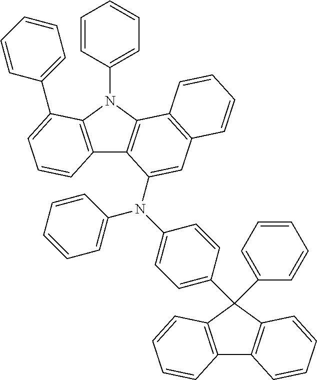 [SMILES: c1ccc(-c2cccc3c4c(N(c5ccccc5)c5ccc(C6(c7ccccc7)c7ccccc7-c7ccccc76)cc5)cc5ccccc5c4n(-c4ccccc4)c23)cc1] | 77% |
| 302 | 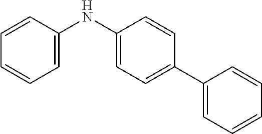 [SMILES: c1ccc(Nc2ccc(-c3ccccc3)cc2)cc1] | 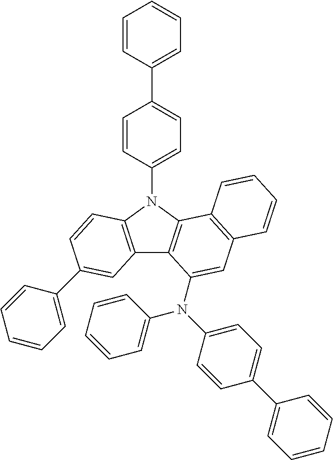 [SMILES: c1ccc(-c2ccc(N(c3ccccc3)c3cc4ccccc4c4c3c3cc(-c5ccccc5)ccc3n4-c3ccc(-c4ccccc4)cc3)cc2)cc1] | 80% |
| 323 | 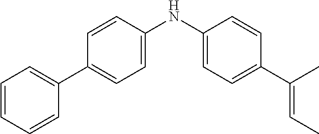 [SMILES: C/C=C(\C)c1ccc(Nc2ccc(-c3ccccc3)cc2)cc1] | 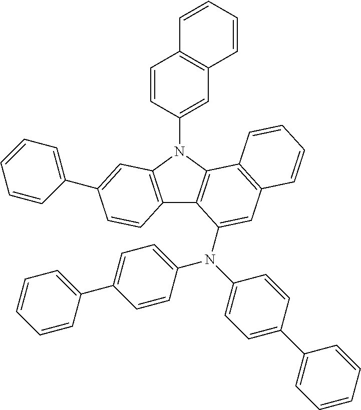 [SMILES: c1ccc(-c2ccc(N(c3ccc(-c4ccccc4)cc3)c3cc4ccccc4c4c3c3ccc(-c5ccccc5)cc3n4-c3ccc4ccccc4c3)cc2)cc1] | 76% |
| 341 | 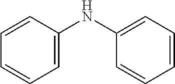 [SMILES: c1ccc(Nc2ccccc2)cc1] | 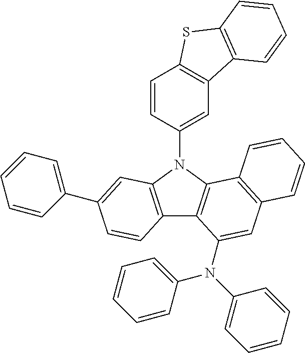 [SMILES: c1ccc(-c2ccc3c4c(N(c5ccccc5)c5ccccc5)cc5ccccc5c4n(-c4ccc5sc6ccccc6c5c4)c3c2)cc1] | 72% |
| 361 | 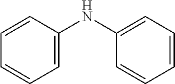 [SMILES: c1ccc(Nc2ccccc2)cc1] | 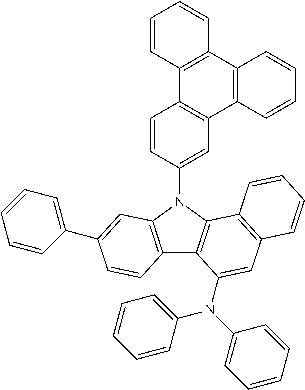 [SMILES: c1ccc(-c2ccc3c4c(N(c5ccccc5)c5ccccc5)cc5ccccc5c4n(-c4ccc5c6ccccc6c6ccccc6c5c4)c3c2)cc1] | 75% |
| 381 | 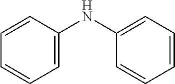 [SMILES: c1ccc(Nc2ccccc2)cc1] | 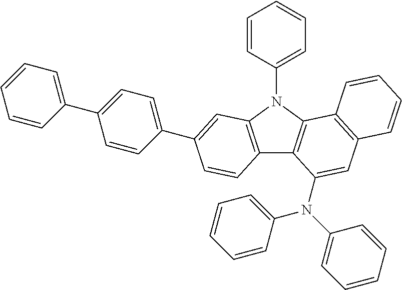 [SMILES: c1ccc(-c2ccc(-c3ccc4c5c(N(c6ccccc6)c6ccccc6)cc6ccccc6c5n(-c5ccccc5)c4c3)cc2)cc1] | 81% |
| 403 | 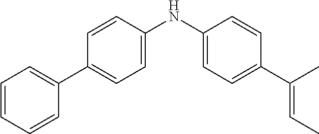 [SMILES: C/C=C(\C)c1ccc(Nc2ccc(-c3ccccc3)cc2)cc1] | 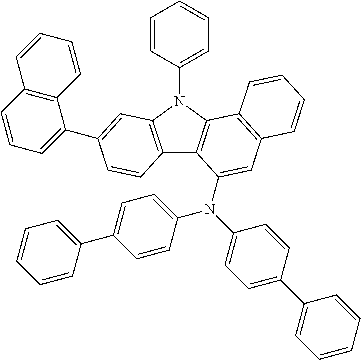 [SMILES: c1ccc(-c2ccc(N(c3ccc(-c4ccccc4)cc3)c3cc4ccccc4c4c3c3ccc(-c5cccc6ccccc56)cc3n4-c3ccccc3)cc2)cc1] | 78% |
| 405 | 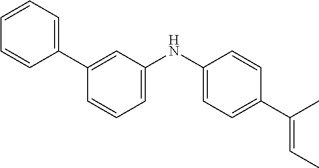 [SMILES: C/C=C(\C)c1ccc(Nc2cccc(-c3ccccc3)c2)cc1] | 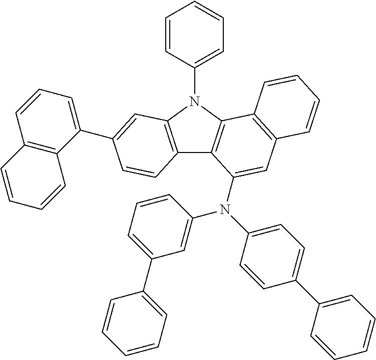 [SMILES: c1ccc(-c2ccc(N(c3cccc(-c4ccccc4)c3)c3cc4ccccc4c4c3c3ccc(-c5cccc6ccccc56)cc3n4-c3ccccc3)cc2)cc1] | 82% |
| 435 | 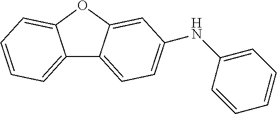 [SMILES: c1ccc(Nc2ccc3c(c2)oc2ccccc23)cc1] | 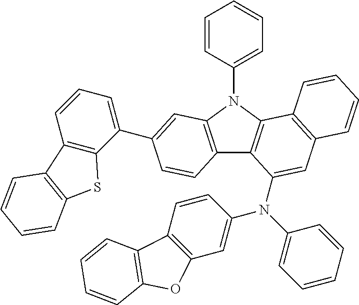 [SMILES: c1ccc(N(c2ccc3c(c2)oc2ccccc23)c2cc3ccccc3c3c2c2ccc(-c4cccc5c4sc4ccccc45)cc2n3-c2ccccc2)cc1] | 74% |
| 442 | 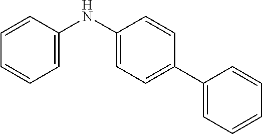 [SMILES: c1ccc(Nc2ccc(-c3ccccc3)cc2)cc1] | 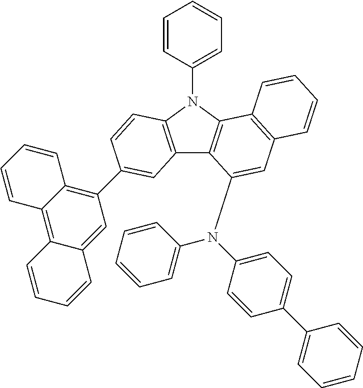 [SMILES: c1ccc(-c2ccc(N(c3ccccc3)c3cc4ccccc4c4c3c3cc(-c5cc6ccccc6c6ccccc56)ccc3n4-c3ccccc3)cc2)cc1] | 76% |
| 488 | 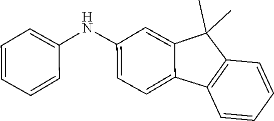 [SMILES: CC1(C)c2ccccc2-c2ccc(Nc3ccccc3)cc21] | 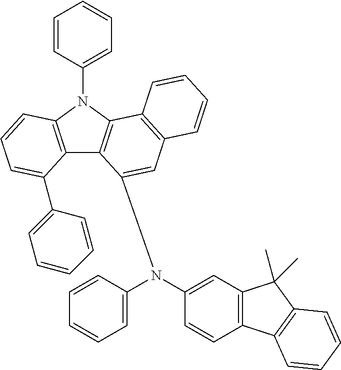 [SMILES: CC1(C)c2ccccc2-c2ccc(N(c3ccccc3)c3cc4ccccc4c4c3c3c(-c5ccccc5)cccc3n4-c3ccccc3)cc21] | 64% |

### [Preparation Example 3] Preparation of Compound 039

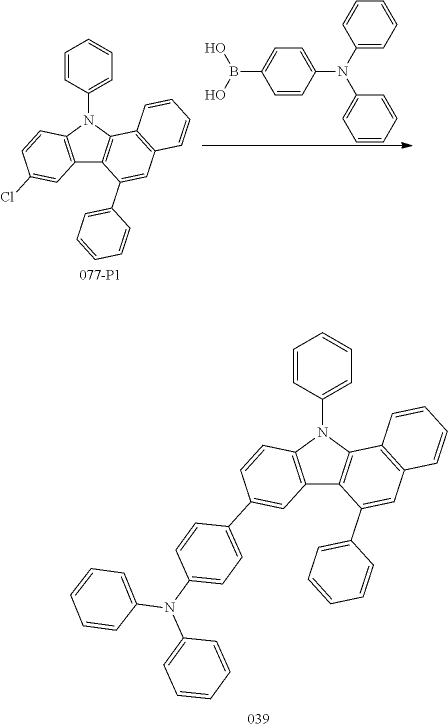 [SMILES: Clc1ccc2c(c1)c1c(-c3ccccc3)cc3ccccc3c1n2-c1ccccc1 ; OB(O)c1ccc(N(c2ccccc2)c2ccccc2)cc1 ; c1ccc(-c2cc3ccccc3c3c2c2cc(-c4ccc(N(c5ccccc5)c5ccccc5)cc4)ccc2n3-c2ccccc2)cc1]

### 1) Preparation of Compound 039

After Compound 007-P1 (10 g, 24.76 mmol) and (4-(diphenylamino)phenyl)boronic acid (7.87 g, 27.23 mmol) were dissolved in 100 ml of 1,4-dioxane and 20 ml of distilled water, Pd(dba)₂ (0.28 g, 0.50 mol), Xphos (0.59 g, 1.24 mmol) and K₂CO₃ (8.55 g, 61.90 mmol) were added thereto, and the resulting mixture was stirred under reflux for 6 hours. After the reaction was completed, the reaction solution was extracted with dichloromethane and distilled water, the organic layer was dried over anhydrous MgSO₄, and then the solvent was removed by a rotary evaporator, and then the residue was purified by column chromatography using dichloromethane and hexane as an eluting solvent, thereby obtaining Compound 039 (12 g, 79%).

In the following Table 3, the target compound was synthesized by performing preparation in the same manner as in Preparation Example 3, except that Compound I was used instead of Compound 007-P1 and Compound J was used instead of (4-(diphenylamino)phenyl)boronic acid.

TABLE 3

| Com- | | |
|---|---|---|
| pound | | |
| No. | Compound I | Compound J |
| 040 | 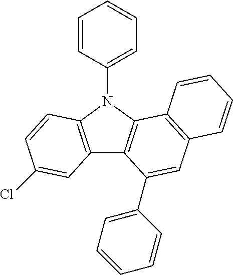 [SMILES: Clc1ccc2c(c1)c1c(-c3ccccc3)cc3ccccc3c1n2-c1ccccc1] | 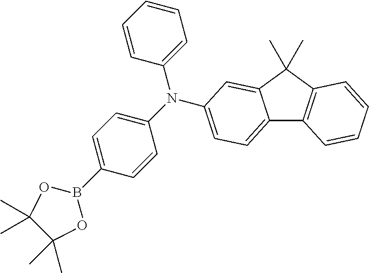 [SMILES: CC1(C)c2ccccc2-c2ccc(N(c3ccccc3)c3ccc(B4OC(C)(C)C(C)(C)O4)cc3)cc21] |
| 079 | 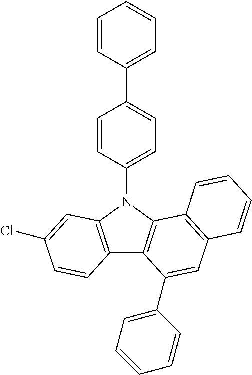 [SMILES: Clc1ccc2c3c(-c4ccccc4)cc4ccccc4c3n(-c3ccc(-c4ccccc4)cc3)c2c1] | 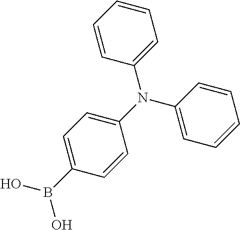 [SMILES: OB(O)c1ccc(N(c2ccccc2)c2ccccc2)cc1] |
| 199 | 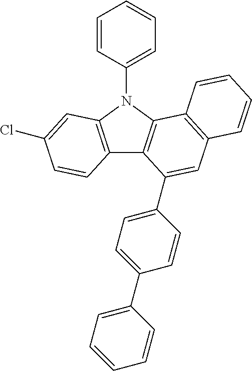 [SMILES: Clc1ccc2c3c(-c4ccc(-c5ccccc5)cc4)cc4ccccc4c3n(-c3ccccc3)c2c1] | 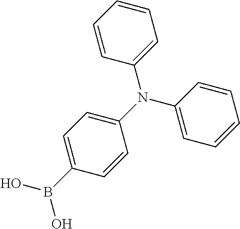 [SMILES: OB(O)c1ccc(N(c2ccccc2)c2ccccc2)cc1] |
| 200 | 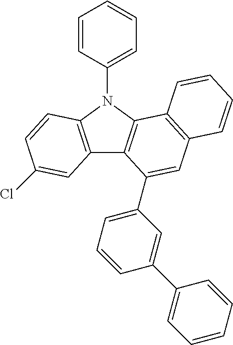 [SMILES: Clc1ccc2c(c1)c1c(-c3cccc(-c4ccccc4)c3)cc3ccccc3c1n2-c1ccccc1] | 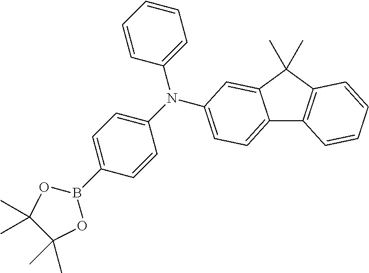 [SMILES: CC1(C)c2ccccc2-c2ccc(N(c3ccccc3)c3ccc(B4OC(C)(C)C(C)(C)O4)cc3)cc21] |
| 318 | 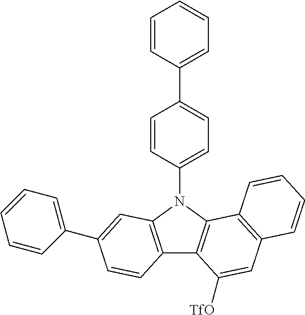 [SMILES: O=S(=O)(Oc1cc2ccccc2c2c1c1ccc(-c3ccccc3)cc1n2-c1ccc(-c2ccccc2)cc1)C(F)(F)F] | 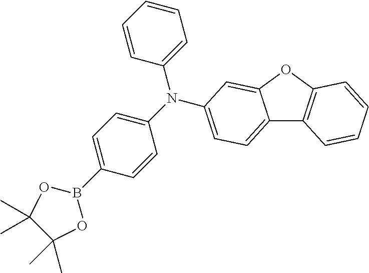 [SMILES: CC1(C)OB(c2ccc(N(c3ccccc3)c3ccc4c(c3)oc3ccccc34)cc2)OC1(C)C] |

| Compound | | |
|---|---|---|
| No. | Target compound | yield |
| 040 | 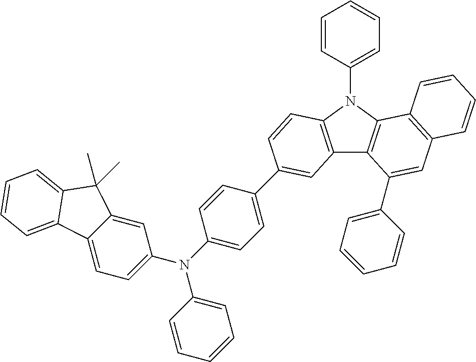 [SMILES: CC1(C)c2ccccc2-c2ccc(N(c3ccccc3)c3ccc(-c4ccc5c(c4)c4c(-c6ccccc6)cc6ccccc6c4n5-c4ccccc4)cc3)cc21] | 79% |
| 079 | 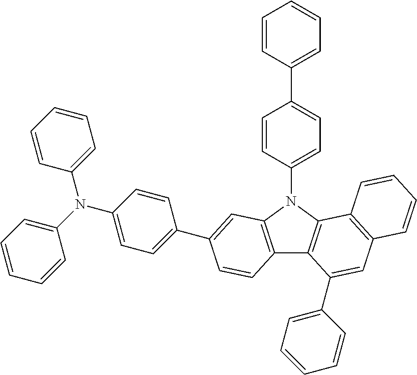 [SMILES: c1ccc(-c2ccc(-n3c4cc(-c5ccc(N(c6ccccc6)c6ccccc6)cc5)ccc4c4c(-c5ccccc5)cc5ccccc5c43)cc2)cc1] | 82% |
| 199 | 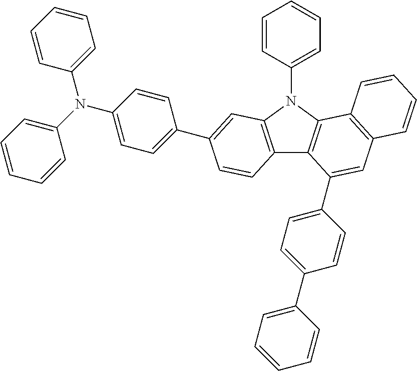 [SMILES: c1ccc(-c2ccc(-c3cc4ccccc4c4c3c3ccc(-c5ccc(N(c6ccccc6)c6ccccc6)cc5)cc3n4-c3ccccc3)cc2)cc1] | 77% |
| 200 | 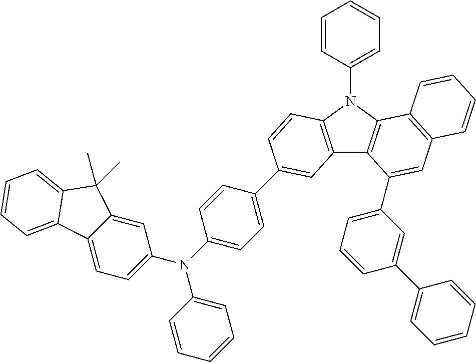 [SMILES: CC1(C)c2ccccc2-c2ccc(N(c3ccccc3)c3ccc(-c4ccc5c(c4)c4c(-c6cccc(-c7ccccc7)c6)cc6ccccc6c4n5-c4ccccc4)cc3)cc21] | 72% |
| 318 | 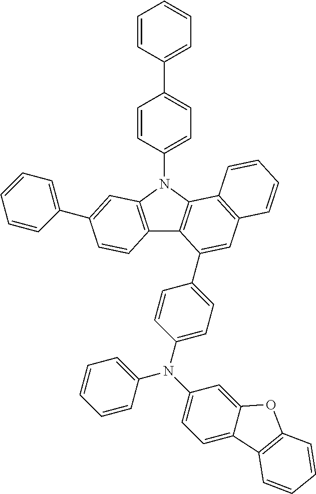 [SMILES: c1ccc(-c2ccc(-n3c4cc(-c5ccccc5)ccc4c4c(-c5ccc(N(c6ccccc6)c6ccc7c(c6)oc6ccccc67)cc5)cc5ccccc5c43)cc2)cc1] | 78% |

### [Preparation Example 4] Preparation of Compound 461

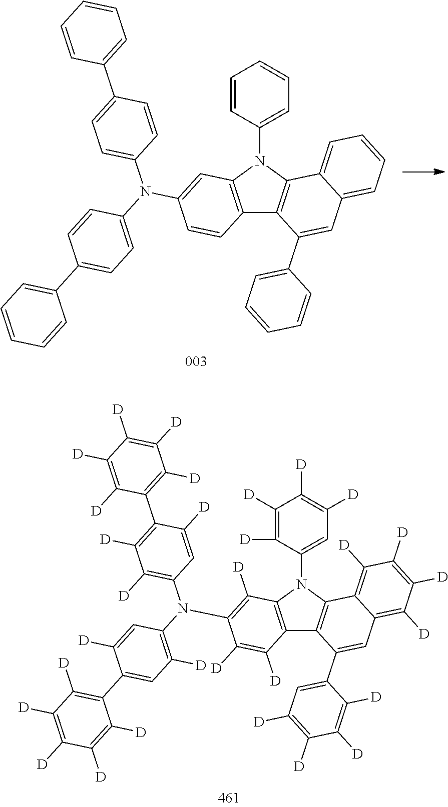 [SMILES: c1ccc(-c2ccc(N(c3ccc(-c4ccccc4)cc3)c3ccc4c5c(-c6ccccc6)cc6ccccc6c5n(-c5ccccc5)c4c3)cc2)cc1 ; [2H]c1cc(N(c2cc([2H])c(-c3c([2H])c([2H])c([2H])c([2H])c3[2H])c([2H])c2[2H])c2c([2H])c([2H])c3c4c(-c5cc([2H])c([2H])c([2H])c5[2H])cc5c([2H])c([2H])c([2H])c([2H])c5c4n(-c4cc([2H])c([2H])c([2H])c4[2H])c3c2[2H])c([2H])cc1-c1c([2H])c([2H])c([2H])c([2H])c1[2H]]

After Compound 003 (10 g, 14.52 mmol), trifluoromethanesulfonic acid (3.27 g, 21.78 mmol) and 100 ml of D6-benzene were put into a reaction flask, the resulting mixture was stirred under reflux for 5 hours. After the reaction was completed, the reaction was terminated by adding water thereto, extraction was performed using dichloromethane and distilled water, the organic layer was dried over anhydrous MgSO₄, and then the solvent was removed by a rotary evaporator, and then, the residue was purified by column chromatography using dichloromethane and hexane as an eluting solvent, thereby obtaining Compound 461 (9 g, 86%).

The compounds synthesized in the Preparation Examples were confirmed through 1H-NMR and FD-mass spectrometry. Table 4 shows the measured values of ¹H NMR (CDCl₃, 300 MHz), and Table 5 shows the measured values of field desorption mass spectrometry (FD-MS).

TABLE 4

| Compound | ¹H NMR(CDCl₃, 300 MHz) |
|---|---|
| 003 | δ = 8.18 (1H, d), 8.00 (2H, d), 7.79 (2H, d), |
| | 7.59-7.41 (24H, m), 7.22 (1H, s), 6.69 (4H, d), |
| | 6.60 (1H, s), 5.85 (1H, d) |
| 006 | δ = 8.18 (1H, d), 8.00 (2H, d), 7.87 (1H, d), 7.79 |
| | (2H, d), 7.62-7.38 (13H, m), 7.28 (1H, t), 7.22 |
| | (1H, s), 7.20 (2H, t), 6.81 (1H, t), 6.75 (1H, |
| | s), 6.63 (2H, d), 6.60 (1H, s), 6.58 (1H, d), |
| | 5.85 (1H, d), 1.72 (6H, s) |
| 007 | δ = 8.18 (1H, d), 8.00 (2H, d), 7.87 (1H, d), 7.79 |
| | (2H, d), 7.62-7.38 (20H, m), 7.28 (1H, t), 7.22 |
| | (1H, s), 6.75 (1H, s), 6.69 (2H, d), 6.60 (1H, |
| | s), 6.58 (1H, d), 5.85 (1H, d), 1.72 (6H, s) |
| 016 | δ = 8.18 (1H, d), 8.00 (2H, d), 7.87 (2H, d), 7.79 |
| | (2H, d), 7.59-7.11 (24H, m), 6.81 (1H, t), 6.76 |
| | (2H, d), 6.63 (2H, d), 6.60 (1H, s), 6.51 (2H, |
| | d), 5.85 (1H, d) |
| 017 | δ = 8.18 (1H, d), 8.00 (2H, d), 7.87 (1H, d), 7.79 |
| | (2H, d), 7.75 (2H, d), 7.59-7.16 (22H, m), 7.03 |
| | (1H, t), 6.91 (1H, d), 6.81 (1H, t), 6.63 (2H, |
| | d), 6.60 (1H, s), 6.58 (1H, d), 5.85 (1H, d) |
| 018 | δ = 8.18 (1H, d), 8.00 (2H, d), 7.87 (1H, d), 7.79 |
| | (2H, d), 7.59-7.03 (27H, m), 6.91 (1H, d), 6.81 |
| | (1H, t), 6.63 (2H, d), 6.60 (1H, s), 6.58 (1H, |
| | d), 5.85 (1H, d) |
| 023 | δ = 8.00 (2H, d), 7.79 (2H, d), 7.69 (1H, d), |
| | 7.63-7.41 (25H, m), 7.22 (1H, s), 6.69 (4H, d), |
| | 5.93 (1H, d) |
| 031 | δ = 8.93 (2H, d), 8.12 (2H, d), 8.00-7.79 (9H, m), |
| | 7.69-7.41 (14H, m), 7.22 (1H, s), 7.20 (2H, t), |
| | 6.81 (1H, t), 6.69 (2H, d), 6.63 (2H, d), 5.93 |
| | (1H, d) |
| 039 | δ = 8.18 (1H, d), 8.00 (3H, d), 7.79 (2H, d), 7.77 |
| | (1H, s), 7.59-7.41 (12H, m), 7.22 (1H, s), 7.20 |
| | (4H, t), 6.81 (2H, t), 6.69 (2H, d), 6.63 (4H, d) |
| 040 | δ = 8.18 (1H, d), 8.00 (3H, d), 7.87 (1H, d), 7.79 |
| | (2H, d), 7.77 (1H, s), 7.59-7.38 (15H, m), 7.28 |
| | (1H, t), 7.22 (1H, s), 7.20 (2H, t), 6.81 (1H, |
| | t), 6.75 (1H, s), 6.69 (2H, d), 6.63 (2H, d), |
| | 6.58 (1H, d), 1.72 (6H, s) |
| 042 | δ = 8.00 (2H, d), 7.79 (2H, d), 7.59-7.41 (18H, |
| | m), 7.22 (1H, s), 7.20 (2H, d), 7.04 (1H, t), |
| | 6.81 (1H, t), 6.77 (1H, d), 6.69 (2H, d), 6.63 |
| | (2H, d) |
| 073 | δ = 8.45 (1H, d), 8.18 (1H, d), 8.00 (2H, d), 7.98 |
| | (1H, d), 7.79 (4H, d), 7.73 (1H, d), 7.68 (2H, |
| | d), 7.59-7.40 (13H, m), 7.22 (1H, s), 7.20 (2H, |
| | t), 6.86 (1H, d), 6.81 (1H, t), 6.63 (2H, d), |
| | 6.60 (1H, s), 5.85 (1H, d) |
| 076 | δ = 8.00 (2H, d), 7.87 (2H, d), 7.79 (4H, d), |
| | 7.68-7.20 (26H, m), 6.81 (1H, t), 6.76 (2H, d), |
| | 6.63 (2H, d), 6.51 (2H, d), 5.93 (1H, d) |
| 079 | δ = 8.49 (1H, d), 8.10 (1H, d), 8.00 (2H, d), 7.79 |
| | (4H, d), 7.68-7.41 (15H, m), 7.22 (1H, s), 7.20 |
| | (4H, t), 6.81 (2H, t), 6.69 (2H, d), 6.63 (4H, d), |
| 089 | δ = 8.93 (2H, d), 8.18 (1H, d), 8.13 (1H, s), 8.12 |
| | (2H, d), 8.00 (5H, d), 7.88-7.79 (8H, m), 7.59 |
| | (4H, t), 7.51 (2H, t), 7.41 (1H, t), 7.36 (1H, |
| | d), 7.22 (1H, s), 7.20 (2H, t), 7.02 (1H, d), |
| | 6.81 (1H, t), 6.63 (2H, d), 6.60 (1H, s), 5.85 |
| | (1H, d) |
| 095 | δ = 8.18 (1H, d), 8.00 (5H, d), 7.89 (1H, d), 7.83 |
| | (1H, s), 7.79 (2H, d), 7.64-7.32 (13H, m), 7.22 |
| | (1H, s), 7.20 (2H, t), 6.81 (1H, t), 6.63 (2H, |
| | d), 6.60 (1H, s), 6.33 (1H, d), 5.85 (1H, d) |
| 105 | δ = 8.18 (1H, d), 8.08 (3H, d), 8.00 (2H, d), 7.79 |
| | (2H, d), 7.60-7.41 (22H, m), 7.22 (1H, s), 6.89 |
| | (1H, s), 6.88 (1H, d), 6.69 (2H, d), 6.60 (1H, |
| | s), 6.59 (1H, d), 5.85 (1H, d) |
| 108 | δ = 8.08 (3H, d), 8.00 (2H, d), 7.88-7.69 (7H, m), |
| | 7.60-7.36 (20H, m), 7.22 (1H, s), 6.69 (2H, d), |
| | 5.93 (1H, d) |
| 122 | δ = 8.00 (2H, d), 7.89 (1H, d), 7.79 (2H, d), |
| | 7.69-7.32 (20H, m), 7.22 (1H, s), 7.20 (2H, t), |
| | 6.81 (1H, t), 6.69 (2H, d), 6.63 (2H, d), 5.93 |
| | (1H, d) |
| 144 | δ = 8.90 (2H, d), 8.10 (2H, d), 8.00 (2H, d), |
| | 7.90-7.79 (6H, m), 7.70-7.41 (19H, m), 7.22 (1H, |
| | s), 7.16 (1H, t), 7.0 (2H, d), 6.87 (1H, t), 6.69 |
| | (3H, d), 5.93 (1H, d) |
| 163 | δ = 8.55 (1H, d), 8.42 (1H, d), 8.18 (1H, d), 8.08 |
| | (1H, d), 8.04 (1H, d), 8.00 (2H, d), 7.61-7.41 |
| | (24H, m), 7.22 (1H, s), 6.69 (4H, d), 6.60 (1H, |
| | s), 5.85 (1H, d) |
| 175 | δ = 8.55 (1H, d), 8.42 (1H, d), 8.18 (1H, d), 8.08 |
| | (1H, d), 8.04 (1H, d), 8.00 (2H, d), 7.89 (1H, |
| | d), 7.66-7.32 (15H, m), 7.22 (1H, s), 7.20 (2H, |
| | t), 6.81 (1H, t), 6.63 (2H, d), 6.60 (1H, s), |
| | 6.33 (1H, d), 5.85 (1H, d) |
| 184 | δ = 8.00 (2H, d), 7.70 (1H, s), 7.69 (1H, d), |
| | 7.63-7.41 (27H, m), 7.22 (1H, s), 7.16 (1H, t), |
| | 7.08 (2H, d), 6.87 (1H, t), 6.69 (3H, d), 5.93 |
| | (1H, d) |
| 199 | δ = 8.49 (1H, d), 8.10 (1H, d), 8.00 (2H, d), |
| | 7.62-7.41 (15H, m), 7.25 (4H, d), 7.22 (1H, s), |
| | 7.20 (4H, t), 6.81 (2H, t), 6.69 (2H, d), 6.63 |
| | (4H, d) |
| 200 | δ = 8.18 (1H, d), 8.00 (3H, d), 7.87 (1H, d), 7.77 |
| | (1H, s), 7.70 (1H, s), 7.62-7.38 (29H, m), 7.28 |
| | (1H, t), 7.22 (1H, s), 7.20 (2H t), 6.81 (1H, t), |
| | 6.75 (1H, s), 6.69 (2H, d), 6.63 (2H, d), 6.58 |
| | (1H, d), 1.72 (6H, s) |
| 202 | δ = 8.45 (1H, d), 8.00 (4H, d), 7.98 (1H, d), 7.86 |
| | (1H, d), 7.69 (1H, d), 7.63-7.41 (17H, m), 7.22 |
| | (1H, s), 7.20 (2H, t), 6.81 (1H, t), 6.69 (2H, |
| | d), 6.63 (2H, d), 5.93 (1H, d) |
| 221 | δ = 9.15 (1H, s), 8.93 (2H, d), 8.18 (2H, d), 8.12 |
| | (2H, d), 8.04 (1H, d), 8.00 (2H, d), 7.88 (2H, |
| | d), 7.82 (2H, d), 7.59-7.45 (7H, m), 7.22 (1H, |
| | s), 7.20 (4H, t), 6.81 (2H, t), 6.63 (4H, d), |
| | 6.60 (1H, s), 5.85 (1H, d) |
| 248 | δ = 8.49 (1H, d), 7.84 (1H, d), 8.00 (2H, d), 7.88 |
| | (1H, d), 7.84 (1H, d), 7.77 (1H, d), 7.74 (1H, |
| | s), 7.62-7.36 (23H, m), 6.69 (2H, d), 6.12 (1H, s) |
| 266 | δ = 8.18 (1H, d), 8.00 (3H, d), 7.87 (1H, d), 7.77 |
| | (1H, s), 7.62-7.38 (15H, m), 7.28 (1H, t), 7.20 |
| | (2H, t), 6.81 (1H, t), 6.75 (1H, s), 6.63 (2H, |
| | d), 6.58 (1H, d), 6.12 (1H, s), 1.72 (6H, s) |
| 296 | δ = 8.51 (1H, d), 8.30 (1H, d), 8.00 (2H, d), 7.87 |
| | (2H, d), 7.58-7.11 (26H, m), 6.81 (1H, t), 6.76 |
| | (2H, d), 6.63 (2H, d), 6.51 (2H, d), 6.12 (1H, s) |
| 302 | δ = 8.18 (1H, d), 8.00 (3H, d), 7.79 (2H, d), 7.77 |
| | (1H, s), 7.68 (2H, d), 7.59-7.41 (19H, m), 7.20 |
| | (2H, t), 6.81 (1H, t), 6.69 (2H, d), 6.63 (2H, |
| | d), 6.12 (1H, s) |
| 318 | δ = 8.49 (1H, d), 8.10 (1H, d), 8.00 (2H, d), 7.89 |
| | (1H, d), 7.79 (2H, d), 7.66-7.41 (22H, m), 7.22 |
| | (1H, s), 7.20 (2H, t), 6.81 (1H, t), 6.69 (2H, |
| | d), 6.63 (2H, d), 6.33 (1H, d) |
| 323 | δ = 8.49 (1H, d), 8.10 (1H, d), 8.00 (5H, d), 7.83 |
| | (1H, s), 7.62-7.36 (25H, m), 6.69 (4H, d), 6.12 |
| | (1H, s) |
| 341 | δ = 8.49 (1H, d), 8.45 (1H, d), 8.10 (1H, d), 8.00 |
| | (2H, d), 7.98 (2H, d), 7.94 (1H, s), 7.62-7.41 |
| | (11H, m), 7.20 (4H, t), 6.81 (2H, t), 6.63 (4H, |
| | d), 6.12 (1H, s) |
| 361 | δ = 8.93 (1H, d), 8.90 (2H, d), 8.49 (1H, d), |
| | 8.12-8.10 (4H, m), 8.00 (2H, d), 7.90-7.80 (5H, |
| | m), 7.62-7.41 (8H, m), 7.20 (4H, t), 6.81 (2H, |
| | t), 6.63 (4H, d), 6.12 (1H, s) |
| 381 | δ = 8.49 (1H, d), 8.10 (1H, d), 8.00 (2H, d), |
| | 7.62-7.41 (13H, m), 7.25 (4H, s), 7.20 (4H, t), |
| | 6.81 (2H, t), 6.63 (4H, d), 6.12 (1H, s) |
| 403 | δ = 8.55 (1H, d), 8.49 (1H, d), 8.42 (1H, d), 8.10 |
| | (1H, d), 8.08 (1H, d), 8.04 (1H, d), 8.00 (2H, |
| | d), 7.62-7.41 (25H, m), 6.69 (4H, d), 6.12 (1H, s) |
| 405 | δ = 8.55 (1H, d), 8.49 (1H, d), 8.42 (1H, d), 8.10 |
| | (1H, d), 8.08 (1H, d), 8.04 (1H, d), 8.00 (2H, |
| | d), 7.62-7.41 (24H, m), 6.89 (1H, s), 6.88 (1H, |
| | d), 6.69 (2H, d), 6.59 (1H, d), 6.12 (1H, s) |
| 435 | δ = 8.49 (1H, d), 8.45 (1H, d), 8.41 (1H, d), 8.20 |
| | (1H, d), 8.10 (1H, d), 8.00 (2H, d), 7.98 (1H, |
| | d), 7.89 (1H, d), 7.66-7.32 (16H, m), 7.20 (2H, |
| | t), 6.81 (1H, t), 6.63 (2H, d), 6.33 (1H, d), |
| | 6.12 (1H, s) |
| 442 | δ = 8.93 (2H, d), 8.18 (1H, d), 8.12 (2H, d), |
| | 8.00-7.77 (9H, m), 7.59-7.41 (14H, m), 7.20 (2H, |
| | t), 6.81 (1H, t), 6.69 (2H, d), 6.63 (2H, d), |
| | 6.12 (1H, s) |
| 461 | δ = 7.66 (1H, s), 7.44 (1H, s), 7.41 (1H, s), 7.22 |
| | (1H, s), 6.70 (2H, s) |
| 468 | δ = 8.55 (1H, d), 8.42 (1H, d), 8.18 (1H, d), 8.08 |
| | (1H, d), 8.04 (1H, d), 8.00 (2H, d), 7.61-7.45 |
| | (10H, m), 7.22 (1H, s), 6.60 (1H, s), 5.85 (1H, d) |
| 474 | δ = 8.00 (2H, d), 7.79 (2H, d), 7.59-7.41 (17H, |
| | m), 7.30 (1H, d), 7.22-7.20 (3H, m), 7.08 (1H, |
| | t), 6.81 (1H, t), 6.69 (2H, d), 6.63 (2H, d), |
| | 5.85 (1H, d) |
| 488 | δ = 8.10 (1H, d), 8.00 (2H, d), 7.90 (1H, d), 7.79 |
| | (2H, d), 7.62-7.38 (14H, m), 7.28 (1H, t), 7.20 |
| | (2H, t), 6.81 (1H, t), 6.75 (1H, s), 6.63 (2H, |
| | d), 6.58 (1H, d), 6.12 (1H, s), 1.72 (6H, s) |

TABLE 5

| Compound | FD-MS |
|---|---|
| 003 | m/z = 688.86 |
| | (C₅₂H₃₆N₂ = 688.29) |
| 006 | m/z = 652.82 |
| | (C₄₉H₃₆N₂ = 652.29) |
| 007 | m/z = 728.92 |
| | (C₅₅H₄₀N₂ = 728.32) |
| 016 | m/z = 776.96 |
| | (C₅₉H₄₀N₂ = 776.32) |
| 017 | m/z = 774.95 |
| | (C₅₉H₃₈N₂ = 774.30) |
| 018 | m/z = 776.96 |
| | (C₅₉H₄₀N₂ = 776.32) |
| 023 | m/z = 688.86 |
| | (C₅₂H₃₆N₂ = 688.29) |
| 031 | m/z = 712.88 |
| | (C₅₄H₃₆N₂ = 712.29) |
| 039 | m/z = 612.76 |
| | (C₄₆H₃₂N₂ = 612.26) |
| 040 | m/z = 728.92 |
| | (C₅₅H₄₀N₂ = 728.32) |
| 042 | m/z = 612.76 |
| | (C₄₆H₃₂N₂ = 612.26) |
| 073 | m/z = 718.90 |
| | (C₅₂H₃₄N₂S = 718.24) |
| 076 | m/z = 853.06 |
| | (C₆₅H₄₄N₂ = 852.35) |
| 079 | m/z = 688.86 |
| | (C₅₂H₃₆N₂ = 688.29) |
| 089 | m/z = 736.90 |
| | (C₅₆H₃₆N₂ = 736.29) |
| 095 | m/z = 676.80 |
| | (C₅₀H₃₂N₂O = 676.25) |
| 105 | m/z = 738.91 |
| | (C₅₆H₃₈N₂ = 738.30) |
| 108 | m/z = 712.88 |
| | (C₅₄H₃₆N₂ = 712.29) |
| 122 | m/z = 702.84 |
| | (C₅₂H₃₄N₂O = 702.27) |
| 144 | m/z = 788.97 |
| | (C₆₀H₄₀N₂ = 788.32) |
| 163 | m/z = 738.91 |
| | (C₅₆H₃₈N₂ = 738.30) |
| 474 | m/z = 612.76 |
| | (C₄₆H₃₂N₂ = 612.26) |
| 175 | m/z = 676.80 |
| | (C₅₀H₃₂N₂O = 676.25) |
| 184 | m/z = 764.95 |
| | (C₅₈H₄₀N₂ = 764.32) |
| 199 | m/z = 688.86 |
| | (C₅₂H₃₆N₂ = 688.29) |
| 200 | m/z = 805.02 |
| | (C₆₁H₄₄N₂ = 804.35) |
| 202 | m/z = 718.90 |
| | (C₅₂H₃₄N₂S = 718.24) |
| 221 | m/z = 686.84 |
| | (C₅₂H₃₄N₂ = 686.27) |
| 248 | m/z = 662.82 |
| | (C₅₀H₃₄N₂ = 662.27) |
| 266 | m/z = 652.82 |
| | (C₄₉H₃₆N₂ = 652.29) |
| 296 | m/z = 776.96 |
| | (C₅₉H₄₀N₂ = 776.32) |
| 302 | m/z = 688.86 |
| | (C₅₂H₃₆N₂ = 688.29) |
| 318 | m/z = 778.94 |
| | (C₅₈H₃₈N₂O = 778.30) |
| 323 | m/z = 738.91 |
| | (C₅₆H₃₈N₂ = 738.30) |
| 341 | m/z = 642.81 |
| | (C₄₆H₃₀N₂S = 642.21) |
| 361 | m/z = 686.84 |
| | (C₅₂H₃₄N₂ = 686.27) |
| 381 | m/z = 612.76 |
| | (C₄₆H₃₂N₂ = 612.26) |
| 403 | m/z = 738.91 |
| | (C₅₆H₃₈N₂ = 738.30) |
| 405 | m/z = 738.91 |
| | (C₅₆H₃₈N₂ = 738.30) |
| 435 | m/z = 732.89 |
| | (C₅₂H₃₂N₂OS = 732.22) |
| 442 | m/z = 712.88 |
| | (C₅₄H₃₆N₂ = 712.29) |
| 461 | m/z = 719.04 |
| | (C₅₂H₆D₃₀N₂ = 718.48) |
| 468 | m/z = 757.03 |
| | (C₅₆H₂₀D₁₈N₂ = 756.42) |
| 488 | m/z = 652.82 |
| | (C₄₉H₃₆N₂ = 652.29) |

### EXPERIMENTAL EXAMPLES

### Experimental Example 1

### (1) Manufacture of Organic Light Emitting Device

A glass substrate, in which indium tin oxide (ITO) was thinly coated to have a thickness of 1,500 Å, was ultrasonically washed with distilled water. When the washing with distilled water was finished, the glass substrate was ultrasonically washed with a solvent such as acetone, methanol, and isopropyl alcohol, dried and then was subjected to UVO treatment for 5 minutes using UV in a UV cleaning machine. Thereafter, the substrate was transferred to a plasma washing machine (PT), and then was subjected to plasma treatment in a vacuum state for an ITO work function and in order to remove a residual film, and was transferred to a thermal deposition apparatus for organic deposition.

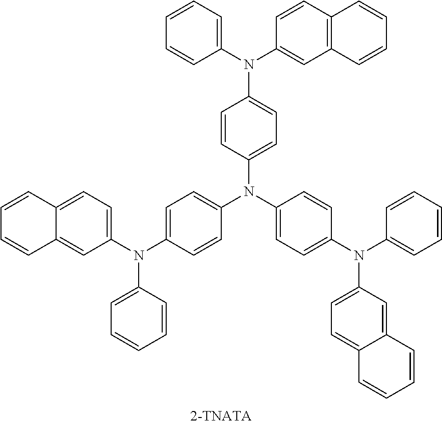 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1]

Subsequently, air in the chamber was evacuated until the degree of vacuum in the chamber reached 10⁻⁶ torr, and then a hole injection layer having a thickness of 600 Å was deposited on the ITO substrate by applying current to the cell to evaporate 2-TNATA. A hole transport layer having a thickness of 1000 Å was deposited on the hole injection layer by placing the following N,N′-bis(α-naphthyl)-N,N′-diphenyl-4,4′-diamine (NPB) in another cell in the vacuum deposition apparatus and applying current to the cell to evaporate NPB.

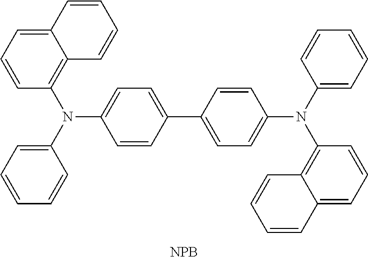 [SMILES: c1ccc(N(c2ccc(-c3ccc(N(c4ccccc4)c4cccc5ccccc45)cc3)cc2)c2cccc3ccccc23)cc1 ; "NPB"]

A light emitting layer was thermally vacuum deposited thereon as follows. The light emitting layer was deposited by depositing a compound of 9-[4-(4,6-diphenyl-1,3,5-triazin-2-yl)phenyl]-9′-phenyl-3,3′-bi-9H-carbazole as a host to have a thickness of 400 Å and doping the deposited layer with a green phosphorescent dopant Ir(ppy)₃ at 7%. Thereafter, bathocuproine (BCP) as a hole blocking layer was deposited to 60 Å, and E1 as an electron transport layer was deposited to 300 Å thereon.

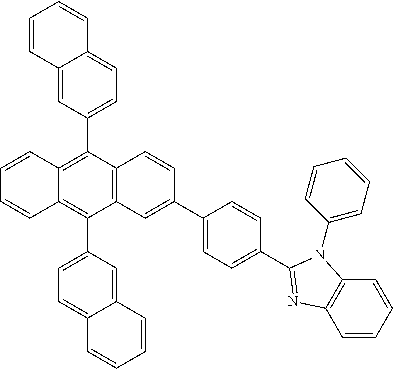 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1]

Finally, lithium fluoride (LiF) was deposited to have a thickness of 10 Å on the electron transport layer to form an electron injection layer, and then an aluminum (Al) negative electrode was deposited to have a thickness of 1,200 Å on the electron injection layer to form a negative electrode, thereby manufacturing an organic electroluminescence device.

Meanwhile, all the organic compounds required for manufacturing an OLED device were subjected to vacuum sublimed purification under 10⁻⁶ to 10⁻⁸ torr for each material, and used for the manufacture of OLED.

The driving voltage and light emitting efficiency of the organic electroluminescence device according to Experimental Example 1 are shown in the following Table 6, except that Compounds shown in the following Table 6 were used instead of the compound NPB used when the hole transport layer was formed in Experimental Example 1.

In this case, the hole transport compounds of Comparative Examples 2 to 4, excluding NPB, are as follows.

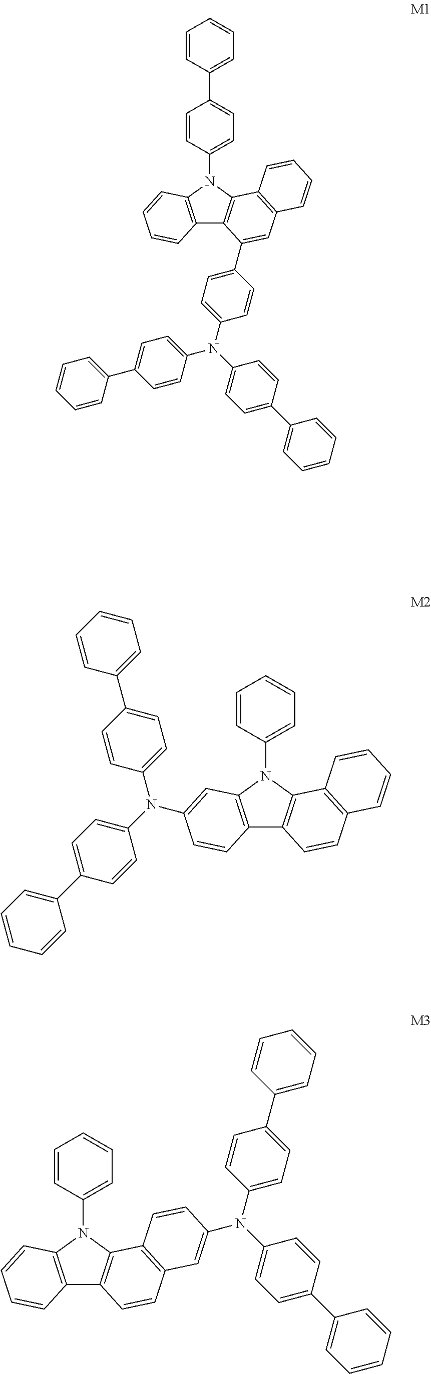 [SMILES: c1ccc(-c2ccc(N(c3ccc(-c4ccccc4)cc3)c3ccc(-c4cc5ccccc5c5c4c4ccccc4n5-c4ccc(-c5ccccc5)cc4)cc3)cc2)cc1 ; c1ccc(-c2ccc(N(c3ccc(-c4ccccc4)cc3)c3ccc4c5ccc6ccccc6c5n(-c5ccccc5)c4c3)cc2)cc1 ; c1ccc(-c2ccc(N(c3ccc(-c4ccccc4)cc3)c3ccc4c(ccc5c6ccccc6n(-c6ccccc6)c45)c3)cc2)cc1]

### (2) Driving Voltage and Light Emitting Efficiency of Organic Light Emitting Device

For the organic electroluminescence device manufactured as described above, electroluminescence (EL) characteristics were measured by M7000 manufactured by McScience Inc., and based on the measurement result thereof, T₉₅ (time taken to decrease from reference luminance to 95%) was measured by a service life measurement equipment (M6000) manufactured by McScience Inc., when the reference luminance was 20,000 cd/m2.

Characteristics of the organic electroluminescence device of the present invention are as described in the following Table 6.

TABLE 6

| | | Driving | Light emitting | Service |
|---|---|---|---|---|
| | | voltage | efficiency | life |
| | Compound | (V) | (cd/A) | (T₉₅) |
| Example 1 | 003 | 4.25 | 122.44 | 149 |
| Example 2 | 006 | 4.30 | 119.68 | 145 |
| Example 3 | 007 | 4.28 | 123.21 | 136 |
| Example 4 | 016 | 4.20 | 120.76 | 139 |
| Example 5 | 017 | 4.13 | 124.53 | 147 |
| Example 6 | 018 | 4.17 | 118.87 | 138 |
| Example 7 | 023 | 4.14 | 120.72 | 139 |
| Example 8 | 031 | 4.19 | 124.35 | 136 |
| Example 9 | 039 | 4.24 | 122.28 | 144 |
| Example 10 | 040 | 4.15 | 124.91 | 147 |
| Example 11 | 042 | 4.11 | 117.77 | 138 |
| Example 12 | 073 | 4.27 | 124.27 | 149 |
| Example 13 | 076 | 4.26 | 116.69 | 134 |
| Example 14 | 079 | 4.37 | 119.53 | 138 |
| Example 15 | 089 | 4.29 | 122.24 | 143 |
| Example 16 | 095 | 4.32 | 116.74 | 137 |
| Example 17 | 105 | 4.11 | 115.55 | 138 |
| Example 18 | 108 | 4.25 | 123.21 | 142 |
| Example 19 | 122 | 4.21 | 120.26 | 146 |
| Example 20 | 144 | 4.17 | 118.75 | 134 |
| Example 21 | 163 | 4.29 | 122.30 | 140 |
| Example 22 | 175 | 4.33 | 116.09 | 134 |
| Example 23 | 184 | 4.26 | 119.15 | 133 |
| Example 24 | 199 | 4.21 | 117.66 | 148 |
| Example 25 | 200 | 4.30 | 120.37 | 136 |
| Example 26 | 202 | 4.33 | 118.49 | 137 |
| Example 27 | 221 | 4.26 | 120.36 | 139 |
| Example 28 | 248 | 4.28 | 122.83 | 130 |
| Example 29 | 266 | 4.17 | 118.55 | 132 |
| Example 30 | 296 | 4.21 | 121.67 | 135 |
| Example 31 | 302 | 4.13 | 119.17 | 133 |
| Example 32 | 318 | 4.12 | 123.56 | 141 |
| Example 33 | 323 | 4.24 | 119.09 | 132 |
| Example 34 | 341 | 4.28 | 117.76 | 138 |
| Example 35 | 361 | 4.26 | 120.57 | 141 |
| Example 36 | 381 | 4.31 | 119.78 | 135 |
| Example 37 | 403 | 4.20 | 125.42 | 138 |
| Example 38 | 405 | 4.23 | 120.18 | 136 |
| Example 39 | 435 | 4.18 | 118.87 | 133 |
| Example 40 | 442 | 4.21 | 117.67 | 147 |
| Example 41 | 461 | 4.27 | 122.24 | 197 |
| Example 42 | 468 | 4.28 | 122.09 | 181 |
| Comparative | NPB | 5.37 | 85.21 | 96 |
| Example 1 | | | | |
| Comparative | M1 | 4.76 | 98.59 | 110 |
| Example 2 | | | | |
| Comparative | M2 | 4.65 | 101.64 | 104 |
| Example 3 | | | | |
| Comparative | M3 | 4.70 | 96.47 | 110 |
| Example 4 | | | | |

As can be seen from the results of Table 6, in Examples 1 to 42, which are organic light emitting devices using the compound represented by Chemical Formula 1 of the present invention as a hole transport layer material, the drive voltage was low, and the light emitting efficiency and service life were improved compared to Comparative Examples 1 to 4, which are organic light emitting devices that do not use the compound represented by Chemical Formula 1 of the present invention as a material for the hole transport layer.

The compounds M1 to M3 used in Comparative Examples 2 to 4 are similar to the present invention in that an arylamine group is introduced as a substituent into the benzocarbazole-type five-ring skeleton, but the present invention has a disubstituted structure in which not only an arylamine group is introduced into the skeleton, but also an aryl group or a heteroaryl group is additionally introduced, for example, a structure in which two substituents are substituted.

Such a disubstituted structure has an advantage in that structural stability is excellent compared to the monosubstituted structures of Comparative Examples 2 to 4, and hole mobility becomes faster than that of the monosubstituted structure. For this reason, it can be confirmed that the compound of the present invention has a low driving voltage and improved efficiency and service life compared to the compounds of Comparative Examples 2 to 4.

### Experimental Example 2

### (1) Manufacture of Organic Light Emitting Device

Trichloroethylene, acetone, ethanol, and distilled water were each sequentially used to ultrasonically wash transparent electrode ITO thin film obtained from glass for OLED (manufactured by Samsung-Corning Co., Ltd.) for 5 minutes, and then the ITO thin film was placed in isopropanol, stored, and then used. Next, the ITO substrate was disposed in a substrate folder of a vacuum deposition apparatus, and the following 4,4′,4″-tris(N,N-(2-naphthyl)-phenylamino)triphenyl amine (2-TNATA) was placed in a cell in the vacuum deposition apparatus.

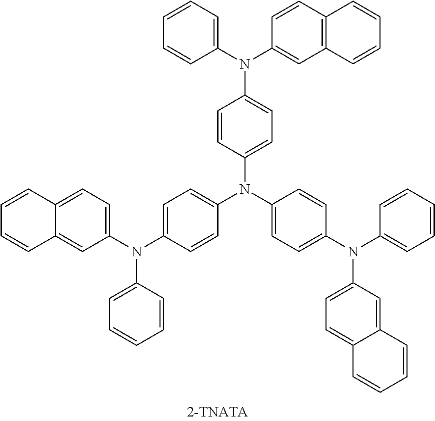 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1]

Subsequently, air in the chamber was evacuated until the degree of vacuum in the chamber reached 10⁻⁶ torr, and then a hole injection layer having a thickness of 600 Å was deposited on the ITO substrate by applying current to the cell to evaporate 2-TNATA. A hole transport layer having a thickness of 1000 Å was deposited on the hole injection layer by placing the following N,N′-bis(α-naphthyl)-N,N′-diphenyl-4,4′-diamine (NPB) in another cell in the vacuum deposition apparatus and applying current to the cell to evaporate NPB.

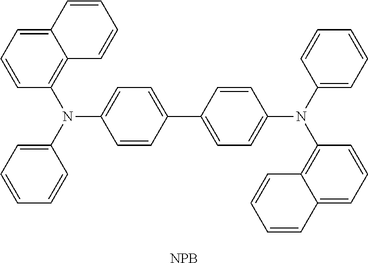 [SMILES: c1ccc(N(c2ccc(-c3ccc(N(c4ccccc4)c4cccc5ccccc45)cc3)cc2)c2cccc3ccccc23)cc1 ; "NPB"]

Subsequently, an electron blocking layer was deposited to have a thickness of 100 Å using a compound having the following structural formula M1.

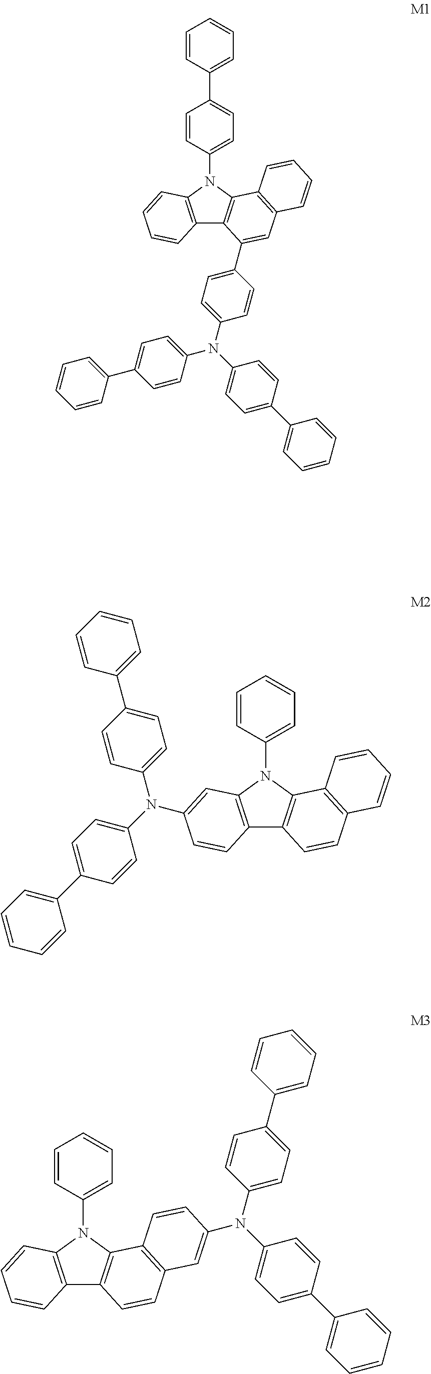 [SMILES: c1ccc(-c2ccc(N(c3ccc(-c4ccccc4)cc3)c3ccc(-c4cc5ccccc5c5c4c4ccccc4n5-c4ccc(-c5ccccc5)cc4)cc3)cc2)cc1 ; c1ccc(-c2ccc(N(c3ccc(-c4ccccc4)cc3)c3ccc4c5ccc6ccccc6c5n(-c5ccccc5)c4c3)cc2)cc1 ; c1ccc(-c2ccc(N(c3ccc(-c4ccccc4)cc3)c3ccc4c(ccc5c6ccccc6n(-c6ccccc6)c45)c3)cc2)cc1]

A blue light emitting material having the following structure was deposited as a light emitting layer thereon. Specifically, a blue light emitting host material H1 was vacuum deposited to have a thickness of 300 Å on one cell in the vacuum deposition apparatus, and a blue light emitting dopant material D1 was vacuum deposited thereon in an amount of 5% based on the host material.

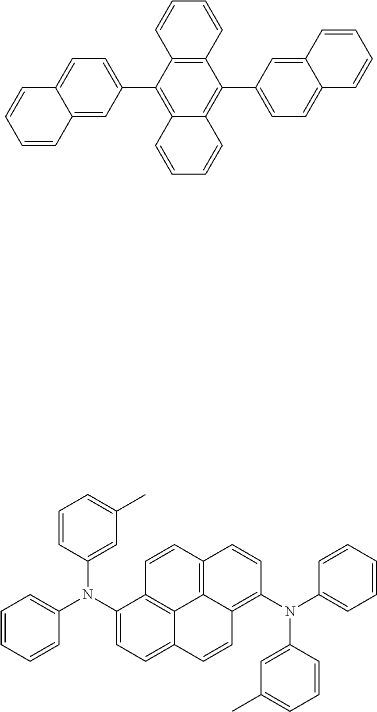

Subsequently, a compound having the following structural formula E1 as an electron transport layer was deposited to have a thickness of 300 Å.

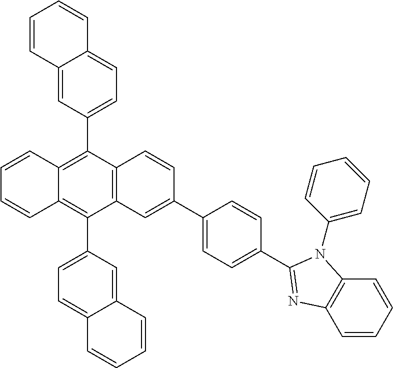 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1]

An OLED device was manufactured by depositing lithium fluoride (LiF) as an electron injection layer to have a thickness of 10 Å and allowing the Al negative electrode to have a thickness of 1,000 Å. Meanwhile, all the organic compounds required for manufacturing an OLED device were subjected to vacuum sublimed purification under 10⁻⁶ to 10⁻⁸ torr for each material, and used for the manufacture of OLED.

### (2) Driving Voltage and Light Emitting Efficiency of Organic Light Emitting Device

The driving voltage and light emitting efficiency of the organic electroluminescence device according to Experimental Example 2 are shown in the following Table 7, except that Compounds shown in the following Table 7 were used instead of the compound M1 used when the electron blocking layer was formed in Experimental Example 2.

TABLE 7

| | | Driving | Light emitting | Service |
|---|---|---|---|---|
| | | voltage | efficiency | life |
| | Compound | (V) | (cd/A) | (T₉₅) |
| Example 43 | 003 | 5.22 | 6.93 | 72 |
| Example 44 | 016 | 5.35 | 7.07 | 69 |
| Example 45 | 017 | 5.28 | 6.99 | 71 |
| Example 46 | 018 | 5.30 | 7.11 | 70 |
| Example 47 | 023 | 5.27 | 6.82 | 73 |
| Example 48 | 027 | 5.33 | 6.93 | 75 |
| Example 49 | 031 | 5.07 | 7.01 | 73 |
| Example 50 | 039 | 5.17 | 6.90 | 75 |
| Example 51 | 040 | 5.41 | 6.84 | 74 |
| Example 52 | 079 | 5.07 | 7.13 | 78 |
| Example 53 | 089 | 5.29 | 6.94 | 70 |
| Example 54 | 163 | 5.30 | 6.96 | 68 |
| Example 55 | 202 | 5.42 | 7.15 | 71 |
| Example 56 | 221 | 5.18 | 6.70 | 68 |
| Example 57 | 248 | 5.34 | 6.87 | 72 |
| Example 58 | 296 | 5.32 | 6.97 | 73 |
| Example 59 | 302 | 5.25 | 6.83 | 76 |
| Example 60 | 323 | 5.36 | 6.97 | 71 |
| Example 61 | 403 | 5.14 | 6.88 | 68 |
| Example 62 | 461 | 5.23 | 6.90 | 102 |
| Example 63 | 468 | 5.32 | 6.94 | 109 |
| Comparative | M1 | 6.02 | 5.88 | 50 |
| Example 5 | | | | |
| Comparative | M2 | 6.21 | 5.70 | 53 |
| Example 6 | | | | |
| Comparative | M3 | 6.16 | 5.93 | 49 |
| Example 7 | | | | |
| Comparative | NPB | 6.40 | 5.66 | 44 |
| Example 8 | | | | |

As can be seen from the results of Table 7, in the organic light emitting devices of Examples 43 to 63 using the compound represented by Chemical Formula 1 of the present invention as an electron blocking layer material, the drive voltage was low, and the light emitting efficiency and service life were remarkably improved compared to the organic light emitting devices of Comparative Examples 5 to 8 using M1 to M3 and NPB compounds as electron blocking layer materials.

### Experimental Example 3

### (1) Manufacture of Organic Light Emitting Device

A glass substrate, in which indium tin oxide (ITO) was thinly coated to have a thickness of 1,500 Å, was ultrasonically washed with distilled water. When the washing with distilled water was finished, the glass substrate was ultrasonically washed with a solvent such as acetone, methanol, and isopropyl alcohol, dried and then was subjected to UVO treatment for 5 minutes using UV in a UV cleaning machine. Thereafter, the substrate was transferred to a plasma washing machine (PT), and then was subjected to plasma treatment in a vacuum state for an ITO work function and in order to remove a residual film, and was transferred to a thermal deposition apparatus for organic deposition.

Subsequently, air in the chamber was evacuated until the degree of vacuum in the chamber reached 10⁻⁶ torr, and then a hole injection layer having a thickness of 600 Å was deposited on the ITO substrate by applying current to the cell to evaporate 2-TNATA. A hole transport layer having a thickness of 1000 Å was deposited on the hole injection layer by placing the following N,N′-bis(α-naphthyl)-N,N′-diphenyl-4,4′-diamine (NPB) in another cell in the vacuum deposition apparatus and applying current to the cell to evaporate NPB.

A light emitting layer was thermally vacuum deposited thereon as follows. The compounds shown in the following Table 8 were used as the host for the light emitting layer by a vapor deposition method, and the host was doped with (piq)₂(Ir)(acac) as a red phosphorescent dopant at 3% based on the weight of the host material and deposited to a thickness of 500 Å.

Thereafter, bathocuproine (BCP) was deposited as a hole blocking layer to have a thickness of 60 Å, and Alq₃ was deposited as an electron transport layer to have a thickness of 200 Å thereon.

Finally, an organic light emitting device was manufactured by depositing lithium fluoride (LiF) to have a thickness of 10 Å on the electron transport layer to form an electron injection layer, and then depositing an aluminum (Al) negative electrode to have a thickness of 1,200 Å on the electron injection layer to form a negative electrode.

Specifically, the compounds used as hosts in Examples 64 to 77 and Comparative Examples 9 to 12 are shown in the following Table 8.

In this case, Compounds M1 to M3 used as hosts in Comparative Examples 9 to 11 of the following Table 8 are as follows.

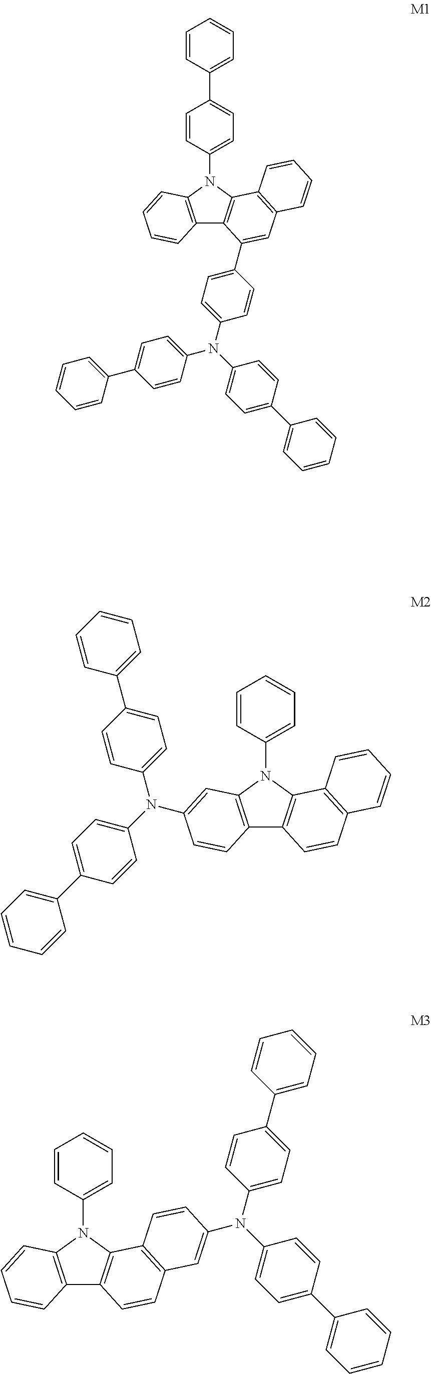 [SMILES: c1ccc(-c2ccc(N(c3ccc(-c4ccccc4)cc3)c3ccc(-c4cc5ccccc5c5c4c4ccccc4n5-c4ccc(-c5ccccc5)cc4)cc3)cc2)cc1 ; c1ccc(-c2ccc(N(c3ccc(-c4ccccc4)cc3)c3ccc4c5ccc6ccccc6c5n(-c5ccccc5)c4c3)cc2)cc1 ; c1ccc(-c2ccc(N(c3ccc(-c4ccccc4)cc3)c3ccc4c(ccc5c6ccccc6n(-c6ccccc6)c45)c3)cc2)cc1]

Meanwhile, all the organic compounds required for manufacturing an organic light emitting device were subjected to vacuum sublimed purification under 10⁻⁶ to 10⁻⁸ torr for each material, and used for the manufacture of the organic light emitting device.

### (2) Driving Voltage and Light Emitting Efficiency of Organic Light Emitting Device

For the organic light emitting device manufactured as described above, electroluminescence (EL) characteristics were measured by M7000 manufactured by McScience Inc., and based on the measurement result thereof, T₉₅ was measured by a service life measurement equipment (M6000) manufactured by McScience Inc., when the reference luminance was 6,000 cd/m². The results of measuring the driving voltage, light emitting efficiency, light emission color and service life of the organic light emitting device manufactured according to the present invention are shown in the following Table 8.

TABLE 8

| | | Driving | Light emitting | Service |
|---|---|---|---|---|
| | | voltage | efficiency | life |
| | Compound | (V) | (cd/A) | (T₉₅) |
| Example 64 | 003 | 4.62 | 22.71 | 52 |
| Example 65 | 039 | 5.03 | 21.52 | 70 |
| Example 66 | 079 | 4.77 | 22.01 | 73 |
| Example 67 | 089 | 4.68 | 24.87 | 59 |
| Example 68 | 122 | 5.13 | 20.76 | 54 |
| Example 69 | 199 | 5.10 | 21.38 | 75 |
| Example 70 | 221 | 4.86 | 23.28 | 62 |
| Example 71 | 266 | 4.55 | 22.41 | 55 |
| Example 72 | 302 | 4.74 | 23.08 | 51 |
| Example 73 | 323 | 4.84 | 22.96 | 57 |
| Example 74 | 435 | 4.75 | 23.28 | 60 |
| Example 75 | 465 | 5.07 | 21.44 | 92 |
| Example 76 | 474 | 4.94 | 22.27 | 55 |
| Example 77 | 488 | 4.83 | 21.79 | 57 |
| Comparative | M1 | 5.88 | 16.43 | 36 |
| Example 9 | | | | |
| Comparative | M2 | 5.79 | 15.87 | 26 |
| Example 10 | | | | |
| Comparative | M3 | 5.62 | 16.38 | 29 |
| Example 11 | | | | |
| Comparative | NPB | 6.09 | 13.74 | 20 |
| Example 12 | | | | |

As can be seen from the results of Table 8, in the organic light emitting devices of Examples 64 to 77 using the compound represented by Chemical Formula 1 of the present invention as a host material for the light emitting layer, the drive voltage was low, and the light emitting efficiency and service life were remarkably improved compared to the organic light emitting devices of Comparative Examples 9 to 12 using M1 to M3 and NPB compounds as host materials for the light emitting layer.

1. A heterocyclic compound represented by the following Chemical Formula 1: 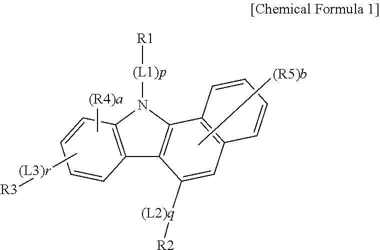
wherein, in Chemical Formula 1,
L1 to L3 are the same as or different from each other, and are each independently a direct bond; a substituted or unsubstituted C6 to C60 arylene group; or a substituted or unsubstituted C2 to C60 heteroarylene group,
R1 is a substituted or unsubstituted C6 to C60 aryl group; or a substituted or unsubstituted C2 to C60 heteroaryl group,
R2 and R3 are the same as or different from each other, and are each independently a substituted or unsubstituted C6 to C60 aryl group; a substituted or unsubstituted C2 to C60 heteroaryl group; or —N(Ra)(Rb), and at least one of R2 and R3 is —N(Ra)(Rb),
Ra and Rb are the same as or different from each other, and are each independently a substituted or unsubstituted C6 to C60 aryl group; or a substituted or unsubstituted C2 to C60 heteroaryl group,
R4 to R5 are the same as or different from each other, and are each independently hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group,
a is an integer from 0 to 3, b is an integer from 0 to 5, and when a and b are each an integer of 2 or higher, substituents in the parenthesis are the same as or different from each other, and
p, q and r are each an integer from 0 to 4, and when p, q and r are each an integer of 2 or higher, substituents in the parenthesis are the same as or different from each other.

2. The heterocyclic compound of claim 1, wherein Chemical Formula 1 is represented by any one of the following Chemical Formulae 1-1 to 1-5: 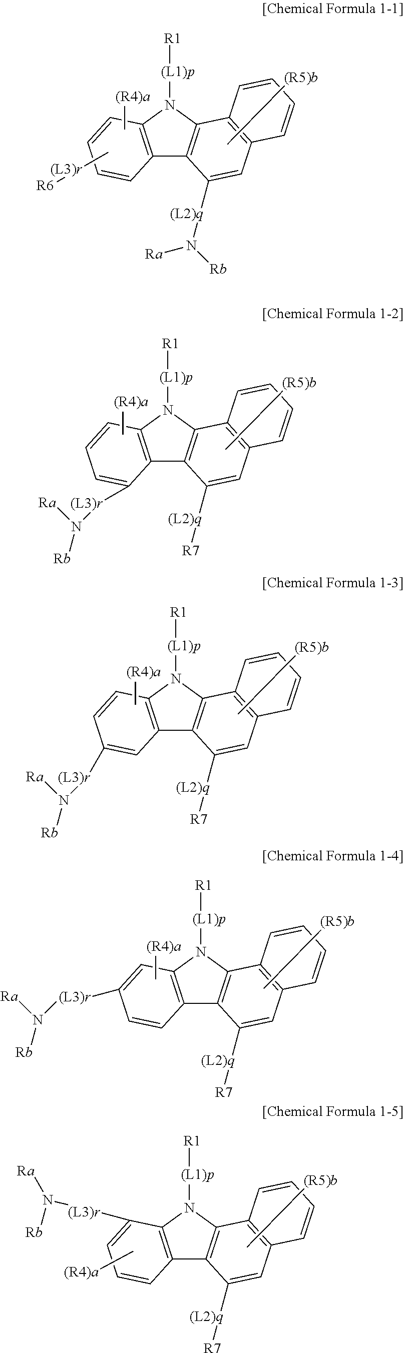
in Chemical Formulae 1-1 to 1-5,
the definitions of L1 to L3, R1, R4, R5, Ra, Rb, a, b, p, q and r are the same as the definitions in Chemical Formula 1, and
R6 and R7 are the same as or different from each other, and are each independently a substituted or unsubstituted C6 to C60 aryl group; or a substituted or unsubstituted C2 to C60 heteroaryl group.

3. The heterocyclic compound of claim 1, wherein Ra and Rb are the same as or different from each other, and are each independently a substituted or unsubstituted C6 to C40 aryl group; or a substituted or unsubstituted C2 to C40 heteroaryl group.

4. The heterocyclic compound of claim 1, wherein R2 and R3 are the same as or different from each other, and are each independently a substituted or unsubstituted C6 to C40 aryl group; a substituted or unsubstituted C2 to C40 heteroaryl group; or —N(Ra)(Rb), and at least one of R2 and R3 is —N(Ra)(Rb).

5. The heterocyclic compound of claim 1, wherein R4 and R5 are the same as or different from each other, and are each independently hydrogen; or deuterium.

6. The heterocyclic compound of claim 1, wherein a deuterium content of the heterocyclic compound of Chemical Formula 1 is 0%, or 30% to 100%.

7. The heterocyclic compound of claim 1, wherein Chemical Formula 1 is represented by any one of the following compounds:

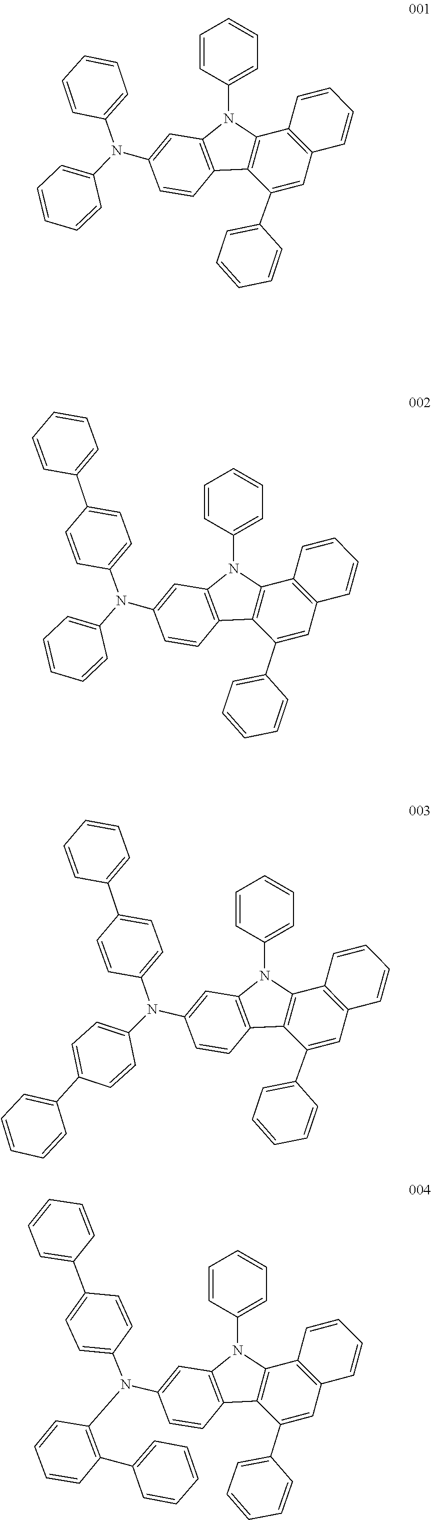

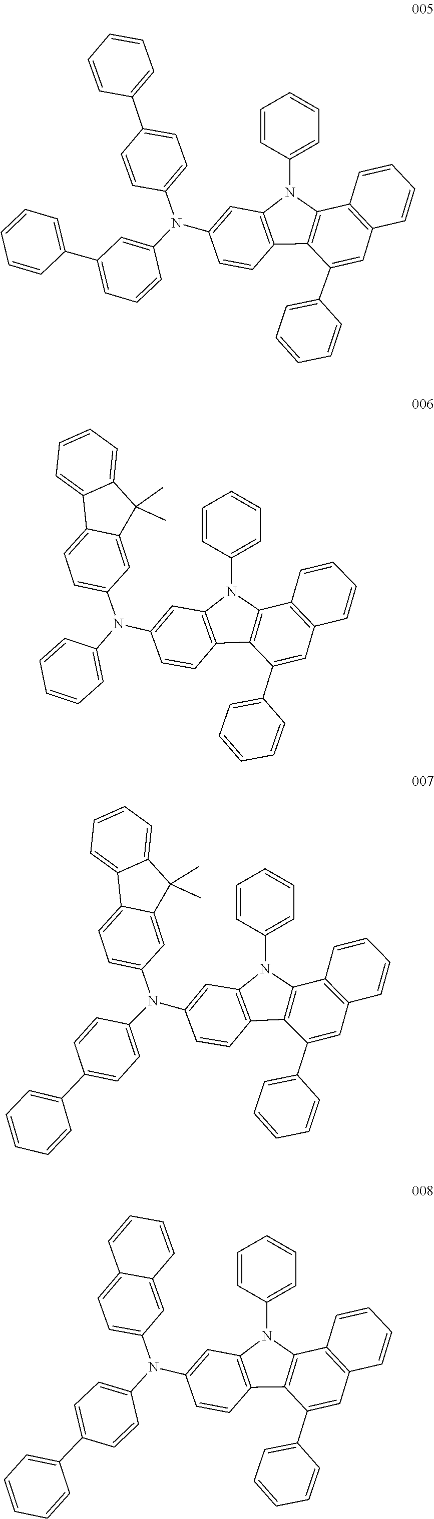 [SMILES: c1ccc(-c2ccc(N(c3cccc(-c4ccccc4)c3)c3ccc4c5c(-c6ccccc6)cc6ccccc6c5n(-c5ccccc5)c4c3)cc2)cc1 ; CC1(C)c2ccccc2-c2ccc(N(c3ccccc3)c3ccc4c5c(-c6ccccc6)cc6ccccc6c5n(-c5ccccc5)c4c3)cc21 ; CC1(C)c2ccccc2-c2ccc(N(c3ccc(-c4ccccc4)cc3)c3ccc4c5c(-c6ccccc6)cc6ccccc6c5n(-c5ccccc5)c4c3)cc21 ; c1ccc(-c2ccc(N(c3ccc4ccccc4c3)c3ccc4c5c(-c6ccccc6)cc6ccccc6c5n(-c5ccccc5)c4c3)cc2)cc1]

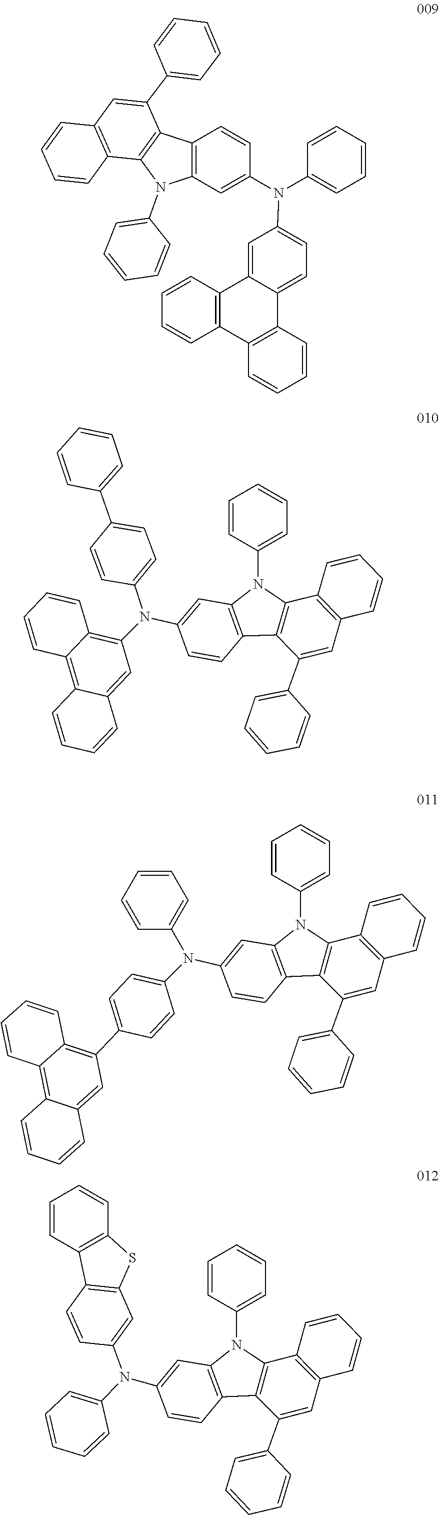

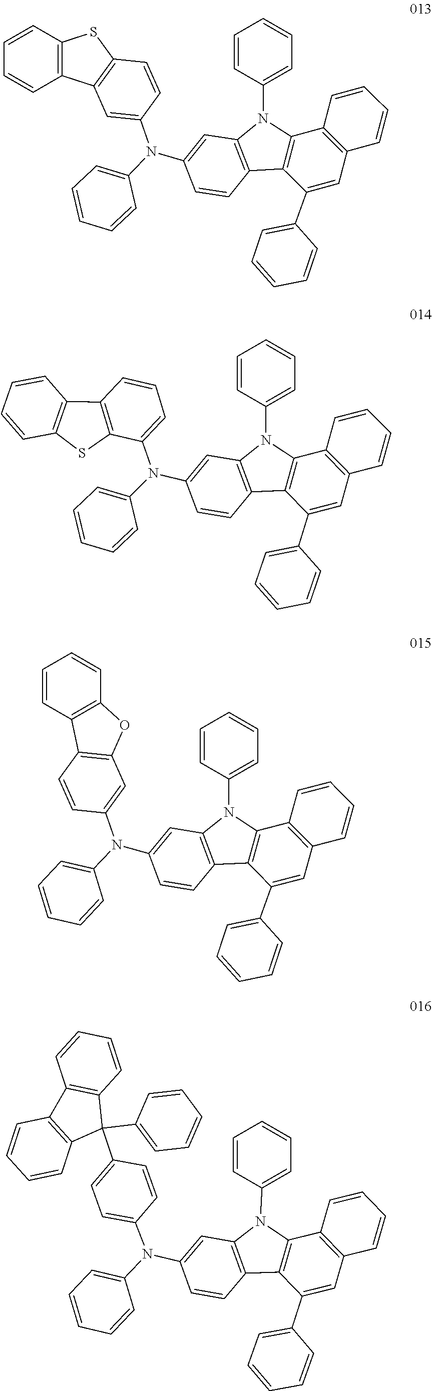 [SMILES: c1ccc(-c2cc3ccccc3c3c2c2ccc(N(c4ccccc4)c4ccc5sc6ccccc6c5c4)cc2n3-c2ccccc2)cc1 ; c1ccc(-c2cc3ccccc3c3c2c2ccc(N(c4ccccc4)c4cccc5c4sc4ccccc45)cc2n3-c2ccccc2)cc1 ; c1ccc(-c2cc3ccccc3c3c2c2ccc(N(c4ccccc4)c4ccc5c(c4)oc4ccccc45)cc2n3-c2ccccc2)cc1 ; c1ccc(-c2cc3ccccc3c3c2c2ccc(N(c4ccccc4)c4ccc(C5(c6ccccc6)c6ccccc6-c6ccccc65)cc4)cc2n3-c2ccccc2)cc1]

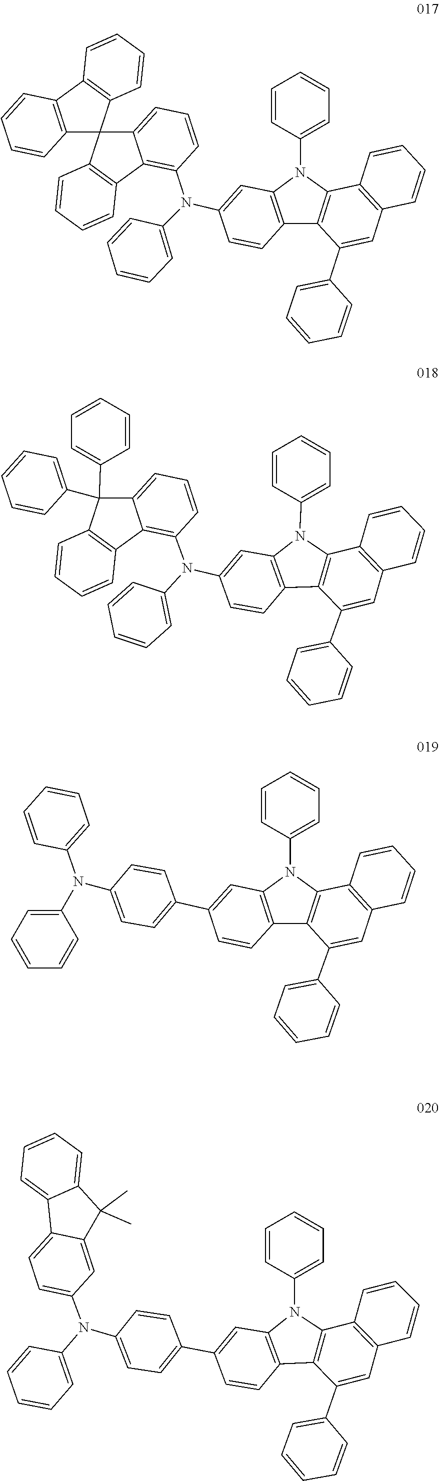 [SMILES: c1ccc(-c2cc3ccccc3c3c2c2ccc(N(c4ccccc4)c4cccc5c4-c4ccccc4C54c5ccccc5-c5ccccc54)cc2n3-c2ccccc2)cc1 ; c1ccc(-c2cc3ccccc3c3c2c2ccc(N(c4ccccc4)c4cccc5c4-c4ccccc4C5(c4ccccc4)c4ccccc4)cc2n3-c2ccccc2)cc1 ; c1ccc(-c2cc3ccccc3c3c2c2ccc(-c4ccc(N(c5ccccc5)c5ccccc5)cc4)cc2n3-c2ccccc2)cc1 ; CC1(C)c2ccccc2-c2ccc(N(c3ccccc3)c3ccc(-c4ccc5c6c(-c7ccccc7)cc7ccccc7c6n(-c6ccccc6)c5c4)cc3)cc21]

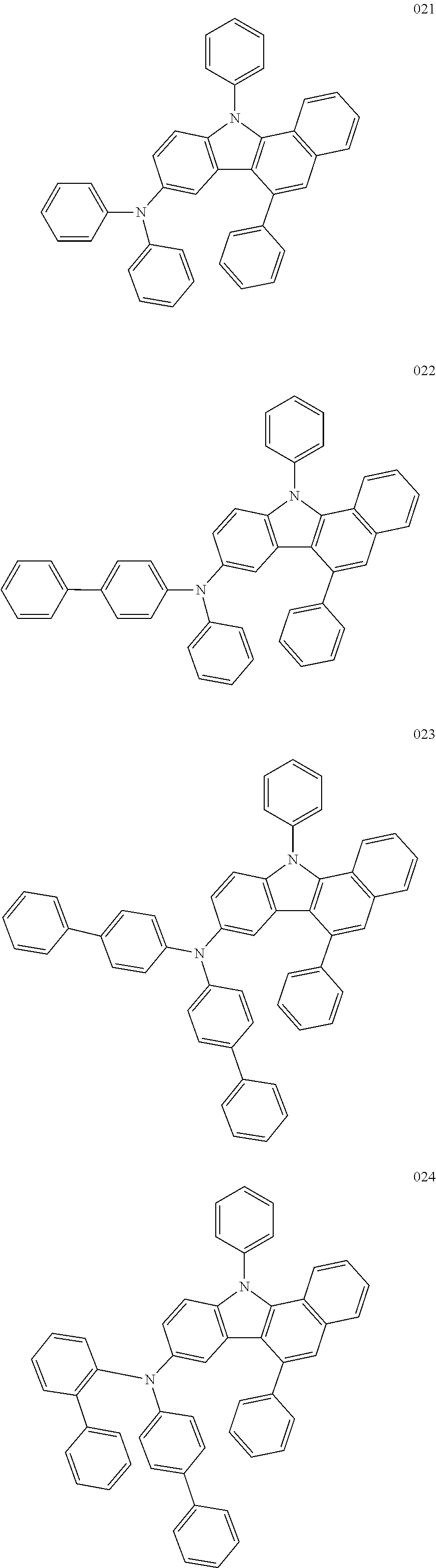 [SMILES: c1ccc(-c2cc3ccccc3c3c2c2cc(N(c4ccccc4)c4ccccc4)ccc2n3-c2ccccc2)cc1 ; c1ccc(-c2ccc(N(c3ccccc3)c3ccc4c(c3)c3c(-c5ccccc5)cc5ccccc5c3n4-c3ccccc3)cc2)cc1 ; c1ccc(-c2ccc(N(c3ccc(-c4ccccc4)cc3)c3ccc4c(c3)c3c(-c5ccccc5)cc5ccccc5c3n4-c3ccccc3)cc2)cc1 ; c1ccc(-c2ccc(N(c3ccc4c(c3)c3c(-c5ccccc5)cc5ccccc5c3n4-c3ccccc3)c3ccccc3-c3ccccc3)cc2)cc1]

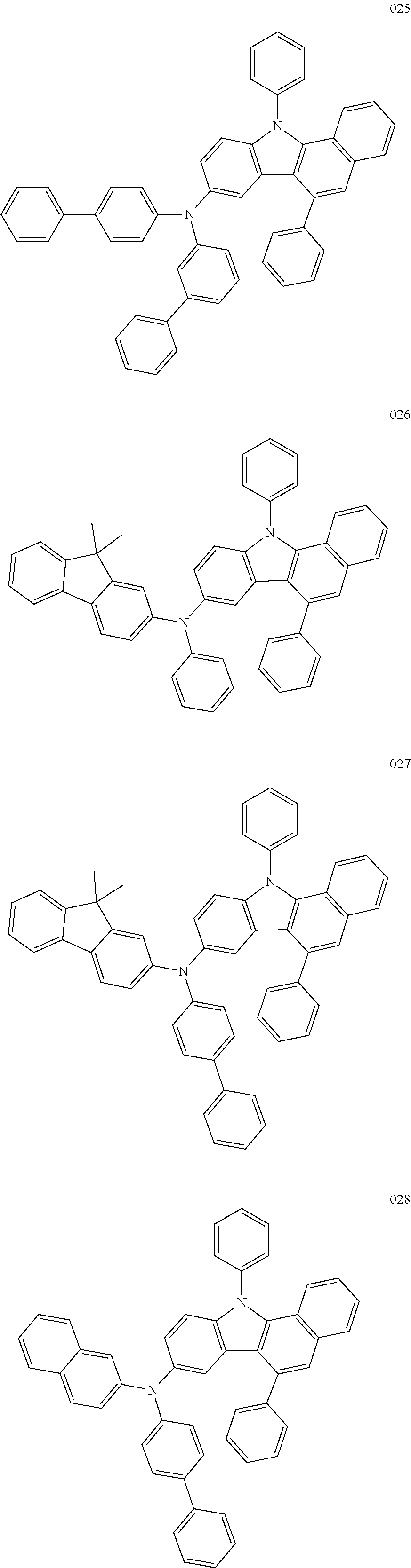 [SMILES: c1ccc(-c2ccc(N(c3cccc(-c4ccccc4)c3)c3ccc4c(c3)c3c(-c5ccccc5)cc5ccccc5c3n4-c3ccccc3)cc2)cc1 ; CC1(C)c2ccccc2-c2ccc(N(c3ccccc3)c3ccc4c(c3)c3c(-c5ccccc5)cc5ccccc5c3n4-c3ccccc3)cc21 ; CC1(C)c2ccccc2-c2ccc(N(c3ccc(-c4ccccc4)cc3)c3ccc4c(c3)c3c(-c5ccccc5)cc5ccccc5c3n4-c3ccccc3)cc21 ; c1ccc(-c2ccc(N(c3ccc4ccccc4c3)c3ccc4c(c3)c3c(-c5ccccc5)cc5ccccc5c3n4-c3ccccc3)cc2)cc1]

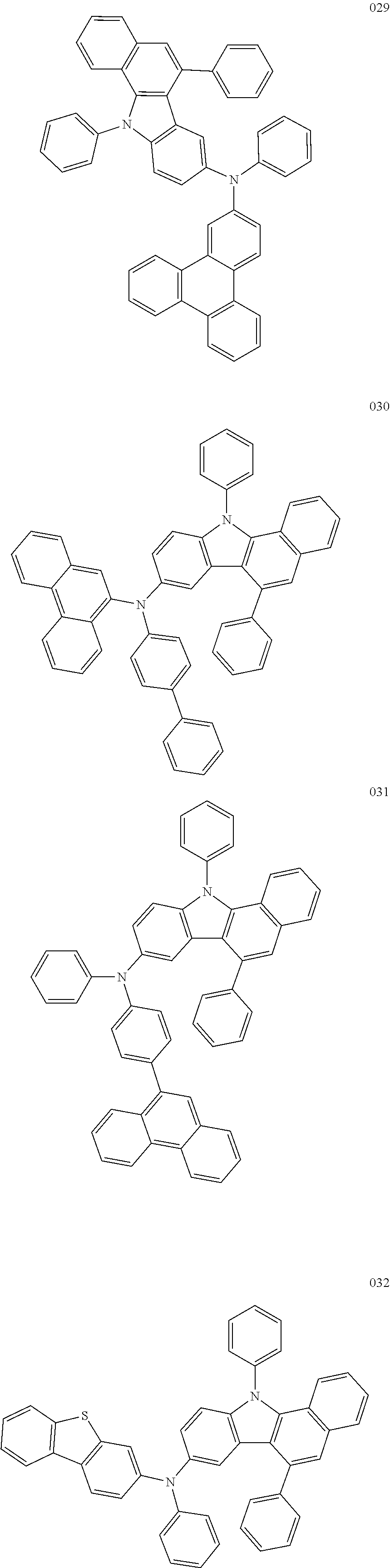

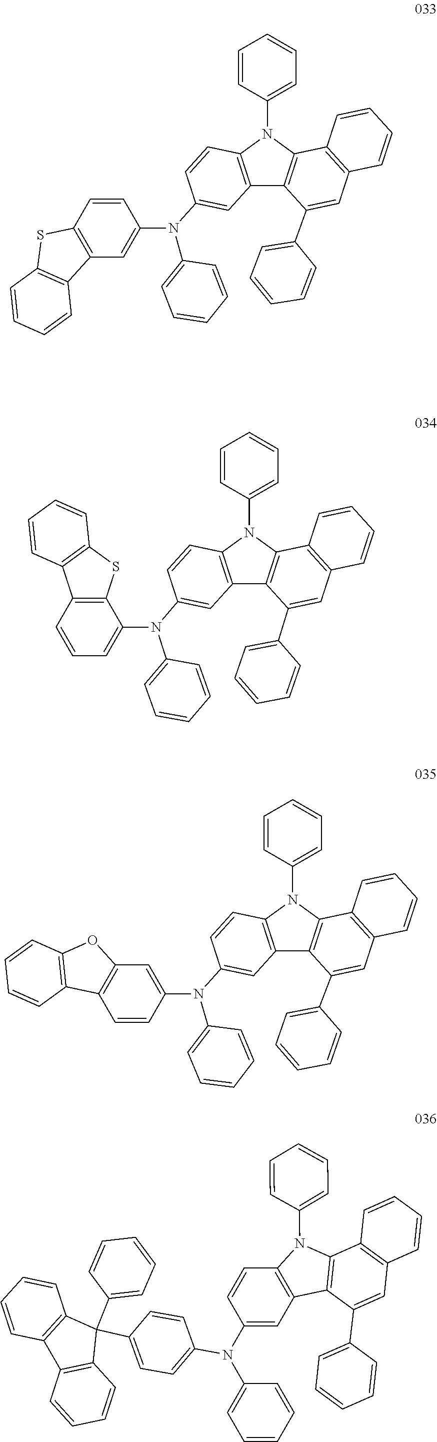 [SMILES: c1ccc(-c2cc3ccccc3c3c2c2cc(N(c4ccccc4)c4ccc5sc6ccccc6c5c4)ccc2n3-c2ccccc2)cc1 ; c1ccc(-c2cc3ccccc3c3c2c2cc(N(c4ccccc4)c4cccc5c4sc4ccccc45)ccc2n3-c2ccccc2)cc1 ; c1ccc(-c2cc3ccccc3c3c2c2cc(N(c4ccccc4)c4ccc5c(c4)oc4ccccc45)ccc2n3-c2ccccc2)cc1 ; c1ccc(-c2cc3ccccc3c3c2c2cc(N(c4ccccc4)c4ccc(C5(c6ccccc6)c6ccccc6-c6ccccc65)cc4)ccc2n3-c2ccccc2)cc1]

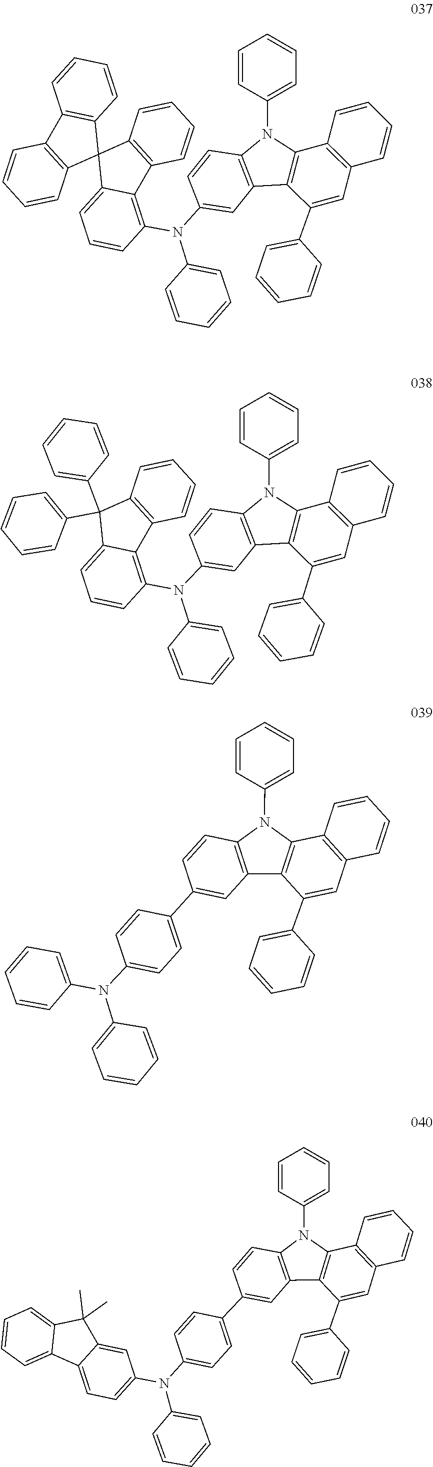 [SMILES: c1ccc(-c2cc3ccccc3c3c2c2cc(N(c4ccccc4)c4cccc5c4-c4ccccc4C54c5ccccc5-c5ccccc54)ccc2n3-c2ccccc2)cc1 ; c1ccc(-c2cc3ccccc3c3c2c2cc(N(c4ccccc4)c4cccc5c4-c4ccccc4C5(c4ccccc4)c4ccccc4)ccc2n3-c2ccccc2)cc1 ; c1ccc(-c2cc3ccccc3c3c2c2cc(-c4ccc(N(c5ccccc5)c5ccccc5)cc4)ccc2n3-c2ccccc2)cc1 ; CC1(C)c2ccccc2-c2ccc(N(c3ccccc3)c3ccc(-c4ccc5c(c4)c4c(-c6ccccc6)cc6ccccc6c4n5-c4ccccc4)cc3)cc21]

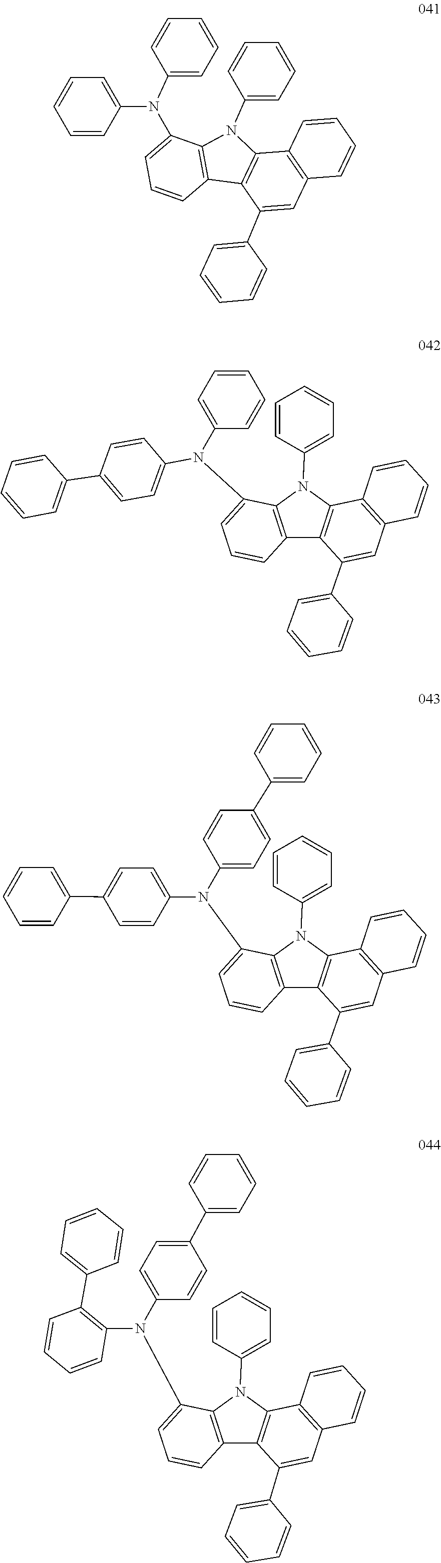 [SMILES: c1ccc(-c2cc3ccccc3c3c2c2cccc(N(c4ccccc4)c4ccccc4)c2n3-c2ccccc2)cc1 ; c1ccc(-c2ccc(N(c3ccccc3)c3cccc4c5c(-c6ccccc6)cc6ccccc6c5n(-c5ccccc5)c34)cc2)cc1 ; c1ccc(-c2ccc(N(c3ccc(-c4ccccc4)cc3)c3cccc4c5c(-c6ccccc6)cc6ccccc6c5n(-c5ccccc5)c34)cc2)cc1 ; c1ccc(-c2ccc(N(c3ccccc3-c3ccccc3)c3cccc4c5c(-c6ccccc6)cc6ccccc6c5n(-c5ccccc5)c34)cc2)cc1]

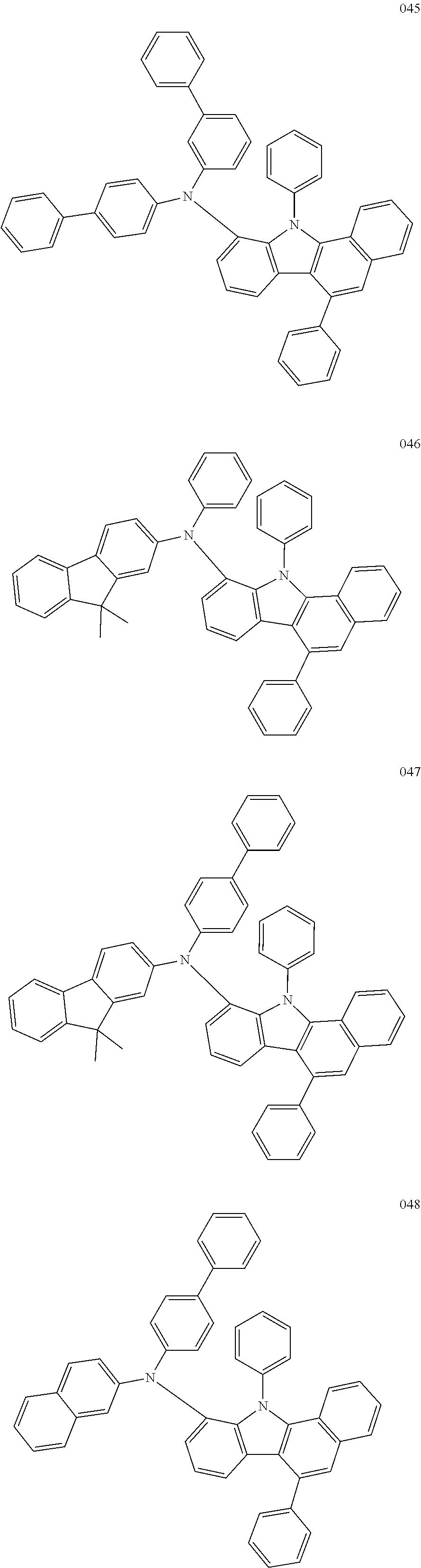 [SMILES: c1ccc(-c2ccc(N(c3cccc(-c4ccccc4)c3)c3cccc4c5c(-c6ccccc6)cc6ccccc6c5n(-c5ccccc5)c34)cc2)cc1 ; CC1(C)c2ccccc2-c2ccc(N(c3ccccc3)c3cccc4c5c(-c6ccccc6)cc6ccccc6c5n(-c5ccccc5)c34)cc21 ; CC1(C)c2ccccc2-c2ccc(N(c3ccc(-c4ccccc4)cc3)c3cccc4c5c(-c6ccccc6)cc6ccccc6c5n(-c5ccccc5)c34)cc21 ; c1ccc(-c2ccc(N(c3ccc4ccccc4c3)c3cccc4c5c(-c6ccccc6)cc6ccccc6c5n(-c5ccccc5)c34)cc2)cc1]

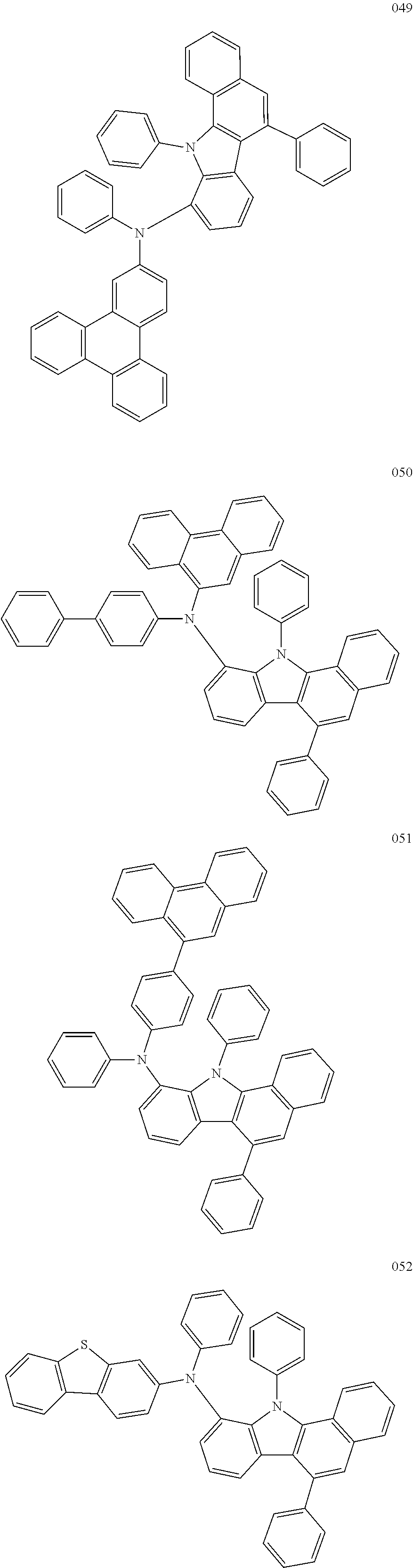 [SMILES: c1ccc(-c2cc3ccccc3c3c2c2cccc(N(c4ccccc4)c4ccc5c6ccccc6c6ccccc6c5c4)c2n3-c2ccccc2)cc1 ; c1ccc(-c2ccc(N(c3cc4ccccc4c4ccccc34)c3cccc4c5c(-c6ccccc6)cc6ccccc6c5n(-c5ccccc5)c34)cc2)cc1 ; c1ccc(-c2cc3ccccc3c3c2c2cccc(N(c4ccccc4)c4ccc(-c5cc6ccccc6c6ccccc56)cc4)c2n3-c2ccccc2)cc1 ; c1ccc(-c2cc3ccccc3c3c2c2cccc(N(c4ccccc4)c4ccc5c(c4)sc4ccccc45)c2n3-c2ccccc2)cc1]

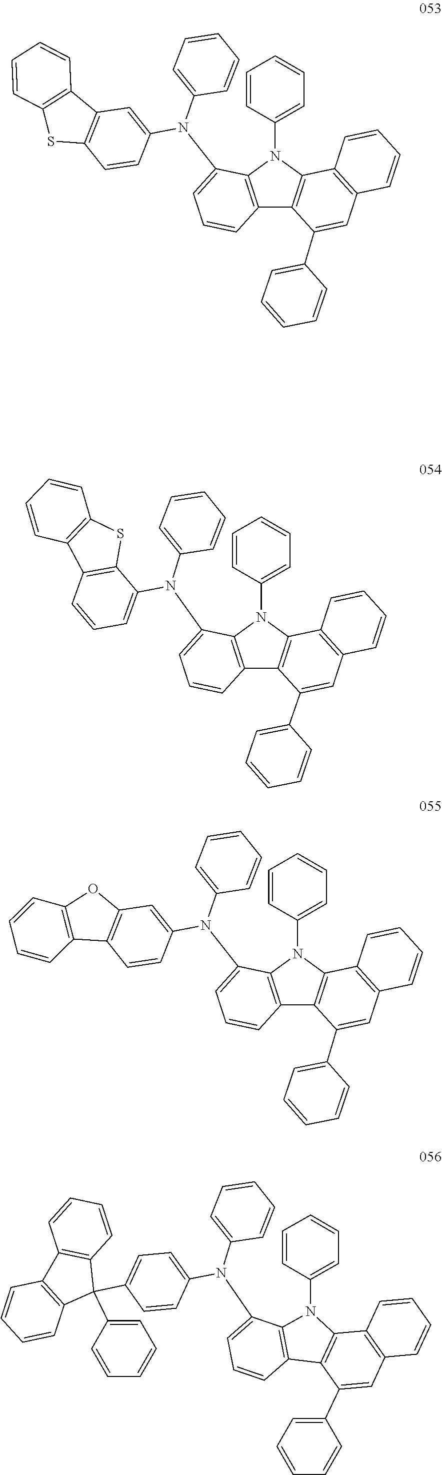 [SMILES: c1ccc(-c2cc3ccccc3c3c2c2cccc(N(c4ccccc4)c4ccc5sc6ccccc6c5c4)c2n3-c2ccccc2)cc1 ; c1ccc(-c2cc3ccccc3c3c2c2cccc(N(c4ccccc4)c4cccc5c4sc4ccccc45)c2n3-c2ccccc2)cc1 ; c1ccc(-c2cc3ccccc3c3c2c2cccc(N(c4ccccc4)c4ccc5c(c4)oc4ccccc45)c2n3-c2ccccc2)cc1 ; c1ccc(-c2cc3ccccc3c3c2c2cccc(N(c4ccccc4)c4ccc(C5(c6ccccc6)c6ccccc6-c6ccccc65)cc4)c2n3-c2ccccc2)cc1]

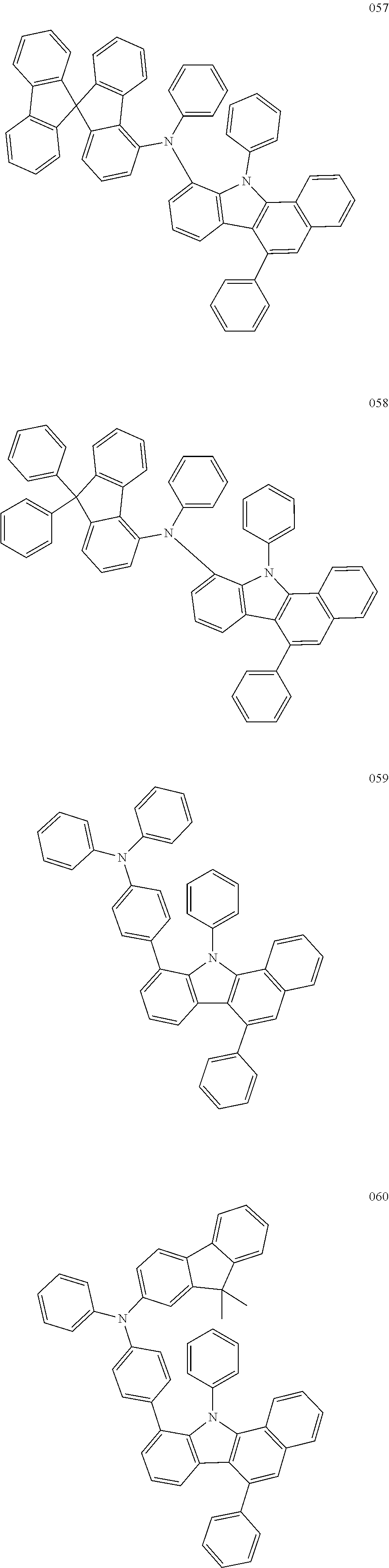 [SMILES: c1ccc(-c2cc3ccccc3c3c2c2cccc(N(c4ccccc4)c4cccc5c4-c4ccccc4C54c5ccccc5-c5ccccc54)c2n3-c2ccccc2)cc1 ; c1ccc(-c2cc3ccccc3c3c2c2cccc(N(c4ccccc4)c4cccc5c4-c4ccccc4C5(c4ccccc4)c4ccccc4)c2n3-c2ccccc2)cc1 ; c1ccc(-c2cc3ccccc3c3c2c2cccc(-c4ccc(N(c5ccccc5)c5ccccc5)cc4)c2n3-c2ccccc2)cc1 ; CC1(C)c2ccccc2-c2ccc(N(c3ccccc3)c3ccc(-c4cccc5c6c(-c7ccccc7)cc7ccccc7c6n(-c6ccccc6)c45)cc3)cc21]

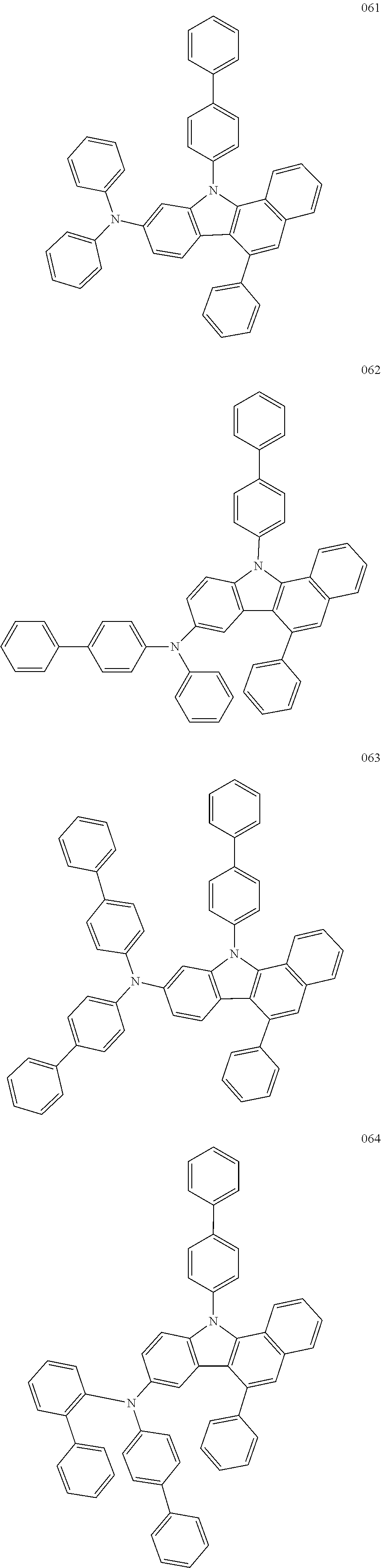 [SMILES: c1ccc(-c2ccc(-n3c4cc(N(c5ccccc5)c5ccccc5)ccc4c4c(-c5ccccc5)cc5ccccc5c43)cc2)cc1 ; c1ccc(-c2ccc(N(c3ccccc3)c3ccc4c(c3)c3c(-c5ccccc5)cc5ccccc5c3n4-c3ccc(-c4ccccc4)cc3)cc2)cc1 ; c1ccc(-c2ccc(N(c3ccc(-c4ccccc4)cc3)c3ccc4c5c(-c6ccccc6)cc6ccccc6c5n(-c5ccc(-c6ccccc6)cc5)c4c3)cc2)cc1 ; c1ccc(-c2ccc(N(c3ccc4c(c3)c3c(-c5ccccc5)cc5ccccc5c3n4-c3ccc(-c4ccccc4)cc3)c3ccccc3-c3ccccc3)cc2)cc1]

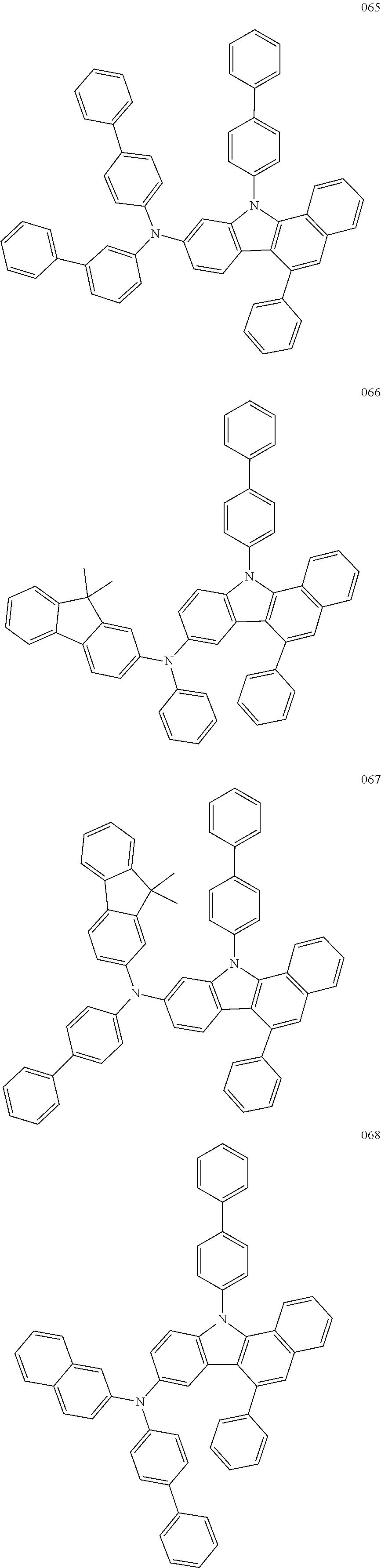

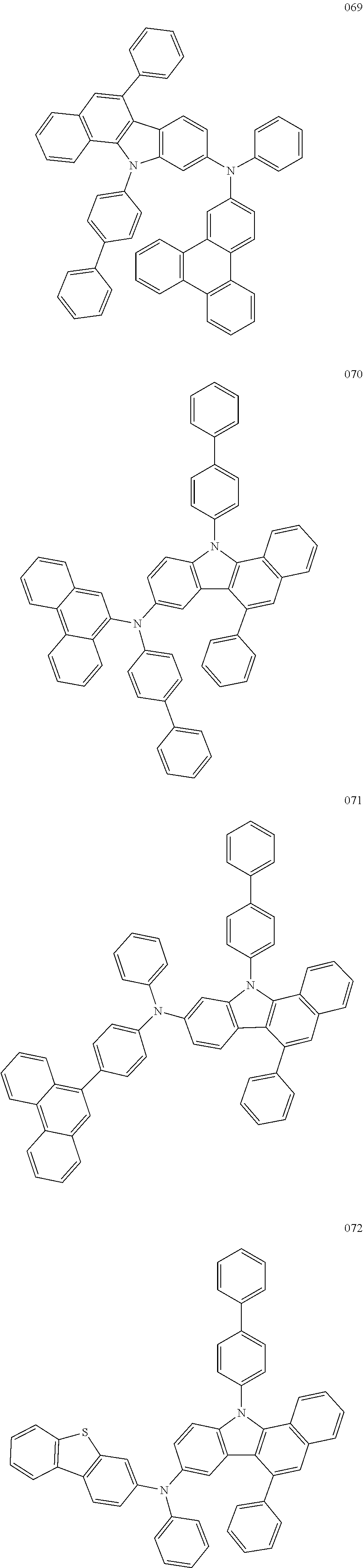 [SMILES: c1ccc(-c2ccc(-n3c4cc(N(c5ccccc5)c5ccc6c7ccccc7c7ccccc7c6c5)ccc4c4c(-c5ccccc5)cc5ccccc5c43)cc2)cc1 ; c1ccc(-c2ccc(N(c3ccc4c(c3)c3c(-c5ccccc5)cc5ccccc5c3n4-c3ccc(-c4ccccc4)cc3)c3cc4ccccc4c4ccccc34)cc2)cc1 ; c1ccc(-c2ccc(-n3c4cc(N(c5ccccc5)c5ccc(-c6cc7ccccc7c7ccccc67)cc5)ccc4c4c(-c5ccccc5)cc5ccccc5c43)cc2)cc1 ; c1ccc(-c2ccc(-n3c4ccc(N(c5ccccc5)c5ccc6c(c5)sc5ccccc56)cc4c4c(-c5ccccc5)cc5ccccc5c43)cc2)cc1]

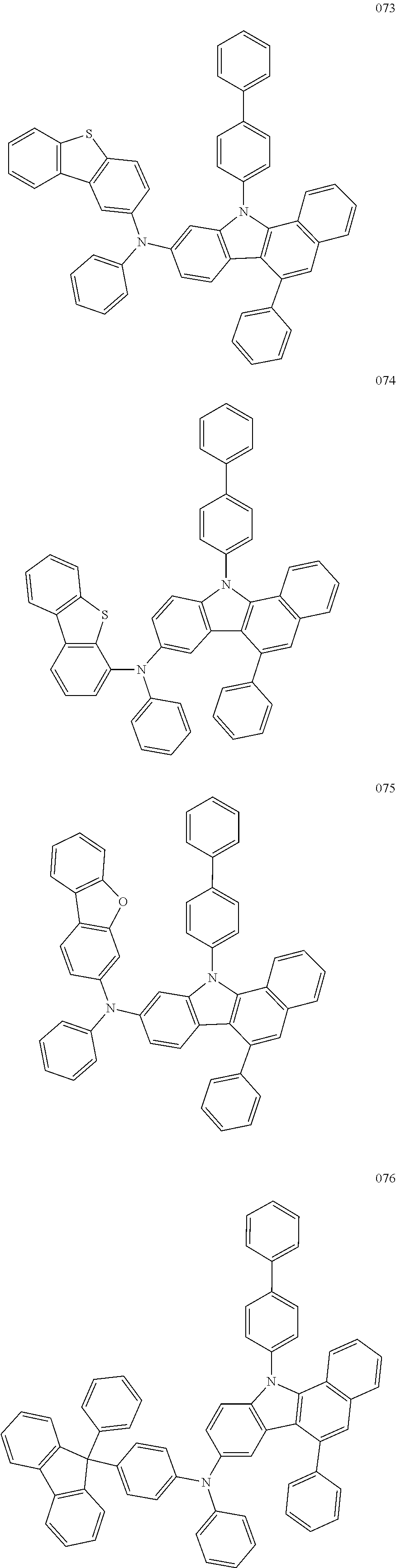 [SMILES: c1ccc(-c2ccc(-n3c4cc(N(c5ccccc5)c5ccc6sc7ccccc7c6c5)ccc4c4c(-c5ccccc5)cc5ccccc5c43)cc2)cc1 ; c1ccc(-c2ccc(-n3c4ccc(N(c5ccccc5)c5cccc6c5sc5ccccc56)cc4c4c(-c5ccccc5)cc5ccccc5c43)cc2)cc1 ; c1ccc(-c2ccc(-n3c4cc(N(c5ccccc5)c5ccc6c(c5)oc5ccccc56)ccc4c4c(-c5ccccc5)cc5ccccc5c43)cc2)cc1 ; c1ccc(-c2ccc(-n3c4ccc(N(c5ccccc5)c5ccc(C6(c7ccccc7)c7ccccc7-c7ccccc76)cc5)cc4c4c(-c5ccccc5)cc5ccccc5c43)cc2)cc1]

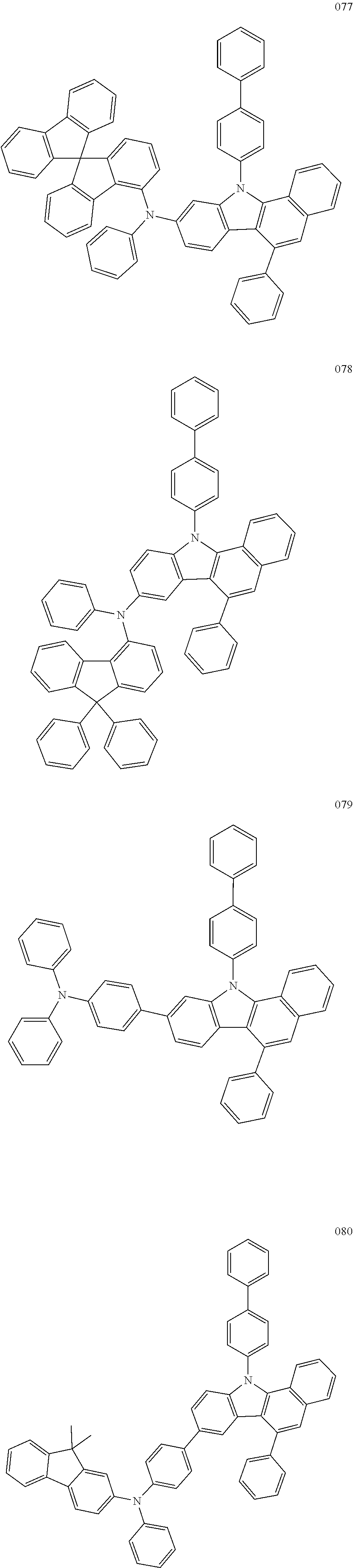 [SMILES: c1ccc(-c2ccc(-n3c4cc(N(c5ccccc5)c5cccc6c5-c5ccccc5C65c6ccccc6-c6ccccc65)ccc4c4c(-c5ccccc5)cc5ccccc5c43)cc2)cc1 ; c1ccc(-c2ccc(-n3c4ccc(N(c5ccccc5)c5cccc6c5-c5ccccc5C6(c5ccccc5)c5ccccc5)cc4c4c(-c5ccccc5)cc5ccccc5c43)cc2)cc1 ; c1ccc(-c2ccc(-n3c4cc(-c5ccc(N(c6ccccc6)c6ccccc6)cc5)ccc4c4c(-c5ccccc5)cc5ccccc5c43)cc2)cc1 ; CC1(C)c2ccccc2-c2ccc(N(c3ccccc3)c3ccc(-c4ccc5c(c4)c4c(-c6ccccc6)cc6ccccc6c4n5-c4ccc(-c5ccccc5)cc4)cc3)cc21]

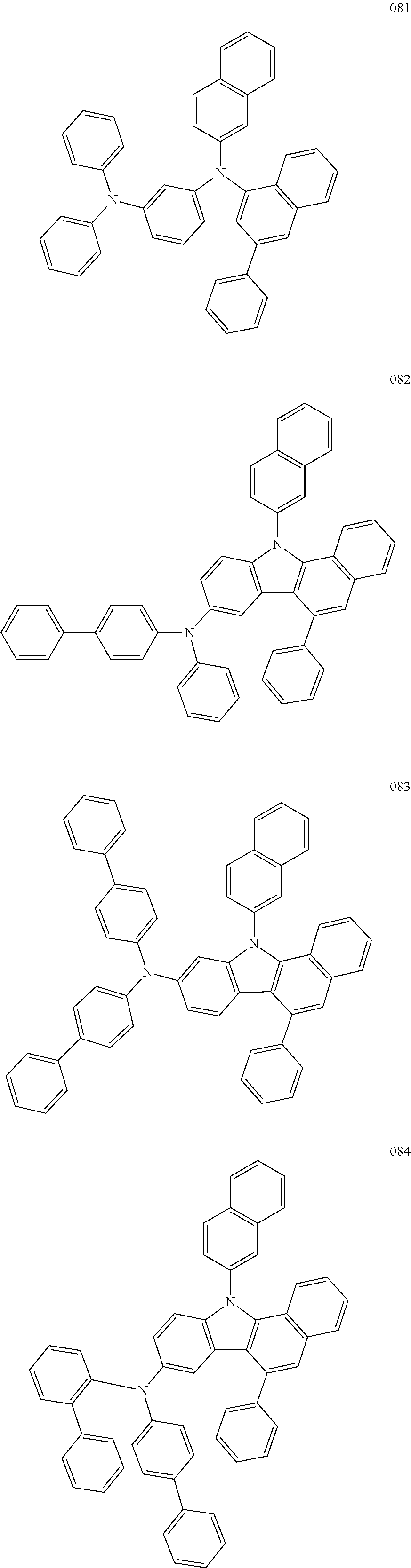 [SMILES: c1ccc(-c2cc3ccccc3c3c2c2ccc(N(c4ccccc4)c4ccccc4)cc2n3-c2ccc3ccccc3c2)cc1 ; c1ccc(-c2ccc(N(c3ccccc3)c3ccc4c(c3)c3c(-c5ccccc5)cc5ccccc5c3n4-c3ccc4ccccc4c3)cc2)cc1 ; c1ccc(-c2ccc(N(c3ccc(-c4ccccc4)cc3)c3ccc4c5c(-c6ccccc6)cc6ccccc6c5n(-c5ccc6ccccc6c5)c4c3)cc2)cc1 ; c1ccc(-c2ccc(N(c3ccc4c(c3)c3c(-c5ccccc5)cc5ccccc5c3n4-c3ccc4ccccc4c3)c3ccccc3-c3ccccc3)cc2)cc1]

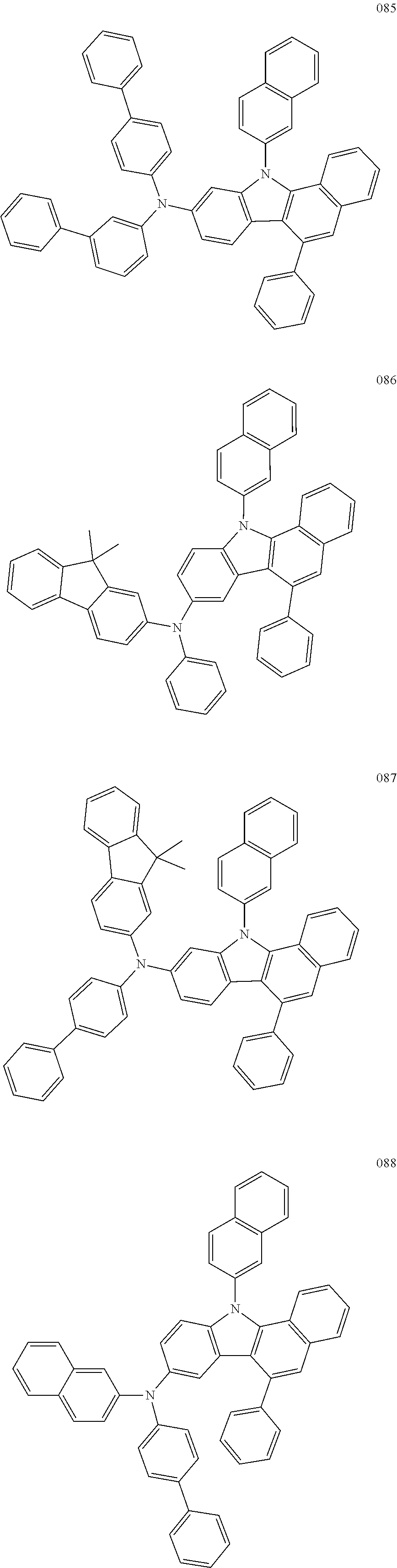 [SMILES: c1ccc(-c2ccc(N(c3cccc(-c4ccccc4)c3)c3ccc4c5c(-c6ccccc6)cc6ccccc6c5n(-c5ccc6ccccc6c5)c4c3)cc2)cc1 ; CC1(C)c2ccccc2-c2ccc(N(c3ccccc3)c3ccc4c(c3)c3c(-c5ccccc5)cc5ccccc5c3n4-c3ccc4ccccc4c3)cc21 ; CC1(C)c2ccccc2-c2ccc(N(c3ccc(-c4ccccc4)cc3)c3ccc4c5c(-c6ccccc6)cc6ccccc6c5n(-c5ccc6ccccc6c5)c4c3)cc21 ; c1ccc(-c2ccc(N(c3ccc4ccccc4c3)c3ccc4c(c3)c3c(-c5ccccc5)cc5ccccc5c3n4-c3ccc4ccccc4c3)cc2)cc1]

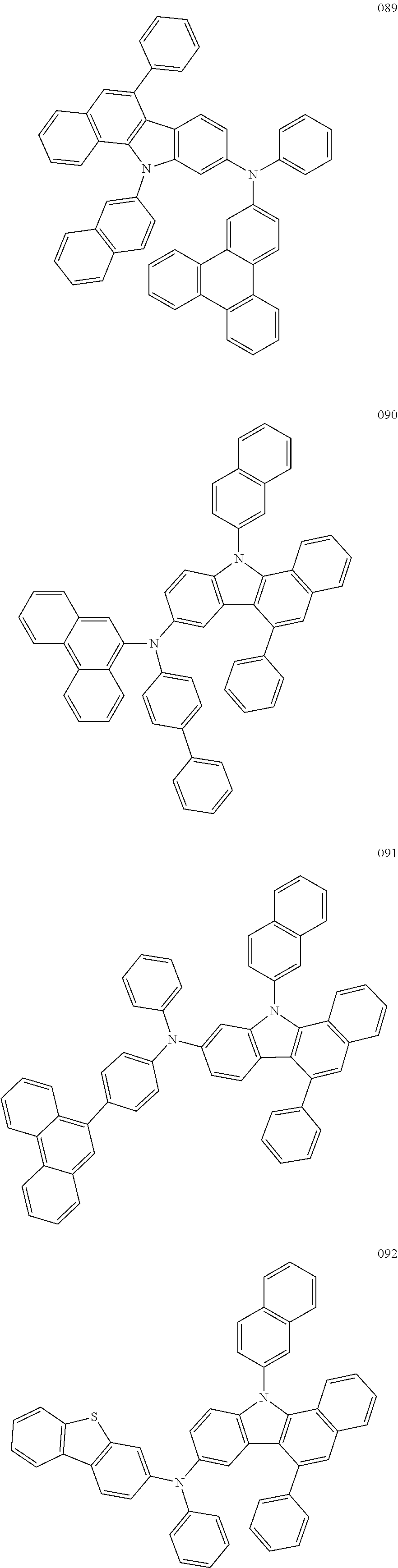 [SMILES: c1ccc(-c2cc3ccccc3c3c2c2ccc(N(c4ccccc4)c4ccc5c6ccccc6c6ccccc6c5c4)cc2n3-c2ccc3ccccc3c2)cc1 ; c1ccc(-c2ccc(N(c3ccc4c(c3)c3c(-c5ccccc5)cc5ccccc5c3n4-c3ccc4ccccc4c3)c3cc4ccccc4c4ccccc34)cc2)cc1 ; c1ccc(-c2cc3ccccc3c3c2c2ccc(N(c4ccccc4)c4ccc(-c5cc6ccccc6c6ccccc56)cc4)cc2n3-c2ccc3ccccc3c2)cc1 ; c1ccc(-c2cc3ccccc3c3c2c2cc(N(c4ccccc4)c4ccc5c(c4)sc4ccccc45)ccc2n3-c2ccc3ccccc3c2)cc1]

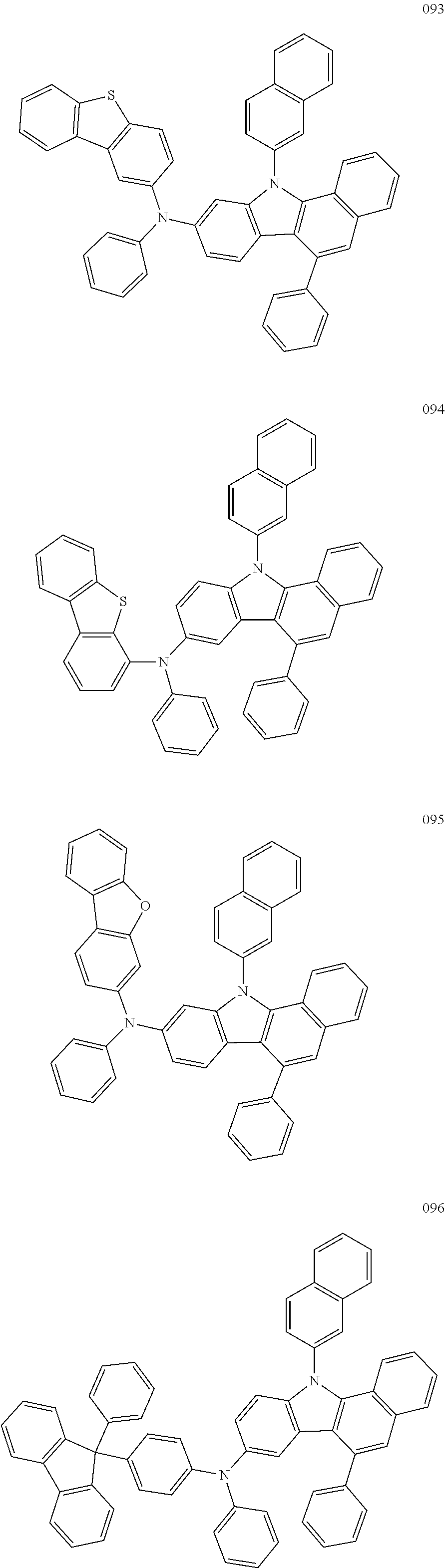 [SMILES: c1ccc(-c2cc3ccccc3c3c2c2ccc(N(c4ccccc4)c4ccc5sc6ccccc6c5c4)cc2n3-c2ccc3ccccc3c2)cc1 ; c1ccc(-c2cc3ccccc3c3c2c2cc(N(c4ccccc4)c4cccc5c4sc4ccccc45)ccc2n3-c2ccc3ccccc3c2)cc1 ; c1ccc(-c2cc3ccccc3c3c2c2ccc(N(c4ccccc4)c4ccc5c(c4)oc4ccccc45)cc2n3-c2ccc3ccccc3c2)cc1 ; c1ccc(-c2cc3ccccc3c3c2c2cc(N(c4ccccc4)c4ccc(C5(c6ccccc6)c6ccccc6-c6ccccc65)cc4)ccc2n3-c2ccc3ccccc3c2)cc1]

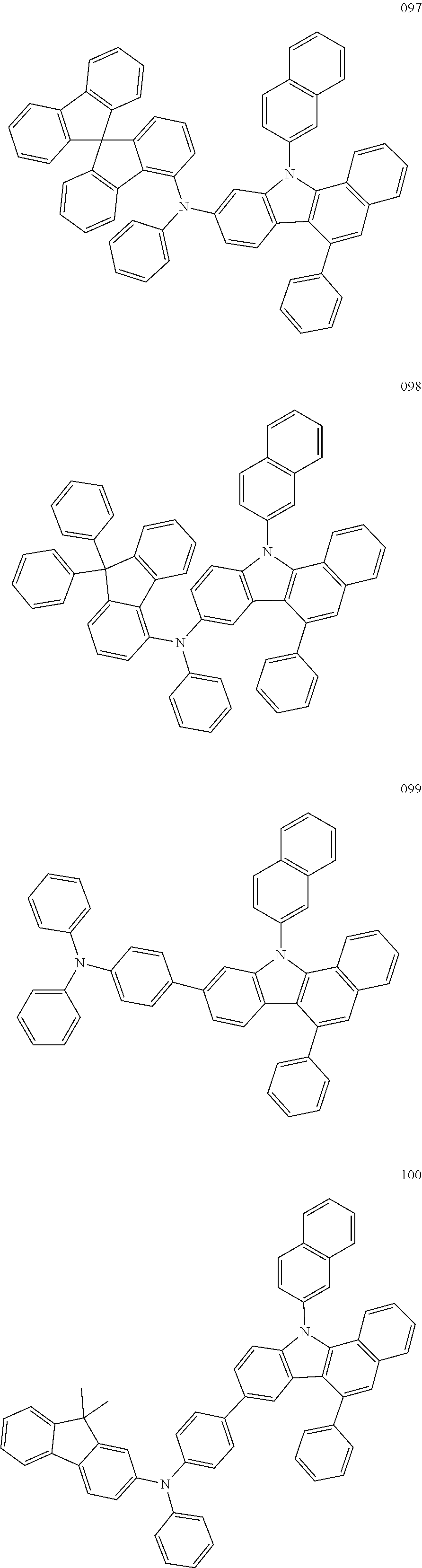 [SMILES: c1ccc(-c2cc3ccccc3c3c2c2ccc(N(c4ccccc4)c4cccc5c4-c4ccccc4C54c5ccccc5-c5ccccc54)cc2n3-c2ccc3ccccc3c2)cc1 ; c1ccc(-c2cc3ccccc3c3c2c2cc(N(c4ccccc4)c4cccc5c4-c4ccccc4C5(c4ccccc4)c4ccccc4)ccc2n3-c2ccc3ccccc3c2)cc1 ; c1ccc(-c2cc3ccccc3c3c2c2ccc(-c4ccc(N(c5ccccc5)c5ccccc5)cc4)cc2n3-c2ccc3ccccc3c2)cc1 ; CC1(C)c2ccccc2-c2ccc(N(c3ccccc3)c3ccc(-c4ccc5c(c4)c4c(-c6ccccc6)cc6ccccc6c4n5-c4ccc5ccccc5c4)cc3)cc21]

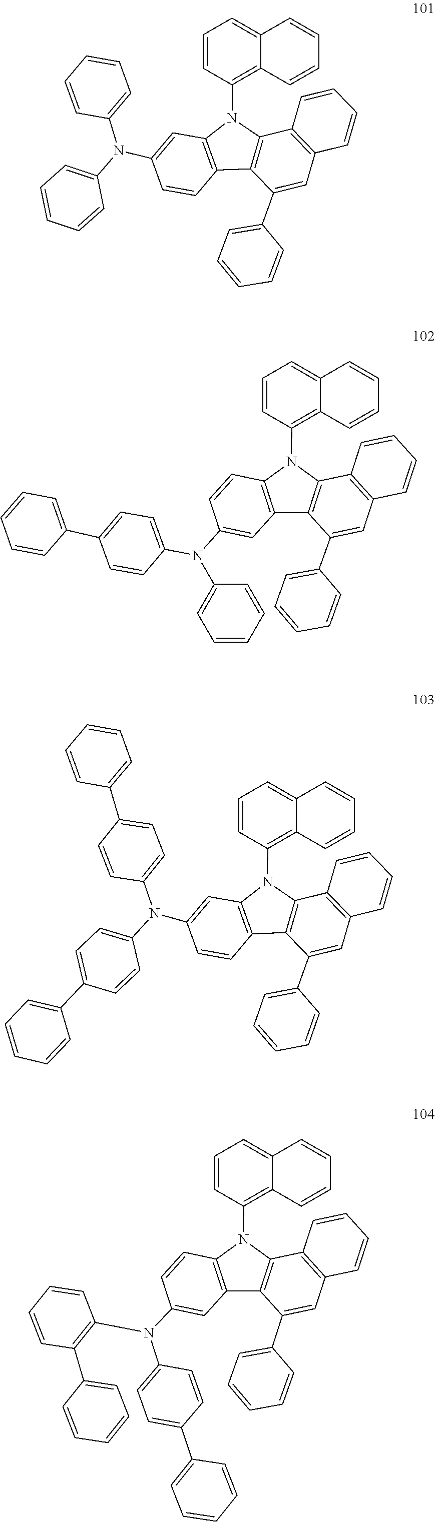 [SMILES: c1ccc(-c2cc3ccccc3c3c2c2ccc(N(c4ccccc4)c4ccccc4)cc2n3-c2cccc3ccccc23)cc1 ; c1ccc(-c2ccc(N(c3ccccc3)c3ccc4c(c3)c3c(-c5ccccc5)cc5ccccc5c3n4-c3cccc4ccccc34)cc2)cc1 ; c1ccc(-c2ccc(N(c3ccc(-c4ccccc4)cc3)c3ccc4c5c(-c6ccccc6)cc6ccccc6c5n(-c5cccc6ccccc56)c4c3)cc2)cc1 ; c1ccc(-c2ccc(N(c3ccc4c(c3)c3c(-c5ccccc5)cc5ccccc5c3n4-c3cccc4ccccc34)c3ccccc3-c3ccccc3)cc2)cc1]

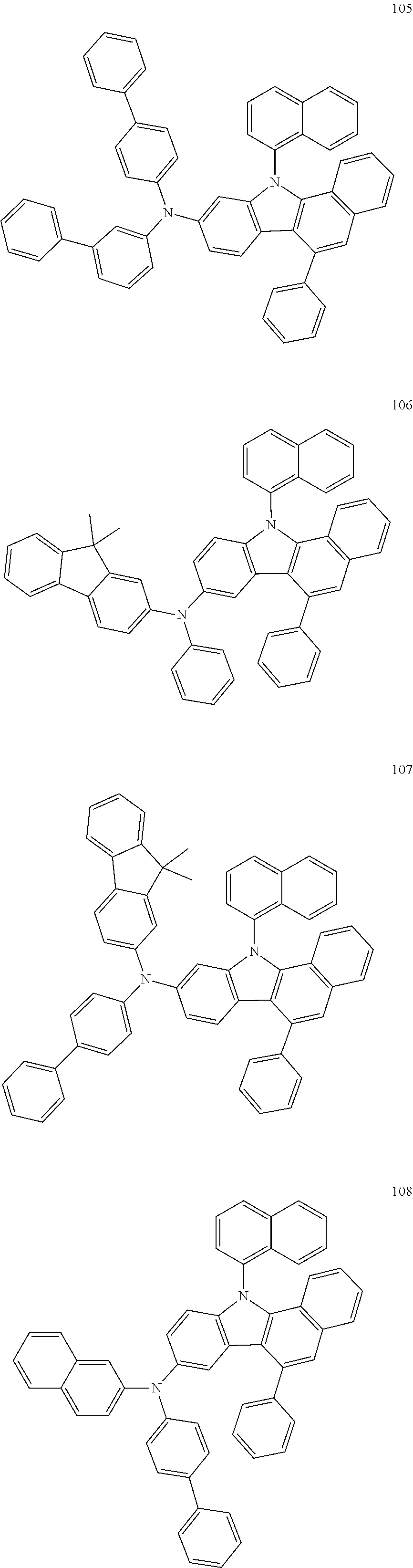 [SMILES: c1ccc(-c2ccc(N(c3cccc(-c4ccccc4)c3)c3ccc4c5c(-c6ccccc6)cc6ccccc6c5n(-c5cccc6ccccc56)c4c3)cc2)cc1 ; CC1(C)c2ccccc2-c2ccc(N(c3ccccc3)c3ccc4c(c3)c3c(-c5ccccc5)cc5ccccc5c3n4-c3cccc4ccccc34)cc21 ; CC1(C)c2ccccc2-c2ccc(N(c3ccc(-c4ccccc4)cc3)c3ccc4c5c(-c6ccccc6)cc6ccccc6c5n(-c5cccc6ccccc56)c4c3)cc21 ; c1ccc(-c2ccc(N(c3ccc4ccccc4c3)c3ccc4c(c3)c3c(-c5ccccc5)cc5ccccc5c3n4-c3cccc4ccccc34)cc2)cc1]

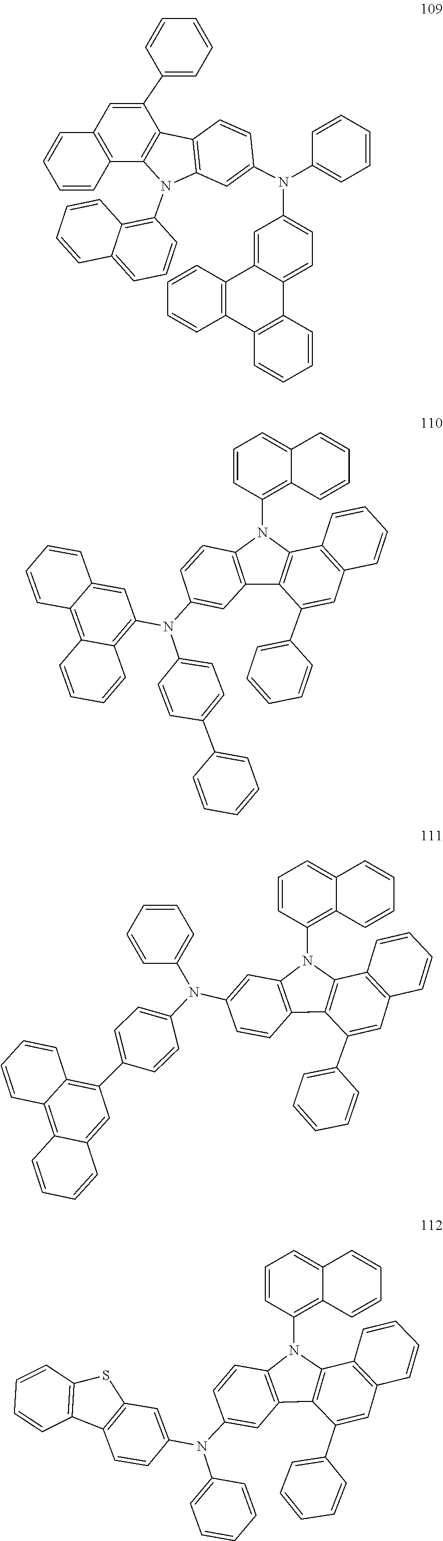 [SMILES: c1ccc(-c2cc3ccccc3c3c2c2ccc(N(c4ccccc4)c4ccc5c6ccccc6c6ccccc6c5c4)cc2n3-c2cccc3ccccc23)cc1 ; c1ccc(-c2ccc(N(c3ccc4c(c3)c3c(-c5ccccc5)cc5ccccc5c3n4-c3cccc4ccccc34)c3cc4ccccc4c4ccccc34)cc2)cc1 ; c1ccc(-c2cc3ccccc3c3c2c2ccc(N(c4ccccc4)c4ccc(-c5cc6ccccc6c6ccccc56)cc4)cc2n3-c2cccc3ccccc23)cc1 ; c1ccc(-c2cc3ccccc3c3c2c2cc(N(c4ccccc4)c4ccc5c(c4)sc4ccccc45)ccc2n3-c2cccc3ccccc23)cc1]

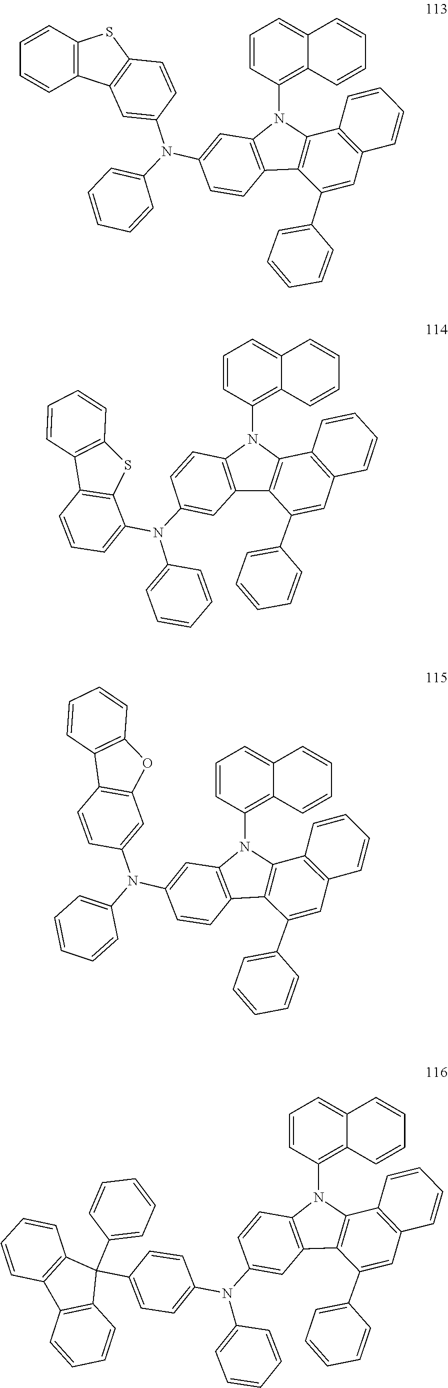 [SMILES: c1ccc(-c2cc3ccccc3c3c2c2ccc(N(c4ccccc4)c4ccc5sc6ccccc6c5c4)cc2n3-c2cccc3ccccc23)cc1 ; c1ccc(-c2cc3ccccc3c3c2c2cc(N(c4ccccc4)c4cccc5c4sc4ccccc45)ccc2n3-c2cccc3ccccc23)cc1 ; c1ccc(-c2cc3ccccc3c3c2c2ccc(N(c4ccccc4)c4ccc5c(c4)oc4ccccc45)cc2n3-c2cccc3ccccc23)cc1 ; c1ccc(-c2cc3ccccc3c3c2c2cc(N(c4ccccc4)c4ccc(C5(c6ccccc6)c6ccccc6-c6ccccc65)cc4)ccc2n3-c2cccc3ccccc23)cc1]

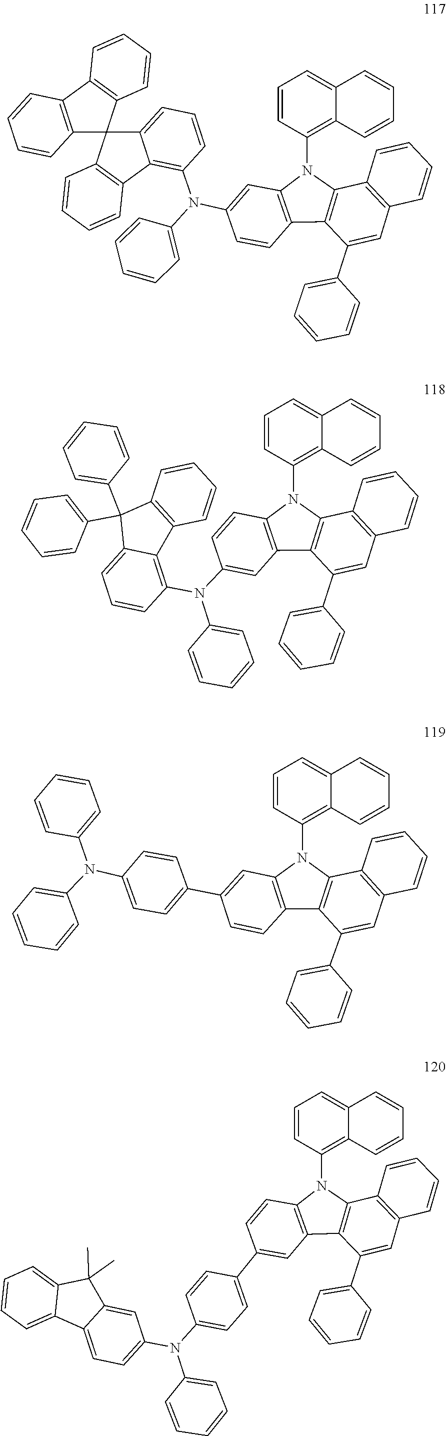 [SMILES: c1ccc(-c2cc3ccccc3c3c2c2ccc(N(c4ccccc4)c4cccc5c4-c4ccccc4C54c5ccccc5-c5ccccc54)cc2n3-c2cccc3ccccc23)cc1 ; c1ccc(-c2cc3ccccc3c3c2c2cc(N(c4ccccc4)c4cccc5c4-c4ccccc4C5(c4ccccc4)c4ccccc4)ccc2n3-c2cccc3ccccc23)cc1 ; c1ccc(-c2cc3ccccc3c3c2c2ccc(-c4ccc(N(c5ccccc5)c5ccccc5)cc4)cc2n3-c2cccc3ccccc23)cc1 ; CC1(C)c2ccccc2-c2ccc(N(c3ccccc3)c3ccc(-c4ccc5c(c4)c4c(-c6ccccc6)cc6ccccc6c4n5-c4cccc5ccccc45)cc3)cc21]

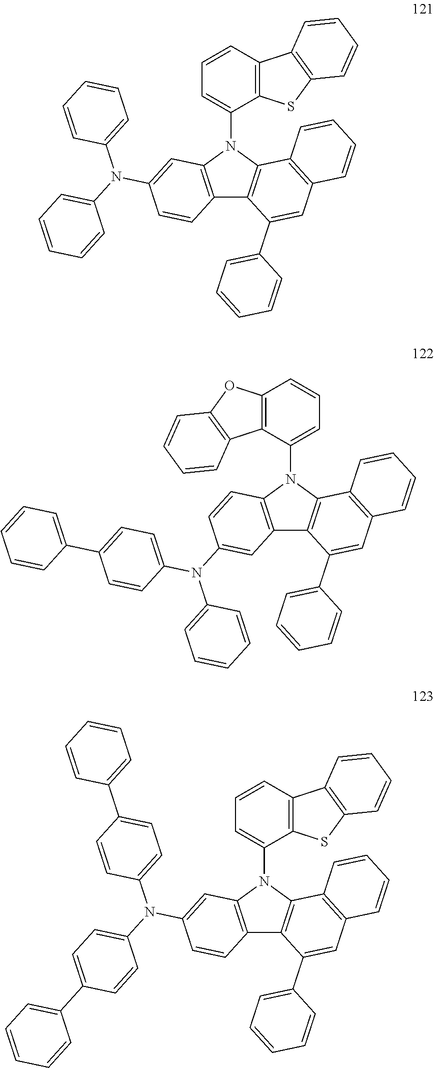 [SMILES: c1ccc(-c2cc3ccccc3c3c2c2ccc(N(c4ccccc4)c4ccccc4)cc2n3-c2cccc3c2sc2ccccc23)cc1 ; c1ccc(-c2ccc(N(c3ccccc3)c3ccc4c(c3)c3c(-c5ccccc5)cc5ccccc5c3n4-c3cccc4oc5ccccc5c34)cc2)cc1 ; c1ccc(-c2ccc(N(c3ccc(-c4ccccc4)cc3)c3ccc4c5c(-c6ccccc6)cc6ccccc6c5n(-c5cccc6c5sc5ccccc56)c4c3)cc2)cc1]

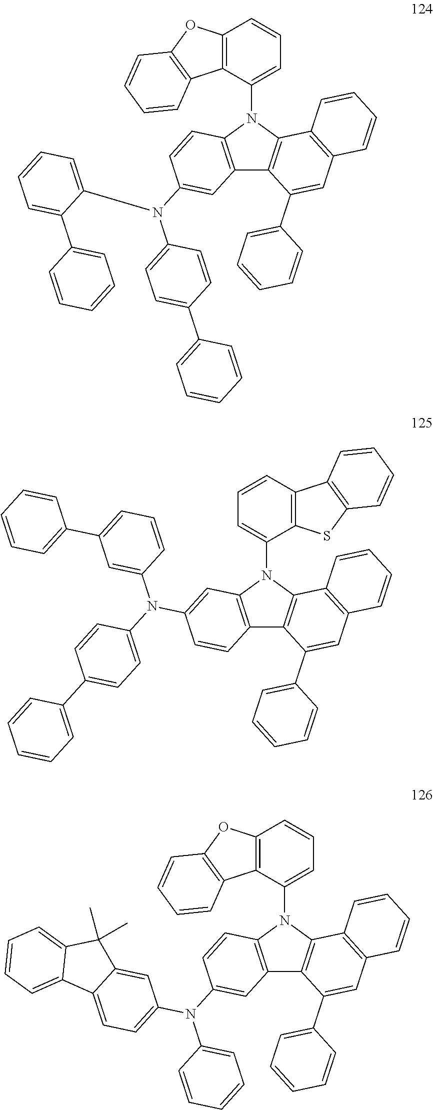 [SMILES: c1ccc(-c2ccc(N(c3ccc4c(c3)c3c(-c5ccccc5)cc5ccccc5c3n4-c3cccc4oc5ccccc5c34)c3ccccc3-c3ccccc3)cc2)cc1 ; c1ccc(-c2ccc(N(c3cccc(-c4ccccc4)c3)c3ccc4c5c(-c6ccccc6)cc6ccccc6c5n(-c5cccc6c5sc5ccccc56)c4c3)cc2)cc1 ; CC1(C)c2ccccc2-c2ccc(N(c3ccccc3)c3ccc4c(c3)c3c(-c5ccccc5)cc5ccccc5c3n4-c3cccc4oc5ccccc5c34)cc21]

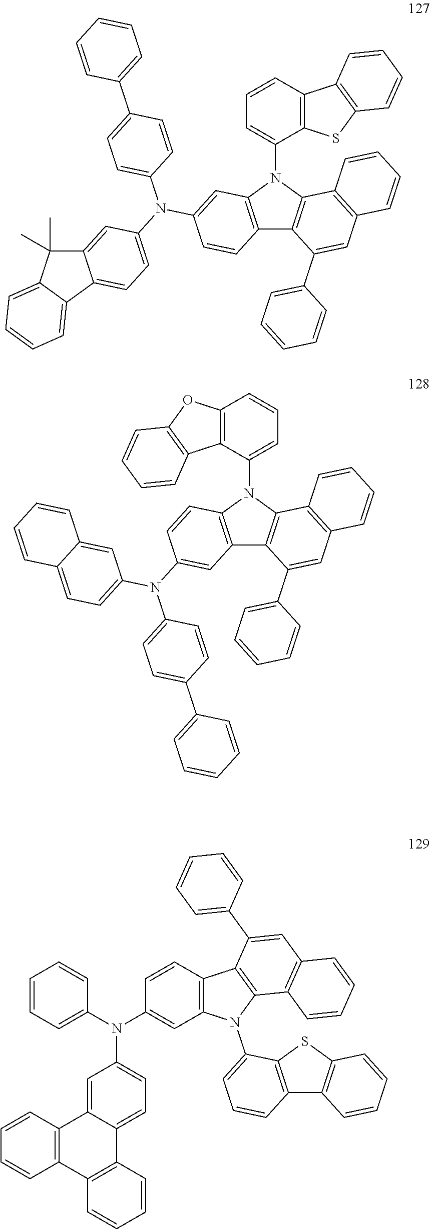 [SMILES: CC1(C)c2ccccc2-c2ccc(N(c3ccc(-c4ccccc4)cc3)c3ccc4c5c(-c6ccccc6)cc6ccccc6c5n(-c5cccc6c5sc5ccccc56)c4c3)cc21 ; c1ccc(-c2ccc(N(c3ccc4ccccc4c3)c3ccc4c(c3)c3c(-c5ccccc5)cc5ccccc5c3n4-c3cccc4oc5ccccc5c34)cc2)cc1 ; c1ccc(-c2cc3ccccc3c3c2c2ccc(N(c4ccccc4)c4ccc5c6ccccc6c6ccccc6c5c4)cc2n3-c2cccc3c2sc2ccccc23)cc1]

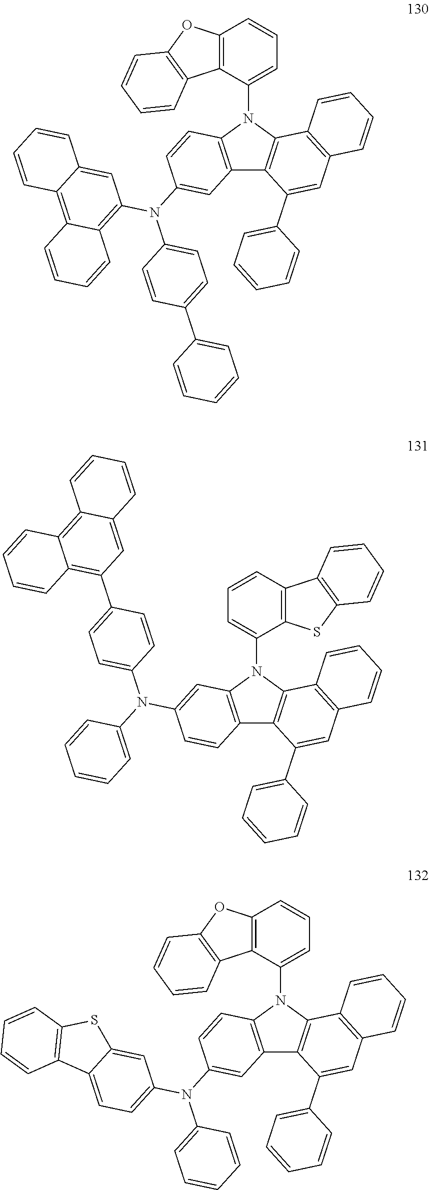 [SMILES: c1ccc(-c2ccc(N(c3ccc4c(c3)c3c(-c5ccccc5)cc5ccccc5c3n4-c3cccc4oc5ccccc5c34)c3cc4ccccc4c4ccccc34)cc2)cc1 ; c1ccc(-c2cc3ccccc3c3c2c2ccc(N(c4ccccc4)c4ccc(-c5cc6ccccc6c6ccccc56)cc4)cc2n3-c2cccc3c2sc2ccccc23)cc1 ; c1ccc(-c2cc3ccccc3c3c2c2cc(N(c4ccccc4)c4ccc5c(c4)sc4ccccc45)ccc2n3-c2cccc3oc4ccccc4c23)cc1]

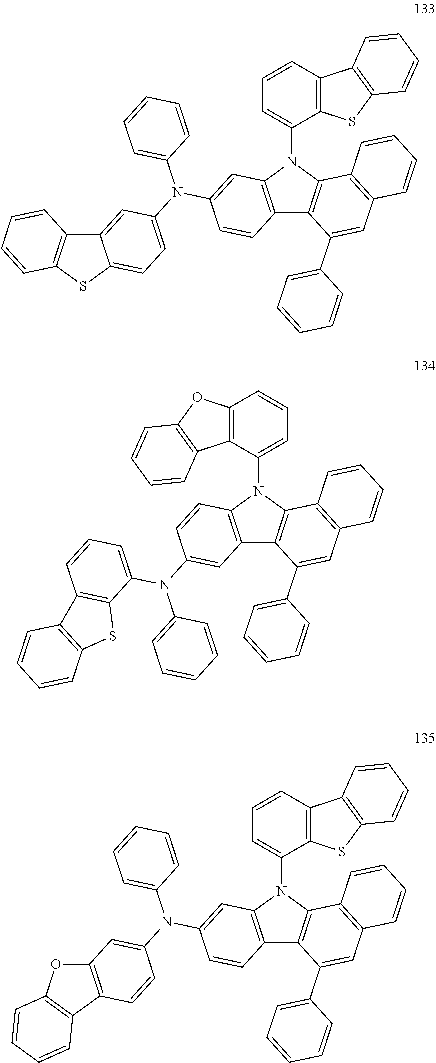 [SMILES: c1ccc(-c2cc3ccccc3c3c2c2ccc(N(c4ccccc4)c4ccc5sc6ccccc6c5c4)cc2n3-c2cccc3c2sc2ccccc23)cc1 ; c1ccc(-c2cc3ccccc3c3c2c2cc(N(c4ccccc4)c4cccc5c4sc4ccccc45)ccc2n3-c2cccc3oc4ccccc4c23)cc1 ; c1ccc(-c2cc3ccccc3c3c2c2ccc(N(c4ccccc4)c4ccc5c(c4)oc4ccccc45)cc2n3-c2cccc3c2sc2ccccc23)cc1]

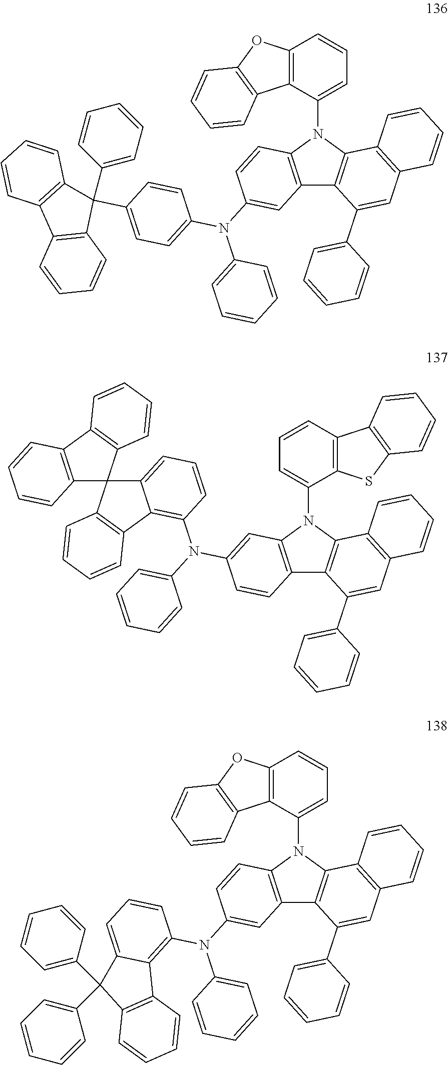 [SMILES: c1ccc(-c2cc3ccccc3c3c2c2cc(N(c4ccccc4)c4ccc(C5(c6ccccc6)c6ccccc6-c6ccccc65)cc4)ccc2n3-c2cccc3oc4ccccc4c23)cc1 ; c1ccc(-c2cc3ccccc3c3c2c2ccc(N(c4ccccc4)c4cccc5c4-c4ccccc4C54c5ccccc5-c5ccccc54)cc2n3-c2cccc3c2sc2ccccc23)cc1 ; c1ccc(-c2cc3ccccc3c3c2c2cc(N(c4ccccc4)c4cccc5c4-c4ccccc4C5(c4ccccc4)c4ccccc4)ccc2n3-c2cccc3oc4ccccc4c23)cc1]

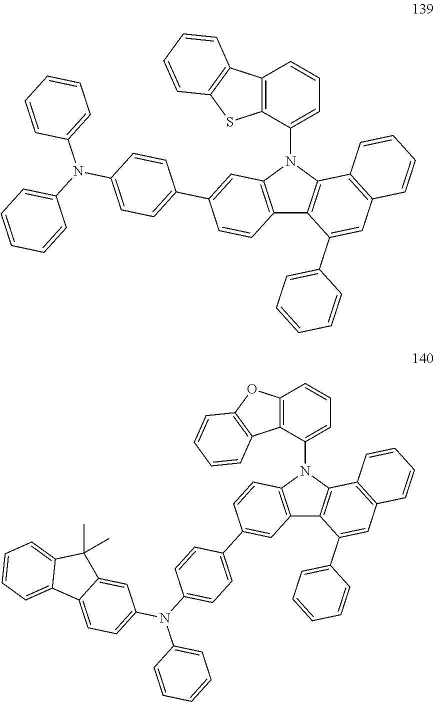 [SMILES: c1ccc(-c2cc3ccccc3c3c2c2ccc(-c4ccc(N(c5ccccc5)c5ccccc5)cc4)cc2n3-c2cccc3c2sc2ccccc23)cc1 ; CC1(C)c2ccccc2-c2ccc(N(c3ccccc3)c3ccc(-c4ccc5c(c4)c4c(-c6ccccc6)cc6ccccc6c4n5-c4cccc5oc6ccccc6c45)cc3)cc21]

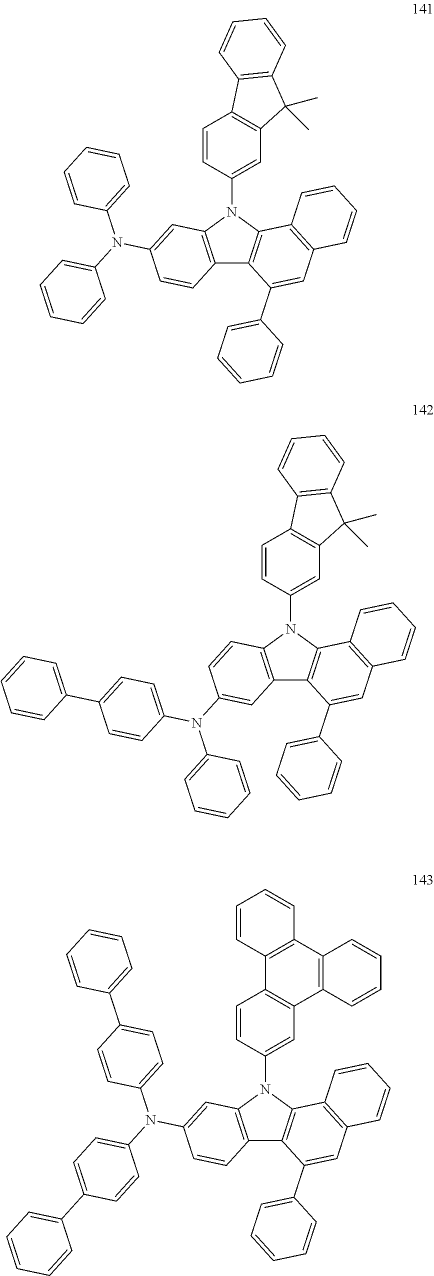 [SMILES: CC1(C)c2ccccc2-c2ccc(-n3c4cc(N(c5ccccc5)c5ccccc5)ccc4c4c(-c5ccccc5)cc5ccccc5c43)cc21 ; CC1(C)c2ccccc2-c2ccc(-n3c4ccc(N(c5ccccc5)c5ccc(-c6ccccc6)cc5)cc4c4c(-c5ccccc5)cc5ccccc5c43)cc21 ; c1ccc(-c2ccc(N(c3ccc(-c4ccccc4)cc3)c3ccc4c5c(-c6ccccc6)cc6ccccc6c5n(-c5ccc6c7ccccc7c7ccccc7c6c5)c4c3)cc2)cc1]

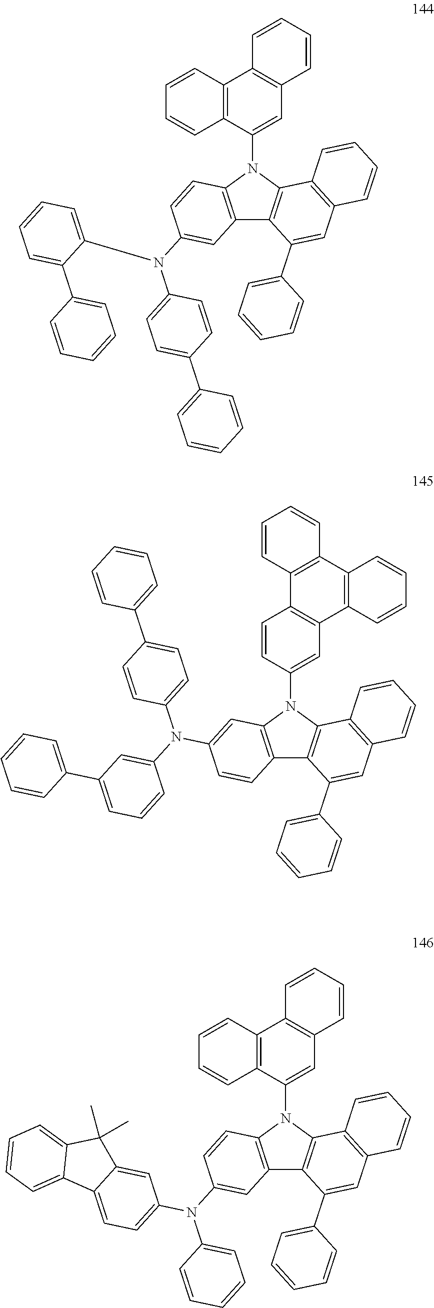 [SMILES: c1ccc(-c2ccc(N(c3ccc4c(c3)c3c(-c5ccccc5)cc5ccccc5c3n4-c3cc4ccccc4c4ccccc34)c3ccccc3-c3ccccc3)cc2)cc1 ; c1ccc(-c2ccc(N(c3cccc(-c4ccccc4)c3)c3ccc4c5c(-c6ccccc6)cc6ccccc6c5n(-c5ccc6c7ccccc7c7ccccc7c6c5)c4c3)cc2)cc1 ; CC1(C)c2ccccc2-c2ccc(N(c3ccccc3)c3ccc4c(c3)c3c(-c5ccccc5)cc5ccccc5c3n4-c3cc4ccccc4c4ccccc34)cc21]

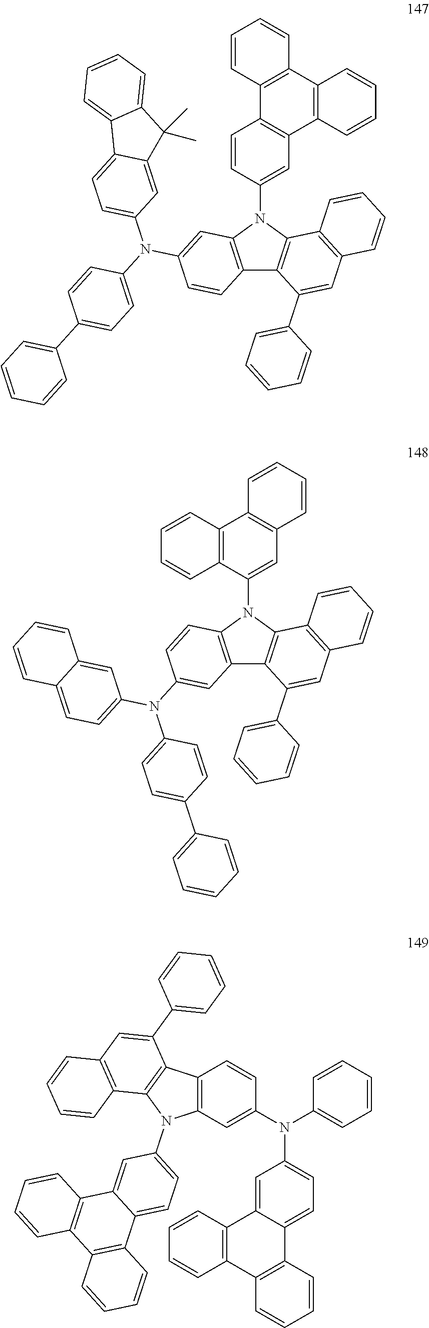 [SMILES: CC1(C)c2ccccc2-c2ccc(N(c3ccc(-c4ccccc4)cc3)c3ccc4c5c(-c6ccccc6)cc6ccccc6c5n(-c5ccc6c7ccccc7c7ccccc7c6c5)c4c3)cc21 ; c1ccc(-c2ccc(N(c3ccc4ccccc4c3)c3ccc4c(c3)c3c(-c5ccccc5)cc5ccccc5c3n4-c3cc4ccccc4c4ccccc34)cc2)cc1 ; c1ccc(-c2cc3ccccc3c3c2c2ccc(N(c4ccccc4)c4ccc5c6ccccc6c6ccccc6c5c4)cc2n3-c2ccc3c4ccccc4c4ccccc4c3c2)cc1]

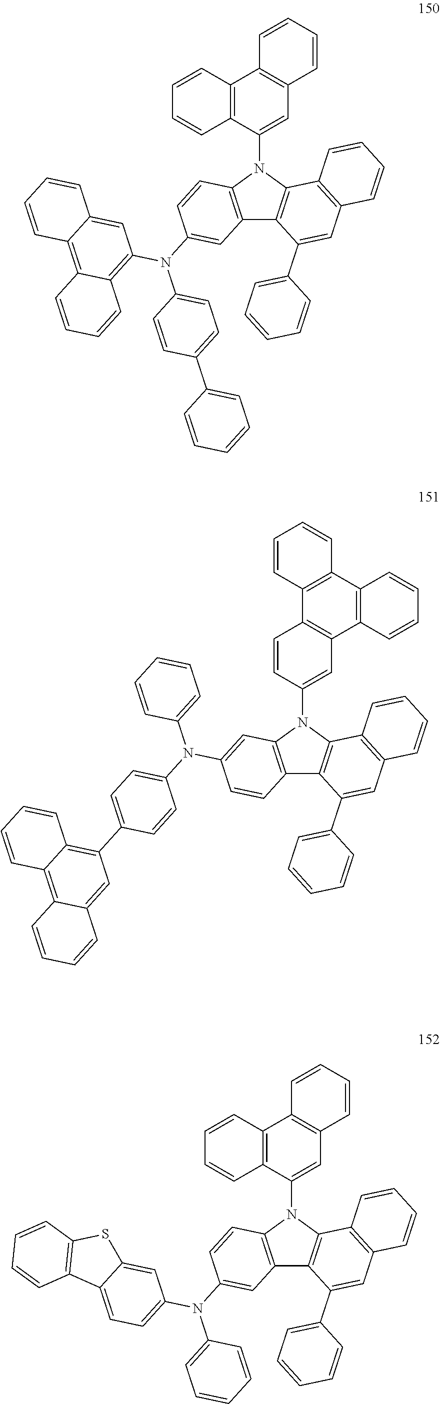 [SMILES: c1ccc(-c2ccc(N(c3ccc4c(c3)c3c(-c5ccccc5)cc5ccccc5c3n4-c3cc4ccccc4c4ccccc34)c3cc4ccccc4c4ccccc34)cc2)cc1 ; c1ccc(-c2cc3ccccc3c3c2c2ccc(N(c4ccccc4)c4ccc(-c5cc6ccccc6c6ccccc56)cc4)cc2n3-c2ccc3c4ccccc4c4ccccc4c3c2)cc1 ; c1ccc(-c2cc3ccccc3c3c2c2cc(N(c4ccccc4)c4ccc5c(c4)sc4ccccc45)ccc2n3-c2cc3ccccc3c3ccccc23)cc1]

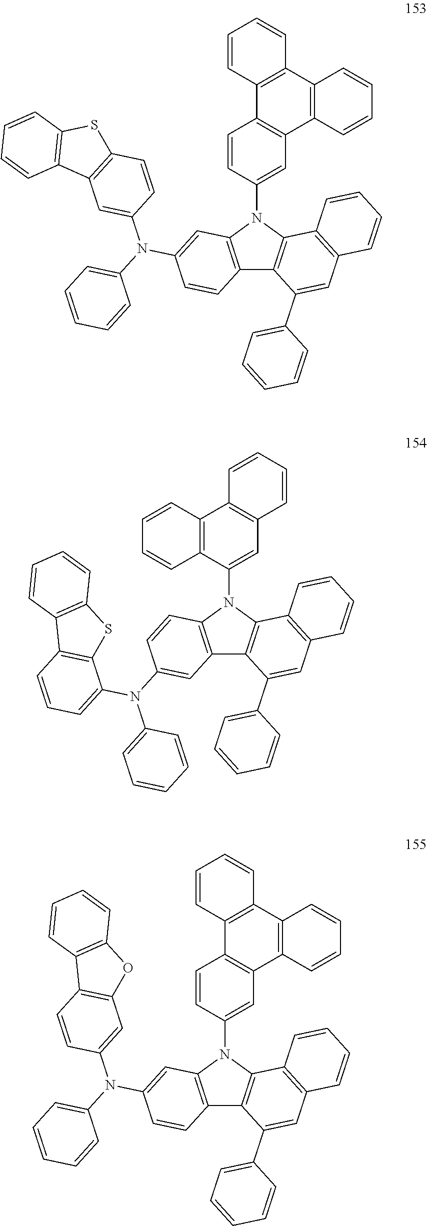 [SMILES: c1ccc(-c2cc3ccccc3c3c2c2ccc(N(c4ccccc4)c4ccc5sc6ccccc6c5c4)cc2n3-c2ccc3c4ccccc4c4ccccc4c3c2)cc1 ; c1ccc(-c2cc3ccccc3c3c2c2cc(N(c4ccccc4)c4cccc5c4sc4ccccc45)ccc2n3-c2cc3ccccc3c3ccccc23)cc1 ; c1ccc(-c2cc3ccccc3c3c2c2ccc(N(c4ccccc4)c4ccc5c(c4)oc4ccccc45)cc2n3-c2ccc3c4ccccc4c4ccccc4c3c2)cc1]

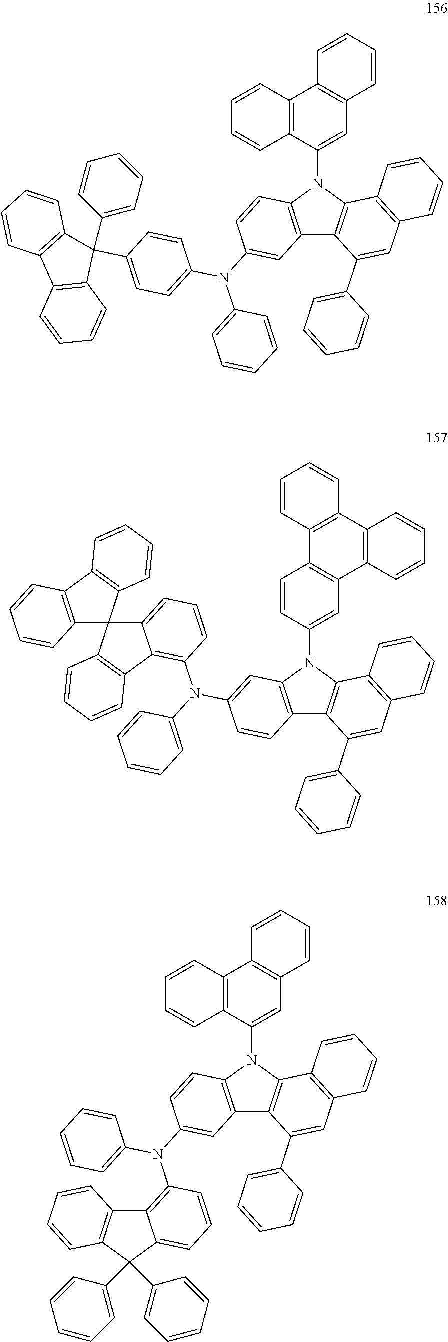 [SMILES: c1ccc(-c2cc3ccccc3c3c2c2cc(N(c4ccccc4)c4ccc(C5(c6ccccc6)c6ccccc6-c6ccccc65)cc4)ccc2n3-c2cc3ccccc3c3ccccc23)cc1 ; c1ccc(-c2cc3ccccc3c3c2c2ccc(N(c4ccccc4)c4cccc5c4-c4ccccc4C54c5ccccc5-c5ccccc54)cc2n3-c2ccc3c4ccccc4c4ccccc4c3c2)cc1 ; c1ccc(-c2cc3ccccc3c3c2c2cc(N(c4ccccc4)c4cccc5c4-c4ccccc4C5(c4ccccc4)c4ccccc4)ccc2n3-c2cc3ccccc3c3ccccc23)cc1]

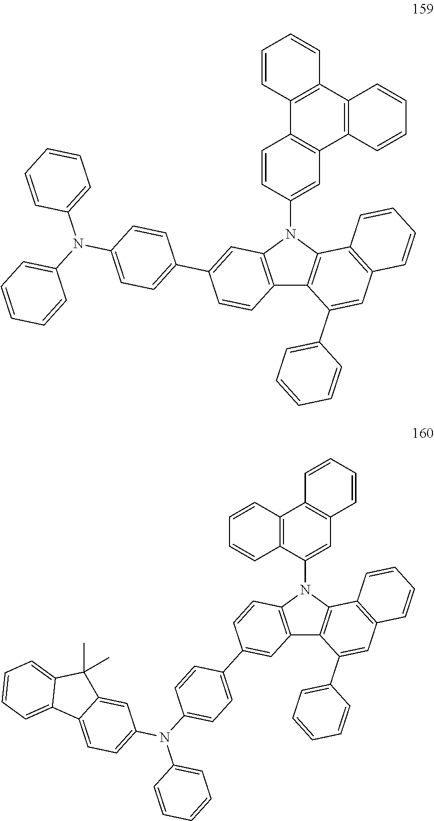 [SMILES: c1ccc(-c2cc3ccccc3c3c2c2ccc(-c4ccc(N(c5ccccc5)c5ccccc5)cc4)cc2n3-c2ccc3c4ccccc4c4ccccc4c3c2)cc1 ; CC1(C)c2ccccc2-c2ccc(N(c3ccccc3)c3ccc(-c4ccc5c(c4)c4c(-c6ccccc6)cc6ccccc6c4n5-c4cc5ccccc5c5ccccc45)cc3)cc21]

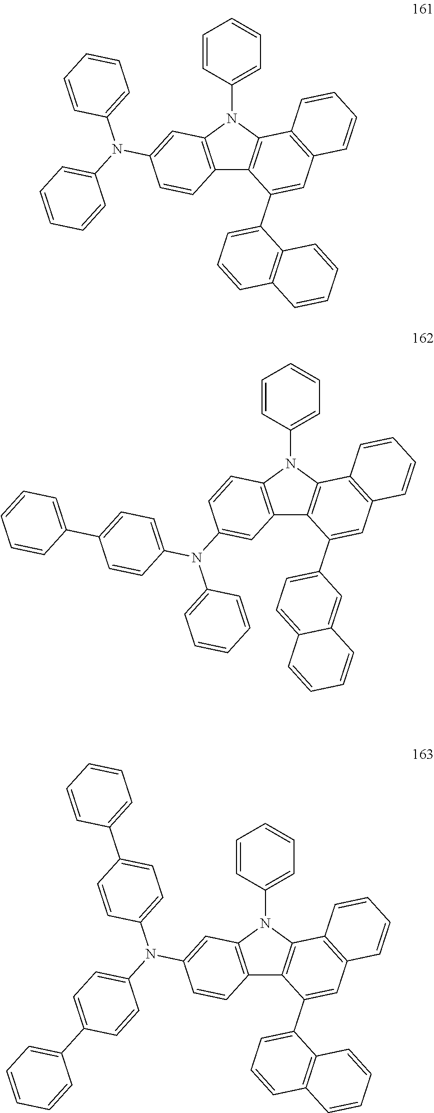 [SMILES: c1ccc(N(c2ccccc2)c2ccc3c4c(-c5cccc6ccccc56)cc5ccccc5c4n(-c4ccccc4)c3c2)cc1 ; c1ccc(-c2ccc(N(c3ccccc3)c3ccc4c(c3)c3c(-c5ccc6ccccc6c5)cc5ccccc5c3n4-c3ccccc3)cc2)cc1 ; c1ccc(-c2ccc(N(c3ccc(-c4ccccc4)cc3)c3ccc4c5c(-c6cccc7ccccc67)cc6ccccc6c5n(-c5ccccc5)c4c3)cc2)cc1]

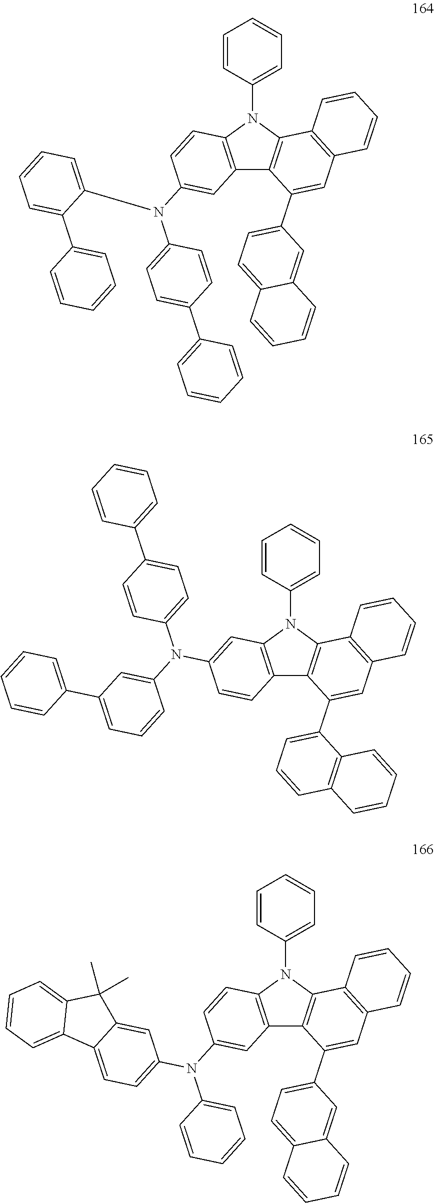 [SMILES: c1ccc(-c2ccc(N(c3ccc4c(c3)c3c(-c5ccc6ccccc6c5)cc5ccccc5c3n4-c3ccccc3)c3ccccc3-c3ccccc3)cc2)cc1 ; c1ccc(-c2ccc(N(c3cccc(-c4ccccc4)c3)c3ccc4c5c(-c6cccc7ccccc67)cc6ccccc6c5n(-c5ccccc5)c4c3)cc2)cc1 ; CC1(C)c2ccccc2-c2ccc(N(c3ccccc3)c3ccc4c(c3)c3c(-c5ccc6ccccc6c5)cc5ccccc5c3n4-c3ccccc3)cc21]

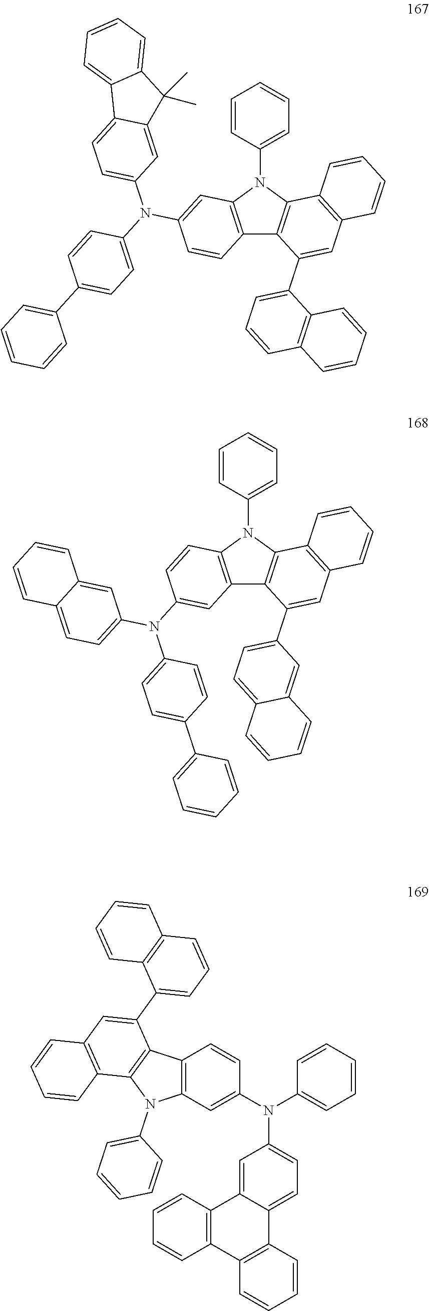 [SMILES: CC1(C)c2ccccc2-c2ccc(N(c3ccc(-c4ccccc4)cc3)c3ccc4c5c(-c6cccc7ccccc67)cc6ccccc6c5n(-c5ccccc5)c4c3)cc21 ; c1ccc(-c2ccc(N(c3ccc4ccccc4c3)c3ccc4c(c3)c3c(-c5ccc6ccccc6c5)cc5ccccc5c3n4-c3ccccc3)cc2)cc1 ; c1ccc(N(c2ccc3c4ccccc4c4ccccc4c3c2)c2ccc3c4c(-c5cccc6ccccc56)cc5ccccc5c4n(-c4ccccc4)c3c2)cc1]

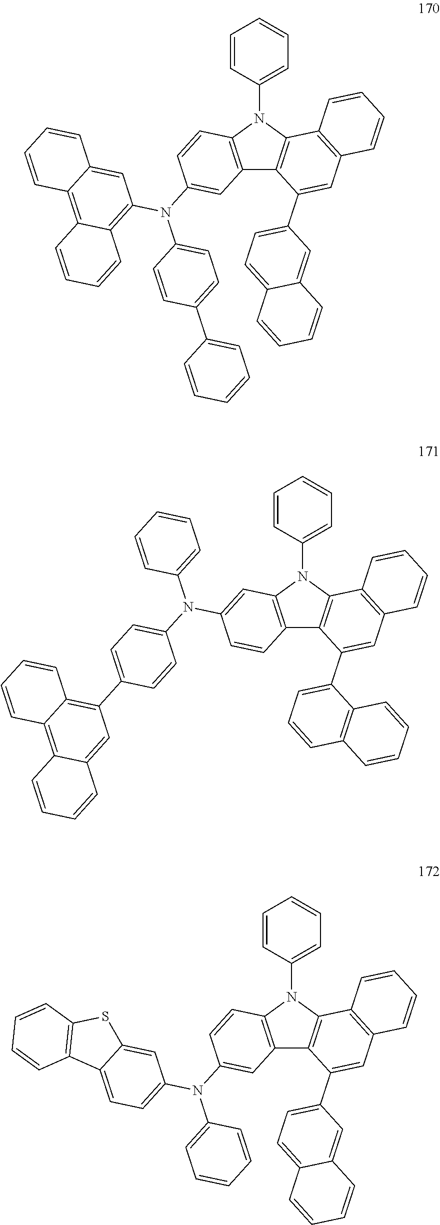 [SMILES: c1ccc(-c2ccc(N(c3ccc4c(c3)c3c(-c5ccc6ccccc6c5)cc5ccccc5c3n4-c3ccccc3)c3cc4ccccc4c4ccccc34)cc2)cc1 ; c1ccc(N(c2ccc(-c3cc4ccccc4c4ccccc34)cc2)c2ccc3c4c(-c5cccc6ccccc56)cc5ccccc5c4n(-c4ccccc4)c3c2)cc1 ; c1ccc(N(c2ccc3c(c2)sc2ccccc23)c2ccc3c(c2)c2c(-c4ccc5ccccc5c4)cc4ccccc4c2n3-c2ccccc2)cc1]

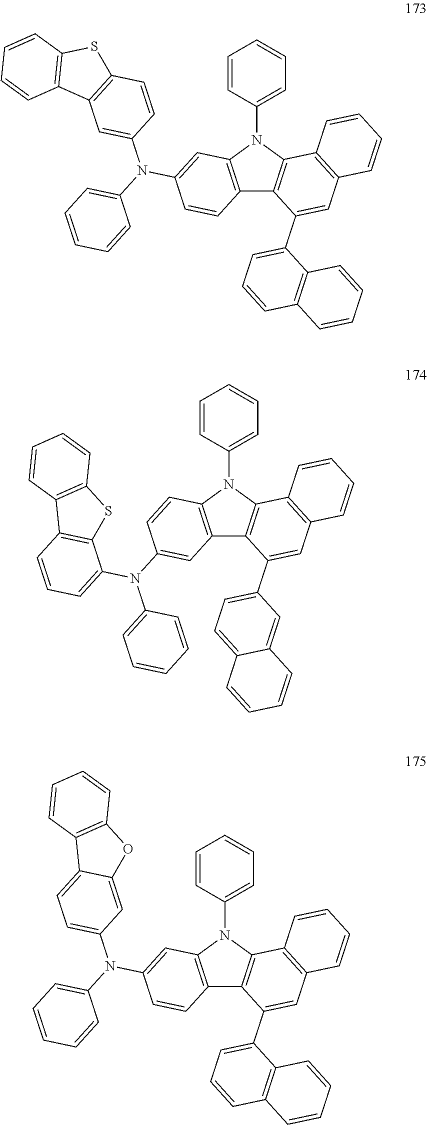 [SMILES: c1ccc(N(c2ccc3sc4ccccc4c3c2)c2ccc3c4c(-c5cccc6ccccc56)cc5ccccc5c4n(-c4ccccc4)c3c2)cc1 ; c1ccc(N(c2ccc3c(c2)c2c(-c4ccc5ccccc5c4)cc4ccccc4c2n3-c2ccccc2)c2cccc3c2sc2ccccc23)cc1 ; c1ccc(N(c2ccc3c(c2)oc2ccccc23)c2ccc3c4c(-c5cccc6ccccc56)cc5ccccc5c4n(-c4ccccc4)c3c2)cc1]

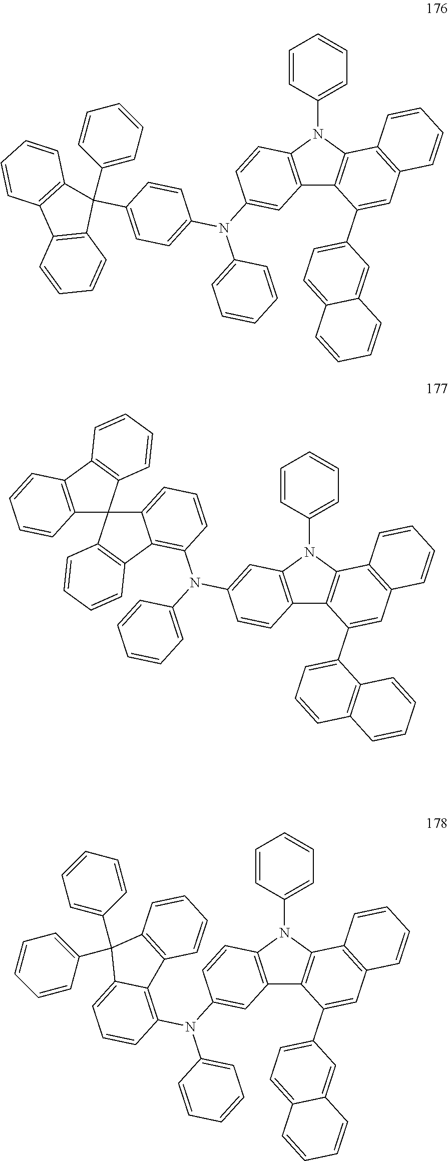 [SMILES: c1ccc(N(c2ccc(C3(c4ccccc4)c4ccccc4-c4ccccc43)cc2)c2ccc3c(c2)c2c(-c4ccc5ccccc5c4)cc4ccccc4c2n3-c2ccccc2)cc1 ; c1ccc(N(c2ccc3c4c(-c5cccc6ccccc56)cc5ccccc5c4n(-c4ccccc4)c3c2)c2cccc3c2-c2ccccc2C32c3ccccc3-c3ccccc32)cc1 ; c1ccc(N(c2ccc3c(c2)c2c(-c4ccc5ccccc5c4)cc4ccccc4c2n3-c2ccccc2)c2cccc3c2-c2ccccc2C3(c2ccccc2)c2ccccc2)cc1]

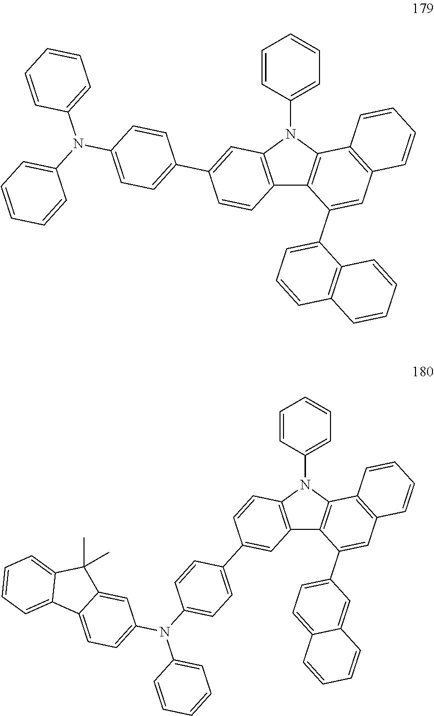 [SMILES: c1ccc(N(c2ccccc2)c2ccc(-c3ccc4c5c(-c6cccc7ccccc67)cc6ccccc6c5n(-c5ccccc5)c4c3)cc2)cc1 ; CC1(C)c2ccccc2-c2ccc(N(c3ccccc3)c3ccc(-c4ccc5c(c4)c4c(-c6ccc7ccccc7c6)cc6ccccc6c4n5-c4ccccc4)cc3)cc21]

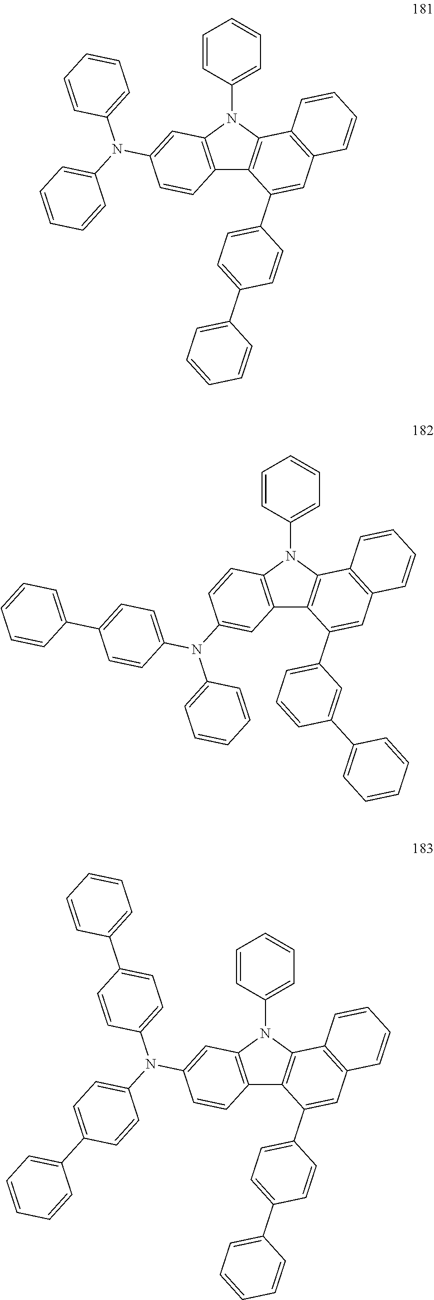 [SMILES: c1ccc(-c2ccc(-c3cc4ccccc4c4c3c3ccc(N(c5ccccc5)c5ccccc5)cc3n4-c3ccccc3)cc2)cc1 ; c1ccc(-c2ccc(N(c3ccccc3)c3ccc4c(c3)c3c(-c5cccc(-c6ccccc6)c5)cc5ccccc5c3n4-c3ccccc3)cc2)cc1 ; c1ccc(-c2ccc(-c3cc4ccccc4c4c3c3ccc(N(c5ccc(-c6ccccc6)cc5)c5ccc(-c6ccccc6)cc5)cc3n4-c3ccccc3)cc2)cc1]

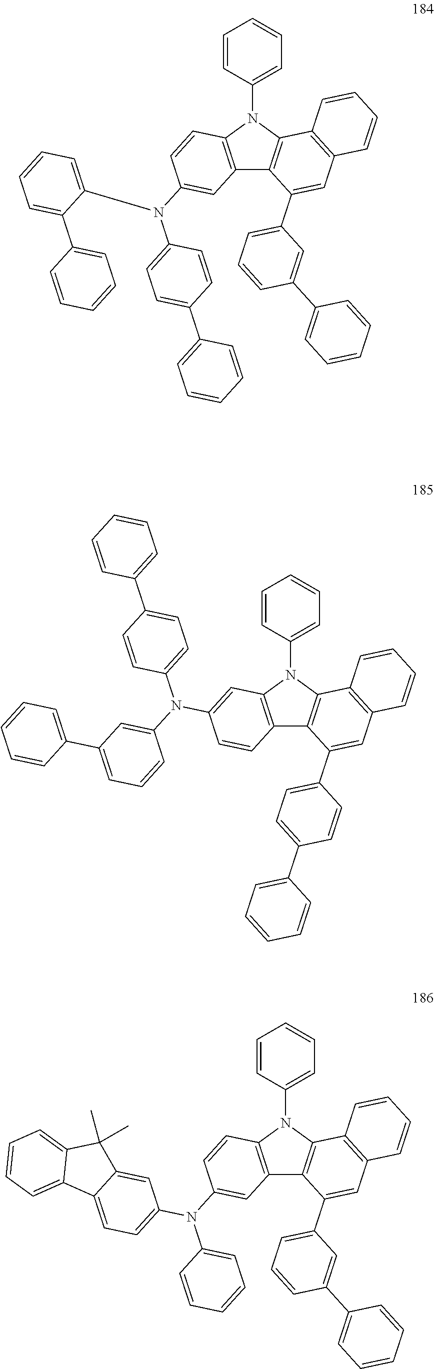 [SMILES: c1ccc(-c2ccc(N(c3ccc4c(c3)c3c(-c5cccc(-c6ccccc6)c5)cc5ccccc5c3n4-c3ccccc3)c3ccccc3-c3ccccc3)cc2)cc1 ; c1ccc(-c2ccc(-c3cc4ccccc4c4c3c3ccc(N(c5ccc(-c6ccccc6)cc5)c5cccc(-c6ccccc6)c5)cc3n4-c3ccccc3)cc2)cc1 ; CC1(C)c2ccccc2-c2ccc(N(c3ccccc3)c3ccc4c(c3)c3c(-c5cccc(-c6ccccc6)c5)cc5ccccc5c3n4-c3ccccc3)cc21]

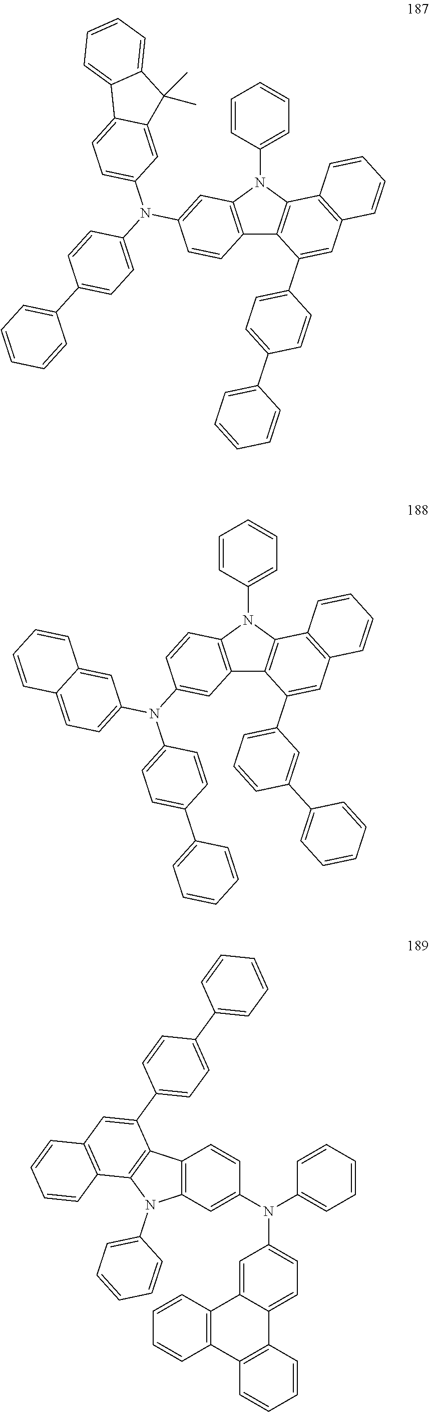 [SMILES: CC1(C)c2ccccc2-c2ccc(N(c3ccc(-c4ccccc4)cc3)c3ccc4c5c(-c6ccc(-c7ccccc7)cc6)cc6ccccc6c5n(-c5ccccc5)c4c3)cc21 ; c1ccc(-c2ccc(N(c3ccc4ccccc4c3)c3ccc4c(c3)c3c(-c5cccc(-c6ccccc6)c5)cc5ccccc5c3n4-c3ccccc3)cc2)cc1 ; c1ccc(-c2ccc(-c3cc4ccccc4c4c3c3ccc(N(c5ccccc5)c5ccc6c7ccccc7c7ccccc7c6c5)cc3n4-c3ccccc3)cc2)cc1]

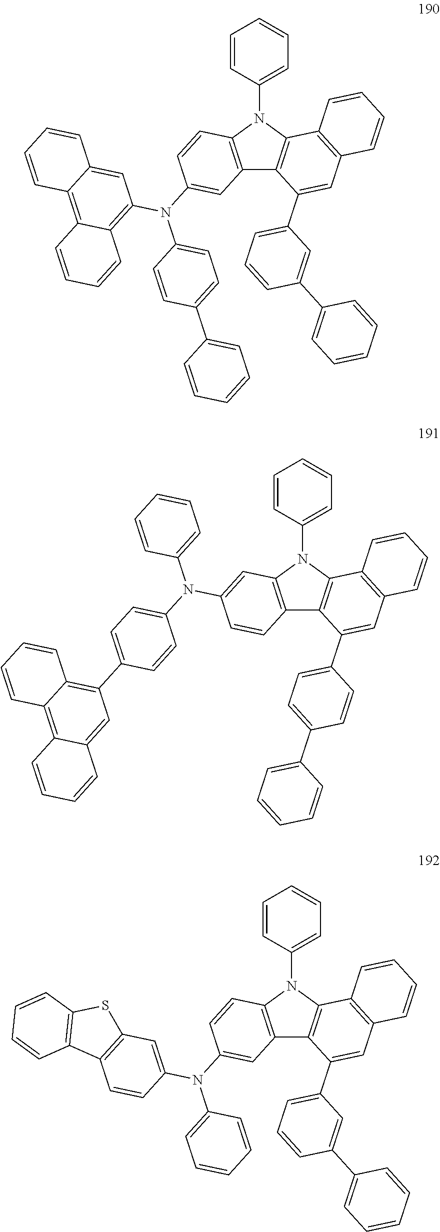 [SMILES: c1ccc(-c2ccc(N(c3ccc4c(c3)c3c(-c5cccc(-c6ccccc6)c5)cc5ccccc5c3n4-c3ccccc3)c3cc4ccccc4c4ccccc34)cc2)cc1 ; c1ccc(-c2ccc(-c3cc4ccccc4c4c3c3ccc(N(c5ccccc5)c5ccc(-c6cc7ccccc7c7ccccc67)cc5)cc3n4-c3ccccc3)cc2)cc1 ; c1ccc(-c2cccc(-c3cc4ccccc4c4c3c3cc(N(c5ccccc5)c5ccc6c(c5)sc5ccccc56)ccc3n4-c3ccccc3)c2)cc1]

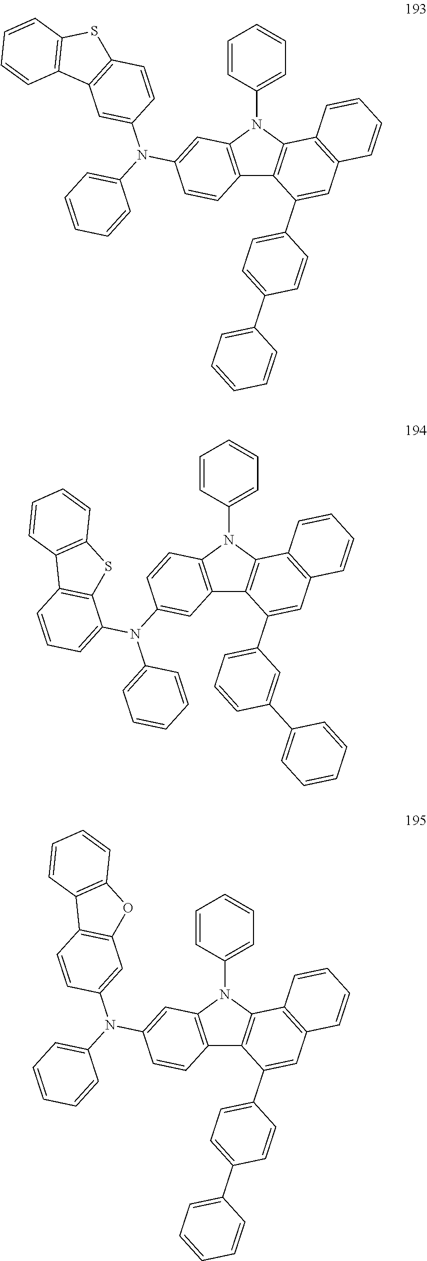 [SMILES: c1ccc(-c2ccc(-c3cc4ccccc4c4c3c3ccc(N(c5ccccc5)c5ccc6sc7ccccc7c6c5)cc3n4-c3ccccc3)cc2)cc1 ; c1ccc(-c2cccc(-c3cc4ccccc4c4c3c3cc(N(c5ccccc5)c5cccc6c5sc5ccccc56)ccc3n4-c3ccccc3)c2)cc1 ; c1ccc(-c2ccc(-c3cc4ccccc4c4c3c3ccc(N(c5ccccc5)c5ccc6c(c5)oc5ccccc56)cc3n4-c3ccccc3)cc2)cc1]

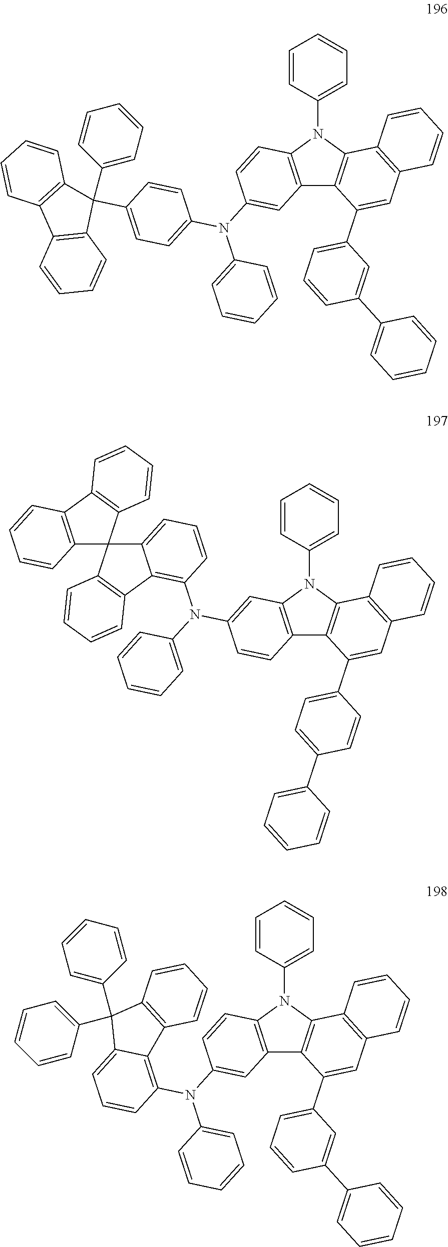 [SMILES: c1ccc(-c2cccc(-c3cc4ccccc4c4c3c3cc(N(c5ccccc5)c5ccc(C6(c7ccccc7)c7ccccc7-c7ccccc76)cc5)ccc3n4-c3ccccc3)c2)cc1 ; c1ccc(-c2ccc(-c3cc4ccccc4c4c3c3ccc(N(c5ccccc5)c5cccc6c5-c5ccccc5C65c6ccccc6-c6ccccc65)cc3n4-c3ccccc3)cc2)cc1 ; c1ccc(-c2cccc(-c3cc4ccccc4c4c3c3cc(N(c5ccccc5)c5cccc6c5-c5ccccc5C6(c5ccccc5)c5ccccc5)ccc3n4-c3ccccc3)c2)cc1]

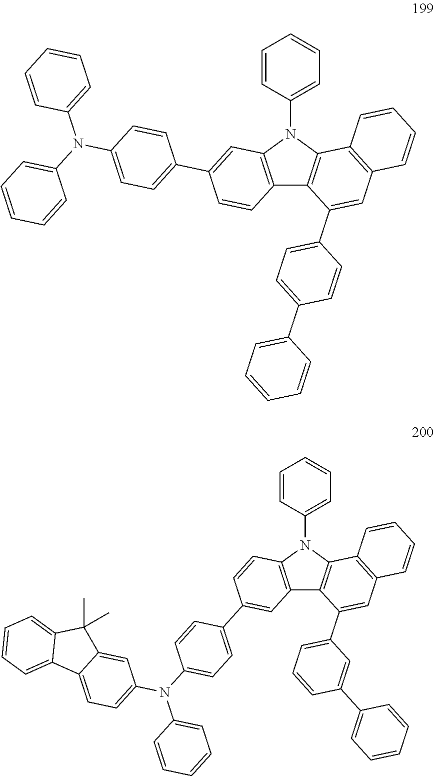 [SMILES: c1ccc(-c2ccc(-c3cc4ccccc4c4c3c3ccc(-c5ccc(N(c6ccccc6)c6ccccc6)cc5)cc3n4-c3ccccc3)cc2)cc1 ; CC1(C)c2ccccc2-c2ccc(N(c3ccccc3)c3ccc(-c4ccc5c(c4)c4c(-c6cccc(-c7ccccc7)c6)cc6ccccc6c4n5-c4ccccc4)cc3)cc21]

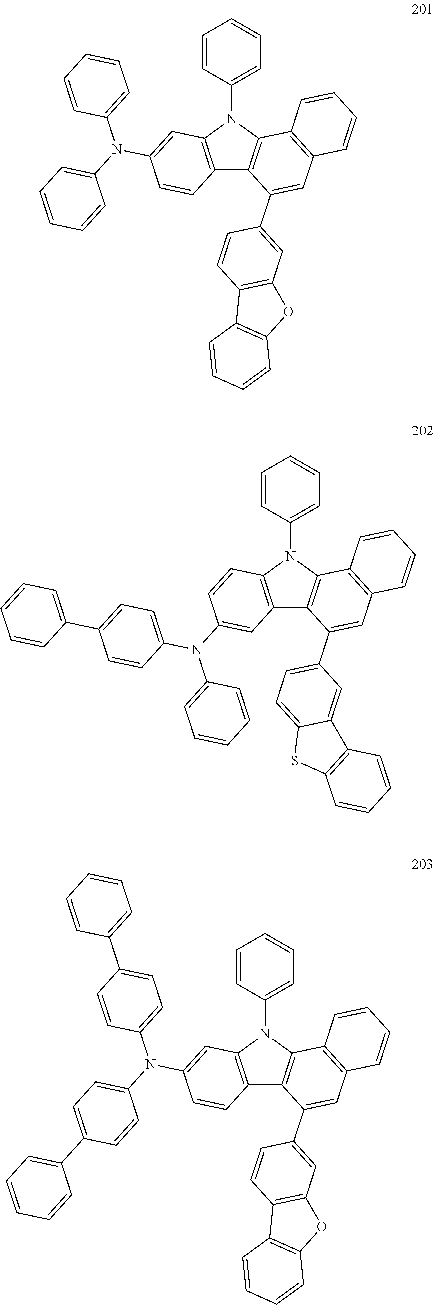 [SMILES: c1ccc(N(c2ccccc2)c2ccc3c4c(-c5ccc6c(c5)oc5ccccc56)cc5ccccc5c4n(-c4ccccc4)c3c2)cc1 ; c1ccc(-c2ccc(N(c3ccccc3)c3ccc4c(c3)c3c(-c5ccc6sc7ccccc7c6c5)cc5ccccc5c3n4-c3ccccc3)cc2)cc1 ; c1ccc(-c2ccc(N(c3ccc(-c4ccccc4)cc3)c3ccc4c5c(-c6ccc7c(c6)oc6ccccc67)cc6ccccc6c5n(-c5ccccc5)c4c3)cc2)cc1]

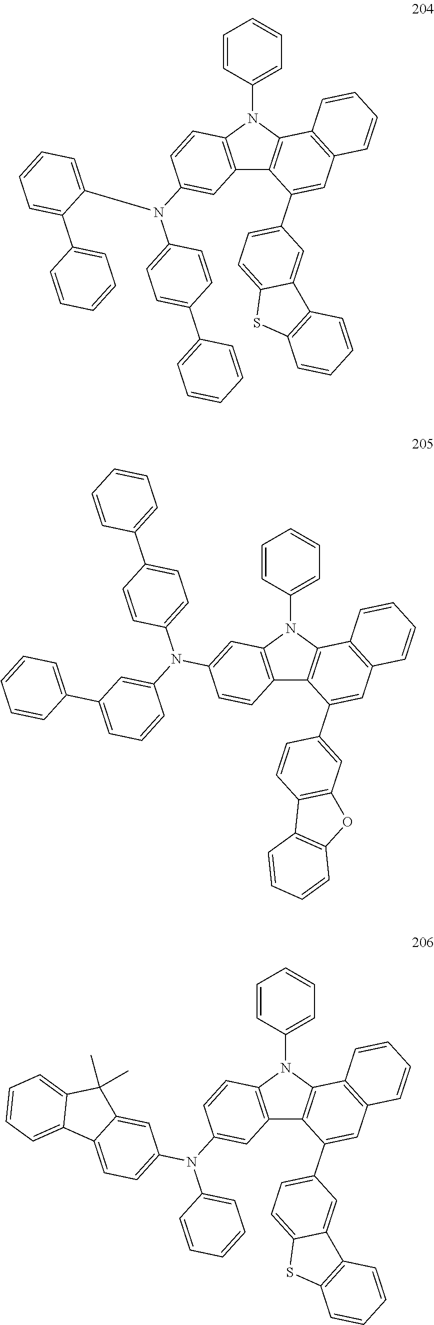 [SMILES: c1ccc(-c2ccc(N(c3ccc4c(c3)c3c(-c5ccc6sc7ccccc7c6c5)cc5ccccc5c3n4-c3ccccc3)c3ccccc3-c3ccccc3)cc2)cc1 ; c1ccc(-c2ccc(N(c3cccc(-c4ccccc4)c3)c3ccc4c5c(-c6ccc7c(c6)oc6ccccc67)cc6ccccc6c5n(-c5ccccc5)c4c3)cc2)cc1 ; CC1(C)c2ccccc2-c2ccc(N(c3ccccc3)c3ccc4c(c3)c3c(-c5ccc6sc7ccccc7c6c5)cc5ccccc5c3n4-c3ccccc3)cc21]

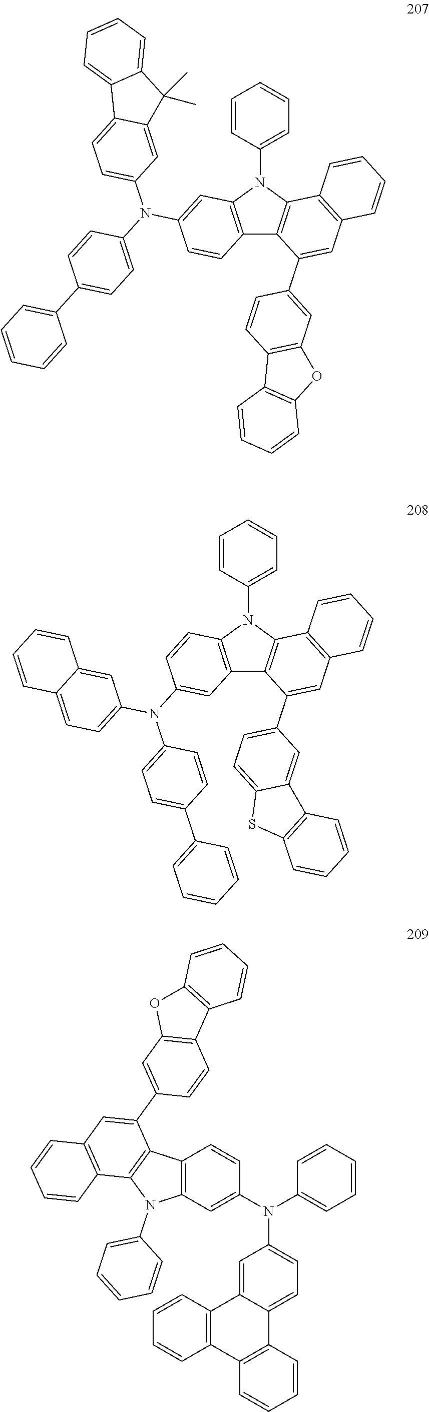

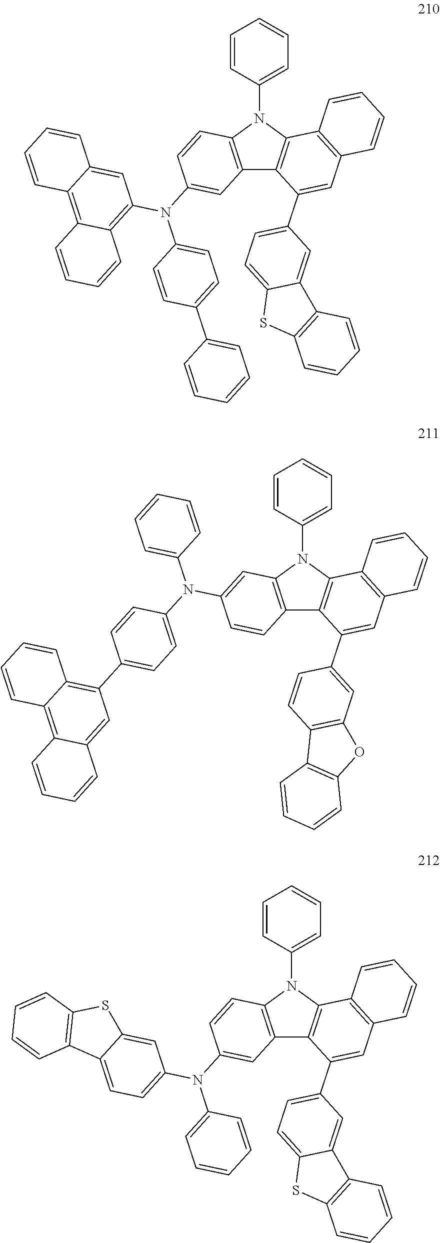 [SMILES: c1ccc(-c2ccc(N(c3ccc4c(c3)c3c(-c5ccc6sc7ccccc7c6c5)cc5ccccc5c3n4-c3ccccc3)c3cc4ccccc4c4ccccc34)cc2)cc1 ; c1ccc(N(c2ccc(-c3cc4ccccc4c4ccccc34)cc2)c2ccc3c4c(-c5ccc6c(c5)oc5ccccc56)cc5ccccc5c4n(-c4ccccc4)c3c2)cc1 ; c1ccc(N(c2ccc3c(c2)sc2ccccc23)c2ccc3c(c2)c2c(-c4ccc5sc6ccccc6c5c4)cc4ccccc4c2n3-c2ccccc2)cc1]

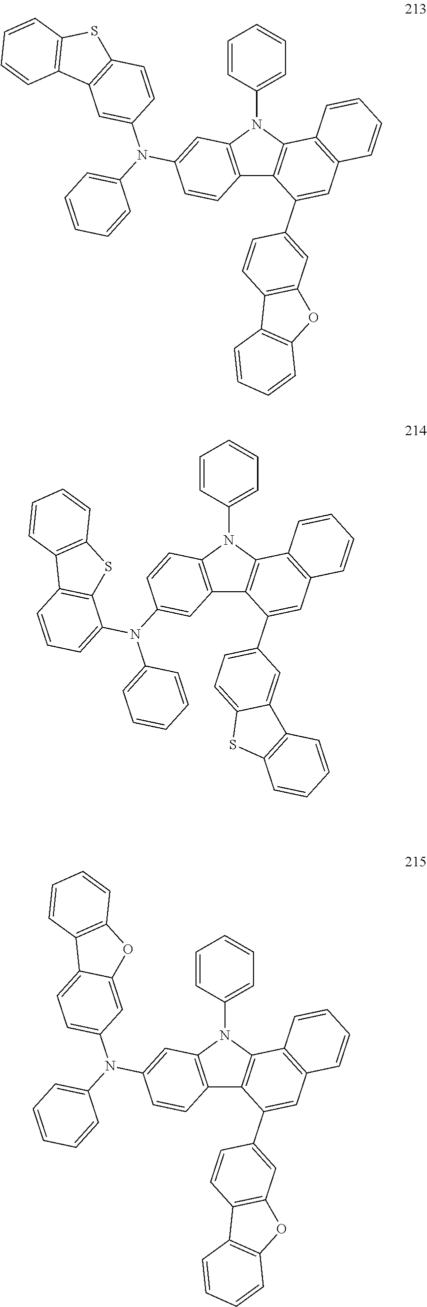 [SMILES: c1ccc(N(c2ccc3sc4ccccc4c3c2)c2ccc3c4c(-c5ccc6c(c5)oc5ccccc56)cc5ccccc5c4n(-c4ccccc4)c3c2)cc1 ; c1ccc(N(c2ccc3c(c2)c2c(-c4ccc5sc6ccccc6c5c4)cc4ccccc4c2n3-c2ccccc2)c2cccc3c2sc2ccccc23)cc1 ; c1ccc(N(c2ccc3c(c2)oc2ccccc23)c2ccc3c4c(-c5ccc6c(c5)oc5ccccc56)cc5ccccc5c4n(-c4ccccc4)c3c2)cc1]

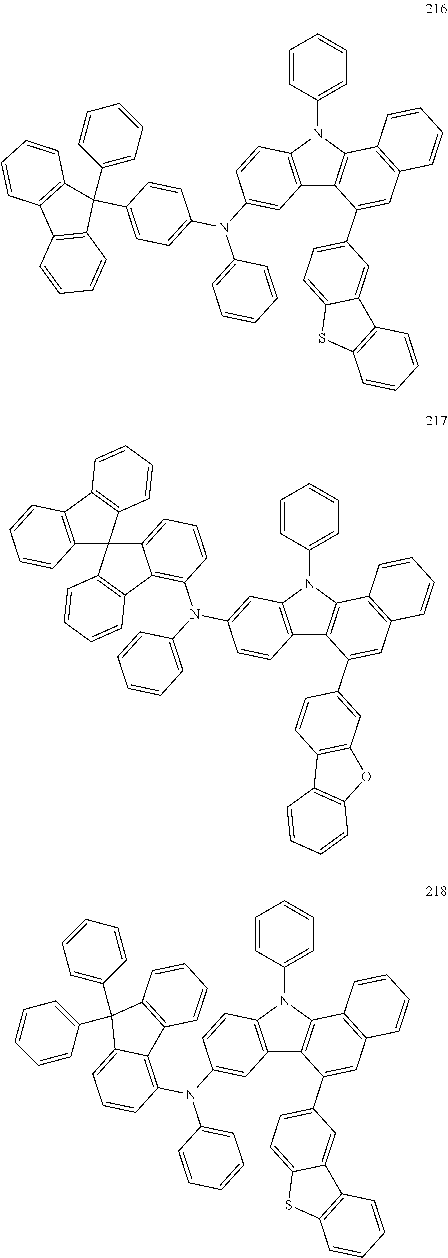 [SMILES: c1ccc(N(c2ccc(C3(c4ccccc4)c4ccccc4-c4ccccc43)cc2)c2ccc3c(c2)c2c(-c4ccc5sc6ccccc6c5c4)cc4ccccc4c2n3-c2ccccc2)cc1 ; c1ccc(N(c2ccc3c4c(-c5ccc6c(c5)oc5ccccc56)cc5ccccc5c4n(-c4ccccc4)c3c2)c2cccc3c2-c2ccccc2C32c3ccccc3-c3ccccc32)cc1 ; c1ccc(N(c2ccc3c(c2)c2c(-c4ccc5sc6ccccc6c5c4)cc4ccccc4c2n3-c2ccccc2)c2cccc3c2-c2ccccc2C3(c2ccccc2)c2ccccc2)cc1]

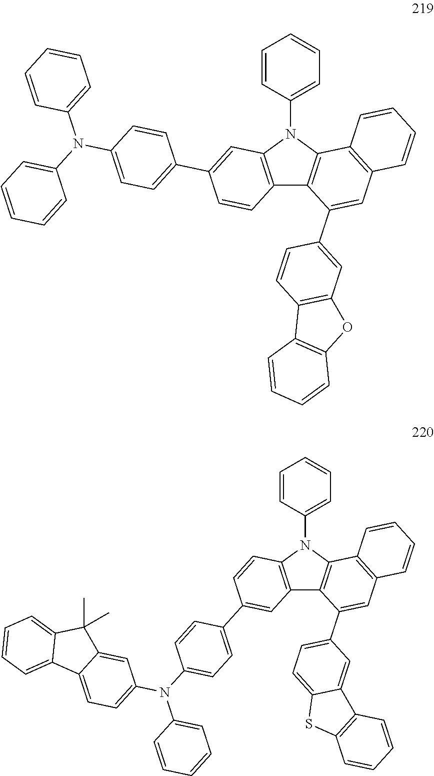 [SMILES: c1ccc(N(c2ccccc2)c2ccc(-c3ccc4c5c(-c6ccc7c(c6)oc6ccccc67)cc6ccccc6c5n(-c5ccccc5)c4c3)cc2)cc1 ; CC1(C)c2ccccc2-c2ccc(N(c3ccccc3)c3ccc(-c4ccc5c(c4)c4c(-c6ccc7sc8ccccc8c7c6)cc6ccccc6c4n5-c4ccccc4)cc3)cc21]

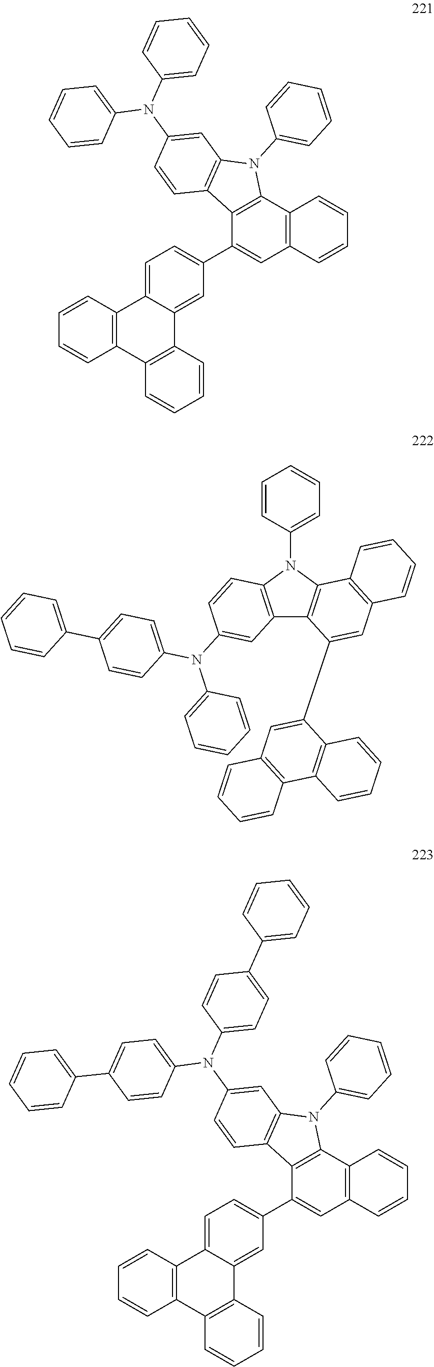 [SMILES: c1ccc(N(c2ccccc2)c2ccc3c4c(-c5ccc6c7ccccc7c7ccccc7c6c5)cc5ccccc5c4n(-c4ccccc4)c3c2)cc1 ; c1ccc(-c2ccc(N(c3ccccc3)c3ccc4c(c3)c3c(-c5cc6ccccc6c6ccccc56)cc5ccccc5c3n4-c3ccccc3)cc2)cc1 ; c1ccc(-c2ccc(N(c3ccc(-c4ccccc4)cc3)c3ccc4c5c(-c6ccc7c8ccccc8c8ccccc8c7c6)cc6ccccc6c5n(-c5ccccc5)c4c3)cc2)cc1]

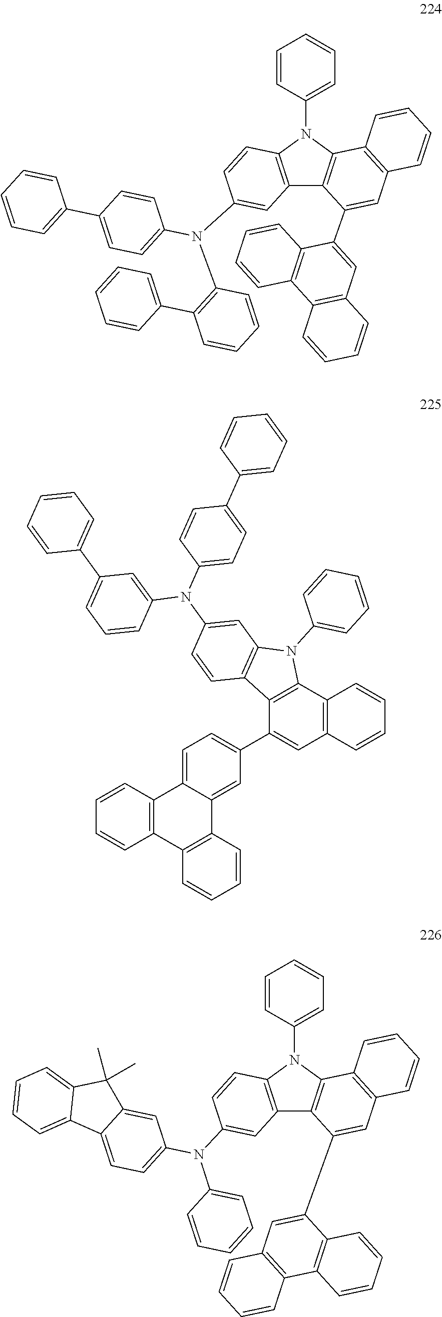 [SMILES: c1ccc(-c2ccc(N(c3ccc4c(c3)c3c(-c5cc6ccccc6c6ccccc56)cc5ccccc5c3n4-c3ccccc3)c3ccccc3-c3ccccc3)cc2)cc1 ; c1ccc(-c2ccc(N(c3cccc(-c4ccccc4)c3)c3ccc4c5c(-c6ccc7c8ccccc8c8ccccc8c7c6)cc6ccccc6c5n(-c5ccccc5)c4c3)cc2)cc1 ; CC1(C)c2ccccc2-c2ccc(N(c3ccccc3)c3ccc4c(c3)c3c(-c5cc6ccccc6c6ccccc56)cc5ccccc5c3n4-c3ccccc3)cc21]

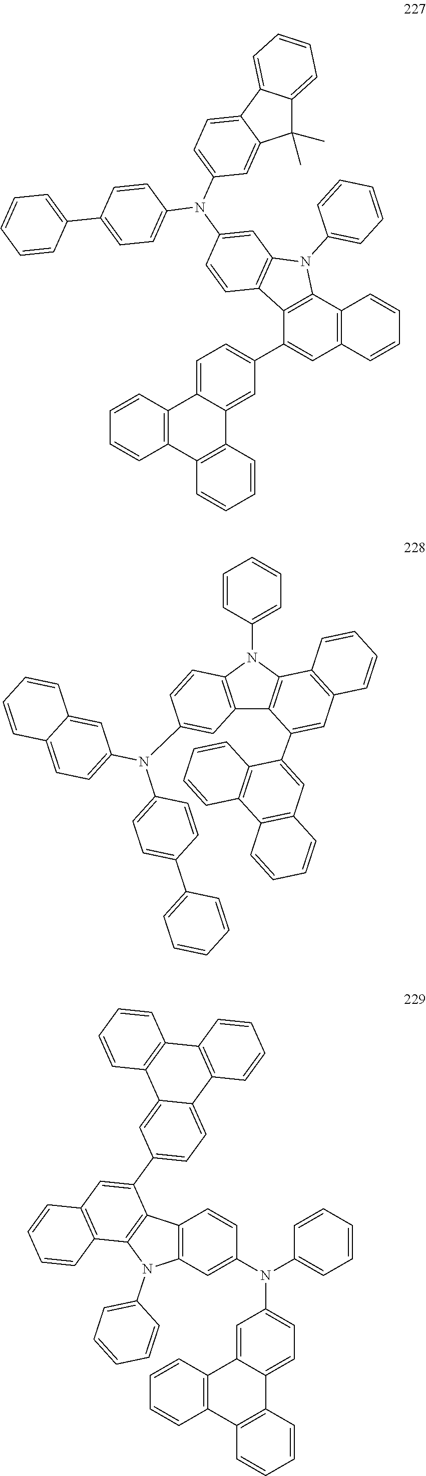 [SMILES: CC1(C)c2ccccc2-c2ccc(N(c3ccc(-c4ccccc4)cc3)c3ccc4c5c(-c6ccc7c8ccccc8c8ccccc8c7c6)cc6ccccc6c5n(-c5ccccc5)c4c3)cc21 ; c1ccc(-c2ccc(N(c3ccc4ccccc4c3)c3ccc4c(c3)c3c(-c5cc6ccccc6c6ccccc56)cc5ccccc5c3n4-c3ccccc3)cc2)cc1 ; c1ccc(N(c2ccc3c4ccccc4c4ccccc4c3c2)c2ccc3c4c(-c5ccc6c7ccccc7c7ccccc7c6c5)cc5ccccc5c4n(-c4ccccc4)c3c2)cc1]

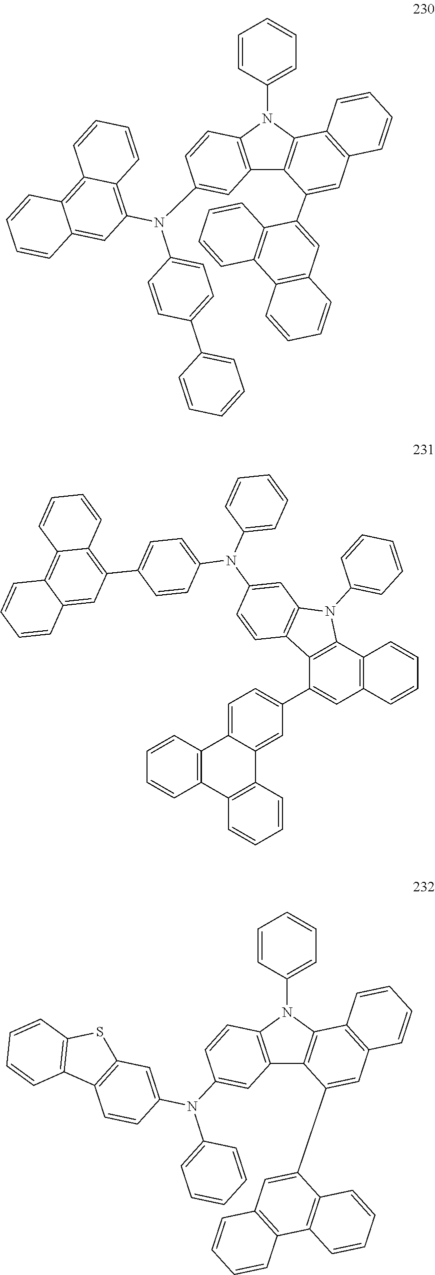 [SMILES: c1ccc(-c2ccc(N(c3ccc4c(c3)c3c(-c5cc6ccccc6c6ccccc56)cc5ccccc5c3n4-c3ccccc3)c3cc4ccccc4c4ccccc34)cc2)cc1 ; c1ccc(N(c2ccc(-c3cc4ccccc4c4ccccc34)cc2)c2ccc3c4c(-c5ccc6c7ccccc7c7ccccc7c6c5)cc5ccccc5c4n(-c4ccccc4)c3c2)cc1 ; c1ccc(N(c2ccc3c(c2)sc2ccccc23)c2ccc3c(c2)c2c(-c4cc5ccccc5c5ccccc45)cc4ccccc4c2n3-c2ccccc2)cc1]

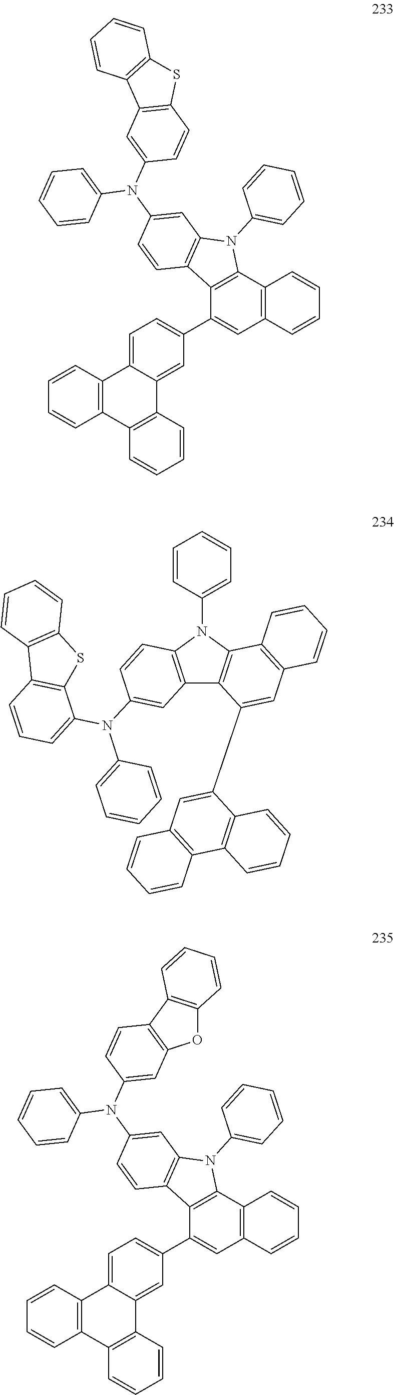 [SMILES: c1ccc(N(c2ccc3sc4ccccc4c3c2)c2ccc3c4c(-c5ccc6c7ccccc7c7ccccc7c6c5)cc5ccccc5c4n(-c4ccccc4)c3c2)cc1 ; c1ccc(N(c2ccc3c(c2)c2c(-c4cc5ccccc5c5ccccc45)cc4ccccc4c2n3-c2ccccc2)c2cccc3c2sc2ccccc23)cc1 ; c1ccc(N(c2ccc3c(c2)oc2ccccc23)c2ccc3c4c(-c5ccc6c7ccccc7c7ccccc7c6c5)cc5ccccc5c4n(-c4ccccc4)c3c2)cc1]

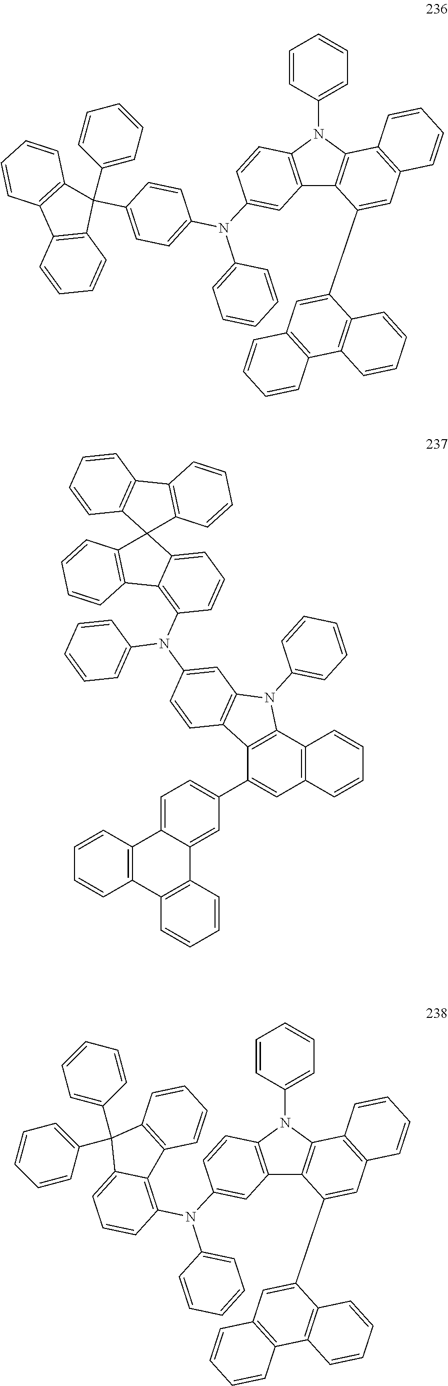 [SMILES: c1ccc(N(c2ccc(C3(c4ccccc4)c4ccccc4-c4ccccc43)cc2)c2ccc3c(c2)c2c(-c4cc5ccccc5c5ccccc45)cc4ccccc4c2n3-c2ccccc2)cc1 ; c1ccc(N(c2ccc3c4c(-c5ccc6c7ccccc7c7ccccc7c6c5)cc5ccccc5c4n(-c4ccccc4)c3c2)c2cccc3c2-c2ccccc2C32c3ccccc3-c3ccccc32)cc1 ; c1ccc(N(c2ccc3c(c2)c2c(-c4cc5ccccc5c5ccccc45)cc4ccccc4c2n3-c2ccccc2)c2cccc3c2-c2ccccc2C3(c2ccccc2)c2ccccc2)cc1]

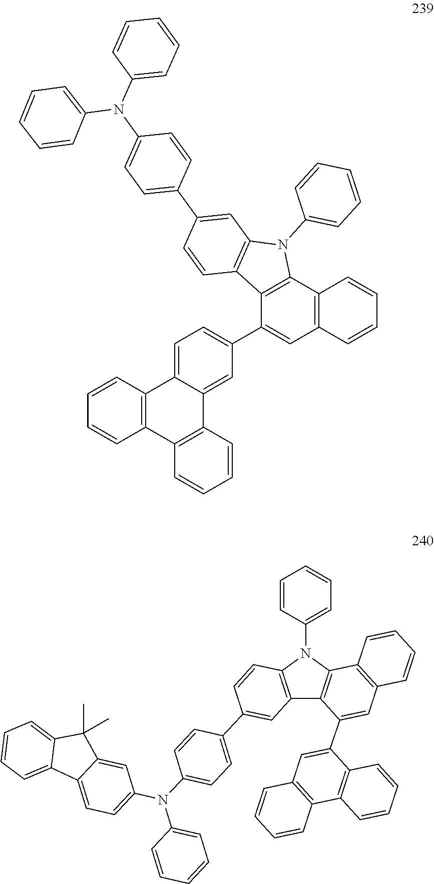 [SMILES: c1ccc(N(c2ccccc2)c2ccc(-c3ccc4c5c(-c6ccc7c8ccccc8c8ccccc8c7c6)cc6ccccc6c5n(-c5ccccc5)c4c3)cc2)cc1 ; CC1(C)c2ccccc2-c2ccc(N(c3ccccc3)c3ccc(-c4ccc5c(c4)c4c(-c6cc7ccccc7c7ccccc67)cc6ccccc6c4n5-c4ccccc4)cc3)cc21]

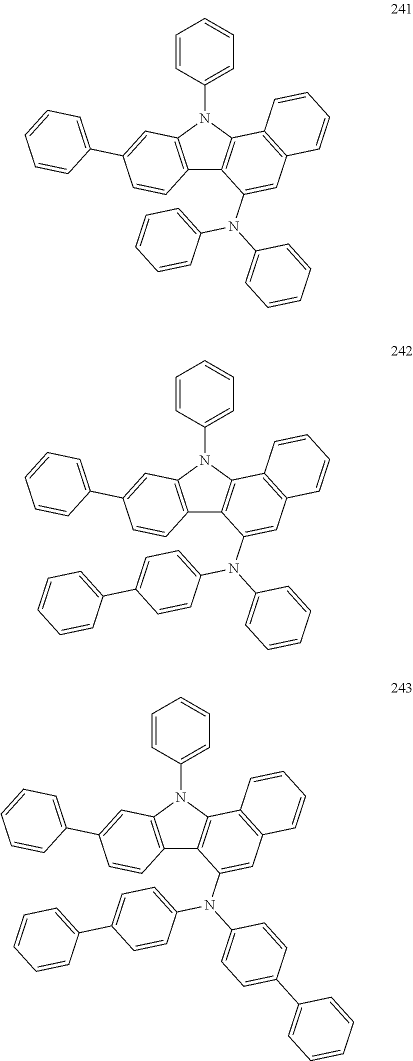 [SMILES: c1ccc(-c2ccc3c4c(N(c5ccccc5)c5ccccc5)cc5ccccc5c4n(-c4ccccc4)c3c2)cc1 ; c1ccc(-c2ccc(N(c3ccccc3)c3cc4ccccc4c4c3c3ccc(-c5ccccc5)cc3n4-c3ccccc3)cc2)cc1 ; c1ccc(-c2ccc(N(c3ccc(-c4ccccc4)cc3)c3cc4ccccc4c4c3c3ccc(-c5ccccc5)cc3n4-c3ccccc3)cc2)cc1]

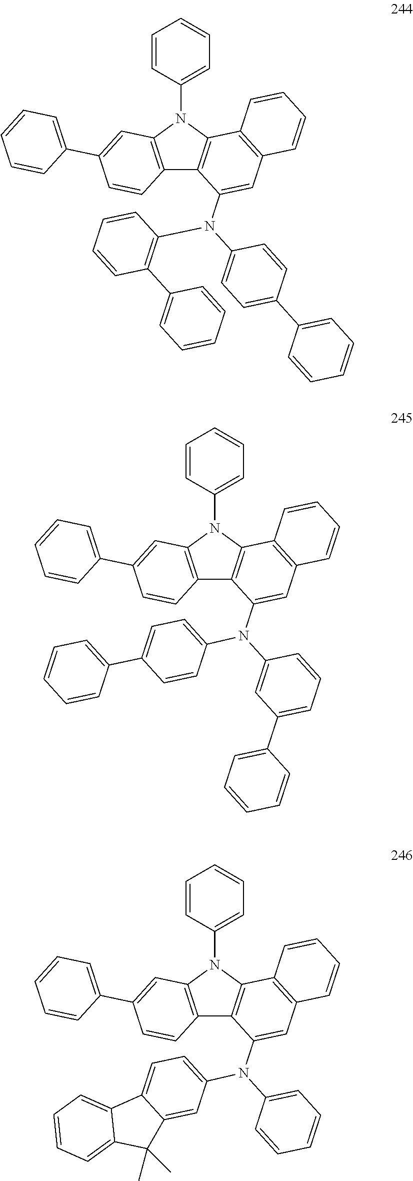 [SMILES: c1ccc(-c2ccc(N(c3ccccc3-c3ccccc3)c3cc4ccccc4c4c3c3ccc(-c5ccccc5)cc3n4-c3ccccc3)cc2)cc1 ; c1ccc(-c2ccc(N(c3cccc(-c4ccccc4)c3)c3cc4ccccc4c4c3c3ccc(-c5ccccc5)cc3n4-c3ccccc3)cc2)cc1 ; CC1(C)c2ccccc2-c2ccc(N(c3ccccc3)c3cc4ccccc4c4c3c3ccc(-c5ccccc5)cc3n4-c3ccccc3)cc21]

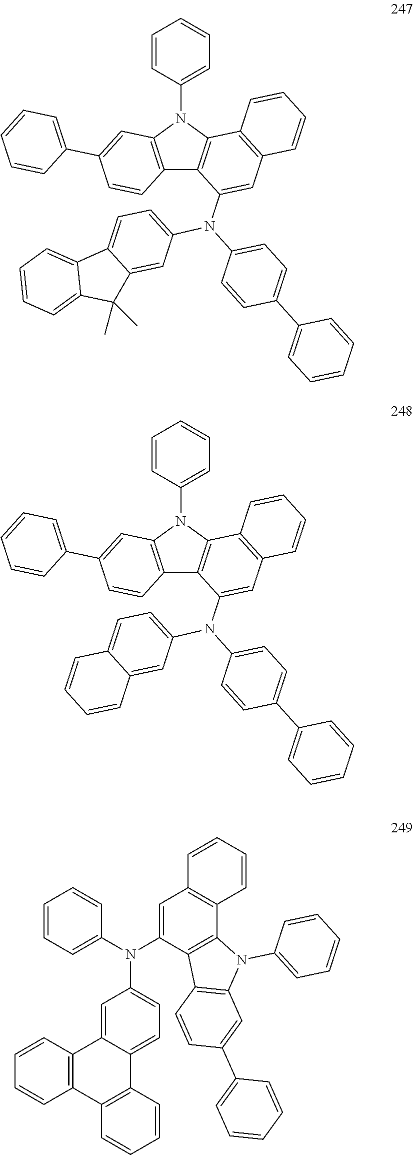 [SMILES: CC1(C)c2ccccc2-c2ccc(N(c3ccc(-c4ccccc4)cc3)c3cc4ccccc4c4c3c3ccc(-c5ccccc5)cc3n4-c3ccccc3)cc21 ; c1ccc(-c2ccc(N(c3ccc4ccccc4c3)c3cc4ccccc4c4c3c3ccc(-c5ccccc5)cc3n4-c3ccccc3)cc2)cc1 ; c1ccc(-c2ccc3c4c(N(c5ccccc5)c5ccc6c7ccccc7c7ccccc7c6c5)cc5ccccc5c4n(-c4ccccc4)c3c2)cc1]

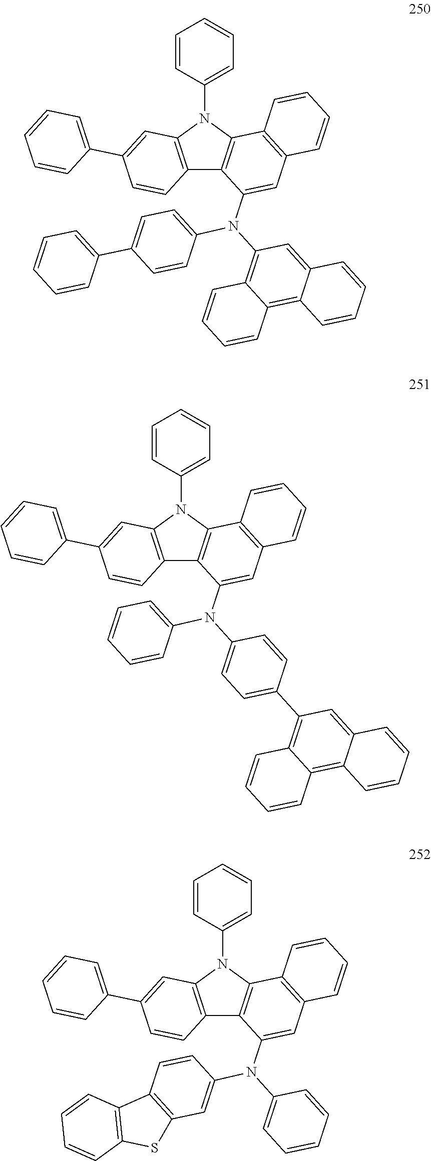 [SMILES: c1ccc(-c2ccc(N(c3cc4ccccc4c4ccccc34)c3cc4ccccc4c4c3c3ccc(-c5ccccc5)cc3n4-c3ccccc3)cc2)cc1 ; c1ccc(-c2ccc3c4c(N(c5ccccc5)c5ccc(-c6cc7ccccc7c7ccccc67)cc5)cc5ccccc5c4n(-c4ccccc4)c3c2)cc1 ; c1ccc(-c2ccc3c4c(N(c5ccccc5)c5ccc6c(c5)sc5ccccc56)cc5ccccc5c4n(-c4ccccc4)c3c2)cc1]

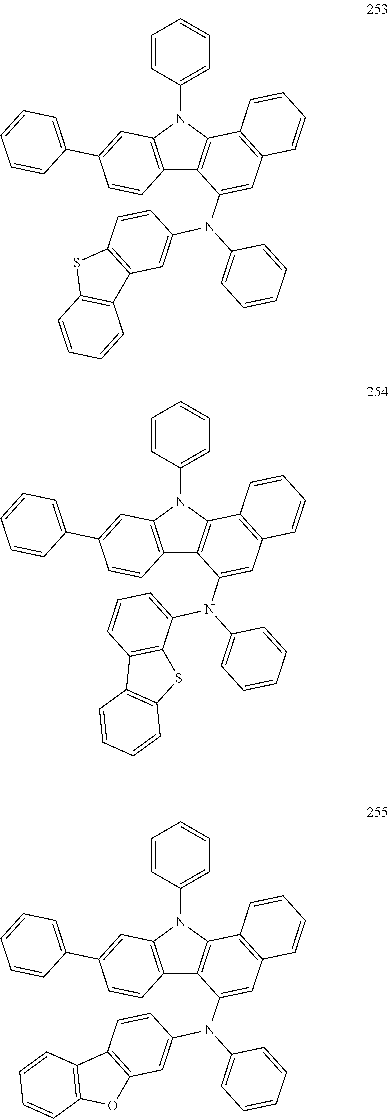 [SMILES: c1ccc(-c2ccc3c4c(N(c5ccccc5)c5ccc6sc7ccccc7c6c5)cc5ccccc5c4n(-c4ccccc4)c3c2)cc1 ; c1ccc(-c2ccc3c4c(N(c5ccccc5)c5cccc6c5sc5ccccc56)cc5ccccc5c4n(-c4ccccc4)c3c2)cc1 ; c1ccc(-c2ccc3c4c(N(c5ccccc5)c5ccc6c(c5)oc5ccccc56)cc5ccccc5c4n(-c4ccccc4)c3c2)cc1]

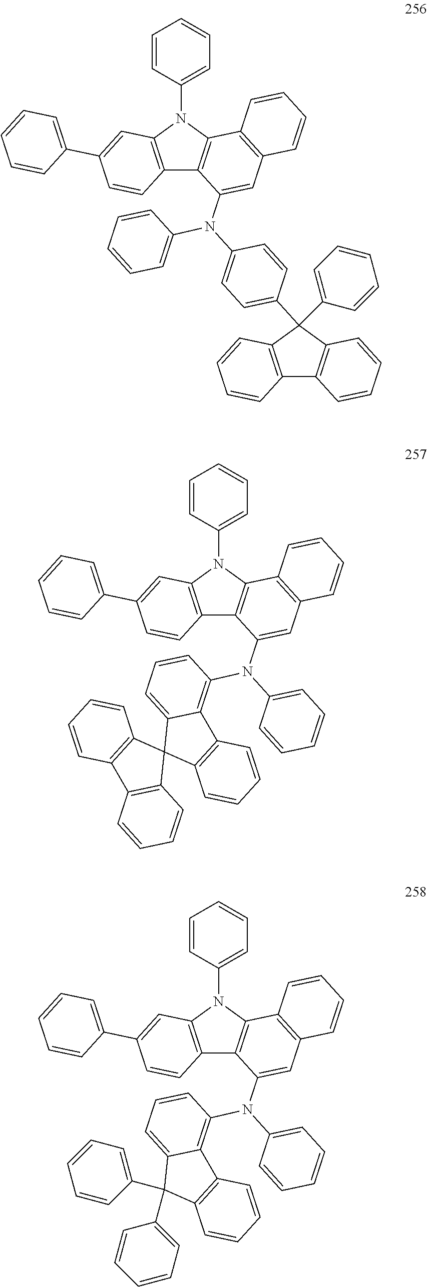 [SMILES: c1ccc(-c2ccc3c4c(N(c5ccccc5)c5ccc(C6(c7ccccc7)c7ccccc7-c7ccccc76)cc5)cc5ccccc5c4n(-c4ccccc4)c3c2)cc1 ; c1ccc(-c2ccc3c4c(N(c5ccccc5)c5cccc6c5-c5ccccc5C65c6ccccc6-c6ccccc65)cc5ccccc5c4n(-c4ccccc4)c3c2)cc1 ; c1ccc(-c2ccc3c4c(N(c5ccccc5)c5cccc6c5-c5ccccc5C6(c5ccccc5)c5ccccc5)cc5ccccc5c4n(-c4ccccc4)c3c2)cc1]

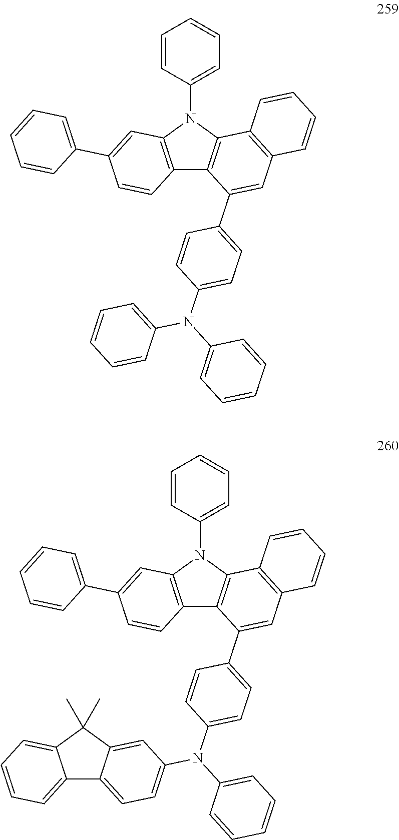 [SMILES: c1ccc(-c2ccc3c4c(-c5ccc(N(c6ccccc6)c6ccccc6)cc5)cc5ccccc5c4n(-c4ccccc4)c3c2)cc1 ; CC1(C)c2ccccc2-c2ccc(N(c3ccccc3)c3ccc(-c4cc5ccccc5c5c4c4ccc(-c6ccccc6)cc4n5-c4ccccc4)cc3)cc21]

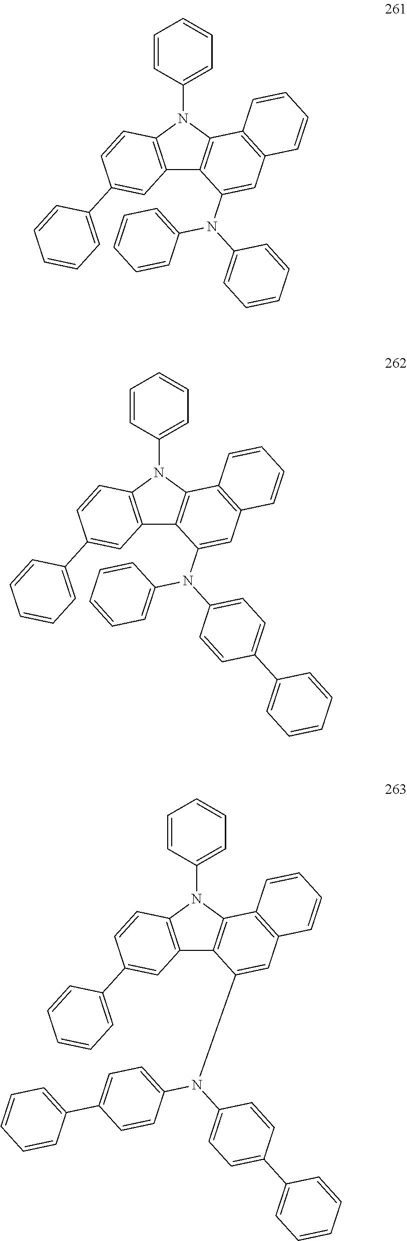 [SMILES: c1ccc(-c2ccc3c(c2)c2c(N(c4ccccc4)c4ccccc4)cc4ccccc4c2n3-c2ccccc2)cc1 ; c1ccc(-c2ccc(N(c3ccccc3)c3cc4ccccc4c4c3c3cc(-c5ccccc5)ccc3n4-c3ccccc3)cc2)cc1 ; c1ccc(-c2ccc(N(c3ccc(-c4ccccc4)cc3)c3cc4ccccc4c4c3c3cc(-c5ccccc5)ccc3n4-c3ccccc3)cc2)cc1]

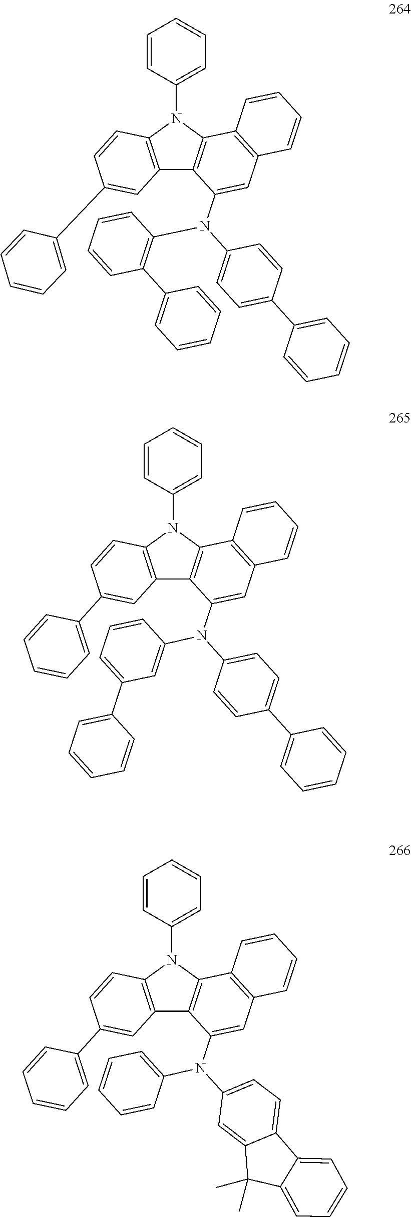 [SMILES: c1ccc(-c2ccc(N(c3ccccc3-c3ccccc3)c3cc4ccccc4c4c3c3cc(-c5ccccc5)ccc3n4-c3ccccc3)cc2)cc1 ; c1ccc(-c2ccc(N(c3cccc(-c4ccccc4)c3)c3cc4ccccc4c4c3c3cc(-c5ccccc5)ccc3n4-c3ccccc3)cc2)cc1 ; CC1(C)c2ccccc2-c2ccc(N(c3ccccc3)c3cc4ccccc4c4c3c3cc(-c5ccccc5)ccc3n4-c3ccccc3)cc21]

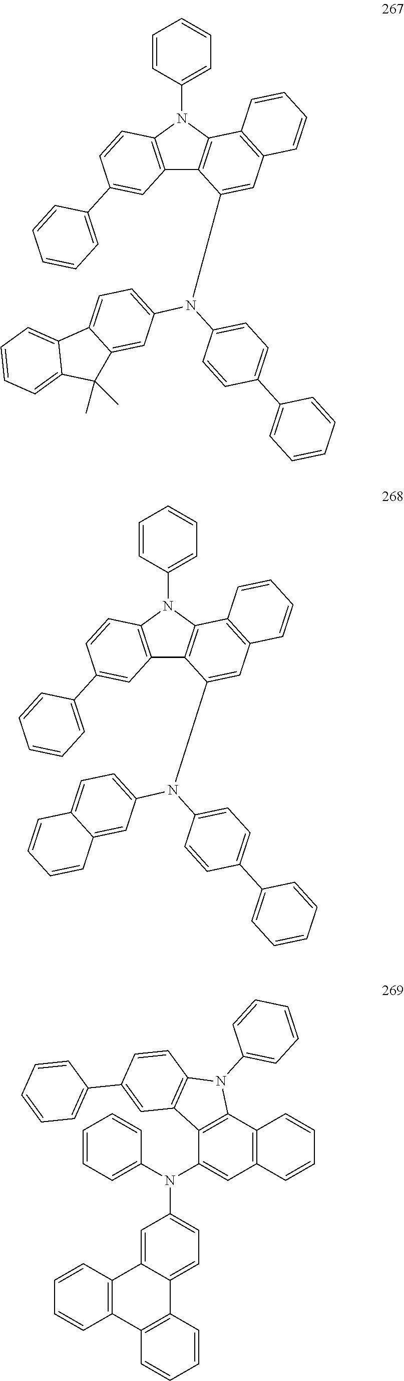 [SMILES: CC1(C)c2ccccc2-c2ccc(N(c3ccc(-c4ccccc4)cc3)c3cc4ccccc4c4c3c3cc(-c5ccccc5)ccc3n4-c3ccccc3)cc21 ; c1ccc(-c2ccc(N(c3ccc4ccccc4c3)c3cc4ccccc4c4c3c3cc(-c5ccccc5)ccc3n4-c3ccccc3)cc2)cc1 ; c1ccc(-c2ccc3c(c2)c2c(N(c4ccccc4)c4ccc5c6ccccc6c6ccccc6c5c4)cc4ccccc4c2n3-c2ccccc2)cc1]

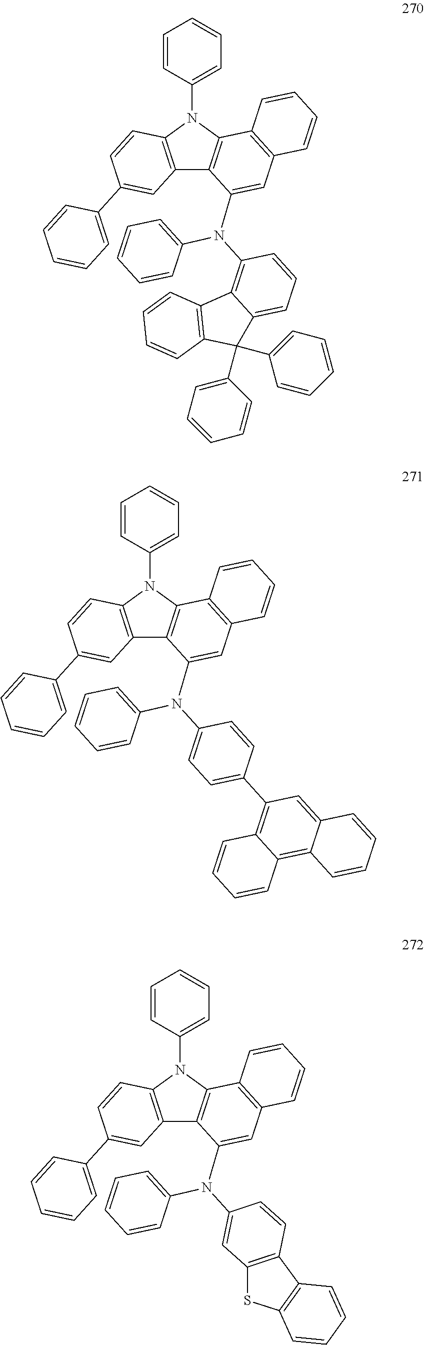 [SMILES: c1ccc(-c2ccc3c(c2)c2c(N(c4ccccc4)c4cccc5c4-c4ccccc4C5(c4ccccc4)c4ccccc4)cc4ccccc4c2n3-c2ccccc2)cc1 ; c1ccc(-c2ccc3c(c2)c2c(N(c4ccccc4)c4ccc(-c5cc6ccccc6c6ccccc56)cc4)cc4ccccc4c2n3-c2ccccc2)cc1 ; c1ccc(-c2ccc3c(c2)c2c(N(c4ccccc4)c4ccc5c(c4)sc4ccccc45)cc4ccccc4c2n3-c2ccccc2)cc1]

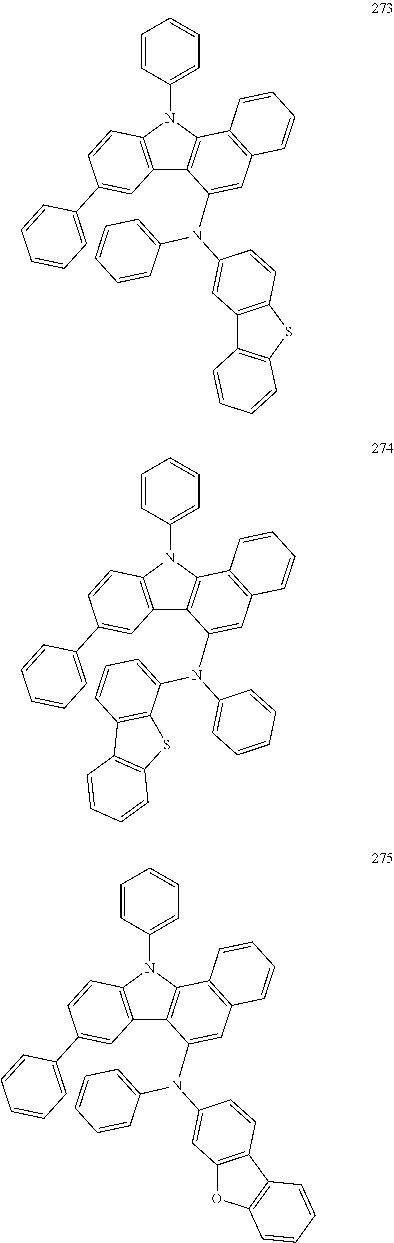 [SMILES: c1ccc(-c2ccc3c(c2)c2c(N(c4ccccc4)c4ccc5sc6ccccc6c5c4)cc4ccccc4c2n3-c2ccccc2)cc1 ; c1ccc(-c2ccc3c(c2)c2c(N(c4ccccc4)c4cccc5c4sc4ccccc45)cc4ccccc4c2n3-c2ccccc2)cc1 ; c1ccc(-c2ccc3c(c2)c2c(N(c4ccccc4)c4ccc5c(c4)oc4ccccc45)cc4ccccc4c2n3-c2ccccc2)cc1]

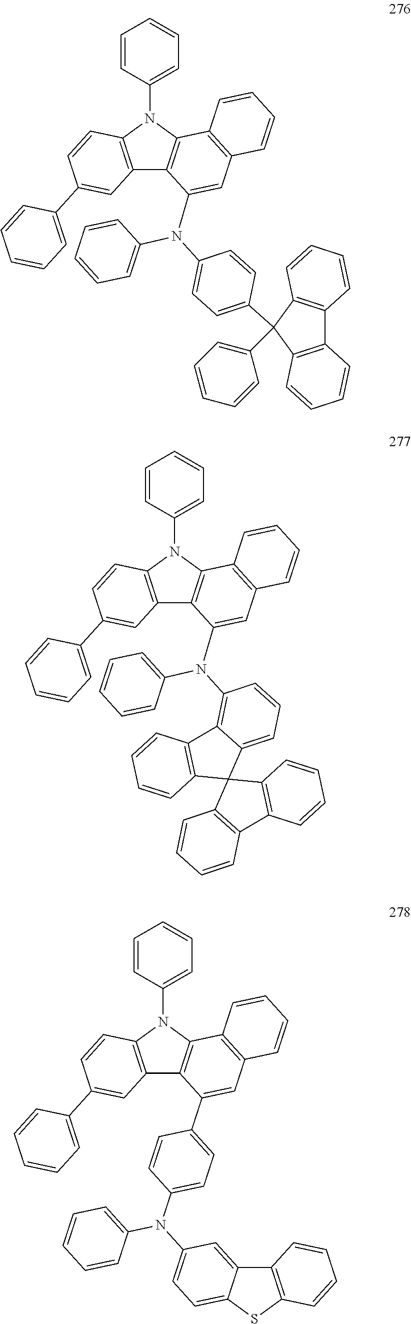 [SMILES: c1ccc(-c2ccc3c(c2)c2c(N(c4ccccc4)c4ccc(C5(c6ccccc6)c6ccccc6-c6ccccc65)cc4)cc4ccccc4c2n3-c2ccccc2)cc1 ; c1ccc(-c2ccc3c(c2)c2c(N(c4ccccc4)c4cccc5c4-c4ccccc4C54c5ccccc5-c5ccccc54)cc4ccccc4c2n3-c2ccccc2)cc1 ; c1ccc(-c2ccc3c(c2)c2c(-c4ccc(N(c5ccccc5)c5ccc6sc7ccccc7c6c5)cc4)cc4ccccc4c2n3-c2ccccc2)cc1]

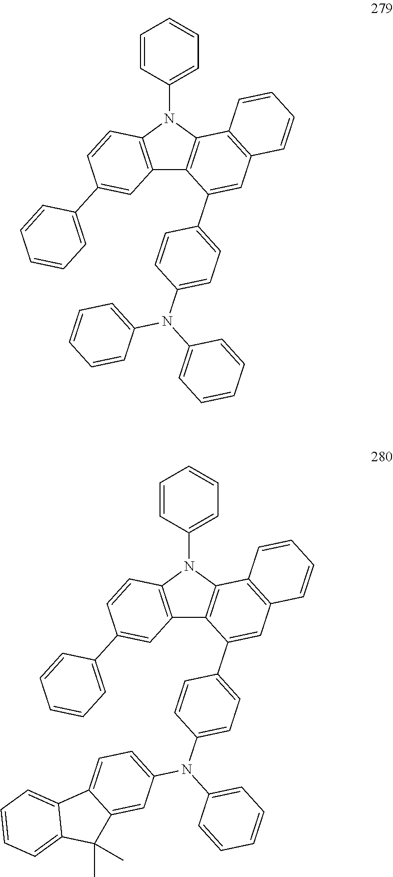 [SMILES: c1ccc(-c2ccc3c(c2)c2c(-c4ccc(N(c5ccccc5)c5ccccc5)cc4)cc4ccccc4c2n3-c2ccccc2)cc1 ; CC1(C)c2ccccc2-c2ccc(N(c3ccccc3)c3ccc(-c4cc5ccccc5c5c4c4cc(-c6ccccc6)ccc4n5-c4ccccc4)cc3)cc21]

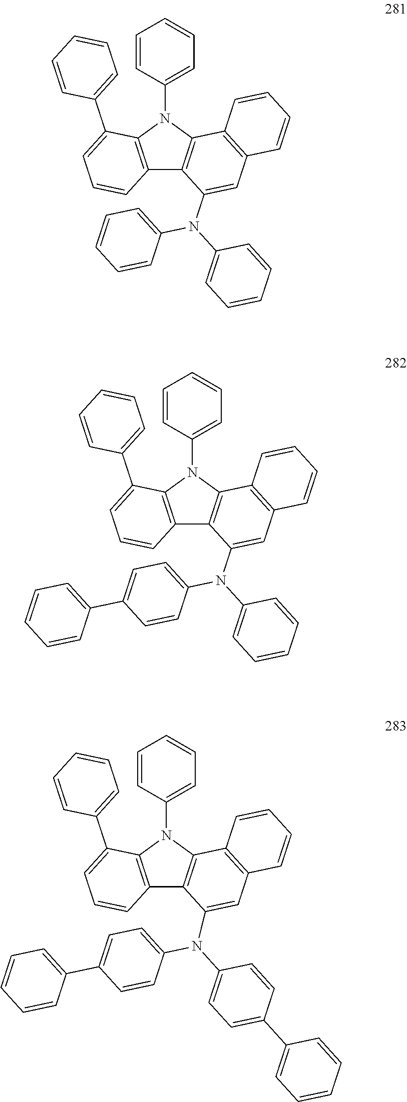 [SMILES: c1ccc(-c2cccc3c4c(N(c5ccccc5)c5ccccc5)cc5ccccc5c4n(-c4ccccc4)c23)cc1 ; c1ccc(-c2ccc(N(c3ccccc3)c3cc4ccccc4c4c3c3cccc(-c5ccccc5)c3n4-c3ccccc3)cc2)cc1 ; c1ccc(-c2ccc(N(c3ccc(-c4ccccc4)cc3)c3cc4ccccc4c4c3c3cccc(-c5ccccc5)c3n4-c3ccccc3)cc2)cc1]

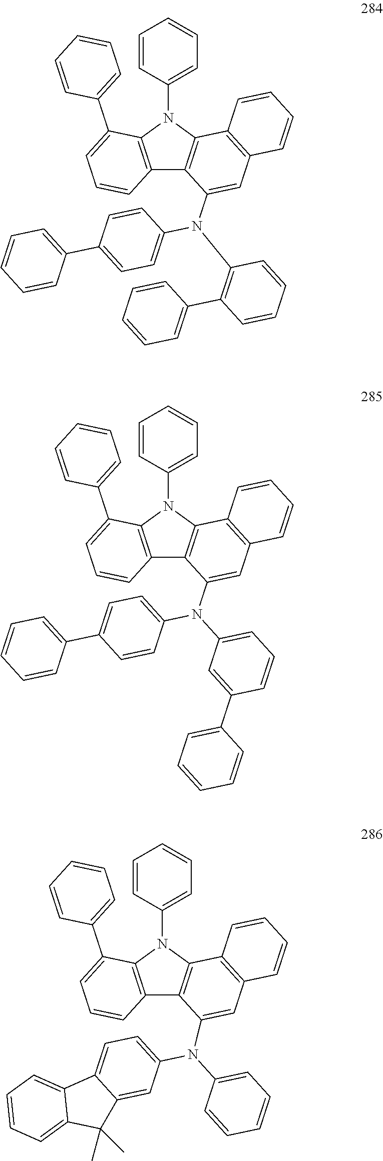 [SMILES: c1ccc(-c2ccc(N(c3ccccc3-c3ccccc3)c3cc4ccccc4c4c3c3cccc(-c5ccccc5)c3n4-c3ccccc3)cc2)cc1 ; c1ccc(-c2ccc(N(c3cccc(-c4ccccc4)c3)c3cc4ccccc4c4c3c3cccc(-c5ccccc5)c3n4-c3ccccc3)cc2)cc1 ; CC1(C)c2ccccc2-c2ccc(N(c3ccccc3)c3cc4ccccc4c4c3c3cccc(-c5ccccc5)c3n4-c3ccccc3)cc21]

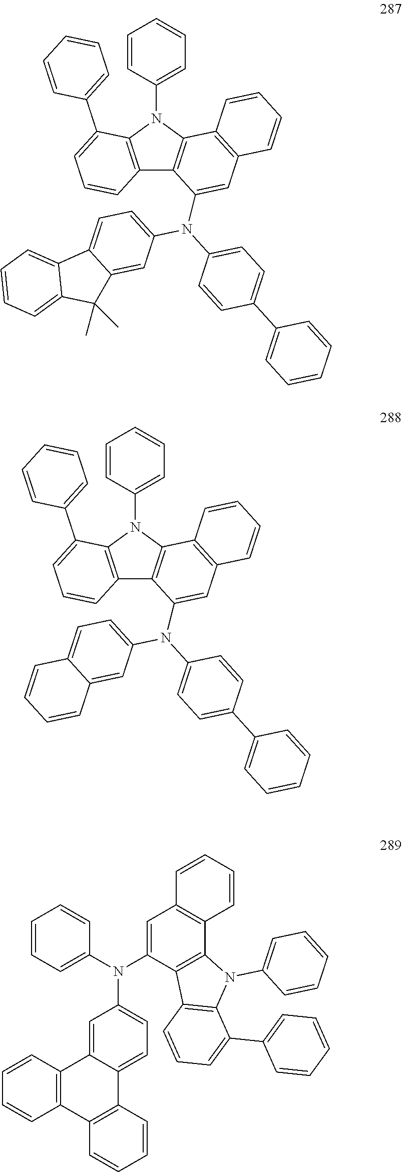 [SMILES: CC1(C)c2ccccc2-c2ccc(N(c3ccc(-c4ccccc4)cc3)c3cc4ccccc4c4c3c3cccc(-c5ccccc5)c3n4-c3ccccc3)cc21 ; c1ccc(-c2ccc(N(c3ccc4ccccc4c3)c3cc4ccccc4c4c3c3cccc(-c5ccccc5)c3n4-c3ccccc3)cc2)cc1 ; c1ccc(-c2cccc3c4c(N(c5ccccc5)c5ccc6c7ccccc7c7ccccc7c6c5)cc5ccccc5c4n(-c4ccccc4)c23)cc1]

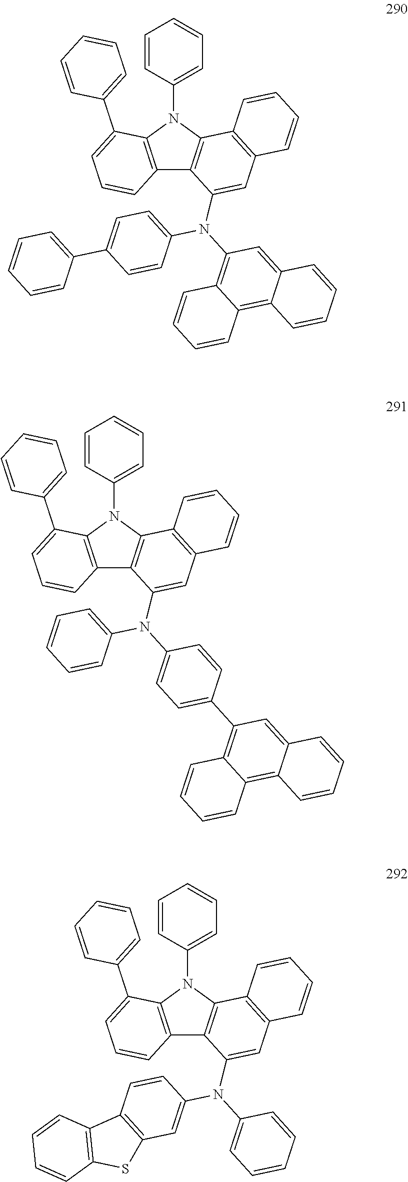 [SMILES: c1ccc(-c2ccc(N(c3cc4ccccc4c4ccccc34)c3cc4ccccc4c4c3c3cccc(-c5ccccc5)c3n4-c3ccccc3)cc2)cc1 ; c1ccc(-c2cccc3c4c(N(c5ccccc5)c5ccc(-c6cc7ccccc7c7ccccc67)cc5)cc5ccccc5c4n(-c4ccccc4)c23)cc1 ; c1ccc(-c2cccc3c4c(N(c5ccccc5)c5ccc6c(c5)sc5ccccc56)cc5ccccc5c4n(-c4ccccc4)c23)cc1]

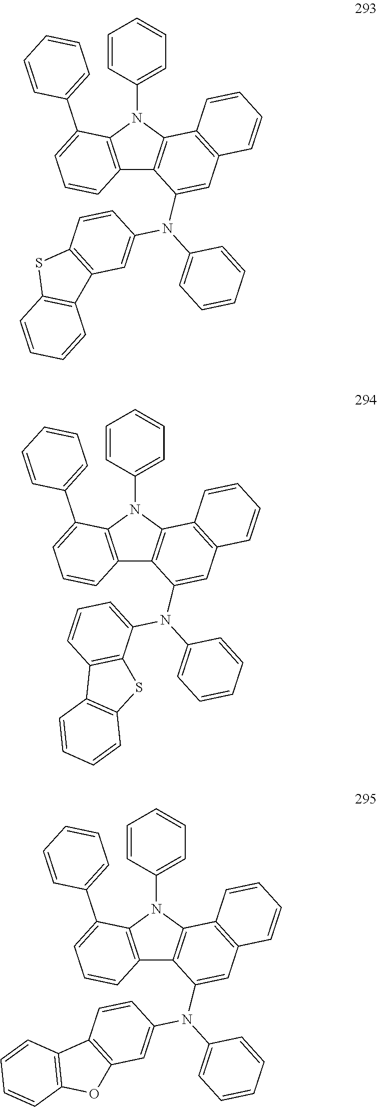 [SMILES: c1ccc(-c2cccc3c4c(N(c5ccccc5)c5ccc6sc7ccccc7c6c5)cc5ccccc5c4n(-c4ccccc4)c23)cc1 ; c1ccc(-c2cccc3c4c(N(c5ccccc5)c5cccc6c5sc5ccccc56)cc5ccccc5c4n(-c4ccccc4)c23)cc1 ; c1ccc(-c2cccc3c4c(N(c5ccccc5)c5ccc6c(c5)oc5ccccc56)cc5ccccc5c4n(-c4ccccc4)c23)cc1]

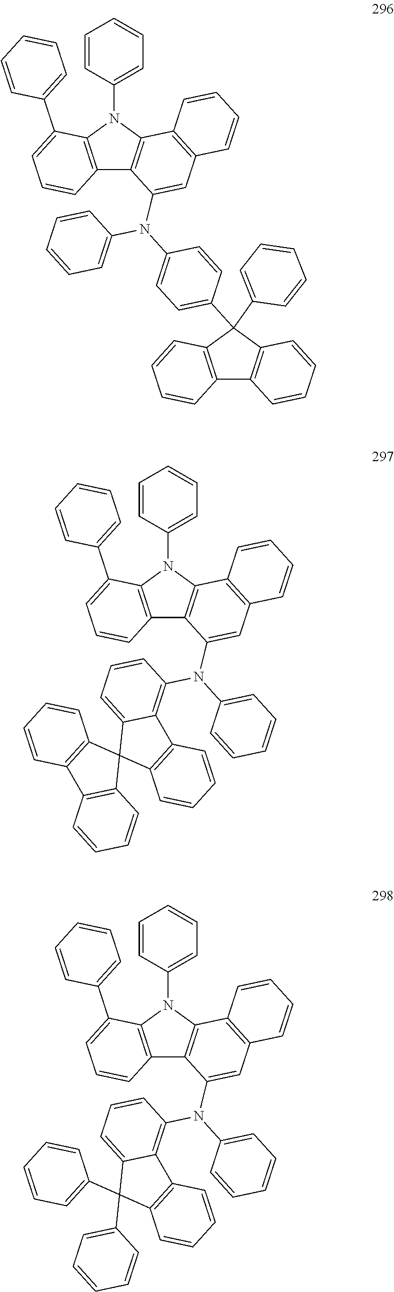 [SMILES: c1ccc(-c2cccc3c4c(N(c5ccccc5)c5ccc(C6(c7ccccc7)c7ccccc7-c7ccccc76)cc5)cc5ccccc5c4n(-c4ccccc4)c23)cc1 ; c1ccc(-c2cccc3c4c(N(c5ccccc5)c5cccc6c5-c5ccccc5C65c6ccccc6-c6ccccc65)cc5ccccc5c4n(-c4ccccc4)c23)cc1 ; c1ccc(-c2cccc3c4c(N(c5ccccc5)c5cccc6c5-c5ccccc5C6(c5ccccc5)c5ccccc5)cc5ccccc5c4n(-c4ccccc4)c23)cc1]

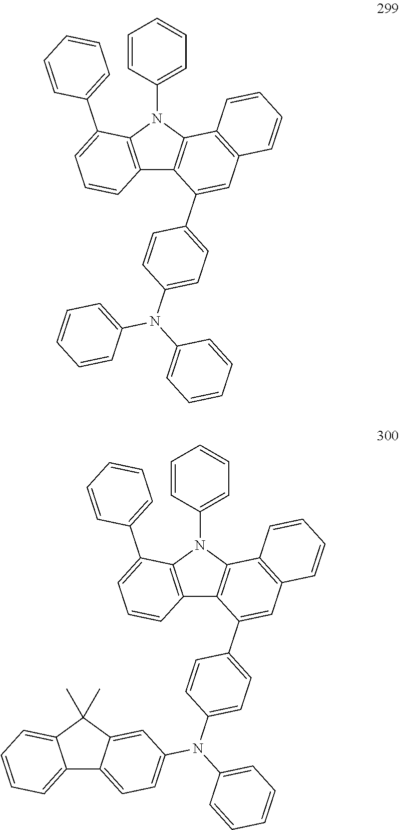

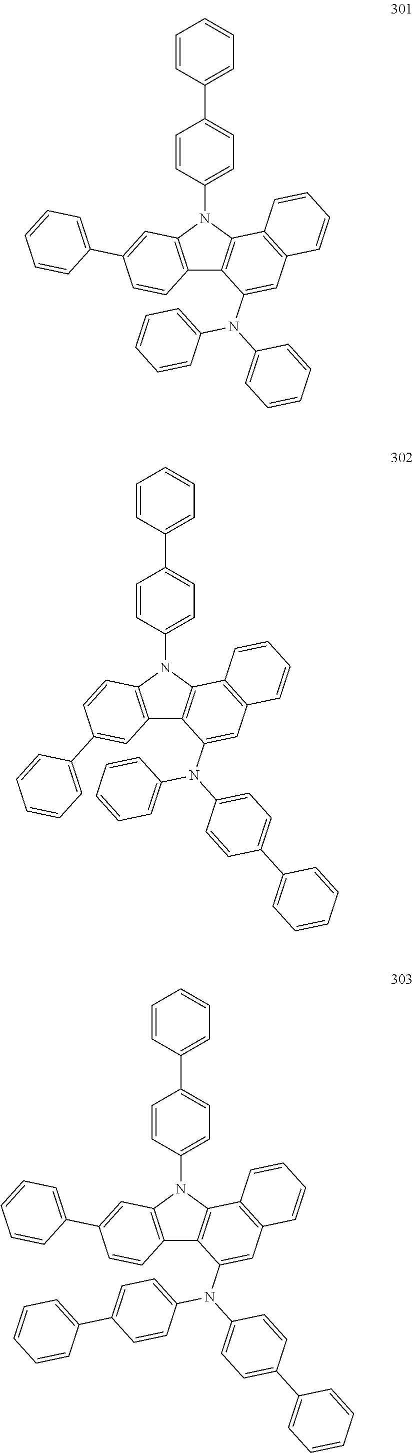 [SMILES: c1ccc(-c2ccc(-n3c4cc(-c5ccccc5)ccc4c4c(N(c5ccccc5)c5ccccc5)cc5ccccc5c43)cc2)cc1 ; c1ccc(-c2ccc(N(c3ccccc3)c3cc4ccccc4c4c3c3cc(-c5ccccc5)ccc3n4-c3ccc(-c4ccccc4)cc3)cc2)cc1 ; c1ccc(-c2ccc(N(c3ccc(-c4ccccc4)cc3)c3cc4ccccc4c4c3c3ccc(-c5ccccc5)cc3n4-c3ccc(-c4ccccc4)cc3)cc2)cc1]

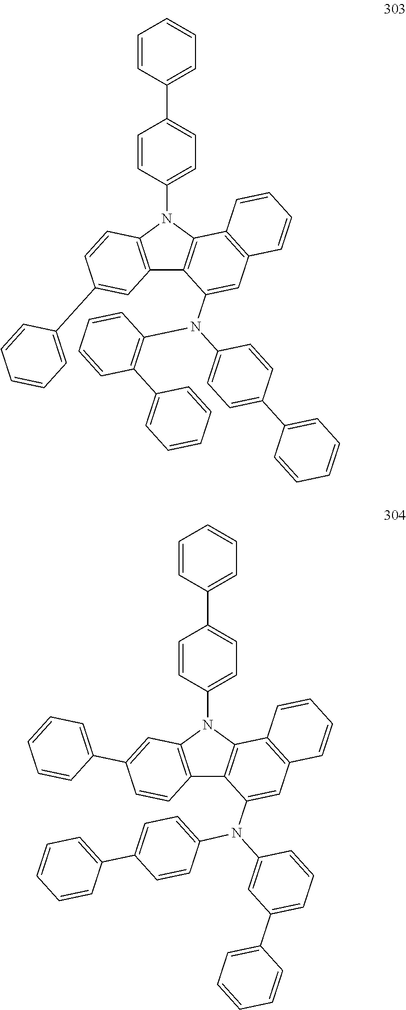 [SMILES: c1ccc(-c2ccc(N(c3ccccc3-c3ccccc3)c3cc4ccccc4c4c3c3cc(-c5ccccc5)ccc3n4-c3ccc(-c4ccccc4)cc3)cc2)cc1 ; c1ccc(-c2ccc(N(c3cccc(-c4ccccc4)c3)c3cc4ccccc4c4c3c3ccc(-c5ccccc5)cc3n4-c3ccc(-c4ccccc4)cc3)cc2)cc1]

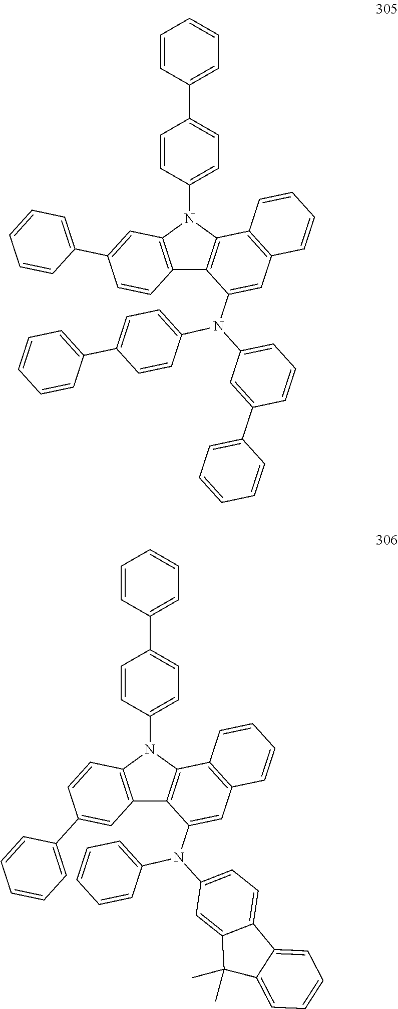 [SMILES: c1ccc(-c2ccc(N(c3cccc(-c4ccccc4)c3)c3cc4ccccc4c4c3c3ccc(-c5ccccc5)cc3n4-c3ccc(-c4ccccc4)cc3)cc2)cc1 ; CC1(C)c2ccccc2-c2ccc(N(c3ccccc3)c3cc4ccccc4c4c3c3cc(-c5ccccc5)ccc3n4-c3ccc(-c4ccccc4)cc3)cc21]

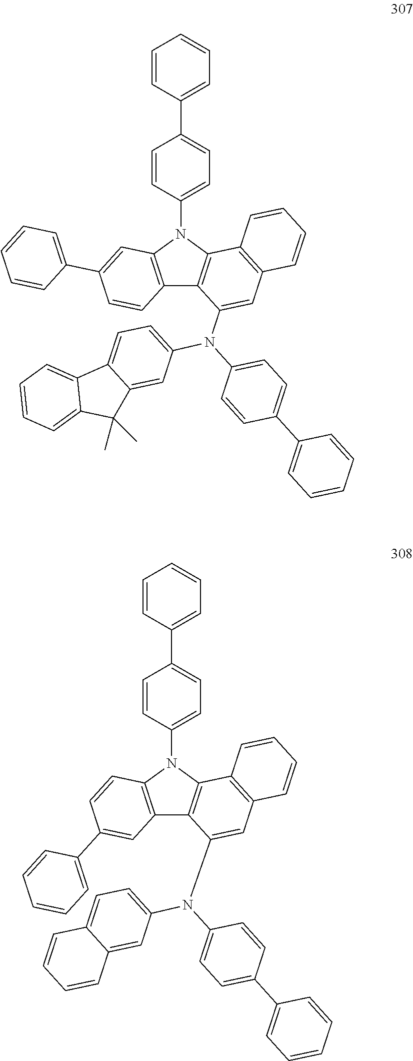 [SMILES: CC1(C)c2ccccc2-c2ccc(N(c3ccc(-c4ccccc4)cc3)c3cc4ccccc4c4c3c3ccc(-c5ccccc5)cc3n4-c3ccc(-c4ccccc4)cc3)cc21 ; c1ccc(-c2ccc(N(c3ccc4ccccc4c3)c3cc4ccccc4c4c3c3cc(-c5ccccc5)ccc3n4-c3ccc(-c4ccccc4)cc3)cc2)cc1]

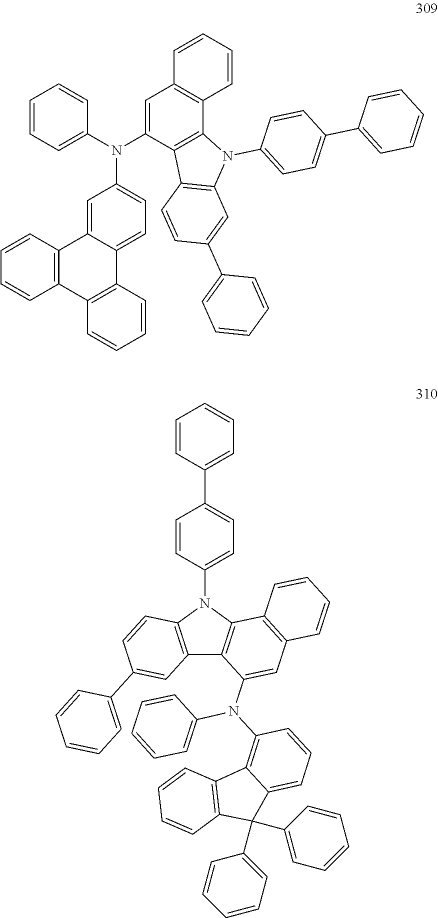 [SMILES: c1ccc(-c2ccc(-n3c4cc(-c5ccccc5)ccc4c4c(N(c5ccccc5)c5ccc6c7ccccc7c7ccccc7c6c5)cc5ccccc5c43)cc2)cc1 ; c1ccc(-c2ccc(-n3c4ccc(-c5ccccc5)cc4c4c(N(c5ccccc5)c5cccc6c5-c5ccccc5C6(c5ccccc5)c5ccccc5)cc5ccccc5c43)cc2)cc1]

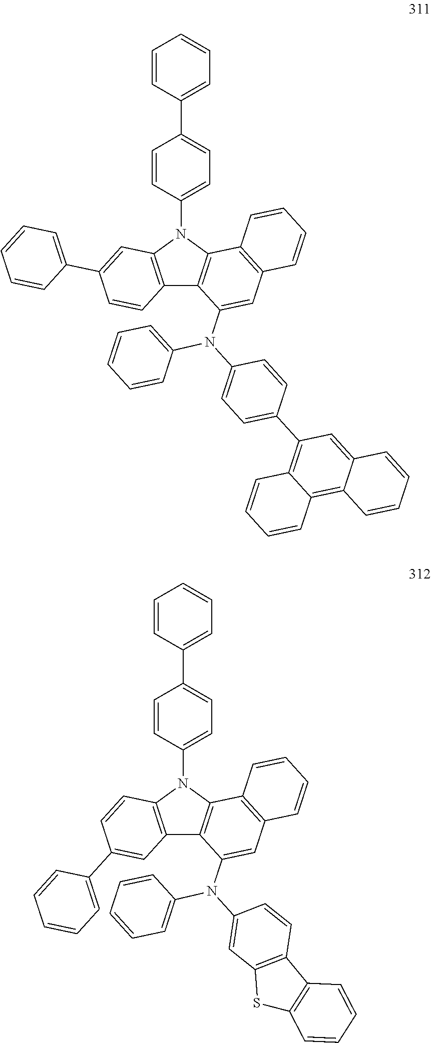 [SMILES: c1ccc(-c2ccc(-n3c4cc(-c5ccccc5)ccc4c4c(N(c5ccccc5)c5ccc(-c6cc7ccccc7c7ccccc67)cc5)cc5ccccc5c43)cc2)cc1 ; c1ccc(-c2ccc(-n3c4ccc(-c5ccccc5)cc4c4c(N(c5ccccc5)c5ccc6c(c5)sc5ccccc56)cc5ccccc5c43)cc2)cc1]

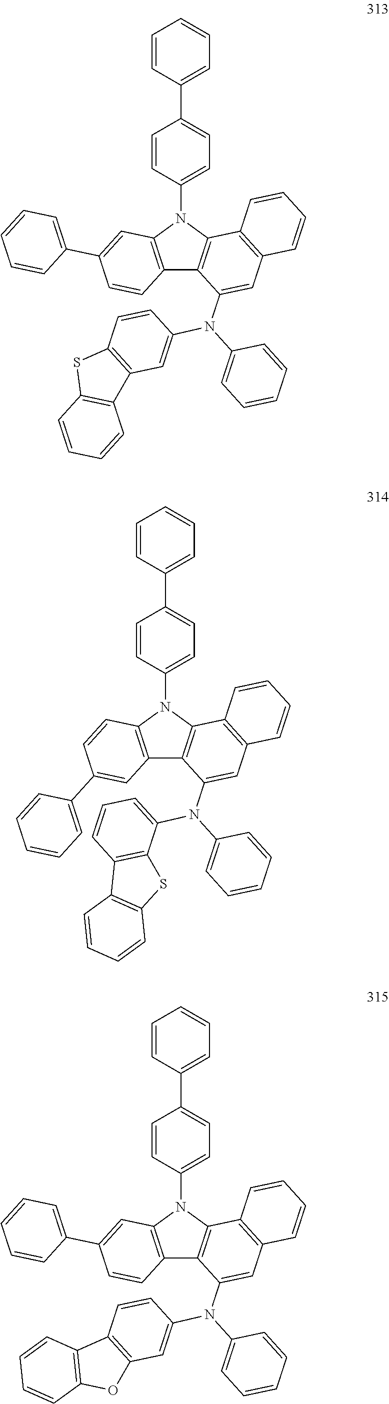 [SMILES: c1ccc(-c2ccc(-n3c4cc(-c5ccccc5)ccc4c4c(N(c5ccccc5)c5ccc6sc7ccccc7c6c5)cc5ccccc5c43)cc2)cc1 ; c1ccc(-c2ccc(-n3c4ccc(-c5ccccc5)cc4c4c(N(c5ccccc5)c5cccc6c5sc5ccccc56)cc5ccccc5c43)cc2)cc1 ; c1ccc(-c2ccc(-n3c4cc(-c5ccccc5)ccc4c4c(N(c5ccccc5)c5ccc6c(c5)oc5ccccc56)cc5ccccc5c43)cc2)cc1]

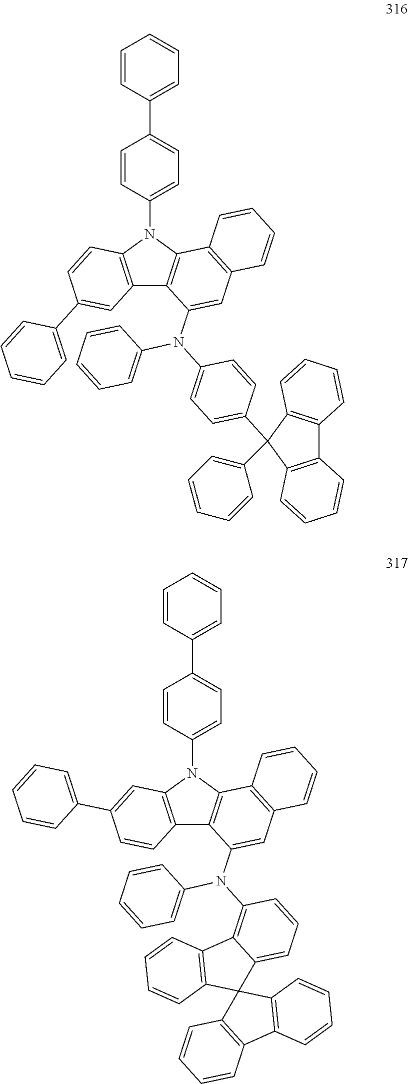 [SMILES: c1ccc(-c2ccc(-n3c4ccc(-c5ccccc5)cc4c4c(N(c5ccccc5)c5ccc(C6(c7ccccc7)c7ccccc7-c7ccccc76)cc5)cc5ccccc5c43)cc2)cc1 ; c1ccc(-c2ccc(-n3c4cc(-c5ccccc5)ccc4c4c(N(c5ccccc5)c5cccc6c5-c5ccccc5C65c6ccccc6-c6ccccc65)cc5ccccc5c43)cc2)cc1]

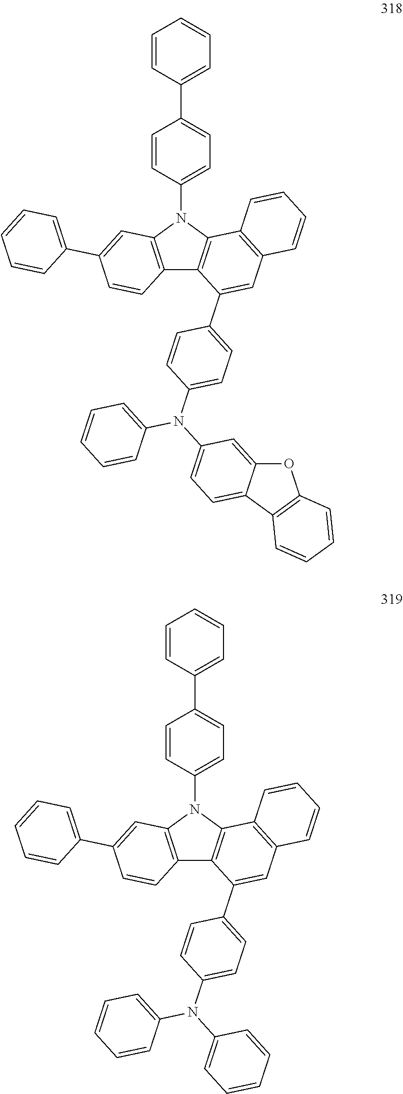 [SMILES: c1ccc(-c2ccc(-n3c4cc(-c5ccccc5)ccc4c4c(-c5ccc(N(c6ccccc6)c6ccc7c(c6)oc6ccccc67)cc5)cc5ccccc5c43)cc2)cc1 ; c1ccc(-c2ccc(-n3c4cc(-c5ccccc5)ccc4c4c(-c5ccc(N(c6ccccc6)c6ccccc6)cc5)cc5ccccc5c43)cc2)cc1]

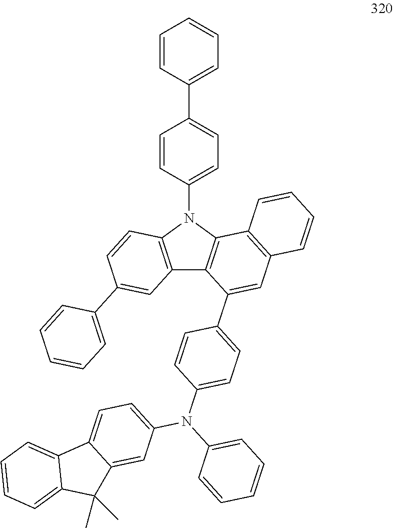 [SMILES: CC1(C)c2ccccc2-c2ccc(N(c3ccccc3)c3ccc(-c4cc5ccccc5c5c4c4cc(-c6ccccc6)ccc4n5-c4ccc(-c5ccccc5)cc4)cc3)cc21]

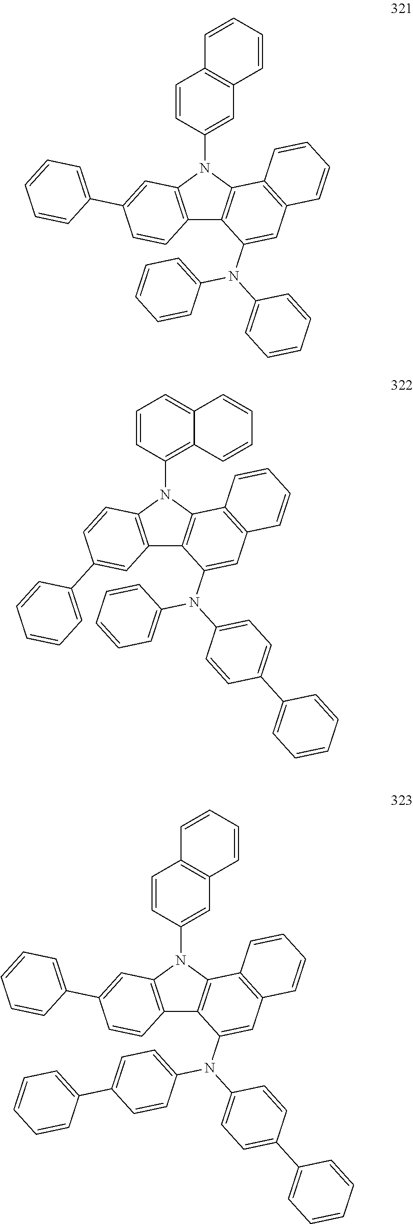 [SMILES: c1ccc(-c2ccc3c4c(N(c5ccccc5)c5ccccc5)cc5ccccc5c4n(-c4ccc5ccccc5c4)c3c2)cc1 ; c1ccc(-c2ccc(N(c3ccccc3)c3cc4ccccc4c4c3c3cc(-c5ccccc5)ccc3n4-c3cccc4ccccc34)cc2)cc1 ; c1ccc(-c2ccc(N(c3ccc(-c4ccccc4)cc3)c3cc4ccccc4c4c3c3ccc(-c5ccccc5)cc3n4-c3ccc4ccccc4c3)cc2)cc1]

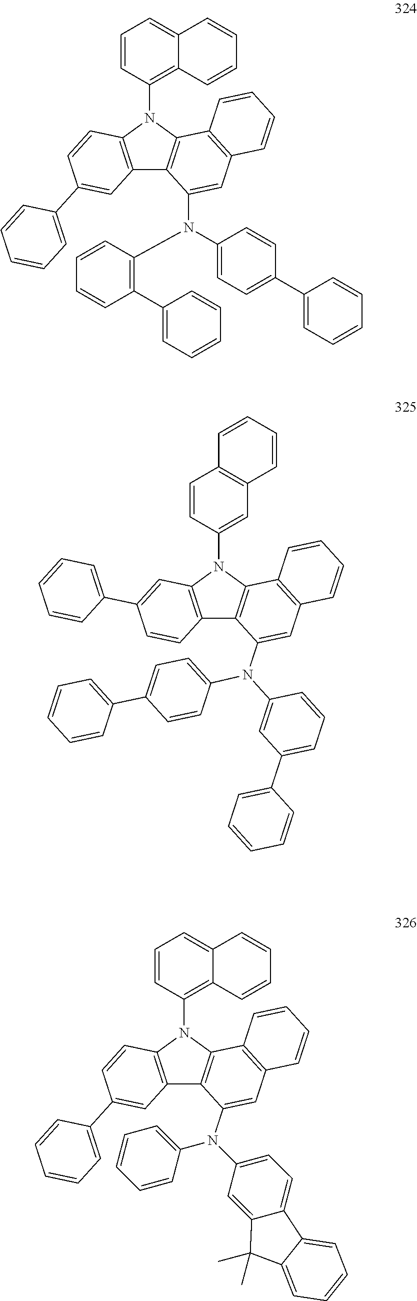 [SMILES: c1ccc(-c2ccc(N(c3ccccc3-c3ccccc3)c3cc4ccccc4c4c3c3cc(-c5ccccc5)ccc3n4-c3cccc4ccccc34)cc2)cc1 ; c1ccc(-c2ccc(N(c3cccc(-c4ccccc4)c3)c3cc4ccccc4c4c3c3ccc(-c5ccccc5)cc3n4-c3ccc4ccccc4c3)cc2)cc1 ; CC1(C)c2ccccc2-c2ccc(N(c3ccccc3)c3cc4ccccc4c4c3c3cc(-c5ccccc5)ccc3n4-c3cccc4ccccc34)cc21]

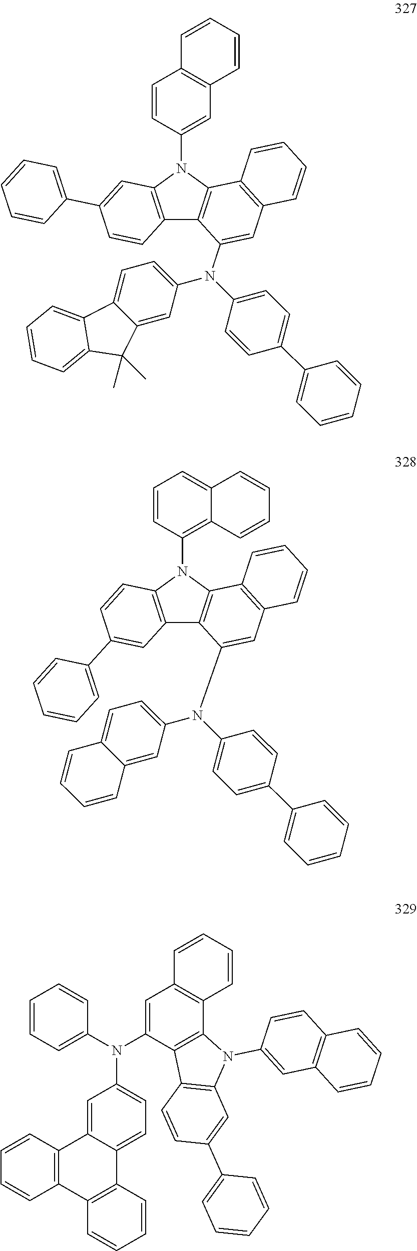 [SMILES: CC1(C)c2ccccc2-c2ccc(N(c3ccc(-c4ccccc4)cc3)c3cc4ccccc4c4c3c3ccc(-c5ccccc5)cc3n4-c3ccc4ccccc4c3)cc21 ; c1ccc(-c2ccc(N(c3ccc4ccccc4c3)c3cc4ccccc4c4c3c3cc(-c5ccccc5)ccc3n4-c3cccc4ccccc34)cc2)cc1 ; c1ccc(-c2ccc3c4c(N(c5ccccc5)c5ccc6c7ccccc7c7ccccc7c6c5)cc5ccccc5c4n(-c4ccc5ccccc5c4)c3c2)cc1]

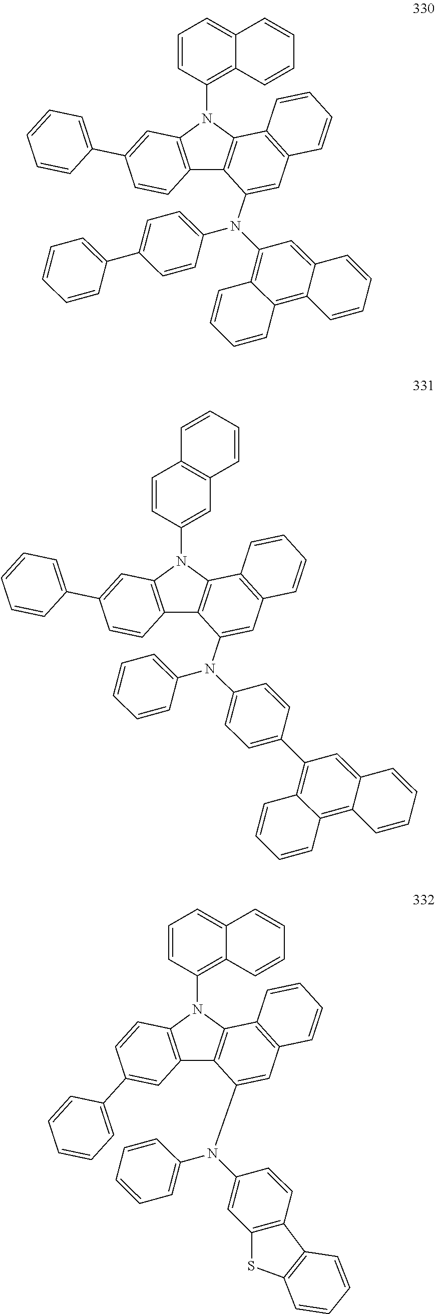 [SMILES: c1ccc(-c2ccc(N(c3cc4ccccc4c4ccccc34)c3cc4ccccc4c4c3c3ccc(-c5ccccc5)cc3n4-c3cccc4ccccc34)cc2)cc1 ; c1ccc(-c2ccc3c4c(N(c5ccccc5)c5ccc(-c6cc7ccccc7c7ccccc67)cc5)cc5ccccc5c4n(-c4ccc5ccccc5c4)c3c2)cc1 ; c1ccc(-c2ccc3c(c2)c2c(N(c4ccccc4)c4ccc5c(c4)sc4ccccc45)cc4ccccc4c2n3-c2cccc3ccccc23)cc1]

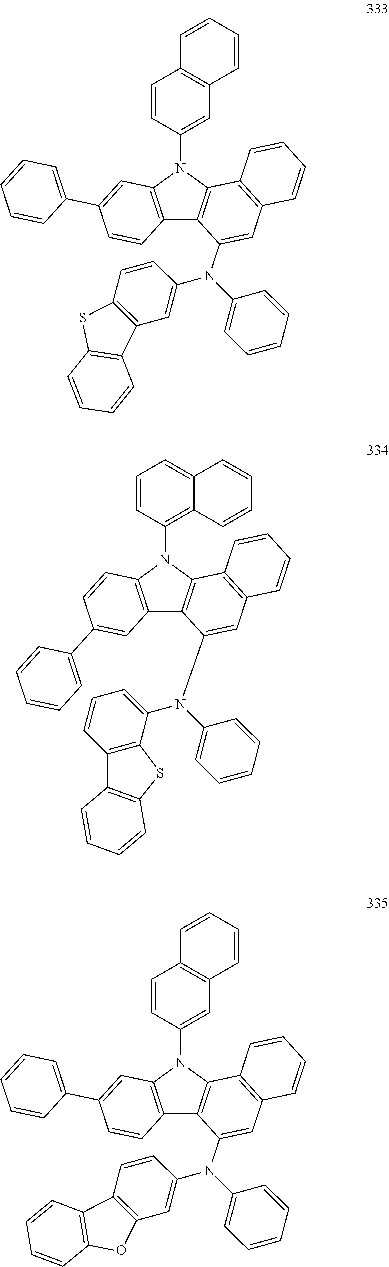 [SMILES: c1ccc(-c2ccc3c4c(N(c5ccccc5)c5ccc6sc7ccccc7c6c5)cc5ccccc5c4n(-c4ccc5ccccc5c4)c3c2)cc1 ; c1ccc(-c2ccc3c(c2)c2c(N(c4ccccc4)c4cccc5c4sc4ccccc45)cc4ccccc4c2n3-c2cccc3ccccc23)cc1 ; c1ccc(-c2ccc3c4c(N(c5ccccc5)c5ccc6c(c5)oc5ccccc56)cc5ccccc5c4n(-c4ccc5ccccc5c4)c3c2)cc1]

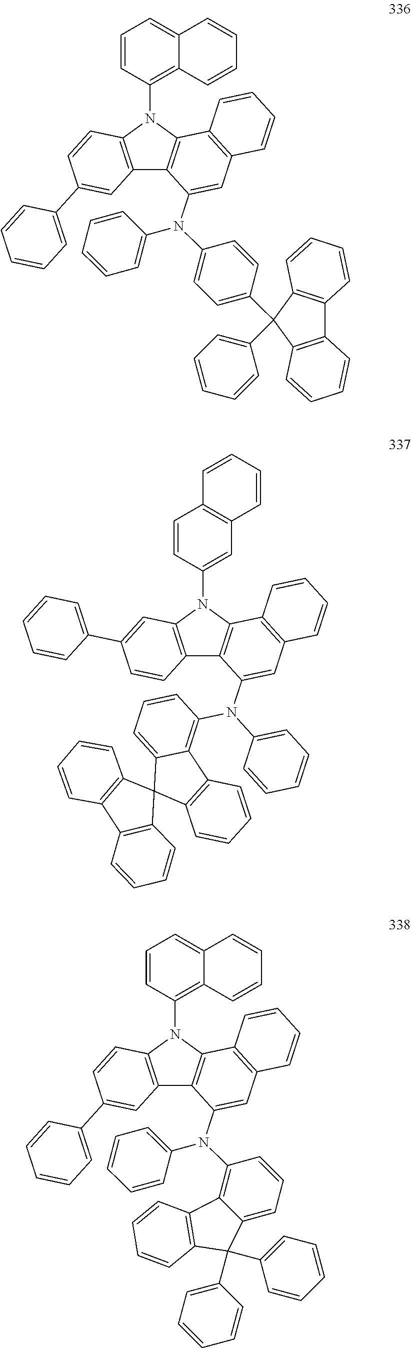 [SMILES: c1ccc(-c2ccc3c(c2)c2c(N(c4ccccc4)c4ccc(C5(c6ccccc6)c6ccccc6-c6ccccc65)cc4)cc4ccccc4c2n3-c2cccc3ccccc23)cc1 ; c1ccc(-c2ccc3c4c(N(c5ccccc5)c5cccc6c5-c5ccccc5C65c6ccccc6-c6ccccc65)cc5ccccc5c4n(-c4ccc5ccccc5c4)c3c2)cc1 ; c1ccc(-c2ccc3c(c2)c2c(N(c4ccccc4)c4cccc5c4-c4ccccc4C5(c4ccccc4)c4ccccc4)cc4ccccc4c2n3-c2cccc3ccccc23)cc1]

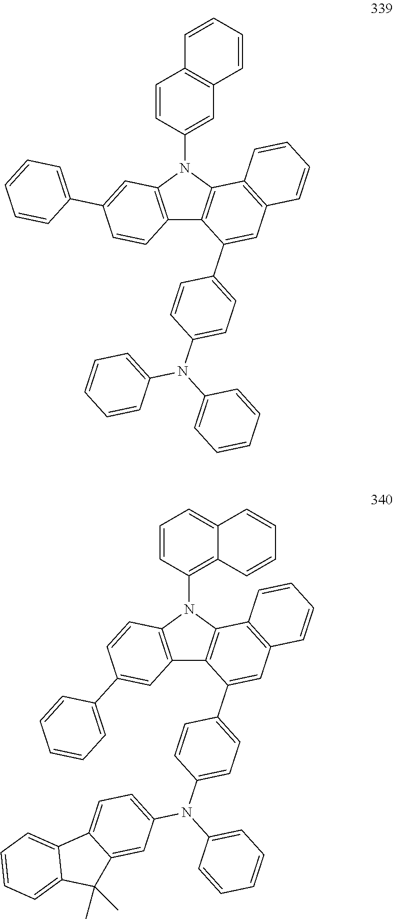 [SMILES: c1ccc(-c2ccc3c4c(-c5ccc(N(c6ccccc6)c6ccccc6)cc5)cc5ccccc5c4n(-c4ccc5ccccc5c4)c3c2)cc1 ; CC1(C)c2ccccc2-c2ccc(N(c3ccccc3)c3ccc(-c4cc5ccccc5c5c4c4cc(-c6ccccc6)ccc4n5-c4cccc5ccccc45)cc3)cc21]

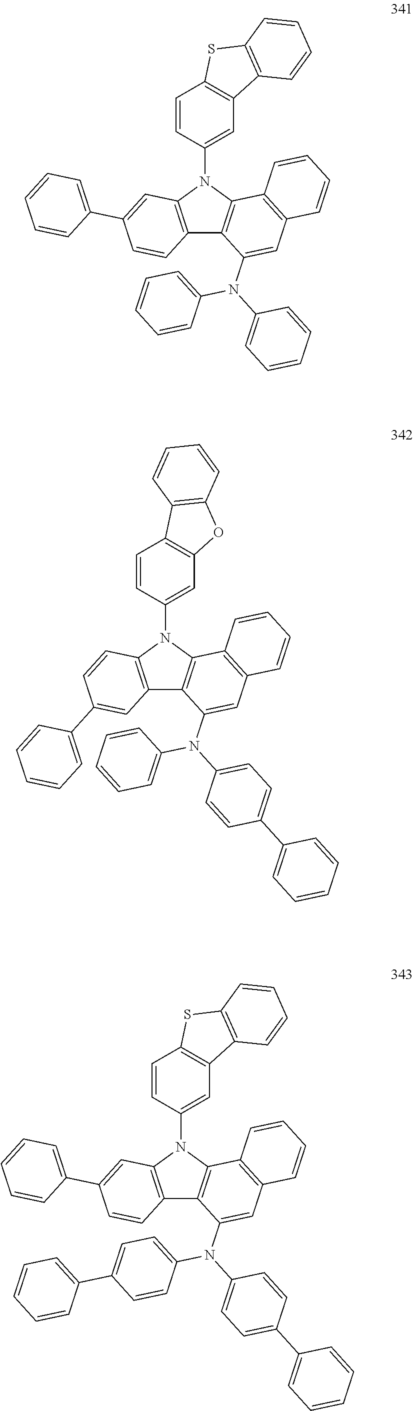 [SMILES: c1ccc(-c2ccc3c4c(N(c5ccccc5)c5ccccc5)cc5ccccc5c4n(-c4ccc5sc6ccccc6c5c4)c3c2)cc1 ; c1ccc(-c2ccc(N(c3ccccc3)c3cc4ccccc4c4c3c3cc(-c5ccccc5)ccc3n4-c3ccc4c(c3)oc3ccccc34)cc2)cc1 ; c1ccc(-c2ccc(N(c3ccc(-c4ccccc4)cc3)c3cc4ccccc4c4c3c3ccc(-c5ccccc5)cc3n4-c3ccc4sc5ccccc5c4c3)cc2)cc1]

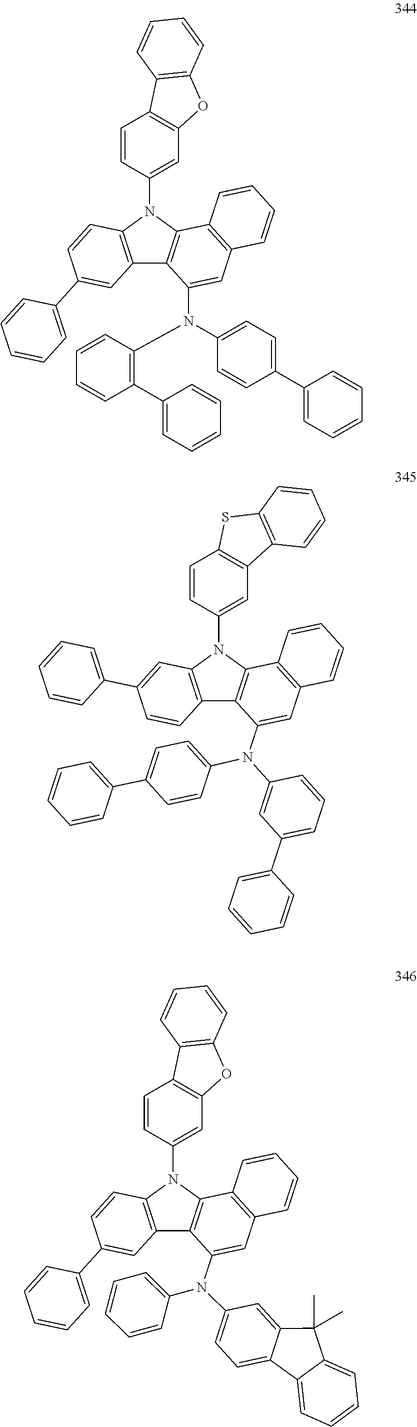 [SMILES: c1ccc(-c2ccc(N(c3ccccc3-c3ccccc3)c3cc4ccccc4c4c3c3cc(-c5ccccc5)ccc3n4-c3ccc4c(c3)oc3ccccc34)cc2)cc1 ; c1ccc(-c2ccc(N(c3cccc(-c4ccccc4)c3)c3cc4ccccc4c4c3c3ccc(-c5ccccc5)cc3n4-c3ccc4sc5ccccc5c4c3)cc2)cc1 ; CC1(C)c2ccccc2-c2ccc(N(c3ccccc3)c3cc4ccccc4c4c3c3cc(-c5ccccc5)ccc3n4-c3ccc4c(c3)oc3ccccc34)cc21]

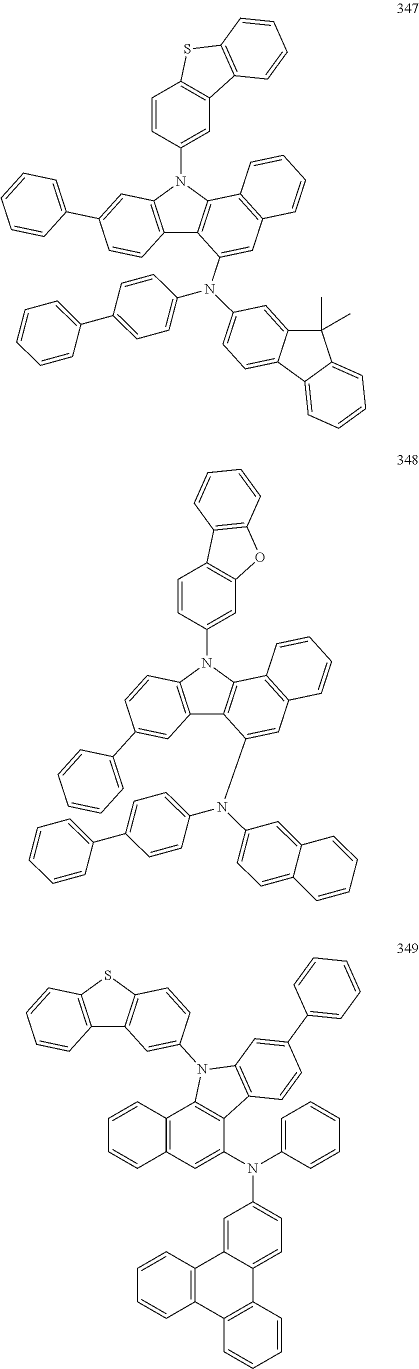 [SMILES: CC1(C)c2ccccc2-c2ccc(N(c3ccc(-c4ccccc4)cc3)c3cc4ccccc4c4c3c3ccc(-c5ccccc5)cc3n4-c3ccc4sc5ccccc5c4c3)cc21 ; c1ccc(-c2ccc(N(c3ccc4ccccc4c3)c3cc4ccccc4c4c3c3cc(-c5ccccc5)ccc3n4-c3ccc4c(c3)oc3ccccc34)cc2)cc1 ; c1ccc(-c2ccc3c4c(N(c5ccccc5)c5ccc6c7ccccc7c7ccccc7c6c5)cc5ccccc5c4n(-c4ccc5sc6ccccc6c5c4)c3c2)cc1]

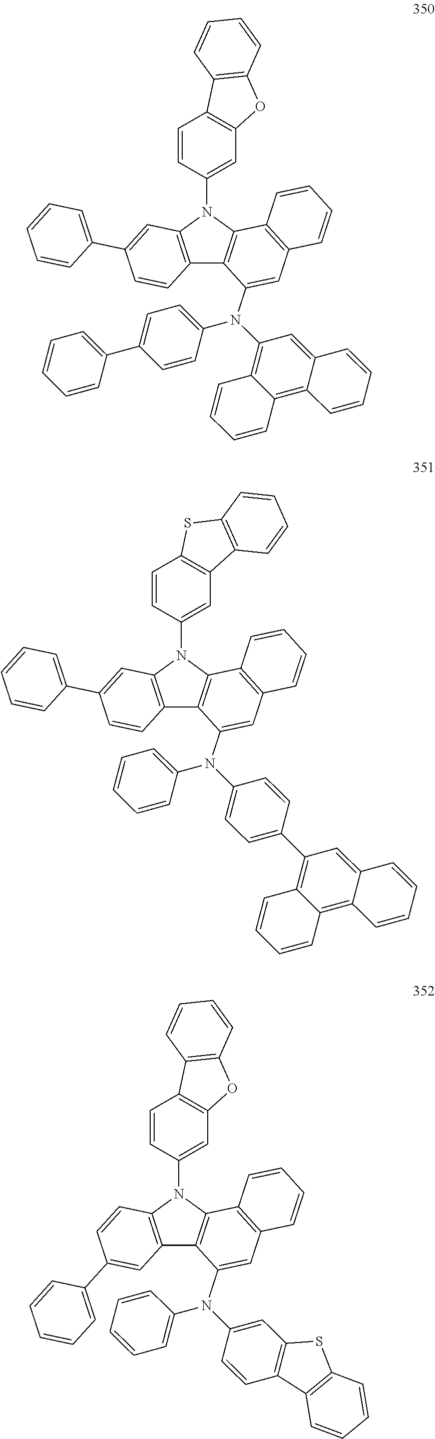 [SMILES: c1ccc(-c2ccc(N(c3cc4ccccc4c4ccccc34)c3cc4ccccc4c4c3c3ccc(-c5ccccc5)cc3n4-c3ccc4c(c3)oc3ccccc34)cc2)cc1 ; c1ccc(-c2ccc3c4c(N(c5ccccc5)c5ccc(-c6cc7ccccc7c7ccccc67)cc5)cc5ccccc5c4n(-c4ccc5sc6ccccc6c5c4)c3c2)cc1 ; c1ccc(-c2ccc3c(c2)c2c(N(c4ccccc4)c4ccc5c(c4)sc4ccccc45)cc4ccccc4c2n3-c2ccc3c(c2)oc2ccccc23)cc1]

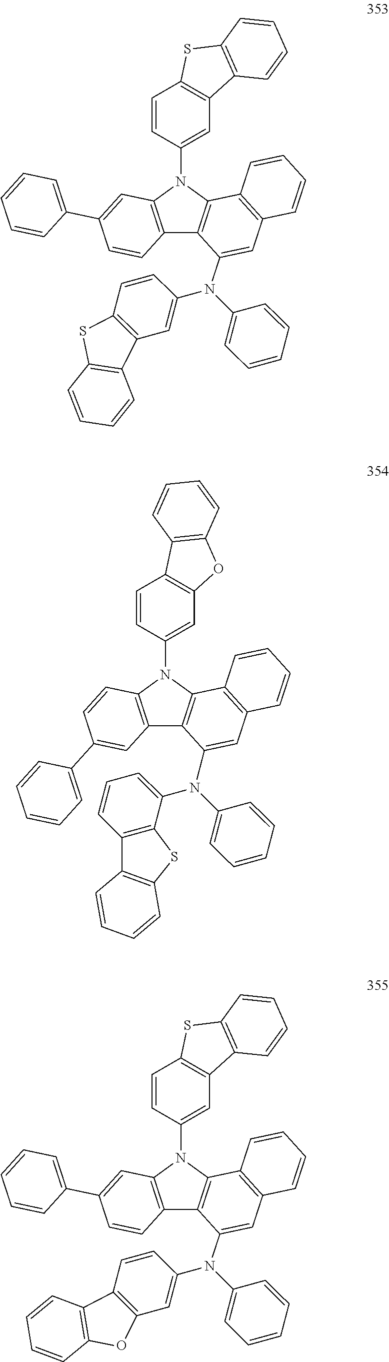 [SMILES: c1ccc(-c2ccc3c4c(N(c5ccccc5)c5ccc6sc7ccccc7c6c5)cc5ccccc5c4n(-c4ccc5sc6ccccc6c5c4)c3c2)cc1 ; c1ccc(-c2ccc3c(c2)c2c(N(c4ccccc4)c4cccc5c4sc4ccccc45)cc4ccccc4c2n3-c2ccc3c(c2)oc2ccccc23)cc1 ; c1ccc(-c2ccc3c4c(N(c5ccccc5)c5ccc6c(c5)oc5ccccc56)cc5ccccc5c4n(-c4ccc5sc6ccccc6c5c4)c3c2)cc1]

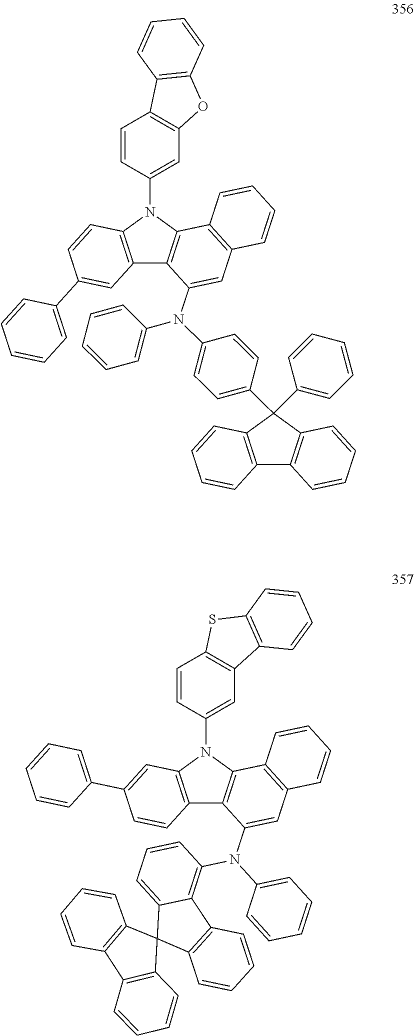 [SMILES: c1ccc(-c2ccc3c(c2)c2c(N(c4ccccc4)c4ccc(C5(c6ccccc6)c6ccccc6-c6ccccc65)cc4)cc4ccccc4c2n3-c2ccc3c(c2)oc2ccccc23)cc1 ; c1ccc(-c2ccc3c4c(N(c5ccccc5)c5cccc6c5-c5ccccc5C65c6ccccc6-c6ccccc65)cc5ccccc5c4n(-c4ccc5sc6ccccc6c5c4)c3c2)cc1]

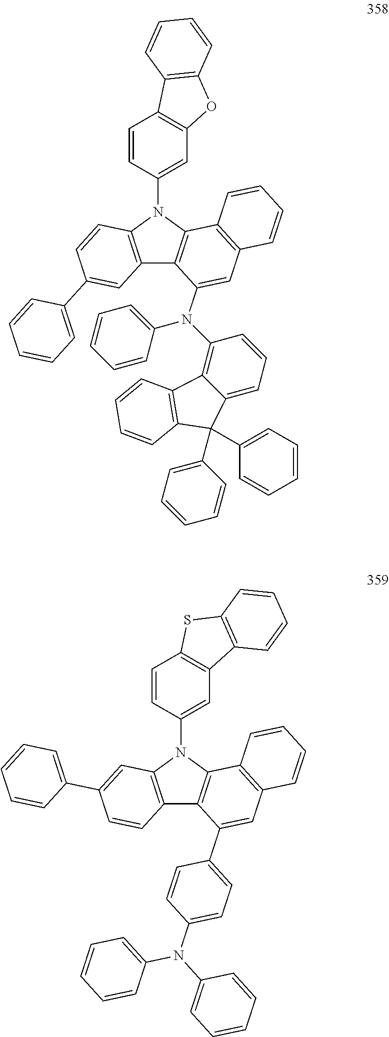 [SMILES: c1ccc(-c2ccc3c(c2)c2c(N(c4ccccc4)c4cccc5c4-c4ccccc4C5(c4ccccc4)c4ccccc4)cc4ccccc4c2n3-c2ccc3c(c2)oc2ccccc23)cc1 ; c1ccc(-c2ccc3c4c(-c5ccc(N(c6ccccc6)c6ccccc6)cc5)cc5ccccc5c4n(-c4ccc5sc6ccccc6c5c4)c3c2)cc1]

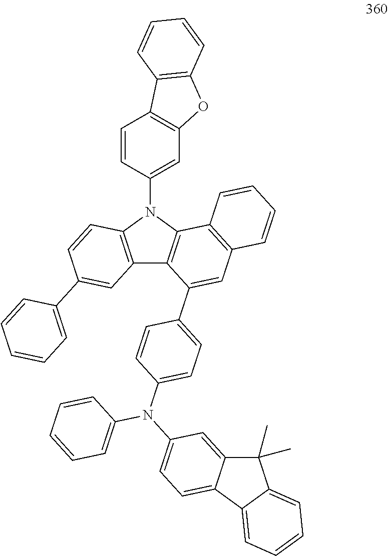 [SMILES: CC1(C)c2ccccc2-c2ccc(N(c3ccccc3)c3ccc(-c4cc5ccccc5c5c4c4cc(-c6ccccc6)ccc4n5-c4ccc5c(c4)oc4ccccc45)cc3)cc21]

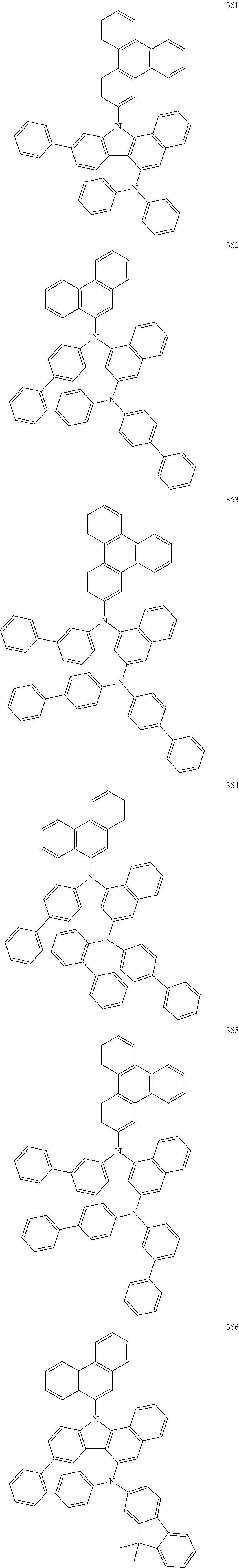

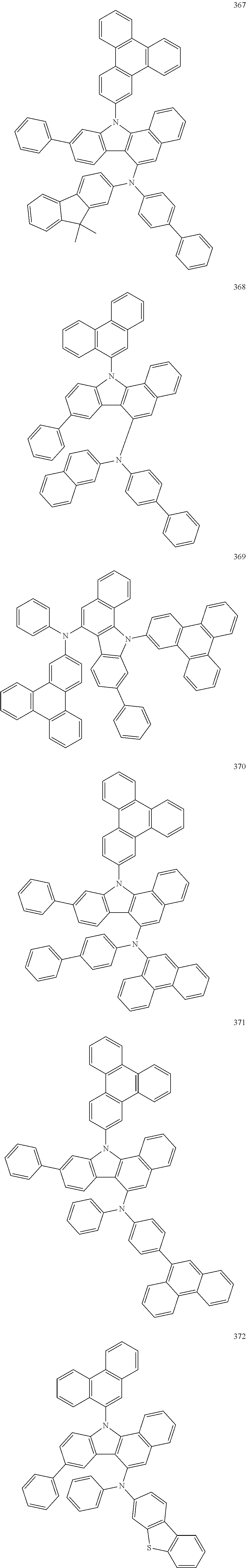

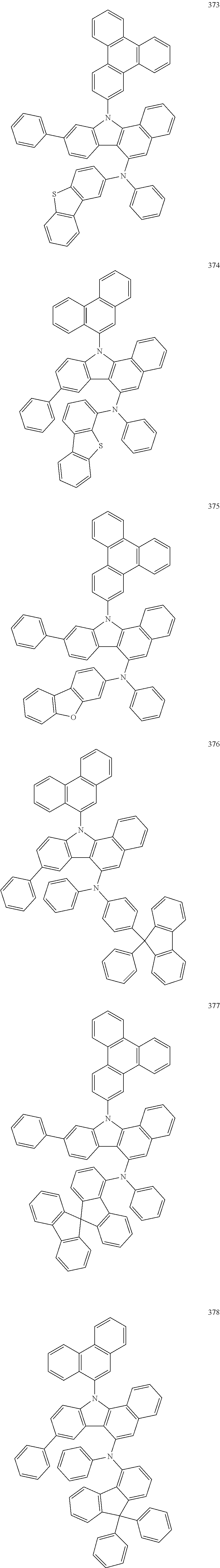

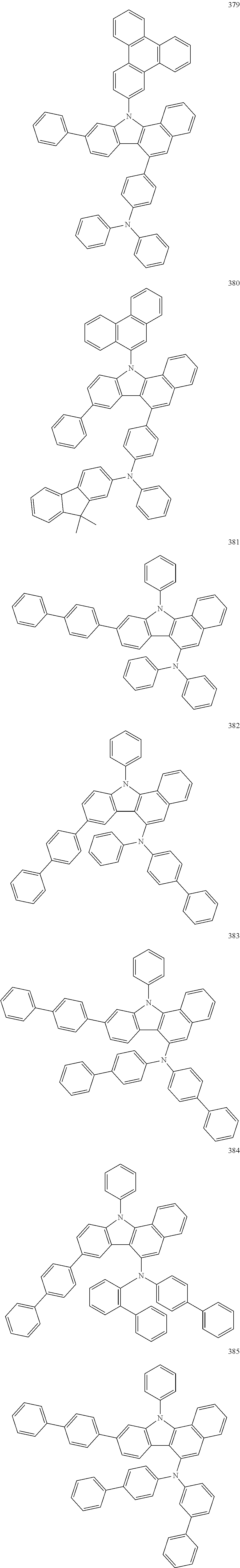

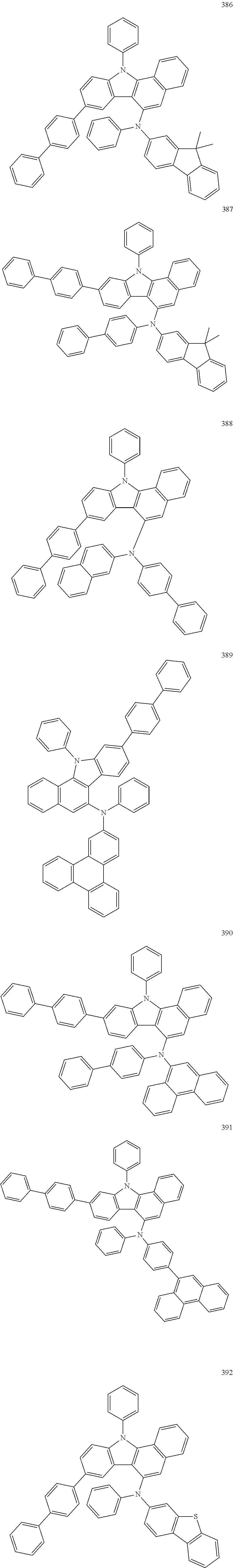

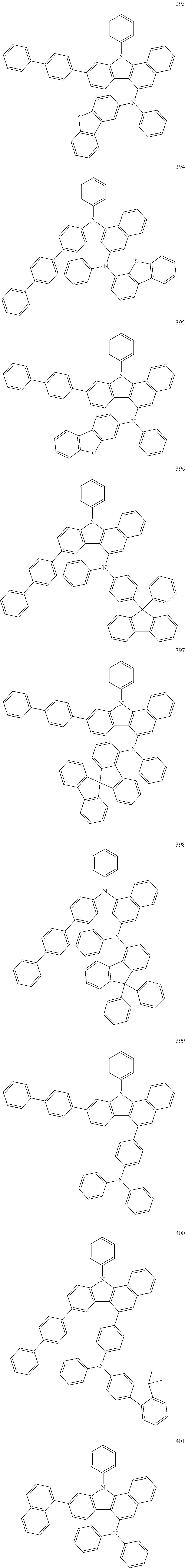

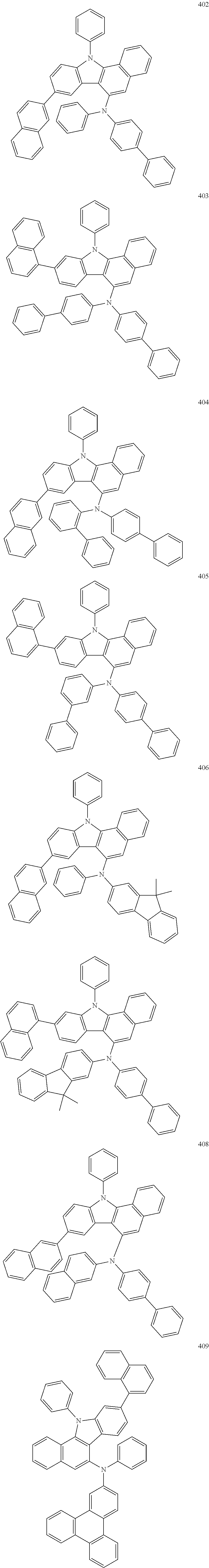

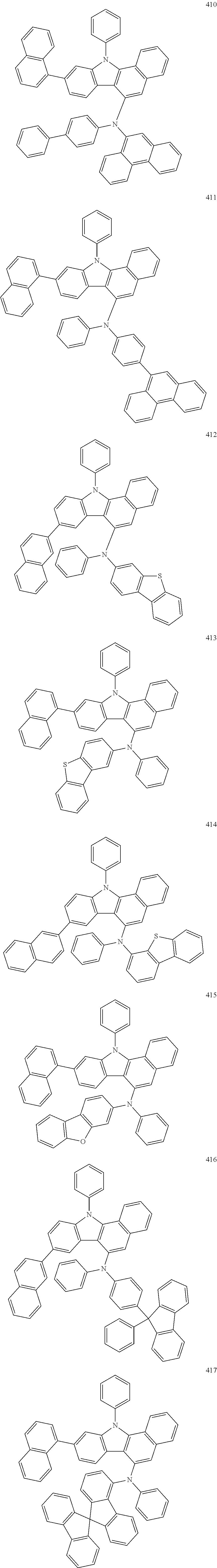

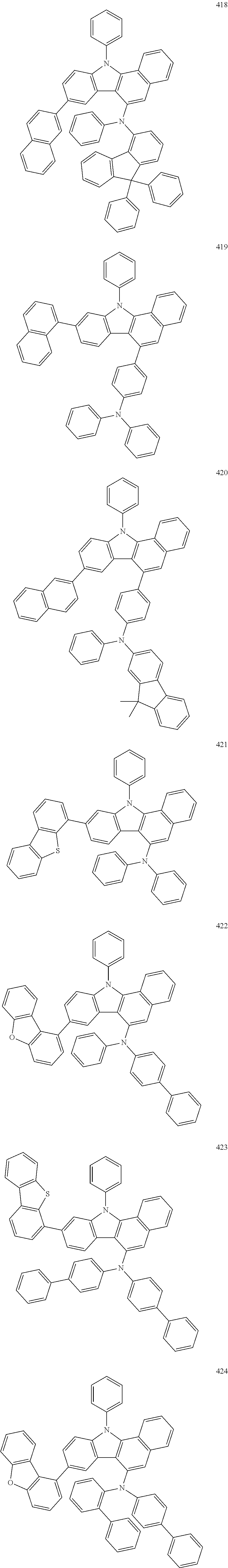

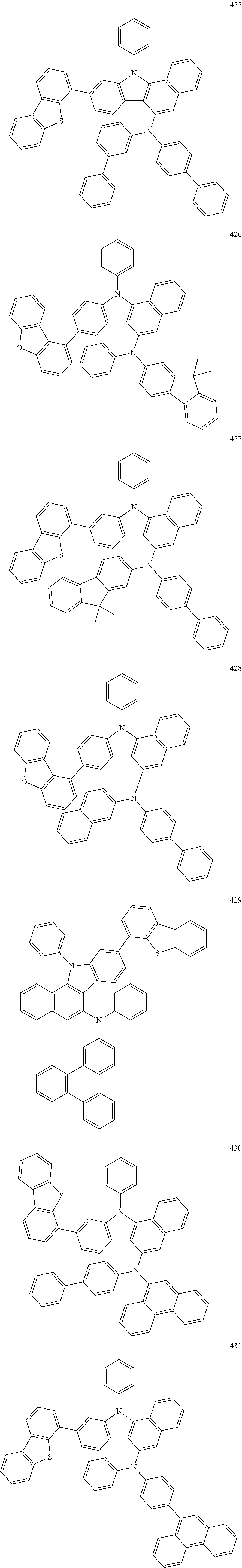

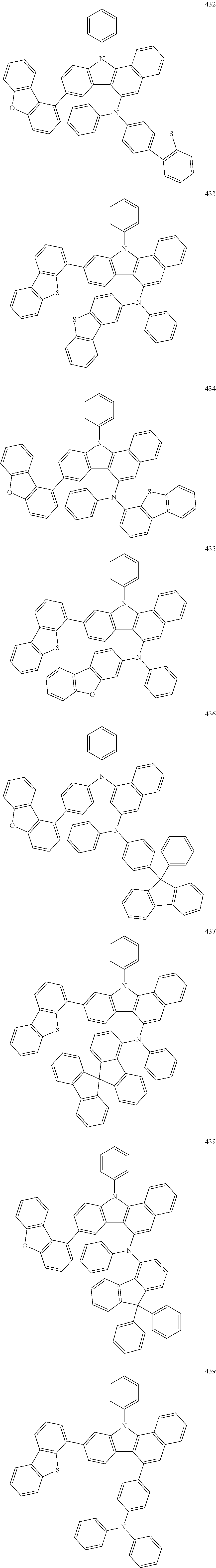

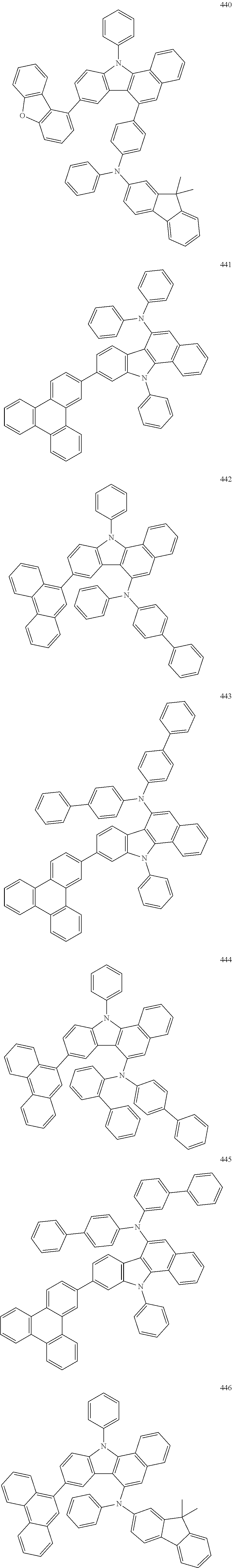

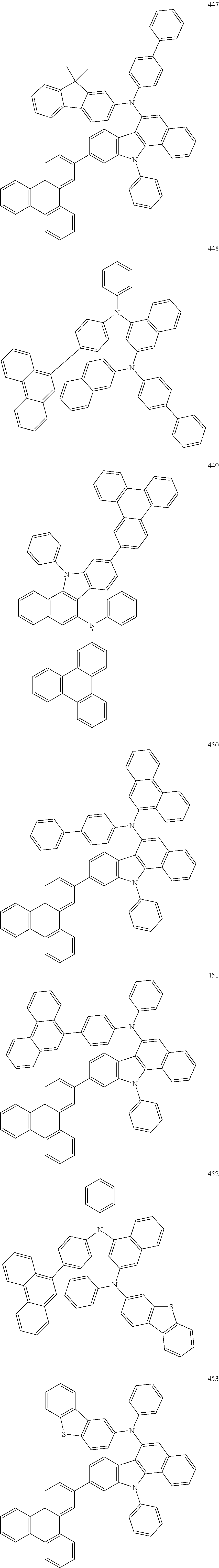

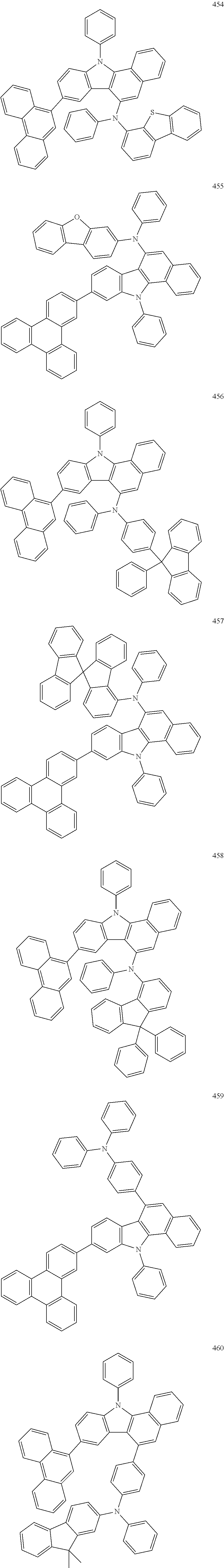

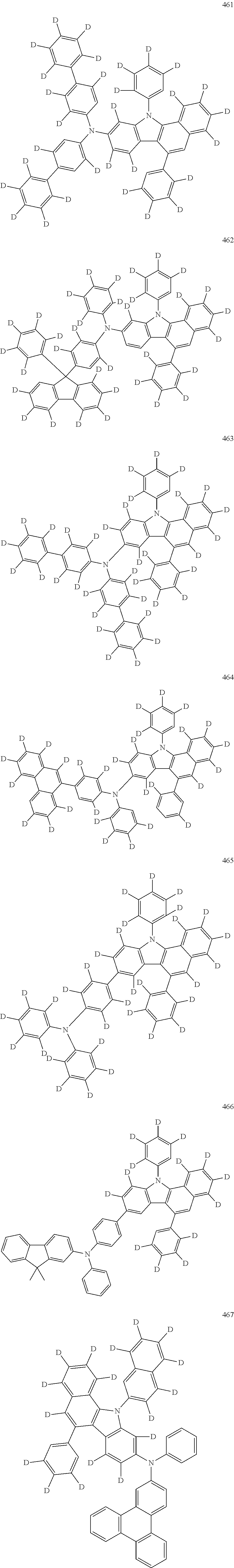

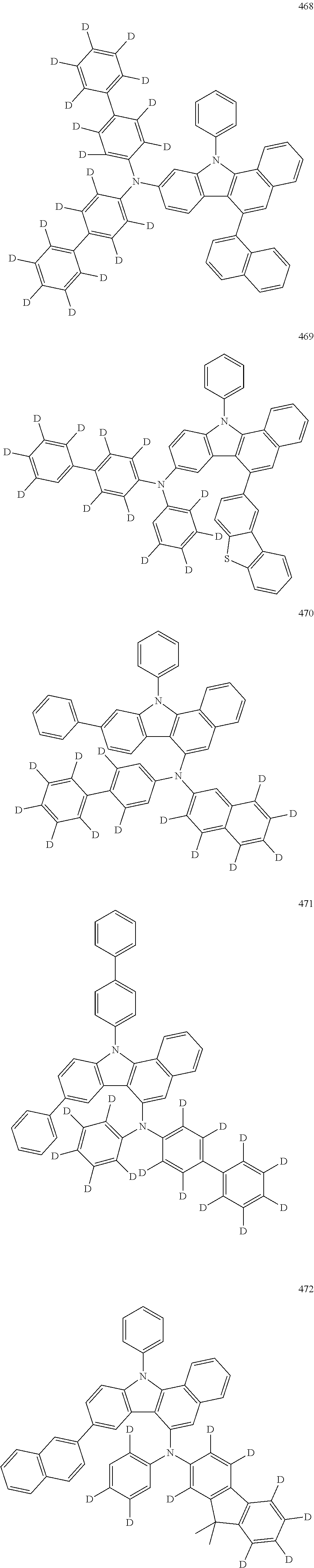

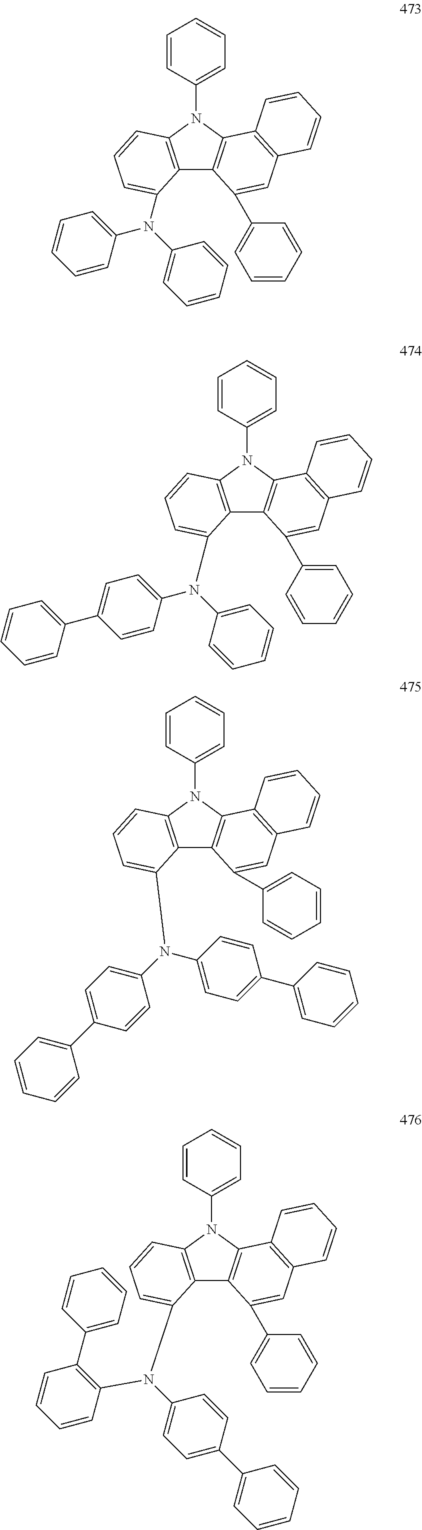

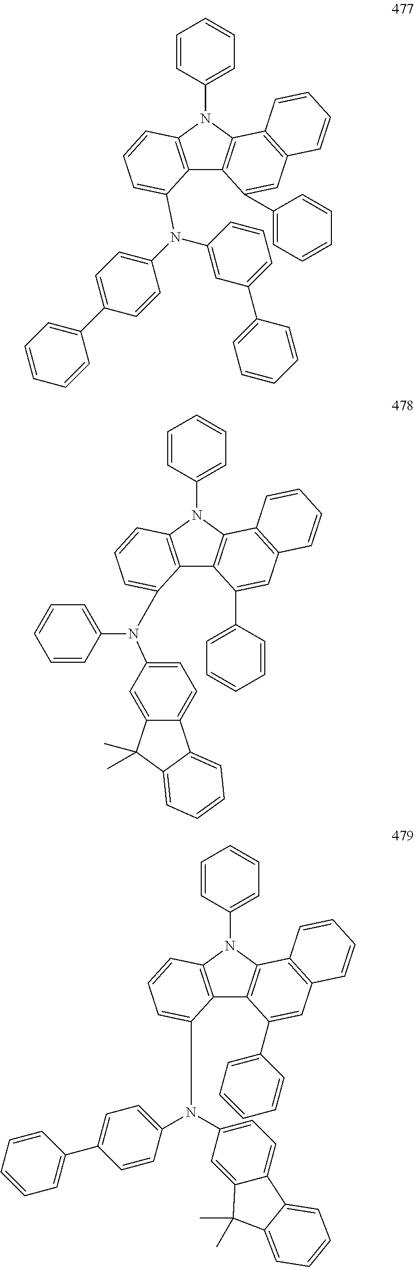

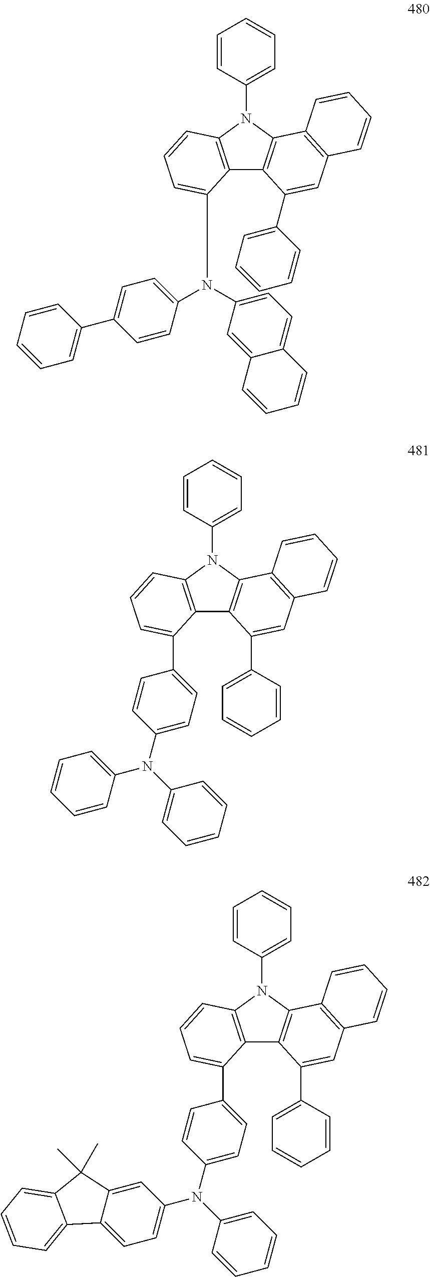 [SMILES: c1ccc(-c2ccc(N(c3ccc4ccccc4c3)c3cccc4c3c3c(-c5ccccc5)cc5ccccc5c3n4-c3ccccc3)cc2)cc1 ; c1ccc(-c2cc3ccccc3c3c2c2c(-c4ccc(N(c5ccccc5)c5ccccc5)cc4)cccc2n3-c2ccccc2)cc1 ; CC1(C)c2ccccc2-c2ccc(N(c3ccccc3)c3ccc(-c4cccc5c4c4c(-c6ccccc6)cc6ccccc6c4n5-c4ccccc4)cc3)cc21]

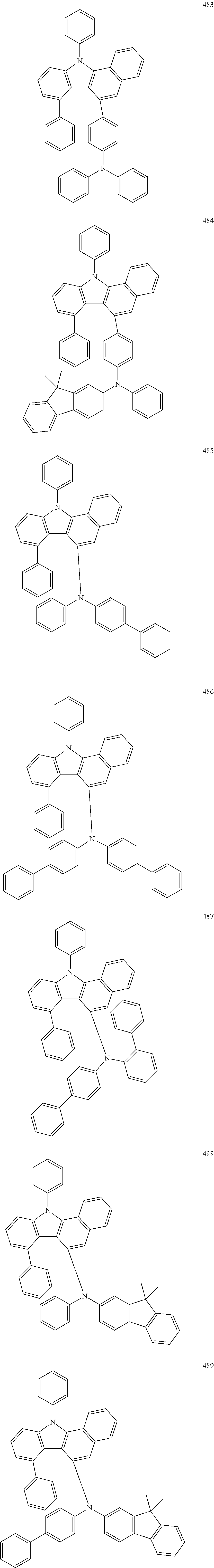

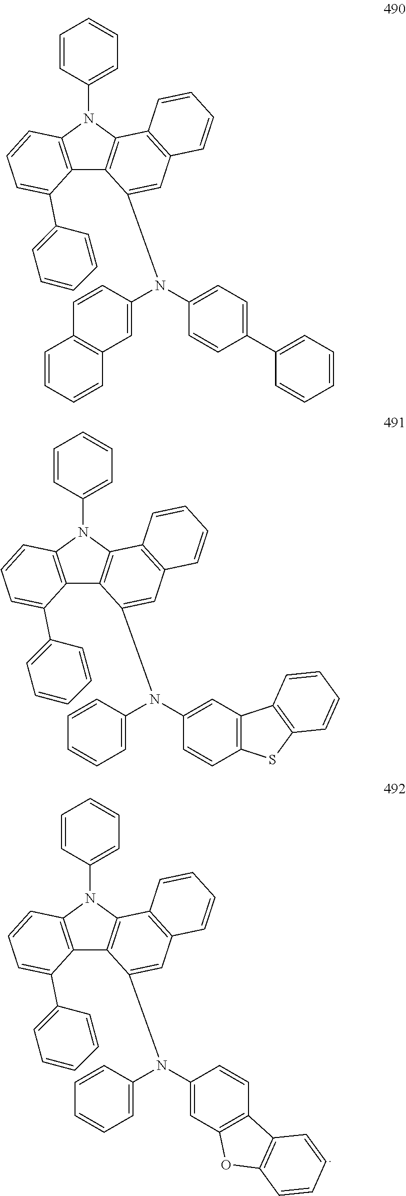

8. An organic light emitting device comprising: a first electrode; a second electrode provided to face the first electrode; and an organic material layer having one or more layers provided between the first electrode and the second electrode, wherein the one or more layers of the organic material layer comprise the heterocyclic compound of claim 1.

9. The organic light emitting device of claim 8, wherein the organic material layer comprises a light emitting layer, and the light emitting layer comprises the heterocyclic compound represented by Chemical Formula 1.

10. The organic light emitting device of claim 8, wherein the organic material layer comprises a light emitting layer, the light emitting layer comprises a host material, and the host material comprises the heterocyclic compound represented by Chemical Formula 1.

11. The organic light emitting device of claim 8, wherein the organic material layer comprises an electron blocking layer, and the electron blocking layer comprises the heterocyclic compound represented by Chemical Formula 1.

12. The organic light emitting device of claim 8, wherein the organic material layer comprises a hole transport layer or a hole transport auxiliary layer, and the hole transport layer or the hole transport auxiliary layer comprises the heterocyclic compound represented by Chemical Formula 1.

13. The organic light emitting device of claim 8, further comprising one or two or more layers selected from the group consisting of a light emitting layer, a hole injection layer, a hole transport layer, a hole transport auxiliary layer, an electron blocking layer, a hole blocking layer, an electron transport layer and an electron injection layer.
